(19) United States
(12) Patent Application Publication
(10) Pub. No.: US 2009/0320718 A1
(43) Pub. Date: Dec. 31, 2009

(54) FLUOROSURFACTANTS

(76) Inventors: Wolfgang Hierse, Gross-Zimmern (DE); Nikolai (Mykola) Ignatyev, Duisburg (DE); Martin Seidel, Darmstadt (DE); Elvira Montenegro, Weinheim (DE); Peer Kirsch, Yokohama Kanagawa (JP); Andreas Bathe, Darmstadt (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/307,218

(22) PCT Filed: Jul. 2, 2007
(86) PCT No.: PCT/EP2007/005842

§ 371 Date: Dec. 31, 2008

(30) Foreign Application Priority Data
Jul. 4, 2006 (DE) 10 2006 031 143.4

(51) Int. Cl. C09D 7/12; C07C 211/03; C07C 303/02

(52) U.S. Cl. 106/287.32; 564/510; 558/48

(57) ABSTRACT

The present invention relates to the use of end groups Y, where Y stands for CF₃(CH₂)ₐS— or CF₃CF₂S— or [CF₃—(CH₂)ₐ]₂N—, where a stands for an integer selected from the range from 0 to 5, as end group in surface-active compounds, to corresponding novel compounds, and to processes for the preparation of these compounds.

The present invention relates to the use of end groups Y, where Y stands for CF₃(CH₂)ₐS— or CF₃CF₂S— or [CF₃—(CH₂)ₐ]₂N—, where a stands for an integer selected from the range from 0 to 5, as end group in surface-active compounds, to corresponding novel compounds, and to processes for the preparation of these compounds.

Fluorosurfactants have an outstanding ability to lower the surface energy, which is utilised, for example, in the hydrophobicisation of surfaces, such as textile impregnation, the hydrophobicisation of glass or the de-icing of air-craft wings.

In general, however, fluorosurfactants contain perfluoroalkyl substituents, which are degraded in the environment by biological and other oxidation processes to give perfluoroalkanecarboxylic acids and -sulfonic acids. These are regarded as persistent and are in some cases suspected of causing health problems (G. L. Kennedy, Jr., J. L. Butenhoff, G. W. Olsen, J. C. O'Connor, A. M. Seacat, R. G. Perkins, L. B. Biegel, S. R. Murphy, D. G. Farrar, Critical Reviews in Toxicology 2004, 34, 351-384). In addition, relatively long-chain perfluoroalkanecarboxylic acids and -sulfonic acids accumulate in the food chain (e.g.: M. Fricke, U. Lahl, Z Umweltchem Ökotox 17 (1) 36-49 (2005) (risk assessment of perfluorosurfactants as contribution to the current debate on the REACH dossier from the EU Commission)).

There is therefore a demand for novel surface-active substances which have a property profile which is comparable to the classical fluorosurfactants and are preferably oxidatively or reductively degradable. Particularly advantageous compounds are those which do not leave behind any persistent organofluorine degradation products on degradation.

The Omnova company markets polymers whose side chains have terminal CF₃ or C₂F₅ groups. International patent application WO 03/010128 describes perfluoroalkyl-substituted amines, acids, amino acids and thioether acids which have a C₃₋₂₀-perfluoroalkyl group.

JP-A-2001/133984 discloses surface-active compounds having perfluoroalkoxy chains which are suitable for use in antireflection coatings. JP-A-09/111 286 discloses the use of perfluoropolyether surfactants in emulsions.

The earlier German patent application DE 102005000858 A describes compounds which carry at least one terminal pentafluorosulfuranyl group or at least one terminal trifluoromethoxy group and have a polar end group, are surface-active and are highly suitable as surfactants.

Nevertheless, there continues to be a demand for novel fluorinated end groups or compounds which contain these end groups. It is advantageous if such compounds can be used as surfactants or precursors for surfactants.

The present invention therefore relates firstly to the use of end groups Y, where Y stands for CF₃(CH₂)ₐS— or CF₃CF₂S— or [CF₃—(CH₂)ₐ]₂N—, where a stands for an integer selected from the range from 0 to 5, as end group in surface-active compounds. A preferred range for a is 0 to 3, in particular 0 to 2.

The end group Y in the surface-active compounds is preferably bonded to a saturated or unsaturated, optionally aromatic, branched or unbranched, optionally substituted, optionally heteroatom-substituted hydrocarbon unit. The hydrocarbon units may be aliphatic or aromatic units, optionally provided with heteroatoms. It is particularly preferred for the hydrocarbon units or the entire molecule to be free from further fluorine atoms.

Besides the said fluorinated end groups, the compounds to be used in accordance with the invention preferably contain no further fluorinated groups.

In a variant of the invention, the end group Y occurs a number of times in the surface-active compound and the surface-active compound is preferably an oligomer or polymer.

In another, likewise preferred variant of the invention, the end group Y occurs only once, twice or three times in the surface-active compound, where compounds in which the end group only occurs once are particularly preferred. The compounds to be used in accordance with the invention are preferably low-molecular-weight compounds of the formula I

Y-spacer-X I

where
Y stands for CF₃(CH₂)ₐS— or CF₃CF₂S— or [CF₃—(CH₂)ₐ]₂N—, where a stands for an integer selected from the range from 0 to 5, preferably 0 to 3, in particular 0 to 2,
spacer stands for a saturated or unsaturated, optionally aromatic, branched or unbranched, optionally substituted hydrocarbon unit,
X stands for a cationic, nonionic, amphoteric or anionic polar group or a polymerisable group.

It is particularly preferred here for the compound of the formula I to be selected from the compounds of the formulae Ia to Ig

Y—(CH₂)ₙ—X Ia

Y—CH₂—CH(Hal)—(CH₂)₍ₙ₋₁₎—X Ib

Y—CH═CH—(CH₂)₍ₙ₋₁₎—X Ic

Y—CH₂CH═CH—(CH₂)₍ₙ₋₁₎—X Id

Y—CH₂—Ar—(CH₂)₍ₙ₋₁₎—X Ie

Y—(CH₂)ₙ₋₁—C≡C—(CH₂)ₙ—X If

Y—(CH₂)ₙ-Q-(CH₂)_{n′}—X Ig

in which Y stands for CF₃(CH₂)ₐS— or CF₃CF₂S— or [CF₃—(CH₂)ₐ]₂N—, where a stands for an integer selected from the range from 0 to 5, n and n′ stand, independently of one another, for an integer from the range 1 to 30, X stands for a cationic, nonionic, amphoteric or anionic polar group or a polymerisable group or a functional group, Ar stands for aryl, Q stands for O, S or N, and (Hal) stands for F, Cl, Br or I, and corresponding salts of the compounds of the formulae Ia to Ig.

A preferred range for a is 0 to 3, in particular 0 to 2. A preferred range for n and/or n′ is 4 to 24, in particular 4 to 18. Compounds where n and/or n′ are in the range from 4 to 16 are especially preferred, in particular in the range from 8 to 16. Z preferably stands for O or S. Particular preference is given to the use of compounds of the formulae Ia to Ig which have a combination of the variables in their preferred ranges.

Very particular preference is given here to the use of compounds of the formula la where n and/or n′ particularly preferably stand for an integer from the range from 4 to 24, in particular 4 to 18, and particularly preferably for an integer from the range 4 to 16, in particular 8 to 16. In a variant of the invention, it is in turn preferred for n and/or n′ to be an even number.

In another, likewise preferred variant of the invention, n in the formula la stands for 1 or 2 and X preferably stands for a functional group, preferably selected from —CH═CH₂, —C≡CH, —CHO, —C(═O)CH₃, —COOH, —COOR, —OH, —SH, —SO₂Cl, —Cl, —Br, —I, in which R stands for C₁₋₃₀-alkyl, Ar or —CH₂Ar, in particular for C₁₋₄-alkyl or —CH₂Ar. These compounds are particularly suitable as intermediates for the synthesis of further compounds according to the invention.

Particular preference is given in accordance with the invention to the use of the above-mentioned compounds as surfactants.

If the compounds of the formula I are anionic compounds or compounds which can be converted into the anions of salts, it is preferred for the counterion to be an alkali metal ion, preferably Li⁺, Na⁺ or K⁺, an alkaline-earth metal ion or NH₄⁺ or tetra-C₁₋₆-alkylammonium or tetra-C₁₋₆-alkylphosphonium. If the compounds of the formula I are cationic compounds or compounds which can be converted into the cations of salts, it is preferred for the counterion to be a halide, such as Cl⁻, Br⁻, I⁻, or CH₃SO₃⁻, CF₃SO₃⁻, CH₃PhSO₃⁻ or PhSO₃⁻.

Advantages of the compounds according to the invention or use according to the invention of the said compounds or the compositions according to the invention may be, in particular:
a surface activity which may be equal or superior to that of conventional hydrocarbon surfactants with respect to efficiency and/or effectiveness, and/or
biological and/or abiotic degradability of the substances without the formation of persistent, perfluorinated degradation products, and/or
good processing properties in formulations, and/or
storage stability.

The present invention furthermore relates to the corresponding novel compounds of the formula I, in particular compounds of the formulae Ia to IIg

[CF₃—(CH₂)ₐ]₂N−(CH₂)ₙ—X IIa

[CF₃—(CH₂)ₐ]₂N—CH₂—CH(Hal)—(CH₂)₍ₙ₋₁₎—X IIb

[CF₃—(CH₂)ₐ]₂N—CH═CH—(CH₂)₍ₙ₋₁₎—X IIc

[CF₃—(CH₂)ₐ]₂N—CH₂CH═CH—(CH₂)₍ₙ₋₁₎—X IId

[CF₃—(CH₂)ₐ]₂N—CH₂—Ar—(CH₂)₍ₙ₋₁₎—X IIe

[CF₃—(CH₂)ₐ]₂N—(CH₂)ₙ₋₁—C≡C—(CH₂)ₙ—X IIf

[CF₃—(CH₂)ₐ]₂N—(CH₂)ₙ-Q-(CH₂)_{n′}—X IIg

in which a stands for an integer selected from the range from 0 to 5, n and n′ stand, independently of one another, for an integer from the range 1 to 30, X stands for a cationic, nonionic, amphoteric or anionic polar group or a polymerisable group or a functional group, Ar stands for aryl, Q stands for O, S or N, (Hal) stands for F, Cl, Br or I, and corresponding salts of the compounds of the formulae Ia to IIg and the compounds of the formulae IIIa to IIIg

CF₃(CH₂)ₐS—(CH₂)ₙ—X IIIa

CF₃(CH₂)ₐS—CH₂—CH(Hal)—(CH₂)₍ₙ₋₁₎—X IIIb

CF₃(CH₂)ₐS—CH═CH—(CH₂)₍ₙ₋₁₎—X IIIc

CF₃(CH₂)ₐS—CH₂CH═CH—(CH₂)₍ₙ₋₁₎—X IIId

CF₃(CH₂)ₐS—CH₂—Ar—(CH₂)₍ₙ₋₁₎—X IIIe

CF₃(CH₂)ₐS—(CH₂)ₙ₋₁—C≡C—(CH₂)ₙ—X IIIf

CF₃(CH₂)ₐS—(CH₂)ₙ-Q-(CH₂)_{n′}—X IIIg

in which a stands for an integer selected from the range from 0 to 5, n and n′ stand, independently of one another, for an integer from the range 1 to 30, X stands for a cationic, nonionic, amphoteric or anionic polar group or a polymerisable group or a functional group, Ar stands for aryl, Q stands for O, S or N, (Hal) stands for F, Cl, Br or I, and corresponding salts of the compounds of the formulae IIIa to IIIg and the compounds of the formulae IVa to IVg

CF₃CF₂S—(CH₂)ₙ—X IVa

CF₃CF₂S—CH₂—CH(Hal)—(CH₂)₍ₙ₋₁₎—X IVb

CF₃CF₂S—CH═CH—(CH₂)₍ₙ₋₁₎—X IVc

CF₃CF₂S—CH₂—CH═CH—(CH₂)₍ₙ₋₁₎—X IVd

CF₃CF₂S—CH₂—Ar—(CH₂)₍ₙ₋₁₎—X IVe

CF₃CF₂S—(CH₂)ₙ₋₁—C≡C—(CH₂)ₙ—X IVf

CF₃CF₂S—(CH₂)ₙ-Q-(CH₂)_{n′}—X IVg

in which n and n′ stand, independently of one another, for an integer from the range 1 to 30, X stands for a cationic, nonionic, amphoteric or anionic polar group or a polymerisable group or a functional group, Ar stands for aryl, Q stands for O, S or N, (Hal) stands for F, Cl, Br or I, and corresponding salts of the compounds of the formulae IVa to IVg.

n and/or n′ in compounds of the formulae II to IV preferably stand, independently of one another, for a number from the range 4 to 28, particularly preferably for a number from the range 4 to 24. Particular preference is given to compounds where n and/or n′ are in the range from 4 to 18. Special preference is given to compounds where n and/or n′ are in the range from 4 to 16, in particular in the range from 8 to 16. Further preferred ranges of the variables of the formulae I to IV are given below. A preferred range for a is 0 to 3, in particular 0 to 2. Z preferably stands for O or S. Particular preference is given to the use of compounds of the formulae II to IV which have a combination of the variables in their preferred ranges.

In a preferred group of compounds of the formula I to be employed in accordance with the invention or compounds of the formulae II to IV according to the invention, X stands for an anionic polar group selected from —COOM, —SO₃M, —OSO₃M, —PO₃M₂, —OPO₃M₂, —(OCH₂CHR)ₘ—O—(CH₂)ₒ—COOM, —(OCH₂CHR)ₘ—O—(CH₂)ₒ—SO₃M, —(OCH₂CHR)ₘ—O—(CH₂)ₒ—OSO₃M, —(OCH₂CHR)ₘ—O—(CH₂)ₒ—PO₃M₂, —(OCH₂CHR)ₘ—O—(CH₂)ₒ—OPO₃M₂, where M stands for H or an alkali metal ion, preferably Li⁺, Na⁺ or K⁺, or NH₄⁺ or tetra-C₁₋₆-alkylammonium or tetra-C₁₋₆-alkylphosphonium, R stands for H or C₁₋₄-alkyl, m stands for an integer from the range from 1 to 1000, and o stands for an integer selected from 1, 2, 3 and 4.

The preferred anionic groups here include, in particular, —COOM, —SO₃M, —OSO₃M, and —(OCH₂CHR)ₘ—O—(CH₂)ₒ—COOM, —(OCH₂CHR)ₘ—O—(CH₂)ₒ—SO₃M and —(OCH₂CHR)ₘ—O—(CH₂)ₒ—OSO₃M, where each individual one of these groups may be preferred per se.

In another, likewise preferred group of compounds of the formula I to be employed in accordance with the invention or compounds of the formula II or III or IV according to the invention, X stands for a cationic polar group selected from —NR¹R²R³⁺Z³¹ , —PR¹R²R³⁺Z⁻,

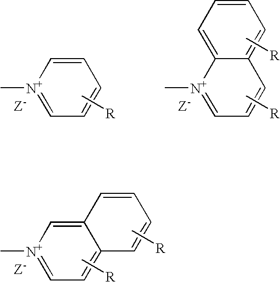

where R stands for H or C₁₋₄-alkyl in any desired position, Z⁻ stands for Cl⁻, Br⁻, I⁻, CH₃SO₃⁻, CF₃SO₃⁻, CH₃PhSO₃⁻, PhSO₃⁻, R¹, R² and R³ each, independently of one another, stand for H, C₁₋₃₀-alkyl, Ar or —CH₂Ar, and
Ar stands for an unsubstituted or mono- or polysubstituted aromatic ring or fused ring system having 6 to 18 C atoms, in which, in addition, one or two CH groups may be replaced by N.

The preferred cationic groups here include, in particular, —NR¹R²R³⁺Z⁻ and

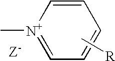

where each individual one of these groups per se may be preferred.

In a further preferred group of compounds of the formula I to be employed in accordance with the invention or compounds of the formulae II to IV according to the invention, X stands for a nonionic polar group selected from —Cl, —Br, —I, —(OCH₂CHR)ₘ—OH, —(OCH₂CHR)ₘ—SH, —O-(glycoside)ₒ, —(OCH₂CHR)ₘ—OCH₂—CHOH—CH₂—OH, —(OCH₂CHR)ₘ—OCH₂Ar(—NCO)ₚ, —(OCH₂CHR)ₘ—OAr(—NCO)ₚ, —SiR¹R²Z, —SiR¹Z₂, —SiZ₃, —COZ, —(OCH₂CHR)ₘ—SO₂CH═CH₂, —SO₂Z,

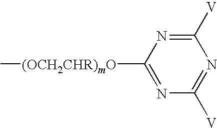

m stands for an integer from the range from 0 to 1000,
n stands for 0 or 1, and
o stands for an integer from the range from 1 to 10,
p stands for 1 or 2,
R stands for H or C₁₋₄-alkyl,
R¹ and R² each, independently of one another, stand for C₁₋₃₀-alkyl,
Ar or —CH₂Ar, and
Ar stands for an unsubstituted, mono- or polysubstituted aromatic ring or fused ring system having 6 to 18 C atoms, in which, in addition, one or two CH groups may be replaced by C═O, and
glycoside stands for an etherified carbohydrate, preferably for a mono-, di-, tri- or oligoglucoside,
all Z each, independently of one another, stand for —H, —Cl, —F, —NR¹R², —OR¹, —N-imidazolyl, and
V stands for Cl or F.

The preferred nonionic polar groups here include, in particular, —(OCH₂CHR)ₘ—OH and —O-(glycoside)o, where each individual one of these groups per se may be preferred.

In addition, preference may be given in accordance with the invention to compounds of the formulae I to IV in which X stands for a polymerisable group selected from —(OCH₂CHR)ₘOCOCR═CH₂, —(OCH₂CHR)ₘ—OCR═CH₂,

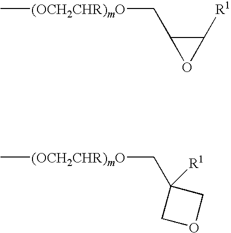

where m stands for an integer from the range from 0 to 1000, R stands for H or C₁₋₄-alkyl, R¹ stands for H or C₁₋₄-alkyl or Y-spacer-—(OCH₂CHR)ₘ—OCH₂—,
and to the use thereof.

These compounds are preferably converted into polymers having corresponding side chains, which may themselves again be employed in the sense according to the invention. The present invention also relates to the use of these polymers.

In addition, preference may be given in accordance with the invention to compounds of the formulae I to IV in which X stands for a functional group selected from —CR²═CR³R⁴, —C≡CR², —CHO, —C(═O)CH₃, —COOH, —OH, —SH, —Cl, —Br, —I, where R², R³ and R⁴ each, independently of one another, stand for H or Y-spacer- or C₁₋₄-alkyl, and to the use thereof.

In addition, preference may be given in accordance with the invention to compounds in which X stands for an amphoteric group selected from the functional groups of the acetyldiamines, the N-alkylamino acids, the betaines, the amine oxides and corresponding derivatives, and the use thereof. X particularly preferably stands for a betaine. In preferred compounds from this class of substances, X is a group selected from

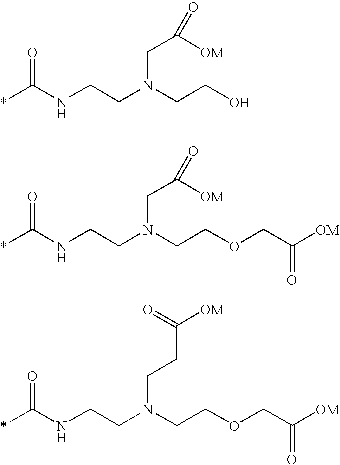

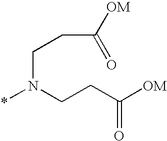

The particularly preferred compounds according to the invention include the compounds shown in the following table. These compounds may themselves be surfactants or they are the corresponding acids of surfactants or the precursors of surfactants. In the table, p stands for 1 to 2, m stands for 0 to 1000, and R stands for H or C₁ to C₄, preferably H or CH₃.

The particularly preferred compounds according to the invention include the compounds in the table in which n stands for an integer from the range 4 to 16.

TABLE
CF₃—S—(CH₂)₄—COOH; CF₃—S—(CH₂)₄—SO₃H; CF₃—S—(CH₂)₄—O—SO₃H; CF₃—S—(CH₂)₄—O—PO₃H
CF₃—S—(CH₂)₅—COOH; CF₃—S—(CH₂)₅—SO₃H; CF₃—S—(CH₂)₅—O—SO₃H; CF₃—S—(CH₂)₅—O—PO₃H
CF₃—S—(CH₂)₆—COOH; CF₃—S—(CH₂)₆—SO₃H; CF₃—S—(CH₂)₆—O—SO₃H; CF₃—S—(CH₂)₆—O—PO₃H
CF₃—S—(CH₂)₇—COOH; CF₃—S—(CH₂)₇—SO₃H; CF₃—S—(CH₂)₇—O—SO₃H; CF₃—S—(CH₂)₇—O—PO₃H
CF₃—S—(CH₂)₈—COOH; CF₃—S—(CH₂)₈—SO₃H; CF₃—S—(CH₂)₈—O—SO₃H; CF₃—S—(CH₂)₈—O—PO₃H
CF₃—S—(CH₂)₉—COOH; CF₃—S—(CH₂)₉—SO₃H; CF₃—S—(CH₂)₉—O—SO₃H; CF₃—S—(CH₂)₉—O—PO₃H
CF₃—S—(CH₂)₁₀—COOH; CF₃—S—(CH₂)₁₀—SO₃H; CF₃—S—(CH₂)₁₀—O—SO₃H; CF₃—S—(CH₂)₁₀—O—PO₃H
CF₃—S—(CH₂)₁₁—COOH; CF₃—S—(CH₂)₁₁—SO₃H; CF₃—S—(CH₂)₁₁—O—SO₃H; CF₃—S—(CH₂)₁₁—O—PO₃H
CF₃—S—(CH₂)₁₂—COOH; CF₃—S—(CH₂)₁₂—SO₃H; CF₃—S—(CH₂)₁₂—O—SO₃H; CF₃—S—(CH₂)₁₂—O—PO₃H
CF₃—S—(CH₂)₁₃—COOH; CF₃—S—(CH₂)₁₃—SO₃H; CF₃—S—(CH₂)₁₃—O—SO₃H; CF₃—S—(CH₂)₁₃—O—PO₃H
CF₃—S—(CH₂)₁₄—COOH; CF₃—S—(CH₂)₁₄—SO₃H; CF₃—S—(CH₂)₁₄—O—SO₃H; CF₃—S—(CH₂)₁₄—O—PO₃H
CF₃—S—(CH₂)₁₅—COOH; CF₃—S—(CH₂)₁₅—SO₃H; CF₃—S—(CH₂)₁₅—O—SO₃H; CF₃—S—(CH₂)₁₅—O—PO₃H
CF₃—S—(CH₂)₁₆—COOH; CF₃—S—(CH₂)₁₆—SO₃H; CF₃—S—(CH₂)₁₆—O—SO₃H; CF₃—S—(CH₂)₁₆—O—PO₃H
CF₃—S—CH₂—Ar—(CH₂)₄—COOH; CF₃—S—CH₂—Ar—(CH₂)₄—SO₃H; CF₃—S—CH₂—Ar—(CH₂)₄—O—SO₃H;
CF₃—S—CH₂—Ar—(CH₂)₄—SH, CF₃—S—CH₂—Ar—(CH₂)₄—OH; CF₃S—CH₂—Ar—(CH₂)₄—N⁺R¹R²R³Z⁻;
CF₃S—CH₂—Ar—(CH₂)₄—P⁺R¹R²R³Z⁻; CF₃S—CH₂—Ar—(CH₂)₄—O-
glucoside; CF₃S—CH₂—Ar—(CH₂)₄—OPO₃H
CF₃—S—CH₂—Ar—(CH₂)₅—COOH; CF₃—S—CH₂—Ar—(CH₂)₅—SO₃H; CF₃—S—CH₂—Ar—(CH₂)₅—O—SO₃H;
CF₃—S—CH₂—Ar—(CH₂)₅—SH, CF₃—S—CH₂—Ar—(CH₂)₅—OH; CF₃S—CH₂—Ar—(CH₂)₅—N⁺R¹R²R³Z⁻;
CF₃S—CH₂—Ar—(CH₂)₅—P⁺R¹R²R³Z⁻; CF₃S—CH₂—Ar—(CH₂)₅—O-
glucoside, CF₃S—CH₂—Ar—(CH₂)₅—O—PO₃H
CF₃—S—CH₂—Ar—(CH₂)₆—COOH; CF₃—S—CH₂—Ar—(CH₂)₆—SO₃H; CF₃—S—CH₂—Ar—(CH₂)₆—O—SO₃H;
CF₃—S—CH₂—Ar—(CH₂)₆—SH, CF₃—S—CH₂—Ar—(CH₂)₆—OH; CF₃S—CH₂—Ar—(CH₂)₆—N⁺R¹R²R³Z⁻;
CF₃S—CH₂—Ar—(CH₂)₆—P⁺R¹R²R³Z⁻; CF₃S—CH₂—Ar—(CH₂)₆—O-
glucoside; CF₃S—CH₂—Ar—(CH₂)₆—OPO₃H
CF₃—S—CH₂—Ar—(CH₂)₇—COOH; CF₃—S—CH₂—Ar—(CH₂)₇—SO₃H; CF₃—S—CH₂—Ar—(CH₂)₇—O—SO₃H;
CF₃—S—CH₂—Ar—(CH₂)₇—SH, CF₃—S—CH₂—Ar—(CH₂)₇—OH; CF₃S—CH₂—Ar—(CH₂)₇—N⁺R¹R²R³Z⁻;
CF₃S—CH₂—Ar—(CH₂)₇—P⁺R¹R²R³Z⁻; CF₃S—CH₂—Ar—(CH₂)₇—O-
glucoside; CF₃S—CH₂—Ar—(CH₂)₇—OPO₃H
CF₃—S—CH₂—Ar—(CH₂)₈—COOH; CF₃—S—CH₂—Ar—(CH₂)₈—SO₃H; CF₃—S—CH₂—Ar—(CH₂)₈—O—SO₃H;
CF₃—S—CH₂—Ar—(CH₂)₈—SH, CF₃—S—CH₂—Ar—(CH₂)₈—OH; CF₃S—CH₂—Ar—(CH₂)₈—N⁺R¹R²R³Z⁻;
CF₃S—CH₂—Ar—(CH₂)₈—P⁺R¹R²R³Z⁻; CF₃S—CH₂—Ar—(CH₂)₈—O-
glucoside; CF₃S—CH₂—Ar—(CH₂)₈—OPO₃H
CF₃—S—CH₂—Ar—(CH₂)₉—COOH; CF₃—S—CH₂—Ar—(CH₂)₉—SO₃H; CF₃—S—CH₂—Ar—(CH₂)₉—O—SO₃H;
CF₃—S—CH₂—Ar—(CH₂)₉—SH, CF₃—S—CH₂—Ar—(CH₂)₉—OH CF₃S—CH₂—Ar—(CH₂)₉—N⁺R¹R²R³Z⁻;
CF₃S—CH₂—Ar—(CH₂)₉—P⁺R¹R²R³Z⁻; CF₃S—CH₂—Ar—(CH₂)₉—O-glucoside;
CF₃S—CH₂—Ar—(CH₂)₉—OPO₃H
CF₃—S—CH₂—Ar—(CH₂)₁₀—COOH; CF₃—S—CH₂—Ar—(CH₂)₁₀—SO₃H; CF₃—S—CH₂—Ar—(CH₂)₁₀—O—SO₃H;
CF₃—S—CH₂—Ar—(CH₂)₁₀—SH, CF₃—S—CH₂—Ar—(CH₂)₁₀—OH; CF₃S—CH₂—Ar—(CH₂)₁₀—N⁺R¹R²R³Z⁻;
CF₃S—CH₂—Ar—(CH₂)₁₀—P⁺R¹R²R³Z⁻; CF₃S—CH₂—Ar—(CH₂)₁₀—O-
glucoside; CF₃S—CH₂—Ar—(CH₂)₁₀—OPO₃H
CF₃—S—CH₂—Ar—(CH₂)₁₁—COOH; CF₃—S—CH₂—Ar—(CH₂)₁₁—SO₃H; CF₃—S—CH₂—Ar—(CH₂)₁₁—O—SO₃H;
CF₃—S—CH₂—Ar—(CH₂)₁₁—SH, CF₃—S—CH₂—Ar—(CH₂)₁₁—OH; CF₃S—CH₂—Ar—(CH₂)₁₁—N⁺R¹R²R³Z⁻;
CF₃S—CH₂—Ar—(CH₂)₁₁—P⁺R¹R²R³Z⁻; CF₃S—CH₂—Ar—(CH₂)₁₁—O-
glucoside; CF₃S—CH₂—Ar—(CH₂)₁₁—OPO₃H
CF₃—S—CH₂—Ar—(CH₂)₁₂—COOH; CF₃—S—CH₂—Ar—(CH₂)₁₂—SO₃H; CF₃—S—CH₂—Ar—(CH₂)₁₂—O—SO₃H;
CF₃—S—CH₂—Ar—(CH₂)₁₂—SH, CF₃—S—CH₂—Ar—(CH₂)₁₂—OH; CF₃S—CH₂—Ar—(CH₂)₁₂—N⁺R¹R²R³Z⁻;
CF₃S—CH₂—Ar—(CH₂)₁₂—P⁺R¹R²R³Z⁻; CF₃S—CH₂—Ar—(CH₂)₁₂—O-
glucoside; CF₃S—CH₂—Ar—(CH₂)₁₂—OPO₃H
CF₃—S—CH₂—Ar—(CH₂)₁₃—COOH; CF₃—S—CH₂—Ar—(CH₂)₁₃—SO₃H; CF₃—S—CH₂—Ar—(CH₂)₁₃—O—SO₃H;
CF₃—S—CH₂—Ar—(CH₂)₁₃—SH; CF₃—S—CH₂—Ar—(CH₂)₁₃—OH; CF₃S—CH₂—Ar—(CH₂)₁₃—N⁺R¹R²R³Z⁻;
CF₃S—CH₂—Ar—(CH₂)₁₃—P⁺R¹R²R³Z⁻; CF₃S—CH₂—Ar—(CH₂)₁₃—O-
glucoside; CF₃S—CH₂—Ar—(CH₂)₁₃—OPO₃H
CF₃—S—CH₂—Ar—(CH₂)₁₄—COOH; CF₃—S—CH₂—Ar—(CH₂)₁₄—SO₃H; CF₃—S—CH₂—Ar—(CH₂)₁₄—O—SO₃H;
CF₃—S—CH₂—Ar—(CH₂)₁₄—SH, CF₃—S—CH₂—Ar—(CH₂)₁₄—OH; CF₃S—CH₂—Ar—(CH₂)₁₄—N⁺R¹R²R³Z⁻;
CF₃S—CH₂—Ar—(CH₂)₁₄—P⁺R¹R²R³Z⁻; CF₃S—CH₂—Ar—(CH₂)₁₄—O-
glucoside; CF₃S—CH₂—Ar—(CH₂)₁₄—OPO₃H
CF₃—S—CH₂—Ar—(CH₂)₁₅—COOH; CF₃—S—CH₂—Ar—(CH₂)₁₅—SO₃H; CF₃—S—CH₂—Ar—(CH₂)₁₅—O—SO₃H;
CF₃—S—CH₂—Ar—(CH₂)₁₅—SH, CF₃—S—CH₂—Ar—(CH₂)₁₅—OH; CF₃S—CH₂—Ar—(CH₂)₁₅—N⁺R¹R²R³Z⁻;
CF₃S—CH₂—Ar—(CH₂)₁₅—P⁺R¹R²R³Z⁻; CF₃S—CH₂—Ar—(CH₂)₁₅—O-
glucoside; CF₃S—CH₂—Ar—(CH₂)₁₅—OPO₃H
CF₃—S—CH₂—Ar—(CH₂)₁₆—COOH; CF₃—S—CH₂—Ar—(CH₂)₁₆—SO₃H; CF₃—S—CH₂—Ar—(CH₂)₁₆—O—SO₃H;
CF₃—S—CH₂—Ar—(CH₂)₁₆—SH, CF₃—S—CH₂—Ar—(CH₂)₁₆—OH; CF₃S—CH₂—Ar—(CH₂)₁₆—N⁺R¹R²R³Z⁻;
CF₃S—CH₂—Ar—(CH₂)₁₆—P⁺R¹R²R³Z⁻; CF₃S—CH₂—Ar—(CH₂)₁₆—O-
glucoside; CF₃S—CH₂—Ar—(CH₂)₁₆—OPO₃H
(CF₃)₂N—CH₂—Ar—(CH₂)₄—COOH; (CF₃)₂N—CH₂—Ar—(CH₂)₄—SO₃H; (CF₃)₂N—CH₂—Ar—(CH₂)₄—O—SO₃H;
(CF₃)₂N—CH₂—Ar—(CH₂)₄—O—PO₃H
(CF₃)₂N—CH₂—Ar—(CH₂)₅—COOH; (CF₃)₂N—CH₂—Ar—(CH₂)₅—SO₃H; (CF₃)₂N—CH₂—Ar—(CH₂)₅—O—SO₃H;
(CF₃)₂N—CH₂—Ar—(CH₂)₅—O—PO₃H
(CF₃)₂N—CH₂—Ar—(CH₂)₆—COOH; (CF₃)₂N—CH₂—Ar—(CH₂)₆—SO₃H; (CF₃)₂N—CH₂—Ar—(CH₂)₆—O—SO₃H;
(CF₃)₂N—CH₂—Ar—(CH₂)₆—O—PO₃H
(CF₃)₂N—CH₂—Ar—(CH₂)₇—COOH; (CF₃)₂N—CH₂—Ar—(CH₂)₇—SO₃H; (CF₃)₂N—CH₂—Ar—(CH₂)₇—O—SO₃H;
(CF₃)₂N—CH₂—Ar—(CH₂)₇—O—PO₃H
(CF₃)₂N—CH₂—Ar—(CH₂)₈—COOH; (CF₃)₂N—CH₂—Ar—(CH₂)₈—SO₃H; (CF₃)₂N—CH₂—Ar—(CH₂)₈—O—SO₃H;
(CF₃)₂N—CH₂—Ar—(CH₂)₈—O—PO₃H
(CF₃)₂N—CH₂—Ar—(CH₂)₉—COOH; (CF₃)₂N—CH₂—Ar—(CH₂)₉—SO₃H; (CF₃)₂N—CH₂—Ar—(CH₂)₉—O—SO₃H;
(CF₃)₂NCH₂—Ar—(CH₂)₉—O—PO₃H
(CF₃)₂N—CH₂—Ar—(CH₂)₁₀—COOH; (CF₃)₂N—CH₂—Ar—(CH₂)₁₀—SO₃H; (CF₃)₂N—CH₂—Ar—(CH₂)₁₀—O—SO₃H;
(CF₃)₂N—CH₂—Ar—(CH₂)₁₀—O—PO₃H
(CF₃)₂N—CH₂—Ar—(CH₂)₁₁—COOH; (CF₃)₂N—CH₂—Ar—(CH₂)₁₁—SO₃H; (CF₃)₂N—CH₂—Ar—(CH₂)₁₁—O—SO₃H;
(CF₃)₂N—CH₂—Ar—(CH₂)₁₁—O—PO₃H
(CF₃)₂N—CH₂—Ar—(CH₂)₁₂—COOH; (CF₃)₂N—CH₂—Ar—(CH₂)₁₂—SO₃H; (CF₃)₂N—CH₂—Ar—(CH₂)₁₂—O—SO₃H;
(CF₃)₂N—CH₂—Ar—(CH₂)₁₂—O—PO₃H
(CF₃)₂N—CH₂—Ar—(CH₂)₁₃—COOH; (CF₃)₂N—CH₂—Ar—(CH₂)₁₃—SO₃H; (CF₃)₂N—CH₂—Ar—(CH₂)₁₃—O—SO₃H;
(CF₃)₂N—CH₂—Ar—(CH₂)₁₃—O—PO₃H;
(CF₃)₂N—CH₂—Ar—(CH₂)₁₄—COOH; (CF₃)₂N—CH₂—Ar—(CH₂)₁₄—SO₃H; (CF₃)₂N—CH₂—Ar—(CH₂)₁₄—O—SO₃H;
(CF₃)₂N—CH₂—Ar—(CH₂)₁₄—O—PO₃H
(CF₃)₂N—CH₂—Ar—(CH₂)₁₅—COOH; (CF₃)₂N—CH₂—Ar—(CH₂)₁₅—SO₃H; (CF₃)₂N—CH₂—Ar—(CH₂)₁₅—O—SO₃H;
(CF₃)₂N—CH₂—Ar—(CH₂)₁₅—O—PO₃H
(CF₃)₂N—CH₂—Ar—(CH₂)₁₆—COOH; (CF₃)₂N—CH₂—Ar—(CH₂)₁₆—SO₃H; (CF₃)₂N—CH₂—Ar—(CH₂)₁₆—O—SO₃H;
(CF₃)₂N—CH₂—Ar—(CH₂)₁₆—O—PO₃H
CF₃—CH₂—CH₂—S—CH₂—Ar—(CH₂)₄—N⁺R¹R²R³Z⁻; CF₃—CH₂—CH₂—S—CH₂—Ar—(CH₂)₄—P⁺R¹R²R³Z⁻;
CF₃—CH₂—CH₂—S—CH₂—Ar—(CH₂)₄—O-glucoside
CF₃—CH₂—CH₂—S—CH₂—Ar—(CH₂)₅—N⁺R¹R²R³Z⁻; CF₃—CH₂—CH₂—S—CH₂—Ar—(CH₂)₅—P⁺R¹R²R³Z⁻;
CF₃—CH₂—CH₂—S—CH₂—Ar—(CH₂)₅—O-glucoside
CF₃—CH₂—CH₂—S—CH₂—Ar—(CH₂)₆—N⁺R¹R²R³Z⁻; CF₃—CH₂—CH₂—S—CH₂—Ar—(CH₂)₆—P⁺R¹R²R³Z⁻;
CF₃—CH₂—CH₂—S—CH₂—Ar—(CH₂)₆—O-glucoside
CF₃—CH₂—CH₂—S—CH₂—Ar—(CH₂)₇—N⁺R¹R²R³Z⁻; CF₃—CH₂—CH₂—S—CH₂—Ar—(CH₂)₇—P⁺R¹R²R³Z⁻;
CF₃—CH₂—CH₂—S—CH₂—Ar—(CH₂)₇—O-glucoside
CF₃—CH₂—CH₂—S—CH₂—Ar—(CH₂)₈—N⁺R¹R²R³Z⁻; CF₃—CH₂—CH₂—S—CH₂—Ar—(CH₂)₈—P⁺R¹R²R³Z⁻;
CF₃—CH₂—CH₂—S—CH₂—Ar—(CH₂)₈—O-glucoside
CF₃—CH₂—CH₂—S—CH₂—Ar—(CH₂)₉—N⁺R¹R²R³Z⁻; CF₃—CH₂—CH₂—S—CH₂—Ar—(CH₂)₉—P⁺R¹R²R³Z⁻;
CF₃—CH₂—CH₂—S—CH₂—Ar—(CH₂)₉—O-glucoside
CF₃—CH₂—CH₂—S—CH₂—Ar—(CH₂)₁₀—N⁺R¹R²R³Z⁻; CF₃—CH₂—CH₂—S—CH₂—Ar—(CH₂)₁₀—P⁺R¹R²R³Z⁻;
CF₃—CH₂—CH₂—S—CH₂—Ar—(CH₂)₁₀—O-glucoside
CF₃—CH₂—CH₂—S—CH₂—Ar—(CH₂)₁₁—N⁺R¹R²R³Z⁻; CF₃—CH₂—CH₂—S—CH₂—Ar—(CH₂)₁₁—P⁺R¹R²R³Z⁻;
CF₃—CH₂—CH₂—S—CH₂—Ar—(CH₂)₁₁—O-glucoside
CF₃—CH₂—CH₂—S—CH₂—Ar—(CH₂)₁₂—N⁺R¹R²R³Z⁻; CF₃—CH₂—CH₂—S—CH₂—Ar—(CH₂)₁₂—P⁺R¹R²R³Z⁻;
CF₃—CH₂—CH₂—S—CH₂—Ar—(CH₂)₁₂—O-glucoside
CF₃—CH₂—CH₂—S—CH₂—Ar—(CH₂)₁₃—N⁺R¹R²R³Z⁻; CF₃—CH₂—CH₂—S—CH₂—Ar—(CH₂)₁₃—P⁺R¹R²R³Z⁻;
CF₃—CH₂—CH₂—S—CH₂—Ar—(CH₂)₁₃—O-glucoside
CF₃—CH₂—CH₂—S—CH₂—Ar—(CH₂)₁₄—N⁺R¹R²R³Z⁻; CF₃—CH₂—CH₂—S—CH₂—Ar—(CH₂)₁₄—P⁺R¹R²R³Z⁻;
CF₃—CH₂—CH₂—S—CH₂—Ar—(CH₂)₁₄—O-glucoside
CF₃—CH₂—CH₂—S—CH₂—Ar—(CH₂)₁₅—N⁺R¹R²R³Z⁻; CF₃—CH₂—CH₂—S—CH₂—Ar—(CH₂)₁₅—P⁺R¹R²R³Z⁻;
CF₃—CH₂—CH₂—S—CH₂—Ar—(CH₂)₁₅—O-glucoside
CF₃—CH₂—CH₂—S—CH₂—Ar—(CH₂)₁₆—N⁺R¹R²R³Z⁻; CF₃—CH₂—CH₂—S—CH₂—Ar—(CH₂)₁₆—P⁺R¹R²R³Z⁻;
CF₃—CH₂—CH₂—S—CH₂—Ar—(CH₂)₁₆—O-glucoside
(CF₃)₂N—CH₂—Ar—(CH₂)₄—OH; (CF₃)₂N—CH₂—Ar—(CH₂)₄—SH; (CF₃)₂N—CH₂—Ar—(CH₂)₄—N⁺R¹R²R³Z⁻;
(CF₃)₂N—CH₂—Ar—(CH₂)₄—P⁺R¹R²R³Z⁻; (CF₃)₂N—CH₂—Ar—(CH₂)₄—O-
glucoside
(CF₃)₂N—CH₂—Ar—(CH₂)₅—OH; (CF₃)₂N—CH₂—Ar—(CH₂)₅—SH; (CF₃)₂N—CH₂—Ar—(CH₂)₅—N⁺R¹R²R³Z⁻;
(CF₃)₂N—CH₂—Ar—(CH₂)₅—P⁺R¹R²R³Z⁻; (CF₃)₂N—CH₂—Ar—(CH₂)₅—O-
glucoside
(CF₃)₂N—CH₂—Ar—(CH₂)₆—OH; (CF₃)₂N—CH₂—Ar—(CH₂)₆—SH; (CF₃)₂N—CH₂—Ar—(CH₂)₆—N⁺R¹R²R³Z⁻;
(CF₃)₂N—CH₂—Ar—(CH₂)₆—P⁺R¹R²R³Z⁻; (CF₃)₂N—CH₂—Ar—(CH₂)₆—O-
glucoside
(CF₃)₂N—CH₂—Ar—(CH₂)₇—OH; (CF₃)₂N—CH₂—Ar—(CH₂)₇—SH; (CF₃)₂N—CH₂—Ar—(CH₂)₇—N⁺R¹R²R³Z⁻;
(CF₃)₂N—CH₂—Ar—(CH₂)₇—P⁺R¹R²R³Z⁻; (CF₃)₂N—CH₂—Ar—(CH₂)₇—O-
glucoside
(CF₃)₂N—CH₂—Ar—(CH₂)₈—OH; (CF₃)₂N—CH₂—Ar—(CH₂)₈—SH; (CF₃)₂N—CH₂—Ar—(CH₂)₈—N⁺R¹R²R³Z⁻;
(CF₃)₂N—CH₂—Ar—(CH₂)₈—P⁺R¹R²R³Z⁻; (CF₃)₂N—CH₂—Ar—(CH₂)₈—O-
glucoside
(CF₃)₂N—CH₂—Ar—(CH₂)₉—OH; (CF₃)₂N—CH₂—Ar—(CH₂)₉—SH; (CF₃)₂N—CH₂—Ar—(CH₂)₉—N⁺R¹R²R³Z⁻;
(CF₃)₂N—CH₂—Ar—(CH₂)₉—P⁺R¹R²R³Z⁻; (CF₃)₂N—CH₂—Ar—(CH₂)₉—O-
glucoside
(CF₃)₂N—CH₂—Ar—(CH₂)₁₀—OH; (CF₃)₂N—CH₂—Ar—(CH₂)₁₀—SH; (CF₃)₂N—CH₂—Ar—(CH₂)₁₀—N⁺R¹R²R³Z⁻;
(CF₃)₂N—CH₂—Ar—(CH₂)₁₀—P⁺R¹R²R³Z⁻; (CF₃)₂N—CH₂—Ar—(CH₂)₁₀—O-
glucoside
(CF₃)₂N—CH₂—Ar—(CH₂)₁₁—OH; (CF₃)₂N—CH₂—Ar—(CH₂)₁₁—SH; (CF₃)₂N—CH₂—Ar—(CH₂)₁₁—N⁺R¹R²R³Z⁻;
(CF₃)₂N—CH₂—Ar—(CH₂)₁₁—P⁺R¹R²R³Z⁻; (CF₃)₂N—CH₂—Ar—(CH₂)₁₁—O-
glucoside
(CF₃)₂N—CH₂—Ar—(CH₂)₁₂—OH; (CF₃)₂N—CH₂—Ar—(CH₂)₁₂—SH; (CF₃)₂N—CH₂—Ar—(CH₂)₁₂—N⁺R¹R²R³Z⁻;
(CF₃)₂N—CH₂—Ar—(CH₂)₁₂—P⁺R¹R²R³Z⁻; (CF₃)₂N—CH₂—Ar—(CH₂)₁₂—O-
glucoside
(CF₃)₂N—CH₂—Ar—(CH₂)₁₃—OH; (CF₃)₂N—CH₂—Ar—(CH₂)₁₃—SH; (CF₃)₂N—CH₂—Ar—(CH₂)₁₃—N⁺R¹R²R³Z⁻;
(CF₃)₂N—CH₂—Ar—(CH₂)₁₃—P⁺R¹R²R³Z⁻; (CF₃)₂N—CH₂—Ar—(CH₂)₁₃—O-
glucoside
(CF₃)₂N—CH₂—Ar—(CH₂)₁₄—OH; (CF₃)₂N—CH₂—Ar—(CH₂)₁₄—SH; (CF₃)₂N—CH₂—Ar—(CH₂)₁₄—N⁺R¹R²R³Z⁻;
(CF₃)₂N—CH₂—Ar—(CH₂)₁₄—P⁺R¹R²R³Z⁻; (CF₃)₂N—CH₂—Ar—(CH₂)₁₄—O-
glucoside
(CF₃)₂N—CH₂—Ar—(CH₂)₁₅—OH; (CF₃)₂N—CH₂—Ar—(CH₂)₁₅—SH; (CF₃)₂N—CH₂—Ar—(CH₂)₁₅—N⁺R¹R²R³Z⁻;
(CF₃)₂N—CH₂—Ar—(CH₂)₁₅—P⁺R¹R²R³Z⁻; (CF₃)₂N—CH₂—Ar—(CH₂)₁₅—O-
glucoside
(CF₃)₂N—CH₂—Ar—(CH₂)₁₆—OH; (CF₃)₂N—CH₂—Ar—(CH₂)₁₆—SH; (CF₃)₂N—CH₂—Ar—(CH₂)₁₆—N⁺R¹R²R³Z⁻;
(CF₃)₂N—CH₂—Ar—(CH₂)₁₆—P⁺R¹R²R³Z⁻; (CF₃)₂N—CH₂—Ar—(CH₂)₁₆—O-
glucoside
CF₃CH₂CH₂S—CH₂—Ar—(CH₂)₄—OH; CF₃CH₂CH₂S—CH₂—Ar—(CH₂)₄—SH; CF₃CH₂CH₂S—CH₂—Ar—(CH₂)₄—O—PO₃H
CF₃CH₂CH₂S—CH₂—Ar—(CH₂)₅—OH; CF₃CH₂CH₂S—CH₂—Ar—(CH₂)₅—SH; CF₃CH₂CH₂S—CH₂—Ar—(CH₂)₅—O—PO₃H
CF₃CH₂CH₂S—CH₂—Ar—(CH₂)₆—OH; CF₃CH₂CH₂S—CH₂—Ar—(CH₂)₆—SH; CF₃CH₂CH₂S—CH₂—Ar—(CH₂)₆—O—PO₃H
CF₃CH₂CH₂S—CH₂—Ar—(CH₂)₇—OH; CF₃CH₂CH₂S—CH₂—Ar—(CH₂)₇—SH; CF₃CH₂CH₂S—CH₂—Ar—(CH₂)₇—O—PO₃H
CF₃CH₂CH₂S—CH₂—Ar—(CH₂)₈—OH; CF₃CH₂CH₂S—CH₂—Ar—(CH₂)₈—SH; CF₃CH₂CH₂S—CH₂—Ar—(CH₂)₈—O—PO₃H
CF₃CH₂CH₂S—CH₂—Ar—(CH₂)₉—OH; CF₃CH₂CH₂S—CH₂—Ar—(CH₂)₉—SH; CF₃CH₂CH₂S—CH₂—Ar—(CH₂)₉—O—PO₃H
CF₃CH₂CH₂S—CH₂—Ar—(CH₂)₁₀—OH; CF₃CH₂CH₂S—CH₂—Ar—(CH₂)₁₀—SH;
CF₃CH₂CH₂S—CH₂—Ar—(CH₂)₁₀—O—PO₃H
CF₃CH₂CH₂S—CH₂—Ar—(CH₂)₁₁—OH CF₃CH₂CH₂S—CH₂—Ar—(CH₂)₁₁—SH CF₃CH₂CH₂S—CH₂—Ar—(CH₂)₁₁—O—PO₃H
CF₃CH₂CH₂S—CH₂—Ar—(CH₂)₁₂—OH CF₃CH₂CH₂S—CH₂—Ar—(CH₂)₁₂—SH; CF₃CH₂CH₂S—CH₂—Ar—(CH₂)₁₂—O—PO₃H
CF₃CH₂CH₂S—CH₂—Ar—(CH₂)₁₃—OH; CF₃CH₂CH₂S—CH₂—Ar—(CH₂)₁₃—SH;
CF₃CH₂CH₂S—CH₂—Ar—(CH₂)₁₃—O—PO₃H;
CF₃CH₂CH₂S—CH₂—Ar—(CH₂)₁₄—OH; CF₃CH₂CH₂S—CH₂—Ar—(CH₂)₁₄—SH;
CF₃CH₂CH₂S—CH₂—Ar—(CH₂)₁₄—O—PO₃H
CF₃CH₂CH₂S—CH₂—Ar—(CH₂)₁₅—OH; CF₃CH₂CH₂S—CH₂—Ar—(CH₂)₁₅—SH;
CF₃CH₂CH₂S—CH₂—Ar—(CH₂)₁₅—O—PO₃H
CF₃CH₂CH₂S—CH₂—Ar—(CH₂)₁₆—OH; CF₃CH₂CH₂S—CH₂—Ar—(CH₂)₁₆—SH;
CF₃CH₂CH₂S—CH₂—Ar—(CH₂)₁₆—O—PO₃H
(CF₃CH₂CH₂)₂N—CH₂—Ar—(CH₂)₄—OH; (CF₃CH₂CH₂)₂N—CH₂—Ar—(CH₂)₄—SH;
(CF₃CH₂CH₂)₂N—CH₂—Ar—(CH₂)₄—N⁺R¹R²R³Z⁻; (CF₃CH₂CH₂)₂N—CH₂—Ar—(CH₂)₄—P⁺R¹R²R³Z⁻;
(CF₃CH₂CH₂)₂N—CH₂—Ar—(CH₂)₄—O-glucoside;
(CF₃CH₂CH₂)₂N—CH₂—Ar—(CH₂)₅—OH; (CF₃CH₂CH₂)₂N—CH₂—Ar—(CH₂)₅—SH;
(CF₃CH₂CH₂)₂N—CH₂—Ar—(CH₂)₅—N⁺R¹R²R³Z⁻; (CF₃CH₂CH₂)₂N—CH₂—Ar—(CH₂)₅—P⁺R¹R²R³Z⁻;
(CF₃CH₂CH₂)₂N—CH₂—Ar—(CH₂)₅—O-glucoside
(CF₃CH₂CH₂)₂N—CH₂—Ar—(CH₂)₆—OH; (CF₃CH₂CH₂)₂N—CH₂—Ar—(CH₂)₆—SH;
(CF₃CH₂CH₂)₂N—CH₂—Ar—(CH₂)₆—N⁺R¹R²R³Z⁻; (CF₃CH₂CH₂)₂N—CH₂—Ar—(CH₂)₆—P⁺R¹R²R³Z⁻;
(CF₃CH₂CH₂)₂N—CH₂—Ar—(CH₂)₆—O-glucoside
(CF₃CH₂CH₂)₂N—CH₂—Ar—(CH₂)₇—OH; (CF₃CH₂CH₂)₂N—CH₂—Ar—(CH₂)₇—SH;
(CF₃CH₂CH₂)₂N—CH₂—Ar—(CH₂)₇—N⁺R¹R²R³Z⁻; (CF₃CH₂CH₂)₂N—CH₂—Ar—(CH₂)₇—P⁺R¹R²R³Z⁻;
(CF₃CH₂CH₂)₂N—CH₂—Ar—(CH₂)₇—O-glucoside
(CF₃CH₂CH₂)₂N—CH₂—Ar—(CH₂)₈—OH; (CF₃CH₂CH₂)₂N—CH₂—Ar—(CH₂)₈—SH;
(CF₃CH₂CH₂)₂N—CH₂—Ar—(CH₂)₈—N⁺R¹R²R³Z⁻; (CF₃CH₂CH₂)₂N—CH₂—Ar—(CH₂)₈—P⁺R¹R²R³Z⁻;
(CF₃CH₂CH₂)₂N—CH₂—Ar—(CH₂)₈—O-glucoside
(CF₃CH₂CH₂)₂N—CH₂—Ar—(CH₂)₉—OH; (CF₃CH₂CH₂)₂N—CH₂—Ar—(CH₂)₉—SH;
(CF₃CH₂CH₂)₂N—CH₂—Ar—(CH₂)₉—N⁺R¹R²R³Z⁻; (CF₃CH₂CH₂)₂N—CH₂—Ar—(CH₂)₉—P⁺R¹R²R³Z⁻;
(CF₃CH₂CH₂)₂N—CH₂—Ar—(CH₂)₉—O-glucoside
(CF₃CH₂CH₂)₂N—CH₂—Ar—(CH₂)₁₀—OH; (CF₃CH₂CH₂)₂N—CH₂—Ar—(CH₂)₁₀—SH;
(CF₃CH₂CH₂)₂N—CH₂—Ar—(CH₂)₁₀—N⁺R¹R²R³Z⁻; (CF₃CH₂CH₂)₂N—CH₂—Ar—(CH₂)₁₀—P⁺R¹R²R³Z⁻;
(CF₃CH₂CH₂)₂N—CH₂—Ar—(CH₂)₁₀—O-glucoside
(CF₃CH₂CH₂)₂N—CH₂—Ar—(CH₂)₁₁—OH; (CF₃CH₂CH₂)₂N—CH₂—Ar—(CH₂)₁₁—SH;
(CF₃CH₂CH₂)₂N—CH₂—Ar—(CH₂)₁₁—N⁺R¹R²R³Z⁻; (CF₃CH₂CH₂)₂N—CH₂—Ar—(CH₂)₁₁—P⁺R¹R²R³Z⁻;
(CF₃CH₂CH₂)₂N—CH₂—Ar—(CH₂)₁₁—O-glucoside
(CF₃CH₂CH₂)₂N—CH₂—Ar—(CH₂)₁₂—OH; (CF₃CH₂CH₂)₂N—CH₂—Ar—(CH₂)₁₂—SH;
(CF₃CH₂CH₂)₂N—CH₂—Ar—(CH₂)₁₂—N⁺R¹R²R³Z⁻; (CF₃CH₂CH₂)₂N—CH₂—Ar—(CH₂)₁₂—P⁺R¹R²R³Z⁻;
(CF₃CH₂CH₂)₂N—CH₂—Ar—(CH₂)₁₂—O-glucoside
(CF₃CH₂CH₂)₂N—CH₂—Ar—(CH₂)₁₃—OH; (CF₃CH₂CH₂)₂N—CH₂—Ar—(CH₂)₁₃—SH;
(CF₃CH₂CH₂)₂N—CH₂—Ar—(CH₂)₁₃—N⁺R¹R²R³Z⁻; (CF₃CH₂CH₂)₂N—CH₂—Ar—(CH₂)₁₃—P⁺R¹R²R³Z⁻;
(CF₃CH₂CH₂)₂N—CH₂—Ar—(CH₂)₁₃—O-glucoside
(CF₃CH₂CH₂)₂N—CH₂—Ar—(CH₂)₁₄—OH; (CF₃CH₂CH₂)₂N—CH₂—Ar—(CH₂)₁₄—SH;
(CF₃CH₂CH₂)₂N—CH₂—Ar—(CH₂)₁₄—N⁺R¹R²R³Z⁻; (CF₃CH₂CH₂)₂N—CH₂—Ar—(CH₂)₁₄—P⁺R¹R²R³Z⁻;
(CF₃CH₂CH₂)₂N—CH₂—Ar—(CH₂)₁₄—O-glucoside
(CF₃CH₂CH₂)₂N—CH₂—Ar—(CH₂)₁₅—OH; (CF₃CH₂CH₂)₂N—CH₂—Ar—(CH₂)₁₅—SH;
(CF₃CH₂CH₂)₂N—CH₂—Ar—(CH₂)₁₅—N⁺R¹R²R³Z⁻; (CF₃CH₂CH₂)₂N—CH₂—Ar—(CH₂)₁₅—P⁺R¹R²R³Z⁻;
(CF₃CH₂CH₂)₂N—CH₂—Ar—(CH₂)₁₅—O-glucoside
(CF₃CH₂CH₂)₂N—CH₂—Ar—(CH₂)₁₆—OH; (CF₃CH₂CH₂)₂N—CH₂—Ar—(CH₂)₁₆—SH;
(CF₃CH₂CH₂)₂N—CH₂—Ar—(CH₂)₁₆—N⁺R¹R²R³Z⁻; (CF₃CH₂CH₂)₂N—CH₂—Ar—(CH₂)₁₆—P⁺R¹R²R³Z⁻;
(CF₃CH₂CH₂)₂N—CH₂—Ar—(CH₂)₁₆—O-glucoside
CF₃CF₂S—CH₂—Ar—(CH₂)₄—OH; CF₃CF₂S—CH₂—Ar—(CH₂)₄—SH; CF₃CF₂S—CH₂—Ar—(CH₂)₄—N⁺R¹R²R³Z⁻;
CF₃CF₂S—CH₂—Ar—(CH₂)₄—P⁺R¹R²R³Z⁻; CF₃CF₂S—CH₂—Ar—(CH₂)₄—O-
glucoside
CF₃CF₂S—CH₂—Ar—(CH₂)₅—OH; CF₃CF₂S—CH₂—Ar—(CH₂)₅—SH; CF₃CF₂S—CH₂—Ar—(CH₂)₅—N⁺R¹R²R³Z⁻;
CF₃CF₂S—CH₂—Ar—(CH₂)₅—P⁺R¹R²R³Z⁻; CF₃CF₂S—CH₂—Ar—(CH₂)₅—O-
glucoside
CF₃CF₂S—CH₂—Ar—(CH₂)₆—OH; CF₃CF₂S—CH₂—Ar—(CH₂)₆—SH; CF₃CF₂S—CH₂—Ar—(CH₂)₆—N⁺R¹R²R³Z⁻;
CF₃CF₂S—CH₂—Ar—(CH₂)₆—P⁺R¹R²R³Z⁻; CF₃CF₂S—CH₂—Ar—(CH₂)₆—O-
glucoside
CF₃CF₂S—CH₂—Ar—(CH₂)₇—OH; CF₃CF₂S—CH₂—Ar—(CH₂)₇—SH; CF₃CF₂S—CH₂—Ar—(CH₂)₇—N⁺R¹R²R³Z⁻;
CF₃CF₂S—CH₂—Ar—(CH₂)₇—P⁺R¹R²R³Z⁻; CF₃CF₂S—CH₂—Ar—(CH₂)₇—O-
glucoside
CF₃CF₂S—CH₂—Ar—(CH₂)₈—OH; CF₃CF₂S—CH₂—Ar—(CH₂)₈—SH; CF₃CF₂S—CH₂—Ar—(CH₂)₈—N⁺R¹R²R³Z⁻;
CF₃CF₂S—CH₂—Ar—(CH₂)₈—P⁺R¹R²R³Z⁻; CF₃CF₂S—CH₂—Ar—(CH₂)₈—O-
glucoside
CF₃CF₂S—CH₂—Ar—(CH₂)₉—OH; CF₃CF₂S—CH₂—Ar—(CH₂)₉—SH; CF₃CF₂S—CH₂—Ar—(CH₂)₉—N⁺R¹R²R³Z⁻;
CF₃CF₂S—CH₂—Ar—(CH₂)₉—P⁺R¹R²R³Z⁻; CF₃CF₂S—CH₂—Ar—(CH₂)₉—O-
glucoside
CF₃CF₂S—CH₂—Ar—(CH₂)₁₀—OH; CF₃CF₂S—CH₂—Ar—(CH₂)₁₀—SH; CF₃CF₂S—CH₂—Ar—(CH₂)₁₀—N⁺R¹R²R³Z⁻;
CF₃CF₂S—CH₂—Ar—(CH₂)₁₀—P⁺R¹R²R³Z⁻; CF₃CF₂S—CH₂—Ar—(CH₂)₁₀—O-
glucoside
CF₃CF₂S—CH₂—Ar—(CH₂)₁₁—OH; CF₃CF₂S—CH₂—Ar—(CH₂)₁₁—SH; CF₃CF₂S—CH₂—Ar—(CH₂)₁₁—N⁺R¹R²R³Z⁻;
CF₃CF₂S—CH₂—Ar—(CH₂)₁₁—P⁺R¹R²R³Z⁻; CF₃CF₂S—CH₂—Ar—(CH₂)₁₁—O-
glucoside
CF₃CF₂S—CH₂—Ar—(CH₂)₁₂—OH; CF₃CF₂S—CH₂—Ar—(CH₂)₁₂—SH; CF₃CF₂S—CH₂—Ar—(CH₂)₁₂—N⁺R¹R²R³Z⁻;
CF₃CF₂S—CH₂—Ar—(CH₂)₁₂—P⁺R¹R²R³Z⁻; CF₃CF₂S—CH₂—Ar—(CH₂)₁₂—O-
glucoside
CF₃CF₂S—CH₂—Ar—(CH₂)₁₃—OH; CF₃CF₂S—CH₂—Ar—(CH₂)₁₃—SH; CF₃CF₂S—CH₂—Ar—(CH₂)₁₃—N⁺R¹R²R³Z⁻;
CF₃CF₂S—CH₂—Ar—(CH₂)₁₃—P⁺R¹R²R³Z⁻; CF₃CF₂S—CH₂—Ar—(CH₂)₁₃—O-
glucoside
CF₃CF₂S—CH₂—Ar—(CH₂)₁₄—OH; CF₃CF₂S—CH₂—Ar—(CH₂)₁₄—SH; CF₃CF₂S—CH₂—Ar—(CH₂)₁₄—N⁺R¹R²R³Z⁻;
CF₃CF₂S—CH₂—Ar—(CH₂)₁₄—P⁺R¹R²R³Z⁻; CF₃CF₂S—CH₂—Ar—(CH₂)₁₄—O-
glucoside
CF₃CF₂S—CH₂—Ar—(CH₂)₁₅—OH; CF₃CF₂S—CH₂—Ar—(CH₂)₁₅—SH; CF₃CF₂S—CH₂—Ar—(CH₂)₁₅—N⁺R¹R²R³Z⁻;
CF₃CF₂S—CH₂—Ar—(CH₂)₁₅—P⁺R¹R²R³Z⁻; CF₃CF₂S—CH₂—Ar—(CH₂)₁₅—O-
glucoside
CF₃CF₂S—CH₂—Ar—(CH₂)₁₆—OH; CF₃CF₂S—CH₂—Ar—(CH₂)₁₆—SH; CF₃CF₂S—CH₂—Ar—(CH₂)₁₆—N⁺R¹R²R³Z⁻;
CF₃CF₂S—CH₂—Ar—(CH₂)₁₆—P⁺R¹R²R³Z⁻; CF₃CF₂S—CH₂—Ar—(CH₂)₁₆—O-
glucoside
CF₃—S—C≡C—(CH₂)₄—COOH; CF₃—S—C≡C—(CH₂)₄—SO₃H; CF₃—S—C≡C—(CH₂)₄—O—SO₃H,
CF₃—S—C≡C—(CH₂)₄—O—PO₃H, CF₃—S—C≡C—(CH₂)₄—N⁺R¹R²R³Z⁻; CF₃—S—C≡C—(CH₂)₄—P⁺R¹R²R³Z⁻;
CF₃—S—C≡C—(CH₂)₄—O-glucoside; CF₃—S—C≡C—(CH₂)₄—OH; CF₃—S—C≡C—(CH₂)₄—SH;
CF₃—S—C≡C—(CH₂)₅—COOH; CF₃—S—C≡C—(CH₂)₅—SO₃H; CF₃—S—C≡C—(CH₂)₅—O—SO₃H;
CF₃—S—C≡C—(CH₂)₅—O—PO₃H; CF₃—S—C≡C—(CH₂)₅—N⁺R¹R²R³Z⁻; CF₃—S—C≡C—(CH₂)₅—P⁺R¹R²R³Z⁻;
CF₃—S—C≡C—(CH₂)₅—O-glucoside; CF₃—S—C≡C—(CH₂)₅—OH; CF₃—S—C≡C—(CH₂)₅—SH;
CF₃—S—C≡C—(CH₂)₆—COOH; CF₃—S—C≡C—(CH₂)₆—SO₃H; CF₃—S—C≡C—(CH₂)₆—O—SO₃H;
CF₃—S—C≡C—(CH₂)₆—O—PO₃H; CF₃—S—C≡C—(CH₂)₆—N⁺R¹R²R³Z⁻; CF₃—S—C≡C—(CH₂)₆—P⁺R¹R²R³Z⁻;
CF₃—S—C≡C—(CH₂)₆—O-glucoside; CF₃—S—C≡C—(CH₂)₆—OH; CF₃—S—C≡C—(CH₂)₆—SH;
CF₃—S—C≡C—(CH₂)₇—COOH; CF₃—S—C≡C—(CH₂)₇—SO₃H; CF₃—S—C≡C—(CH₂)₇—O—SO₃H;
CF₃—S—C≡C—(CH₂)₇—O—PO₃H; CF₃—S—C≡C—(CH₂)₇—N⁺R¹R²R³Z⁻; CF₃—S—C≡C—(CH₂)₇—P⁺R¹R²R³Z⁻;
CF₃—S—C≡C—(CH₂)₇—O-glucoside; CF₃—S—C≡C—(CH₂)₇—OH; CF₃—S—C≡C—(CH₂)₇—SH;
CF₃—S—C≡C—(CH₂)₈—COOH; CF₃—S—C≡C—(CH₂)₈—SO₃H; CF₃—S—C≡C—(CH₂)₈—O—SO₃H;
CF₃—S—C≡C—(CH₂)₈—O—PO₃H; CF₃—S—C≡C—(CH₂)₈—N⁺R¹R²R³Z⁻; CF₃—S—C≡C—(CH₂)₈—P⁺R¹R²R³Z⁻;
CF₃—S—C≡C—(CH₂)₈—O-glucoside; CF₃—S—C≡C—(CH₂)₈—OH; CF₃—S—C≡C—(CH₂)₈—SH;
CF₃—S—C≡C—(CH₂)₉—COOH; CF₃—S—C≡C—(CH₂)₉—SO₃H; CF₃—S—C≡C—(CH₂)₉—O—SO₃H;
CF₃—S—C≡C—(CH₂)₉—O—PO₃H; CF₃—S—C≡C—(CH₂)₉—N⁺R¹R²R³Z⁻; CF₃—S—C≡C—(CH₂)₉—P⁺R¹R²R³Z⁻;
CF₃—S—C≡C—(CH₂)₉—O-glucoside; CF₃—S—C≡C—(CH₂)₉—OH; CF₃—S—C≡C—(CH₂)₉—SH;
CF₃—S—C≡C—(CH₂)₁₀—COOH; CF₃—S—C≡C—(CH₂)₁₀—SO₃H; CF₃—S—C≡C—(CH₂)₁₀—O—SO₃H;
CF₃—S—C≡C—(CH₂)₁₀—O—PO₃H; CF₃—S—C≡C—(CH₂)₁₀—N⁺R¹R²R³Z⁻; CF₃—S—C≡C—(CH₂)₁₀—P⁺R¹R²R³Z⁻;
CF₃—S—C≡C—(CH₂)₁₀—O-glucoside; CF₃—S—C≡C—(CH₂)₁₀—OH;
CF₃—S—C≡C—(CH₂)₁₀—SH;
CF₃—S—C≡C—(CH₂)₁₁—COOH; CF₃—S—C≡C—(CH₂)₁₁—SO₃H; CF₃—S—C≡C—(CH₂)₁₁—O—SO₃H;
CF₃—S—C≡C—(CH₂)₁₁—O—PO₃H; CF₃—S—C≡C—(CH₂)₁₁—N⁺R¹R²R³Z⁻; CF₃—S—C≡C—(CH₂)₁₁—P⁺R¹R²R³Z⁻;
CF₃—S—C≡C—(CH₂)₁₁—O-glucoside; CF₃—S—C≡C—(CH₂)₁₁—OH;
CF₃—S—C≡C—(CH₂)₁₁—SH;
CF₃—S—C≡C—(CH₂)₁₂—COOH; CF₃—SC≡C—(CH₂)₁₂—SO₃H; CF₃—S—C≡C—(CH₂)₁₂—O—SO₃H;
CF₃—S—C≡C—(CH₂)₁₂—O—PO₃H, CF₃—S—C≡C—(CH₂)₁₂—N⁺R¹R²R³Z⁻; CF₃—S—C≡C—(CH₂)₁₂—P⁺R¹R²R³Z⁻;
CF₃—S—C≡C—(CH₂)₁₂—O-glucoside; CF₃—S—C≡C—(CH₂)₁₂—OHCF₃—S—C≡C—(CH₂)₁₂—SH
CF₃—S—C≡C—(CH₂)₁₃—COOH; CF₃—S—C≡C—(CH₂)₁₃—SO₃H; CF₃—S—C≡C—(CH₂)₁₃—O—SO₃H;
CF₃—S—C≡C—(CH₂)₁₃—O—PO₃H; CF₃—S—C≡C—(CH₂)₁₃—N⁺R¹R²R³Z⁻; CF₃—S—C≡C—(CH₂)₁₃—P⁺R¹R²R³Z⁻;
CF₃—S—C≡C—(CH₂)₁₃—O-glucoside; CF₃—S—C≡C—(CH₂)₁₃—OH;
CF₃—S—C≡C—(CH₂)₁₃—SH;
CF₃—S—C≡C—(CH₂)₁₄—COOH; CF₃—S—C≡C—(CH₂)₁₄—SO₃H; CF₃—S—C≡C—(CH₂)₁₄—O—SO₃H;
CF₃—S—C≡C—(CH₂)₁₄—O—PO₃H, CF₃—S—C≡C—(CH₂)₁₄—N⁺R¹R²R³Z⁻; CF₃—S—C≡C—(CH₂)₁₄—P⁺R¹R²R³Z⁻;
CF₃—S—C≡C—(CH₂)₁₄—O-glucoside; CF₃—S—C≡C—(CH₂)₁₄—OH;
CF₃—S—C≡C—(CH₂)₁₄—SH;
CF₃—S—C≡C—(CH₂)₁₅—COOH; CF₃—S—C≡C—(CH₂)₁₅—SO₃H; CF₃—S—C≡C—(CH₂)₁₅—O—SO₃H;
CF₃—S—C≡C—(CH₂)₁₅—O—PO₃H, CF₃—S—C≡C—(CH₂)₁₅—N⁺R¹R²R³Z⁻; CF₃—S—C≡C—(CH₂)₁₅—P⁺R¹R²R³Z⁻;
CF₃—S—C≡C—(CH₂)₁₅—O-glucoside; CF₃—S—C≡C—(CH₂)₁₅—OH;
CF₃—S—C≡C—(CH₂)₁₅—SH;
CF₃—S—C≡C—(CH₂)₁₆—COOH; CF₃—S—C≡C—(CH₂)₁₆—SO₃H; CF₃—S—C≡C—(CH₂)₁₆—O—SO₃H;
CF₃—S—C≡C—(CH₂)₁₆—O—PO₃H; CF₃—S—C≡C—(CH₂)₁₆—N⁺R¹R²R³Z⁻; CF₃—S—C≡C—(CH₂)₁₆—P⁺R¹R²R³Z⁻;
CF₃—S—C≡C—(CH₂)₁₆—O-glucoside; CF₃—S—C≡C—(CH₂)₁₆—OH;
CF₃—S—C≡C—(CH₂)₁₆—SH;
(CF₃)₂N—CH₂—C≡C—(CH₂)₄—COOH; (CF₃)₂N—CH₂—C≡C—(CH₂)₄—SO₃H; (CF₃)₂N—CH₂—C≡C—(CH₂)₄—O—SO₃H,
(CF₃)₂N—CH₂—C≡C—(CH₂)₄—O—PO₃H, (CF₃)₂N—CH₂—C≡C—(CH₂)₄—N⁺R¹R²R³Z⁻;
(CF₃)₂N—CH₂—C≡C—(CH₂)₄—P⁺R¹R²R³Z⁻; (CF₃)₂N—CH₂—C≡C—(CH₂)₄—O-
glucoside; (CF₃)₂N—CH₂—C≡C—(CH₂)₄—OH; (CF₃)₂N—CH₂—C≡C—(CH₂)₄—SH;
(CF₃)₂N—CH₂—C≡C—(CH₂)₅—COOH; (CF₃)₂N—CH₂—C≡C—(CH₂)₅—SO₃H; (CF₃)₂N—CH₂—C≡C—(CH₂)₅—O—SO₃H;
(CF₃)₂N—CH₂—C≡C—(CH₂)₅—O—PO₃H; (CF₃)₂N—CH₂—C≡C—(CH₂)₅—N⁺R¹R²R³Z⁻;
(CF₃)₂N—CH₂—C≡C—(CH₂)₅—P⁺R¹R²R³Z⁻; (CF₃)₂N—CH₂—C≡C—(CH₂)₅—O-
glucoside; (CF₃)₂N—CH₂—C≡C—(CH₂)₅—OH; (CF₃)₂N—CH₂—C≡C—(CH₂)₅—SH;
(CF₃)₂N—CH₂—C≡C—(CH₂)₆—COOH; (CF₃)₂N—CH₂—C≡C—(CH₂)₆—SO₃H; (CF₃)₂N—CH₂—C≡C—(CH₂)₆—O—SO₃H;
(CF₃)₂N—CH₂—C≡C—(CH₂)₆—O—PO₃H; (CF₃)₂N—CH₂—C≡C—(CH₂)₆—N⁺R¹R²R³Z⁻;
(CF₃)₂N—CH₂—C≡C—(CH₂)₆—P⁺R¹R²R³Z⁻; (CF₃)₂N—CH₂—C≡C—(CH₂)₆—O-
glucoside; (CF₃)₂N—CH₂—C≡C—(CH₂)₆—OH; (CF₃)₂N—CH₂—C≡C—(CH₂)₆—SH;
(CF₃)₂N—CH₂—C≡C—(CH₂)₇—COOH; (CF₃)₂N—CH₂—C≡C—(CH₂)₇—SO₃H; (CF₃)₂N—CH₂—C≡C—(CH₂)₇—O—SO₃H;
(CF₃)₂N—CH₂—C≡C—(CH₂)₇—O—PO₃H; (CF₃)₂N—CH₂—C≡C—(CH₂)₇—N⁺R¹R²R³Z⁻;
(CF₃)₂N—CH₂—C≡C—(CH₂)₇—P⁺R¹R²R³Z⁻; (CF₃)₂N—CH₂—C≡C—(CH₂)₇—O-
glucoside; (CF₃)₂N—CH₂—C≡C—(CH₂)₇—OH; (CF₃)₂N—CH₂—C≡C—(CH₂)₇—SH;
(CF₃)₂N—CH₂—C≡C—(CH₂)₈—COOH; (CF₃)₂N—CH₂—C≡C—(CH₂)₈—SO₃H; (CF₃)₂N—CH₂—C≡C—(CH₂)₈—O—SO₃H;
(CF₃)₂N—CH₂—C≡C—(CH₂)₈—O—PO₃H; (CF₃)₂N—CH₂—C≡C—(CH₂)₈—N⁺R¹R²R³Z⁻;
(CF₃)₂N—CH₂—C≡C—(CH₂)₈—P⁺R¹R²R³Z⁻; (CF₃)₂N—CH₂—C≡C—(CH₂)₈—O-
glucoside; (CF₃)₂N—CH₂—C≡C—(CH₂)₈—OH; (CF₃)₂N—CH₂—C≡C—(CH₂)₈—SH;
(CF₃)₂N—CH₂—C≡C—(CH₂)₉—COOH; (CF₃)₂N—CH₂—C≡C—(CH₂)₉—SO₃H; (CF₃)₂N—CH₂—C≡C—(CH₂)₉—O—SO₃H;
(CF₃)₂N—CH₂—C≡C—(CH₂)₉—O—PO₃H; (CF₃)₂N—CH₂—C≡C—(CH₂)₉—N⁺R¹R²R³Z⁻;
(CF₃)₂N—CH₂—C≡C—(CH₂)₉—P⁺R¹R²R³Z⁻; (CF₃)₂N—CH₂—C≡C—(CH₂)₉—O-
glucoside; (CF₃)₂N—CH₂—C≡C—(CH₂)₉—OH; (CF₃)₂N—CH₂—C≡C—(CH₂)₉—SH;
(CF₃)₂N—CH₂—C≡C—(CH₂)₁₀—COOH; (CF₃)₂N—CH₂—C≡C—(CH₂)₁₀—SO₃H; (CF₃)₂N—CH₂—C≡C—(CH₂)₁₀—O—SO₃H;
(CF₃)₂N—CH₂—C≡C—(CH₂)₁₀—O—PO₃H; (CF₃)₂N—CH₂—C≡C—(CH₂)₁₀—N⁺R¹R²R³Z⁻;
(CF₃)₂N—CH₂—C≡C—(CH₂)₁₀—P⁺R¹R²R³Z⁻; (CF₃)₂N—CH₂—C≡C—(CH₂)₁₀—O-
glucoside; (CF₃)₂N—CH₂—C≡C—(CH₂)₁₀—OH; (CF₃)₂N—CH₂—C≡C—(CH₂)₁₀—SH;
(CF₃)₂N—CH₂—C≡C—(CH₂)₁₁—COOH; (CF₃)₂N—CH₂—C≡C—(CH₂)₁₁—SO₃H; (CF₃)₂N—CH₂—C≡C—(CH₂)₁₁—O—SO₃H;
(CF₃)₂N—CH₂—C≡C—(CH₂)₁₁—O—PO₃H; (CF₃)₂N—CH₂—C≡C—(CH₂)₁₁—N⁺R¹R²R³Z⁻;
(CF₃)₂N—CH₂—C≡C—(CH₂)₁₁—P⁺R¹R²R³Z⁻; (CF₃)₂N—CH₂—C≡C—(CH₂)₁₁—O-
glucoside; (CF₃)₂N—CH₂—C≡C—(CH₂)₁₁—OH; (CF₃)₂N—CH₂—C≡C—(CH₂)₁₁—SH;
(CF₃)₂N—CH₂—C≡C—(CH₂)₁₂—COOH; (CF₃)₂N—CH₂—C≡C—(CH₂)₁₂—SO₃H; (CF₃)₂N—CH₂—C≡C—(CH₂)₁₂—O—SO₃H;
(CF₃)₂N—CH₂—C≡C—(CH₂)₁₂—O—PO₃H, (CF₃)₂N—CH₂—C≡C—(CH₂)₁₂—N⁺R¹R²R³Z⁻;
(CF₃)₂N—CH₂—C≡C—(CH₂)₁₂—P⁺R¹R²R³Z⁻; (CF₃)₂N—CH₂—C≡C—(CH₂)₁₂—O-
glucoside; (CF₃)₂N—CH₂—C≡C—(CH₂)₁₂—OH (CF₃)₂N—CH₂—C≡C—(CH₂)₁₂—SH
(CF₃)₂N—CH₂—C≡C—(CH₂)₁₃—COOH; (CF₃)₂N—CH₂—C≡C—(CH₂)₁₃—SO₃H; (CF₃)₂N—CH₂—C≡C—(CH₂)₁₃—O—SO₃H;
(CF₃)₂N—CH₂—C≡C—(CH₂)₁₃—O—PO₃H; (CF₃)₂N—CH₂—C≡C—(CH₂)₁₃—N⁺R¹R²R³Z⁻;
(CF₃)₂N—CH₂—C≡C—(CH₂)₁₃—P⁺R¹R²R³Z⁻; (CF₃)₂N—CH₂—C≡C—(CH₂)₁₃—O-
glucoside; (CF₃)₂N—CH₂—C≡C—(CH₂)₁₃—OH; (CF₃)₂N—CH₂—C≡C—(CH₂)₁₃—SH;
(CF₃)₂N—CH₂—C≡C—(CH₂)₁₄—COOH; (CF₃)₂N—CH₂—C≡C—(CH₂)₁₄—SO₃H; (CF₃)₂N—CH₂—C≡C—(CH₂)₁₄—O—SO₃H;
(CF₃)₂N—CH₂—C≡C—(CH₂)₁₄—O—PO₃H, (CF₃)₂N—CH₂—C≡C—(CH₂)₁₄—N⁺R¹R²R³Z⁻;
(CF₃)₂N—CH₂—C≡C—(CH₂)₁₄—P⁺R¹R²R³Z⁻; (CF₃)₂N—CH₂—C≡C—(CH₂)₁₄—O-
glucoside; (CF₃)₂N—CH₂—C≡C—(CH₂)₁₄—OH; (CF₃)₂N—CH₂—C≡C—(CH₂)₁₄—SH;
(CF₃)₂N—CH₂—C≡C—(CH₂)₁₅—COOH; (CF₃)₂N—CH₂—C≡C—(CH₂)₁₅—SO₃H; (CF₃)₂N—CH₂—C≡C—(CH₂)₁₅—O—SO₃H;
(CF₃)₂N—CH₂—C≡C—(CH₂)₁₅—O—PO₃H, (CF₃)₂N—CH₂—C≡C—(CH₂)₁₅—N⁺R¹R²R³Z⁻;
(CF₃)₂N—CH₂—C≡C—(CH₂)₁₅—P⁺R¹R²R³Z⁻; (CF₃)₂N—CH₂—C≡C—(CH₂)₁₅—O-
glucoside; (CF₃)₂N—CH₂—C≡C—(CH₂)₁₅—OH; (CF₃)₂N—CH₂—C≡C—(CH₂)₁₅—SH;
(CF₃)₂N—CH₂—C≡C—(CH₂)₁₆—COOH; (CF₃)₂N—CH₂—C≡C—(CH₂)₁₆—SO₃H; (CF₃)₂N—CH₂—C≡C—(CH₂)₁₆—O—SO₃H;
(CF₃)₂N—CH₂—C≡C—(CH₂)₁₆—O—PO₃H; (CF₃)₂N—CH₂—C≡C—(CH₂)₁₆—N⁺R¹R²R³Z⁻;
(CF₃)₂N—CH₂—C≡C—(CH₂)₁₆—P⁺R¹R²R³Z⁻; (CF₃)₂N—CH₂—C≡C—(CH₂)₁₆—O-
glucoside; (CF₃)₂N—CH₂—C≡C—(CH₂)₁₆—OH; (CF₃)₂N—CH₂—C≡C—(CH₂)₁₆—SH;
(CF₃)₂N—(CH₂)₄—COOH; (CF₃)₂N—(CH₂)₄—SO₃H; (CF₃)₂N—(CH₂)₄—O—SO₃H
(CF₃)₂N—(CH₂)₅—COOH; (CF₃)₂N—(CH₂)₅—SO₃H; (CF₃)₂N—(CH₂)₅—O—SO₃H
(CF₃)₂N—(CH₂)₆—COOH; (CF₃)₂N—(CH₂)₆—SO₃H; (CF₃)₂N—(CH₂)₆—O—SO₃H
(CF₃)₂N—(CH₂)₇—COOH; (CF₃)₂N—(CH₂)₇—SO₃H; (CF₃)₂N—(CH₂)₇—O—SO₃H
(CF₃)₂N—(CH₂)₈—COOH; (CF₃)₂N—(CH₂)₈—SO₃H; (CF₃)₂N—(CH₂)₈—O—SO₃H
(CF₃)₂N—(CH₂)₉—COOH; (CF₃)₂N—(CH₂)₉—SO₃H; (CF₃)₂N—(CH₂)₉—O—SO₃H
(CF₃)₂N—(CH₂)₁₀—COOH; (CF₃)₂N—(CH₂)₁₀—SO₃H; (CF₃)₂N—(CH₂)₁₀—O—SO₃H
(CF₃)₂N—(CH₂)₁₁—COOH; (CF₃)₂N—(CH₂)₁₁—SO₃H; (CF₃)₂N—(CH₂)₁₁—O—SO₃H
(CF₃)₂N—(CH₂)₁₂—COOH; (CF₃)₂N—(CH₂)₁₂—SO₃H; (CF₃)₂N—(CH₂)₁₂—O—SO₃H
(CF₃)₂N—(CH₂)₁₃—COOH; (CF₃)₂N—(CH₂)₁₃—SO₃H; (CF₃)₂N—(CH₂)₁₃—O—SO₃H
(CF₃)₂N—(CH₂)₁₄—COOH; (CF₃)₂N—(CH₂)₁₄—SO₃H; (CF₃)₂N—(CH₂)₁₄—O—SO₃H
(CF₃)₂N—(CH₂)₁₅—COOH; (CF₃)₂N—(CH₂)₁₅—SO₃H; (CF₃)₂N—(CH₂)₁₅—O—SO₃H
(CF₃)₂N—(CH₂)₁₆—COOH; (CF₃)₂N—(CH₂)₁₆—SO₃H; (CF₃)₂N—(CH₂)₁₆—O—SO₃H
CF₃—CH₂—CH₂S—CH₂—CH═CH—(CH₂)₄—COOH; CF₃—CH₂—CH₂S—CH₂—CH═CH—(CH₂)₄—SO₃H;
CF₃—CH₂—CH₂S—CH₂—CH═CH—(CH₂)₄—O—SO₃H; CF₃—CH₂—CH₂S—CH₂—CH═CH—(CH₂)₄—OPO₃H;
CF₃—CH₂—CH₂S—CH₂—CH═CH—(CH₂)₅—COOH; CF₃—CH₂—CH₂S—CH₂—CH═CH—(CH₂)₅—SO₃H;
CF₃—CH₂—CH₂S—CH₂—CH═CH—(CH₂)₅—O—SO₃H; CF₃—CH₂—CH₂S—CH₂—CH═CH—(CH₂)₅—OPO₃H
CF₃—CH₂—CH₂S—CH₂—CH═CH—(CH₂)₆—COOH; CF₃—CH₂—CH₂S—CH₂—CH═CH—(CH₂)₆—SO₃H;
CF₃—CH₂—CH₂S—CH₂—CH═CH—(CH₂)₆—O—SO₃H; CF₃—CH₂—CH₂S—CH₂—CH═CH—(CH₂)₆—OPO₃
CF₃—CH₂—CH₂S—CH₂—CH═CH—(CH₂)₇—COOH; CF₃—CH₂—CH₂S—CH₂—CH═CH—(CH₂)₇—SO₃H;
CF₃—CH₂—CH₂S—CH₂—CH═CH—(CH₂)₇—O—SO₃H; CF₃—CH₂—CH₂S—CH₂—CH═CH—(CH₂)₇—OPO₃H
CF₃—CH₂—CH₂S—CH₂—CH═CH—(CH₂)₈—COOH; CF₃—CH₂—CH₂S—CH₂—CH═CH—(CH₂)₈—SO₃H;
CF₃—CH₂—CH₂S—CH₂—CH═CH—(CH₂)₈—O—SO₃H; CF₃—CH₂—CH₂S—CH₂—CH═CH—(CH₂)₈—OPO₃H
CF₃—CH₂—CH₂S—CH₂—CH═CH—(CH₂)₉—COOH; CF₃—CH₂—CH₂S—CH₂—CH═CH—(CH₂)₉—SO₃H;
CF₃—CH₂—CH₂S—CH₂—CH═CH—(CH₂)₉—O—SO₃H; CF₃—CH₂—CH₂S—CH₂—CH═CH—(CH₂)₉—OPO₃H
CF₃—CH₂—CH₂S—CH₂—CH═CH—(CH₂)₁₀—COOH; CF₃—CH₂—CH₂S—CH₂—CH═CH—(CH₂)₁₀—SO₃H;
CF₃—CH₂—CH₂S—CH₂—CH═CH—(CH₂)₁₀—O—SO₃H; CF₃—CH₂—CH₂S—CH₂—CH═CH—(CH₂)₁₀—OPO₃H;
CF₃—CH₂—CH₂S—CH₂—CH═CH—(CH₂)₁₁—COOH; CF₃—CH₂—CH₂S—CH₂—CH═CH—(CH₂)₁₁—SO₃H;
CF₃—CH₂—CH₂S—CH₂—CH═CH—(CH₂)₁₁—O—SO₃H; CF₃—CH₂—CH₂S—CH₂—CH═CH—(CH₂)₁₁—OPO₃H
CF₃—CH₂—CH₂S—CH₂—CH═CH—(CH₂)₁₂—COOH; CF₃—CH₂—CH₂S—CH₂—CH═CH—(CH₂)₁₂—SO₃H;
CF₃—CH₂—CH₂S—CH₂—CH═CH—(CH₂)₁₂—O—SO₃H; CF₃—CH₂—CH₂S—CH₂—CH═CH—(CH₂)₁₂—OPO₃H
CF₃—CH₂—CH₂S—CH₂—CH═CH—(CH₂)₁₃—COOH; CF₃—CH₂—CH₂S—CH₂—CH═CH—(CH₂)₁₃—SO₃H;
CF₃—CH₂—CH₂S—CH₂—CH═CH—(CH₂)₁₃—O—SO₃H; CF₃—CH₂—CH₂S—CH₂—CH═CH—(CH₂)₁₃—OPO₃H
CF₃—CH₂—CH₂S—CH₂—CH═CH—(CH₂)₁₄—COOH; CF₃—CH₂—CH₂S—CH₂—CH═CH—(CH₂)₁₄—SO₃H;
CF₃—CH₂—CH₂S—CH₂—CH═CH—(CH₂)₁₄—O—SO₃H; CF₃—CH₂—CH₂S—CH₂—CH═CH—(CH₂)₁₄—OPO₃H
CF₃—CH₂—CH₂S—CH₂—CH═CH—(CH₂)₁₅—COOH; CF₃—CH₂—CH₂S—CH₂—CH═CH—(CH₂)₁₅—SO₃H;
CF₃—CH₂—CH₂S—CH₂—CH═CH—(CH₂)₁₅—O—SO₃H; CF₃—CH₂—CH₂S—CH₂—CH═CH—(CH₂)₁₅—OPO₃H
CF₃—CH₂—CH₂S—CH₂—CH═CH—(CH₂)₁₆—COOH; CF₃—CH₂—CH₂S—CH₂—CH═CH—(CH₂)₁₆—SO₃H;
CF₃—CH₂—CH₂S—CH₂—CH═CH—(CH₂)₁₆—O—SO₃H; CF₃—CH₂—CH₂S—CH₂—CH═CH—(CH₂)₁₆—OPO₃H
(CF₃—CH₂—CH₂)₂N—CH₂—CH═CH—(CH₂)₄—COOH; (CF₃—CH₂—CH₂)₂N—CH₂—CH═CH—(CH₂)₄—SO₃H;
(CF₃—CH₂—CH₂)₂N—CH₂—CH═CH—(CH₂)₄—O—SO₃H; (CF₃—CH₂—CH₂)₂N—CH₂—CH═CH—(CH₂)₄—OPO₃H;
(CF₃—CH₂—CH₂)₂N—CH₂—CH═CH—(CH₂)₅—COOH; (CF₃—CH₂—CH₂)₂N—CH₂—CH═CH—(CH₂)₅—SO₃H;
(CF₃—CH₂—CH₂)₂N—CH₂—CH═CH—(CH₂)₅—O—SO₃H; (CF₃—CH₂—CH₂)₂N—CH₂—CH═CH—(CH₂)₅—OPO₃H
(CF₃—CH₂—CH₂)₂N—CH₂—CH═CH—(CH₂)₆—COOH; (CF₃—CH₂—CH₂)₂N—CH₂—CH═CH—(CH₂)₆—SO₃H;
(CF₃—CH₂—CH₂)₂N—CH₂—CH═CH—(CH₂)₆—O—SO₃H; (CF₃—CH₂—CH₂)₂N—CH₂—CH═CH—(CH₂)₆—OPO₃
(CF₃—CH₂—CH₂)₂N—CH₂—CH═CH—(CH₂)₇—COOH; (CF₃—CH₂—CH₂)₂N—CH₂—CH═CH—(CH₂)₇—SO₃H;
(CF₃—CH₂—CH₂)₂N—CH₂—CH═CH—(CH₂)₇—O—SO₃H; (CF₃—CH₂—CH₂)₂N—CH₂—CH═CH—(CH₂)₇—OPO₃H
(CF₃—CH₂—CH₂)₂N—CH₂—CH═CH—(CH₂)₈—COOH; (CF₃—CH₂—CH₂)₂N—CH₂—CH═CH—(CH₂)₈—SO₃H;
(CF₃—CH₂—CH₂)₂NCH₂—CH═CH—(CH₂)₈—O—SO₃H; (CF₃—CH₂—CH₂)₂N—CH₂—CH═CH—(CH₂)₈—OPO₃H
(CF₃—CH₂—CH₂)₂N—CH₂—CH═CH—(CH₂)₉—COOH; (CF₃—CH₂—CH₂)₂N—CH₂—CH═CH—(CH₂)₉—SO₃H;
(CF₃—CH₂—CH₂)₂N—CH₂—CH═CH—(CH₂)₉—O—SO₃H; (CF₃—CH₂—CH₂)₂N—CH₂—CH═CH—(CH₂)₉—OPO₃H
(CF₃—CH₂—CH₂)₂N—CH₂—CH═CH—(CH₂)₁₀—COOH; (CF₃—CH₂—CH₂)₂N—CH₂—CH═CH—(CH₂)₁₀—SO₃H;
(CF₃—CH₂—CH₂)₂N—CH₂—CH═CH—(CH₂)₁₀—O—SO₃H; (CF₃—CH₂—CH₂)₂N—CH₂—CH═CH—(CH₂)₁₀—OPO₃H;
(CF₃—CH₂—CH₂)₂N—CH₂—CH═CH—(CH₂)₁₁—COOH; (CF₃—CH₂—CH₂)₂N—CH₂—CH═CH—(CH₂)₁₁—SO₃H;
(CF₃—CH₂—CH₂)₂N—CH₂—CH═CH—(CH₂)₁₁—O—SO₃H; (CF₃—CH₂—CH₂)₂N—CH₂—CH═CH—(CH₂)₁₁—OPO₃H
(CF₃—CH₂—CH₂)₂N—CH₂—CH═CH—(CH₂)₁₂—COOH; (CF₃—CH₂—CH₂)₂N—CH₂—CH═CH—(CH₂)₁₂—SO₃H;
(CF₃—CH₂—CH₂)₂N—CH₂—CH═CH—(CH₂)₁₂—O—SO₃H; (CF₃—CH₂—CH₂)₂N—CH₂—CH═CH—(CH₂)₁₂—OPO₃H
(CF₃—CH₂—CH₂)₂N—CH₂—CH═CH—(CH₂)₁₃—COOH; (CF₃—CH₂—CH₂)₂NCH₂—CH═CH—(CH₂)₁₃—SO₃H;
(CF₃—CH₂—CH₂)₂N—CH₂—CH═CH—(CH₂)₁₃—O—SO₃H; (CF₃—CH₂—CH₂)₂N—CH₂—CH═CH—(CH₂)₁₃—OPO₃H
(CF₃—CH₂—CH₂)₂N—CH₂—CH═CH—(CH₂)₁₄—COOH; (CF₃—CH₂—CH₂)₂N—CH₂—CH═CH—(CH₂)₁₄—SO₃H;
(CF₃—CH₂—CH₂)₂N—CH₂—CH═CH—(CH₂)₁₄—O—SO₃H; (CF₃—CH₂—CH₂)₂N—CH₂—CH═CH—(CH₂)₁₄—OPO₃H
(CF₃—CH₂—CH₂)₂N—CH₂—CH═CH—(CH₂)₁₅—COOH; (CF₃—CH₂—CH₂)₂N—CH₂—CH═CH—(CH₂)₁₅—SO₃H;
(CF₃—CH₂—CH₂)₂N—CH₂—CH═CH—(CH₂)₁₅—O—SO₃H; (CF₃—CH₂—CH₂)₂NCH₂—CH═CH—(CH₂)₁₅—OPO₃H
(CF₃—CH₂—CH₂)₂NCH₂—CH═CH—(CH₂)₁₆—COOH; (CF₃—CH₂—CH₂)₂N—CH₂—CH═CH—(CH₂)₁₆—SO₃H;
(CF₃—CH₂—CH₂)₂N—CH₂—CH═CH—(CH₂)₁₆—O—SO₃H; (CF₃—CH₂—CH₂)₂N—CH₂—CH═CH—(CH₂)₁₆—OPO₃H
(CF₃)₂N—CH₂—CH═CH—(CH₂)₄—COOH; (CF₃)₂N—CH₂—CH═CH—(CH₂)₄—SO₃H; (CF₃)₂N—CH₂—CH═CH—(CH₂)₄—O—SO₃H;
(CF₃)₂N—CH₂—CH═CH—(CH₂)₄—SH;
(CF₃)₂N—CH₂—CH═CH—(CH₂)₅—COOH; (CF₃)₂N—CH₂—CH═CH—(CH₂)₅—SO₃H; (CF₃)₂N—CH₂—CH═CH—(CH₂)₅—O—SO₃H;
(CF₃)₂N—CH₂—CH═CH—(CH₂)₅—SH
(CF₃)₂N—CH₂—CH═CH—(CH₂)₆—COOH; (CF₃)₂N—CH₂—CH═CH—(CH₂)₆—SO₃H; (CF₃)₂N—CH₂—CH═CH—(CH₂)₆—O—SO₃H;
(CF₃)₂N—CH₂—CH═CH—(CH₂)₆—SH
(CF₃)₂N—CH₂—CH═CH—(CH₂)₇—COOH; (CF₃)₂N—CH₂—CH═CH—(CH₂)₇—SO₃H; (CF₃)₂N—CH₂—CH═CH—(CH₂)₇—O—SO₃H;
(CF₃)₂N—CH₂—CH═CH—(CH₂)₇—SH
(CF₃)₂N—CH₂—CH═CH—(CH₂)₈—COOH; (CF₃)₂N—CH₂—CH═CH—(CH₂)₈—SO₃H; (CF₃)₂N—CH₂—CH═CH—(CH₂)₈—O—SO₃H;
(CF₃)₂N—CH₂—CH═CH—(CH₂)₈—SH
(CF₃)₂N—CH₂—CH═CH—(CH₂)₉—COOH; (CF₃)₂N—CH₂—CH═CH—(CH₂)₉—SO₃H; (CF₃)₂N—CH₂—CH═CH—(CH₂)₉—O—SO₃H;
(CF₃)₂N—CH₂—CH═CH—(CH₂)₉—SH
(CF₃)₂N—CH₂—CH═CH—(CH₂)₁₀—COOH; (CF₃)₂N—CH₂—CH═CH—(CH₂)₁₀—SO₃H;
(CF₃)₂N—CH₂—CH═CH—(CH₂)₁₀—O—SO₃H; (CF₃)₂N—CH₂—CH═CH—(CH₂)₁₀—SH;
(CF₃)₂N—CH₂—CH═CH—(CH₂)₁₁—COOH; (CF₃)₂N—CH₂—CH═CH—(CH₂)₁₁—SO₃H;
(CF₃)₂N—CH₂—CH═CH—(CH₂)₁₁—O—SO₃H; (CF₃)₂N—CH₂—CH═CH—(CH₂)₁₁—SH
(CF₃)₂N—CH₂—CH═CH—(CH₂)₁₂—COOH; (CF₃)₂N—CH₂—CH═CH—(CH₂)₁₂—SO₃H;
(CF₃)₂N—CH₂—CH═CH—(CH₂)₁₂—O—SO₃H; (CF₃)₂N—CH₂—CH═CH—(CH₂)₁₂—SH
(CF₃)₂N—CH₂—CH═CH—(CH₂)₁₃—COOH; (CF₃)₂N—CH₂—CH═CH—(CH₂)₁₃—SO₃H;
(CF₃)₂N—CH₂—CH═CH—(CH₂)₁₃—O—SO₃H; (CF₃)₂N—CH₂—CH═CH—(CH₂)₁₃—SH
(CF₃)₂N—CH₂—CH═CH—(CH₂)₁₄—COOH; (CF₃)₂N—CH₂—CH═CH—(CH₂)₁₄—SO₃H;
(CF₃)₂N—CH₂—CH═CH—(CH₂)₁₄—O—SO₃H; (CF₃)₂N—CH₂—CH═CH—(CH₂)₁₄—SH
(CF₃)₂N—CH₂—CH═CH—(CH₂)₁₅—COOH; (CF₃)₂N—CH₂—CH═CH—(CH₂)₁₅—SO₃H;
(CF₃)₂N—CH₂—CH═CH—(CH₂)₁₅—O—SO₃H; (CF₃)₂N—CH₂—CH═CH—(CH₂)₁₅—SH
(CF₃)₂N—CH₂—CH═CH—(CH₂)₁₆—COOH; (CF₃)₂N—CH₂—CH═CH—(CH₂)₁₆—SO₃H;
(CF₃)₂N—CH₂—CH═CH—(CH₂)₁₆—O—SO₃H; (CF₃)₂N—CH₂—CH═CH—(CH₂)₁₆—SH
CF₃S—CH₂—CH═CH—(CH₂)₄—COOH; CF₃S—CH₂—CH═CH—(CH₂)₄—SO₃H; CF₃S—CH₂—CH═CH—(CH₂)₄—O—SO₃H;
CF₃S—CH₂—CH═CH—(CH₂)₄—SH;
CF₃S—CH₂—CH═CH—(CH₂)₅—COOH; CF₃S—CH₂—CH═CH—(CH₂)₅—SO₃H; CF₃S—CH₂—CH═CH—(CH₂)₅—O—SO₃H;
CF₃S—CH₂—CH═CH—(CH₂)₅—SH
CF₃S—CH₂—CH═CH—(CH₂)₆—COOH; CF₃S—CH₂—CH═CH—(CH₂)₆—SO₃H; CF₃S—CH₂—CH═CH—(CH₂)₆—O—SO₃H;
CF₃S—CH₂—CH═CH—(CH₂)₆—SH
CF₃S—CH₂—CH═CH—(CH₂)₇—COOH; CF₃S—CH₂—CH═CH—(CH₂)₇—SO₃H; CF₃S—CH₂—CH═CH—(CH₂)₇—O—SO₃H;
CF₃S—CH₂—CH═CH—(CH₂)₇—SH
CF₃S—CH₂—CH═CH—(CH₂)₈—COOH; CF₃S—CH₂—CH═CH—(CH₂)₈—SO₃H; CF₃S—CH₂—CH═CH—(CH₂)₈—O—SO₃H;
CF₃S—CH₂—CH═CH—(CH₂)₈—SH
CF₃S—CH₂—CH═CH—(CH₂)₉—COOH; CF₃S—CH₂—CH═CH—(CH₂)₉—SO₃H; CF₃S—CH₂—CH═CH—(CH₂)₉—O—SO₃H;
CF₃S—CH₂—CH═CH—(CH₂)₉—SH
CF₃S—CH₂—CH═CH—(CH₂)₁₀—COOH; CF₃S—CH₂—CH═CH—(CH₂)₁₀—SO₃H; CF₃S—CH₂—CH═CH—(CH₂)₁₀—O—SO₃H;
CF₃S—CH₂—CH═CH—(CH₂)₁₀—SH;
CF₃S—CH₂—CH═CH—(CH₂)₁₁—COOH; CF₃S—CH₂—CH═CH—(CH₂)₁₁—SO₃H; CF₃S—CH₂—CH═CH—(CH₂)₁₁—O—SO₃H;
CF₃S—CH₂—CH═CH—(CH₂)₁₁—SH
CF₃S—CH₂—CH═CH—(CH₂)₁₂—COOH; CF₃S—CH₂—CH═CH—(CH₂)₁₂—SO₃H; CF₃S—CH₂—CH═CH—(CH₂)₁₂—O—SO₃H;
CF₃S—CH₂—CH═CH—(CH₂)₁₂—SH
CF₃S—CH₂—CH═CH—(CH₂)₁₃—COOH; CF₃S—CH₂—CH═CH—(CH₂)₁₃—SO₃H; CF₃S—CH₂—CH═CH—(CH₂)₁₃—O—SO₃H;
CF₃S—CH₂—CH═CH—(CH₂)₁₃—SH
CF₃S—CH₂—CH═CH—(CH₂)₁₄—COOH; CF₃S—CH₂—CH═CH—(CH₂)₁₄—SO₃H; CF₃S—CH₂—CH═CH—(CH₂)₁₄—O—SO₃H;
CF₃S—CH₂—CH═CH—(CH₂)₁₄—SH
CF₃S—CH₂—CH═CH—(CH₂)₁₅—COOH; CF₃S—CH₂—CH═CH—(CH₂)₁₅—SO₃H; CF₃S—CH₂—CH═CH—(CH₂)₁₅—O—SO₃H;
CF₃S—CH₂—CH═CH—(CH₂)₁₅—SH
CF₃S—CH₂—CH═CH—(CH₂)₁₆—COOH; CF₃S—CH₂—CH═CH—(CH₂)₁₆—SO₃H; CF₃S—CH₂—CH═CH—(CH₂)₁₆—O—SO₃H;
CF₃S—CH₂—CH═CH—(CH₂)₁₆—SH
CF₃S—CH₂—CH═CH—(CH₂)₄—OPO₃H; CF₃S—CH₂—CH═CH—(CH₂)₄—N⁺R¹R²R³Z⁻; CF₃S—CH₂—CH═CH—(CH₂)₄—P⁺R¹R²R³Z⁻;
CF₃S—CH₂—CH═CH—(CH₂)₄—O-glucoside; CF₃S—CH₂—CH═CH—(CH₂)₄—OH
CF₃S—CH₂—CH═CH—(CH₂)₅—OPO₃H; CF₃S—CH₂—CH═CH—(CH₂)₅—N⁺R¹R²R³Z⁻; CF₃S—CH₂—CH═CH—(CH₂)₅—P⁺R¹R²R³Z⁻;
CF₃S—CH₂—CH═CH—(CH₂)₅—O-glucoside; CF₃S—CH₂—CH═CH—(CH₂)₅—OH
CF₃S—CH₂—CH═CH—(CH₂)₆—OPO₃H; CF₃S—CH₂—CH═CH—(CH₂)₆—N⁺R¹R²R³Z⁻; CF₃S—CH₂—CH═CH—(CH₂)₆—P⁺R¹R²R³Z⁻;
CF₃S—CH₂—CH═CH—(CH₂)₆—O-glucoside; CF₃S—CH₂—CH═CH—(CH₂)₆—OH
CF₃S—CH₂—CH═CH—(CH₂)₇—OPO₃H; CF₃S—CH₂—CH═CH—(CH₂)₇—N⁺R¹R²R³Z⁻; CF₃S—CH₂—CH═CH—(CH₂)₇—P⁺R¹R²R³Z⁻;
CF₃S—CH₂—CH═CH—(CH₂)₇—O-glucoside; CF₃S—CH₂—CH═CH—(CH₂)₇—OH
CF₃S—CH₂—CH═CH—(CH₂)₈—OPO₃H; CF₃S—CH₂—CH═CH—(CH₂)₈N⁺R¹R²R³Z⁻; CF₃S—CH₂—CH═CH—(CH₂)₈—P⁺R¹R²R³Z⁻;
CF₃S—CH₂—CH═CH—(CH₂)₈O-glucoside; CF₃S—CH₂—CH═CH—(CH₂)₈—OH
CF₃S—CH₂—CH═CH—(CH₂)₉—OPO₃H; CF₃S—CH₂—CH═CH—(CH₂)₉N⁺R¹R²R³Z⁻; CF₃S—CH₂—CH═CH—(CH₂)₉—P⁺R¹R²R³Z⁻;
CF₃S—CH₂—CH═CH—(CH₂)₉O-glucoside; CF₃S—CH₂—CH═CH—(CH₂)₉—OH
CF₃S—CH₂—CH═CH—(CH₂)₁₀—OPO₃H; CF₃S—CH₂—CH═CH—(CH₂)₁₀⁺R¹R²R³Z⁻; CF₃S—CH₂—CH═CH—(CH₂)₁₀—P⁺R¹R²R³Z⁻;
CF₃S—CH₂—CH═CH—(CH₂)₁₀-glucoside; CF₃S—CH₂—CH═CH—(CH₂)₁₀—OH
CF₃S—CH₂—CH═CH—(CH₂)₁₁—OPO₃H; CF₃S—CH₂—CH═CH—(CH₂)₁₁⁺R¹R²R³Z⁻; CF₃S—CH₂—CH═CH—(CH₂)₁₁—P⁺R¹R²R³Z⁻;
CF₃S—CH₂—CH═CH—(CH₂)₁₁-glucoside; CF₃S—CH₂—CH═CH—(CH₂)₁₁—OH
CF₃S—CH₂—CH═CH—(CH₂)₁₂—OPO₃H; CF₃S—CH₂—CH═CH—(CH₂)₁₂⁺R¹R²R³Z⁻; CF₃S—CH₂—CH═CH—(CH₂)₁₂—P⁺R¹R²R³Z⁻;
CF₃S—CH₂—CH═CH—(CH₂)₁₂-glucoside; CF₃S—CH₂—CH═CH—(CH₂)₁₂—OH
CF₃S—CH₂—CH═CH—(CH₂)₁₃—OPO₃H; CF₃S—CH₂—CH═CH—(CH₂)₁₃⁺R¹R²R³Z⁻; CF₃S—CH₂—CH═CH—(CH₂)₁₃—P⁺R¹R²R³Z⁻;
CF₃S—CH₂—CH═CH—(CH₂)₁₃-glucoside; CF₃S—CH₂—CH═CH—(CH₂)₁₃—OH
CF₃S—CH₂—CH═CH—(CH₂)₁₄—OPO₃H; CF₃S—CH₂—CH═CH—(CH₂)₁₄⁺R¹R²R³Z⁻; CF₃S—CH₂—CH═CH—(CH₂)₁₄—P⁺R¹R²R³Z⁻;
CF₃S—CH₂—CH═CH—(CH₂)₁₄-glucoside; CF₃S—CH₂—CH═CH—(CH₂)₁₄—OH
CF₃S—CH₂—CH═CH—(CH₂)₁₅—OPO₃H; CF₃S—CH₂—CH═CH—(CH₂)₁₅⁺R¹R²R³Z⁻; CF₃S—CH₂—CH═CH—(CH₂)₁₅—P⁺R¹R²R³Z⁻;
CF₃S—CH₂—CH═CH—(CH₂)₁₅-glucoside; CF₃S—CH₂—CH═CH—(CH₂)₁₅—OH
CF₃S—CH₂—CH═CH—(CH₂)₁₆—OPO₃H; CF₃S—CH₂—CH═CH—(CH₂)₁₆⁺R¹R²R³Z⁻; CF₃S—CH₂—CH═CH—(CH₂)₁₆—P⁺R¹R²R³Z⁻;
CF₃S—CH₂—CH═CH—(CH₂)₁₆-glucoside; CF₃S—CH₂—CH═CH—(CH₂)₁₆—OH
(CF₃)₂N—CH═CH—(CH₂)₄—COOH; (CF₃)₂N—CH═CH—(CH₂)₄—SO₃H; (CF₃)₂N—CH═CH—(CH₂)₄—O—SO₃H;
(CF₃)₂N—CH═CH—(CH₂)₄—O—PO₃H; (CF₃)₂N—CH═CH—(CH₂)₄—OH
(CF₃)₂N—CH═CH—(CH₂)₅—COOH; (CF₃)₂N—CH═CH—(CH₂)₅—SO₃H; (CF₃)₂N—CH═CH—(CH₂)₅—O—SO₃H;
(CF₃)₂N—CH═CH—(CH₂)₅—O—PO₃H; (CF₃)₂N—CH═CH—(CH₂)₅—OH
(CF₃)₂N—CH═CH—(CH₂)₆—COOH; (CF₃)₂N—CH═CH—(CH₂)₆—SO₃H; (CF₃)₂N—CH═CH—(CH₂)₆—O—SO₃H;
(CF₃)₂N—CH═CH—(CH₂)₆—O—PO₃H; (CF₃)₂N—CH═CH—(CH₂)₆—OH
(CF₃)₂N—CH═CH—(CH₂)₇—COOH; (CF₃)₂N—CH═CH—(CH₂)₇—SO₃H; (CF₃)₂N—CH═CH—(CH₂)₇—O—SO₃H;
(CF₃)₂N—CH═CH—(CH₂)₇—O—PO₃H; (CF₃)₂N—CH═CH—(CH₂)₇—OH
(CF₃)₂N—CH═CH—(CH₂)₈—COOH; (CF₃)₂N—CH═CH—(CH₂)₈—SO₃H; (CF₃)₂N—CH═CH—(CH₂)₈—O—SO₃H;
(CF₃)₂N—CH═CH—(CH₂)₈—O—PO₃H; (CF₃)₂N—CH═CH—(CH₂)₈—OH
(CF₃)₂N—CH═CH—(CH₂)₉—COOH; (CF₃)₂N—CH═CH—(CH₂)₉—SO₃H; (CF₃)₂N—CH═CH—(CH₂)₉—O—SO₃H;
(CF₃)₂N—CH═CH—(CH₂)₉—O—PO₃H; (CF₃)₂N—CH═CH—(CH₂)₉—OH
(CF₃)₂N—CH═CH—(CH₂)₁₀—COOH; (CF₃)₂N—CH═CH—(CH₂)₁₀—SO₃H; (CF₃)₂N—CH═CH—(CH₂)₁₀—O—SO₃H;
(CF₃)₂N—CH═CH—(CH₂)₁₀—O—PO₃H; (CF₃)₂N—CH═CH—(CH₂)₁₀—OH
(CF₃)₂N—CH═CH—(CH₂)₁₁—COOH; (CF₃)₂N—CH═CH—(CH₂)₁₁—SO₃H; (CF₃)₂N—CH═CH—(CH₂)₁₁—O—SO₃H;
(CF₃)₂N—CH═CH—(CH₂)₁₁—O—PO₃H; (CF₃)₂N—CH═CH—(CH₂)₁₁—OH
(CF₃)₂N—CH═CH—(CH₂)₁₂—COOH; (CF₃)₂N—CH═CH—(CH₂)₁₂—SO₃H; (CF₃)₂N—CH═CH—(CH₂)₁₂—O—SO₃H;
(CF₃)₂N—CH═CH—(CH₂)₁₂—O—PO₃H; (CF₃)₂N—CH═CH—(CH₂)₁₂—OH
(CF₃)₂N—CH═CH—(CH₂)₁₃—COOH; (CF₃)₂N—CH═CH—(CH₂)₁₃—SO₃H; (CF₃)₂N—CH═CH—(CH₂)₁₃—O—SO₃H;
(CF₃)₂N—CH═CH—(CH₂)₁₃—O—PO₃H; (CF₃)₂N—CH═CH—(CH₂)₁₃—OH
(CF₃)₂N—CH═CH—(CH₂)₁₄—COOH; (CF₃)₂N—CH═CH—(CH₂)₁₄—SO₃H; (CF₃)₂N—CH═CH—(CH₂)₁₄—O—SO₃H;
(CF₃)₂N—CH═CH—(CH₂)₁₄—O—PO₃H; (CF₃)₂N—CH═CH—(CH₂)₁₄—OH
(CF₃)₂N—CH═CH—(CH₂)₁₅—COOH; (CF₃)₂N—CH═CH—(CH₂)₁₅—SO₃H; (CF₃)₂N—CH═CH—(CH₂)₁₅—O—SO₃H;
(CF₃)₂N—CH═CH—(CH₂)₁₅—O—PO₃H; (CF₃)₂N—CH═CH—(CH₂)₁₅—OH
(CF₃)₂N—CH═CH—(CH₂)₁₆—COOH; (CF₃)₂N—CH═CH—(CH₂)₁₆—SO₃H; (CF₃)₂N—CH═CH—(CH₂)₁₆—O—SO₃H;
(CF₃)₂N—CH═CH—(CH₂)₁₆—O—PO₃H; (CF₃)₂N—CH═CH—(CH₂)₁₆—OH
CF₃S—CH₂CHBr—(CH₂)₄—COOH; CF₃S—CH₂CHBr—(CH₂)₄—SO₃H; CF₃S—CH₂CHBr—(CH₂)₄—O—SO₃H;
CF₃S—CH₂CHBr—(CH₂)₄—O—PO₃H; CF₃S—CH₂CHBr—(CH₂)₄—OH
CF₃S—CH₂CHBr—(CH₂)₅—COOH; CF₃S—CH₂CHBr—(CH₂)₅—SO₃H; CF₃S—CH₂CHBr—(CH₂)₅—O—SO₃H;
CF₃S—CH₂CHBr—(CH₂)₅—O—PO₃H; CF₃S—CH₂CHBr—(CH₂)₅—OH
CF₃S—CH₂CHBr—(CH₂)₆—COOH; CF₃S—CH₂CHBr—(CH₂)₆—SO₃H; CF₃S—CH₂CHBr—(CH₂)₆—O—SO₃H;
CF₃S—CH₂CHBr—(CH₂)₆—O—PO₃H; CF₃S—CH₂CHBr—(CH₂)₆—OH
CF₃S—CH₂CHBr—(CH₂)₇—COOH; CF₃S—CH₂CHBr—(CH₂)₇—SO₃H; CF₃S—CH₂CHBr—(CH₂)₇—O—SO₃H;
CF₃S—CH₂CHBr—(CH₂)₇—O—PO₃H; CF₃S—CH₂CHBr—(CH₂)₇—OH
CF₃S—CH₂CHBr—(CH₂)₈—COOH; CF₃S—CH₂CHBr—(CH₂)₈—SO₃H; CF₃S—CH₂CHBr—(CH₂)₈—O—SO₃H;
CF₃S—CH₂CHBr—(CH₂)₈—O—PO₃H; CF₃S—CH₂CHBr—(CH₂)₈—OH
CF₃S—CH₂CHBr—(CH₂)₉—COOH; CF₃S—CH₂CHBr—(CH₂)₉—SO₃H; CF₃S—CH₂CHBr—(CH₂)₉—O—SO₃H;
CF₃S—CH₂CHBr—(CH₂)₉—O—PO₃H; CF₃S—CH₂CHBr—(CH₂)₉—OH
CF₃S—CH₂CHBr—(CH₂)₁₀—COOH; CF₃S—CH₂CHBr—(CH₂)₁₀—SO₃H; CF₃S—CH₂CHBr—(CH₂)₁₀—O—SO₃H;
CF₃S—CH₂CHBr—(CH₂)₁₀—O—PO₃H; CF₃S—CH₂CHBr—(CH₂)₁₀—OH
CF₃S—CH₂CHBr—(CH₂)₁₁—COOH; CF₃S—CH₂CHBr—(CH₂)₁₁—SO₃H; CF₃S—CH₂CHBr—(CH₂)₁₁—O—SO₃H;
CF₃S—CH₂CHBr—(CH₂)₁₁—O—PO₃H; CF₃S—CH₂CHBr—(CH₂)₁₁—OH
CF₃S—CH₂CHBr—(CH₂)₁₂—COOH; CF₃S—CH₂CHBr—(CH₂)₁₂—SO₃H; CF₃S—CH₂CHBr—(CH₂)₁₂—O—SO₃H;
CF₃S—CH₂CHBr—(CH₂)₁₂—O—PO₃H; CF₃S—CH₂CHBr—(CH₂)₁₂—OH
CF₃S—CH₂CHBr—(CH₂)₁₃—COOH; CF₃S—CH₂CHBr—(CH₂)₁₃—SO₃H; CF₃S—CH₂CHBr—(CH₂)₁₃—O—SO₃H;
CF₃S—CH₂CHBr—(CH₂)₁₃—O—PO₃H; CF₃S—CH₂CHBr—(CH₂)₁₃—OH
CF₃S—CH₂CHBr—(CH₂)₁₄—COOH; CF₃S—CH₂CHBr—(CH₂)₁₄—SO₃H; CF₃S—CH₂CHBr—(CH₂)₁₄—O—SO₃H;
CF₃S—CH₂CHBr—(CH₂)₁₄—O—PO₃H;
CF₃S—CH₂CHBr—(CH₂)₁₅—COOH; CF₃S—CH₂CHBr—(CH₂)₁₅—SO₃H; CF₃S—CH₂CHBr—(CH₂)₁₅—O—SO₃H;
CF₃S—CH₂CHBr—(CH₂)₁₅—O—PO₃H; CF₃S—CH₂CHBr—(CH₂)₁₅—OH
CF₃S—CH₂CHBr—(CH₂)₁₆—COOH; CF₃S—CH₂CHBr—(CH₂)₁₆—SO₃H; CF₃S—CH₂CHBr—(CH₂)₁₆—O—SO₃H;
CF₃S—CH₂CHBr—(CH₂)₁₆—O—PO₃H; CF₃S—CH₂CHBr—(CH₂)₁₆—OH
(CF₃—CH₂—CH₂)₂N—CH₂CHBr—(CH₂)₄—COOH; (CF₃—CH₂—CH₂)₂N—CH₂CHBr—(CH₂)₄—SO₃H;
(CF₃—CH₂—CH₂)₂N—CH₂CHBr—(CH₂)₄—O—SO₃H; (CF₃—CH₂—CH₂)₂N—CH₂CHBr—(CH₂)₄—O—PO₃H;
(CF₃—CH₂—CH₂)₂N—CH₂CHBr—(CH₂)₄—OH; (CF₃—CH₂—CH₂)₂N—CH₂CHBr—(CH₂)₄—SH
(CF₃—CH₂—CH₂)₂N—CH₂CHBr—(CH₂)₅—COOH; (CF₃—CH₂—CH₂)₂N—CH₂CHBr—(CH₂)₅—SO₃H;
(CF₃—CH₂—CH₂)₂N—CH₂CHBr—(CH₂)₅—O—SO₃H; (CF₃—CH₂—CH₂)₂N—CH₂CHBr—(CH₂)₅—O—PO₃H;
(CF₃—CH₂—CH₂)₂N—CH₂CHBr—(CH₂)₅—OH; (CF₃—CH₂—CH₂)₂N—CH₂CHBr—(CH₂)₅—SH;
(CF₃—CH₂—CH₂)₂N—CH₂CHBr—(CH₂)₆—COOH; (CF₃—CH₂—CH₂)₂N—CH₂CHBr—(CH₂)₆—SO₃H;
(CF₃—CH₂—CH₂)₂N—CH₂CHBr—(CH₂)₆—O—SO₃H; (CF₃—CH₂—CH₂)₂N—CH₂CHBr—(CH₂)₆—O—PO₃H;
(CF₃—CH₂—CH₂)₂N—CH₂CHBr—(CH₂)₆—OH; (CF₃—CH₂—CH₂)₂N—CH₂CHBr—(CH₂)₆—SH
(CF₃—CH₂—CH₂)₂N—CH₂CHBr—(CH₂)₇—COOH; (CF₃—CH₂—CH₂)₂N—CH₂CHBr—(CH₂)₇—SO₃H;
(CF₃—CH₂—CH₂)₂N—CH₂CHBr—(CH₂)₇—O—SO₃H; (CF₃—CH₂—CH₂)₂N—CH₂CHBr—(CH₂)₇—O—PO₃H;
(CF₃—CH₂—CH₂)₂N—CH₂CHBr—(CH₂)₇—OH; (CF₃—CH₂—CH₂)₂N—CH₂CHBr—(CH₂)₇—SH
(CF₃—CH₂—CH₂)₂N—CH₂CHBr—(CH₂)₈—COOH; (CF₃—CH₂—CH₂)₂N—CH₂CHBr—(CH₂)₈—SO₃H;
(CF₃—CH₂—CH₂)₂N—CH₂CHBr—(CH₂)₈—O—SO₃H; (CF₃—CH₂—CH₂)₂N—CH₂CHBr—(CH₂)₈—O—PO₃H;
(CF₃—CH₂—CH₂)₂N—CH₂CHBr—(CH₂)₈—OH; (CF₃—CH₂—CH₂)₂N—CH₂CHBr—(CH₂)₈—SH
(CF₃—CH₂—CH₂)₂N—CH₂CHBr—(CH₂)₉—COOH; (CF₃—CH₂—CH₂)₂N—CH₂CHBr—(CH₂)₉—SO₃H;
(CF₃—CH₂—CH₂)₂N—CH₂CHBr—(CH₂)₉—O—SO₃H; (CF₃—CH₂—CH₂)₂N—CH₂CHBr—(CH₂)₉—O—PO₃H;
(CF₃—CH₂—CH₂)₂N—CH₂CHBr—(CH₂)₉—OH; (CF₃—CH₂—CH₂)₂N—CH₂CHBr—(CH₂)₉—SH
(CF₃—CH₂—CH₂)₂N—CH₂CHBr—(CH₂)₁₀—COOH; (CF₃—CH₂—CH₂)₂N—CH₂CHBr—(CH₂)₁₀—SO₃H;
(CF₃—CH₂—CH₂)₂N—CH₂CHBr—(CH₂)₁₀—O—SO₃H; (CF₃—CH₂—CH₂)₂N—CH₂CHBr—(CH₂)₁₀—O—PO₃H;
(CF₃—CH₂—CH₂)₂N—CH₂CHBr—(CH₂)₁₀—OH; (CF₃—CH₂—CH₂)₂N—CH₂CHBr—(CH₂)₁₀—SH
(CF₃—CH₂—CH₂)₂N—CH₂CHBr—(CH₂)₁₁—COOH; (CF₃—CH₂—CH₂)₂N—CH₂CHBr—(CH₂)₁₁—SO₃H;
(CF₃—CH₂—CH₂)₂N—CH₂CHBr—(CH₂)₁₁—O—SO₃H; (CF₃—CH₂—CH₂)₂N—CH₂CHBr—(CH₂)₁₁—O—PO₃H;
(CF₃—CH₂—CH₂)₂N—CH₂CHBr—(CH₂)₁₁—OH; (CF₃—CH₂—CH₂)₂N—CH₂CHBr—(CH₂)₁₁—SH
(CF₃—CH₂—CH₂)₂N—CH₂CHBr—(CH₂)₁₂—COOH; (CF₃—CH₂—CH₂)₂N—CH₂CHBr—(CH₂)₁₂—SO₃H;
(CF₃—CH₂—CH₂)₂N—CH₂CHBr—(CH₂)₁₂—O—SO₃H; (CF₃—CH₂—CH₂)₂N—CH₂CHBr—(CH₂)₁₂—O—PO₃H;
(CF₃—CH₂—CH₂)₂N—CH₂CHBr—(CH₂)₁₂—OH; (CF₃—CH₂—CH₂)₂N—CH₂CHBr—(CH₂)₁₂—SH;
(CF₃—CH₂—CH₂)₂N—CH₂CHBr—(CH₂)₁₃—COOH; (CF₃—CH₂—CH₂)₂N—CH₂CHBr—(CH₂)₁₃—SO₃H;
(CF₃—CH₂—CH₂)₂N—CH₂CHBr—(CH₂)₁₃—O—SO₃H; (CF₃—CH₂—CH₂)₂N—CH₂CHBr—(CH₂)₁₃—O—PO₃H;
(CF₃—CH₂—CH₂)₂N—CH₂CHBr—(CH₂)₁₃—OH; ((CF₃—CH₂—CH₂)₂N—CH₂CHBr—(CH₂)₁₃—SH
(CF₃—CH₂—CH₂)₂N—CH₂CHBr—(CH₂)₁₄—COOH; (CF₃—CH₂—CH₂)₂N—CH₂CHBr—(CH₂)₁₄—SO₃H;
(CF₃—CH₂—CH₂)₂N—CH₂CHBr—(CH₂)₁₄—O—SO₃H; (CF₃—CH₂—CH₂)₂N—CH₂CHBr—(CH₂)₁₄—O—PO₃H;
(CF₃—CH₂—CH₂)₂N—CH₂CHBr—(CH₂)₁₄—OH; (CF₃—CH₂—CH₂)₂N—CH₂CHBr—(CH₂)₁₄—SH;
(CF₃—CH₂—CH₂)₂N—CH₂CHBr—(CH₂)₁₅—COOH; (CF₃—CH₂—CH₂)₂N—CH₂CHBr—(CH₂)₁₅—SO₃H;
(CF₃—CH₂—CH₂)₂N—CH₂CHBr—(CH₂)₁₅—O—SO₃H; (CF₃—CH₂—CH₂)₂N—CH₂CHBr—(CH₂)₁₅—O—PO₃H;
(CF₃—CH₂—CH₂)₂N—CH₂CHBr—(CH₂)₁₅—OH; (CF₃—CH₂—CH₂)₂N—CH₂CHBr—(CH₂)₁₅—SH;
(CF₃—CH₂—CH₂)₂N—CH₂CHBr—(CH₂)₁₆—COOH; (CF₃—CH₂—CH₂)₂N—CH₂CHBr—(CH₂)₁₆—SO₃H;
(CF₃—CH₂—CH₂)₂N—CH₂CHBr—(CH₂)₁₆—O—SO₃H; (CF₃—CH₂—CH₂)₂N—CH₂CHBr—(CH₂)₁₆—O—PO₃H;
(CF₃—CH₂—CH₂)₂N—CH₂CHBr—(CH₂)₁₆—OH; (CF₃—CH₂—CH₂)₂N—CH₂CHBr—(CH₂)₁₆—SH
CF₃—CH₂—CH₂—S—CH₂CHBr—(CH₂)₄—COOH; CF₃—CH₂—CH₂—S—CH₂CHBr—(CH₂)₄—SO₃H;
CF₃—CH₂—CH₂)—S—CH₂CHBr—(CH₂)₄—O—SO₃H; CF₃—CH₂—CH₂—S—CH₂CHBr—(CH₂)₄—O—PO₃H;
CF₃—CH₂—CH₂—S—CH₂CHBr—(CH₂)₄—OH; CF₃—CH₂—CH₂—S—CH₂CHBr—(CH₂)₄—SH
CF₃—CH₂—CH₂—S—CH₂CHBr—(CH₂)₅—COOH; CF₃—CH₂—CH₂—S—CH₂CHBr—(CH₂)₅—SO₃H;
CF₃—CH₂—CH₂—S—CH₂CHBr—(CH₂)₅—O—SO₃H; CF₃—CH₂—CH₂—S—CH₂CHBr—(CH₂)₅—O—PO₃H;
CF₃—CH₂—CH₂—S—CH₂CHBr—(CH₂)₅—OH; CF₃—CH₂—CH₂—S—CH₂CHBr—(CH₂)₅—SH;
CF₃—CH₂—CH₂—S—CH₂CHBr—(CH₂)₆—COOH; CF₃—CH₂—CH₂—S—CH₂CHBr—(CH₂)₆—SO₃H;
CF₃—CH₂—CH₂—S—CH₂CHBr—(CH₂)₆—O—SO₃H; CF₃—CH₂—CH₂—S—CH₂CHBr—(CH₂)₆—O—PO₃H;
CF₃—CH₂—CH₂—S—CH₂CHBr—(CH₂)₆—OH; CF₃—CH₂—CH₂—S—CH₂CHBr—(CH₂)₆—SH
CF₃—CH₂—CH₂—S—CH₂CHBr—(CH₂)₇—COOH; CF₃—CH₂—CH₂—S—CH₂CHBr—(CH₂)₇—SO₃H;
CF₃—CH₂—CH₂—S—CH₂CHBr—(CH₂)₇—O—SO₃H; CF₃—CH₂—CH₂—S—CH₂CHBr—(CH₂)₇—O—PO₃H;
CF₃—CH₂—CH₂—S—CH₂CHBr—(CH₂)₇—OH; CF₃—CH₂—CH₂—S—CH₂CHBr—(CH₂)₇—SH
CF₃—CH₂—CH₂—S—CH₂CHBr—(CH₂)₈—COOH; CF₃—CH₂—CH₂—S—CH₂CHBr—(CH₂)₈—SO₃H;
CF₃—CH₂—CH₂—S—CH₂CHBr—(CH₂)₈—O—SO₃H; CF₃—CH₂—CH₂—S—CH₂CHBr—(CH₂)₈—O—PO₃H;
CF₃—CH₂—CH₂—S—CH₂CHBr—(CH₂)₈—OH; CF₃—CH₂—CH₂—S—CH₂CHBr—(CH₂)₈—SH
CF₃—CH₂—CH₂—S—CH₂CHBr—(CH₂)₉—COOH; CF₃—CH₂—CH₂—S—CH₂CHBr—(CH₂)₉—SO₃H;
CF₃—CH₂—CH₂—S—CH₂CHBr—(CH₂)₉—O—SO₃H; CF₃—CH₂—CH₂—S—CH₂CHBr—(CH₂)₉—O—PO₃H;
CF₃—CH₂—CH₂—S—CH₂CHBr—(CH₂)₉—OH; CF₃—CH₂—CH₂—S—CH₂CHBr—(CH₂)₉—SH
CF₃—CH₂—CH₂—S—CH₂CHBr—(CH₂)₁₀—COOH; CF₃—CH₂—CH₂—S—CH₂CHBr—(CH₂)₁₀—SO₃H;
CF₃—CH₂—CH₂—S—CH₂CHBr—(CH₂)₁₀—O—SO₃H; CF₃—CH₂—CH₂—S—CH₂CHBr—(CH₂)₁₀—O—PO₃H;
CF₃—CH₂—CH₂—S—CH₂CHBr—(CH₂)₁₀—OHCF₃—CH₂—CH₂—S—CH₂CHBr—(CH₂)₁₀—SH
CF₃—CH₂—CH₂—S—CH₂CHBr—(CH₂)₁₁—COOH; CF₃—CH₂—CH₂—S—CH₂CHBr—(CH₂)₁₁—SO₃H;
CF₃—CH₂—CH₂—S—CH₂CHBr—(CH₂)₁₁—O—SO₃H; CF₃—CH₂—CH₂—S—CH₂CHBr—(CH₂)₁₁—O—PO₃H;
CF₃—CH₂—CH₂—S—CH₂CHBr—(CH₂)₁₁—OH; CF₃—CH₂—CH₂—S—CH₂CHBr—(CH₂)₁₁—SH
CF₃—CH₂—CH₂—S—CH₂CHBr—(CH₂)₁₂—COOH; CF₃—CH₂—CH₂—S—CH₂CHBr—(CH₂)₁₂—SO₃H;
CF₃—CH₂—CH₂—S—CH₂CHBr—(CH₂)₁₂—O—SO₃H; CF₃—CH₂—CH₂—S—CH₂CHBr—(CH₂)₁₂—O—PO₃H;
CF₃—CH₂—CH₂—S—CH₂CHBr—(CH₂)₁₂—OH; CF₃—CH₂—CH₂—S—CH₂CHBr—(CH₂)₁₂—SH;
CF₃—CH₂—CH₂—S—CH₂CHBr—(CH₂)₁₃—COOH; CF₃—CH₂—CH₂—S—CH₂CHBr—(CH₂)₁₃—SO₃H;
CF₃—CH₂—CH₂—S—CH₂CHBr—(CH₂)₁₃—O—SO₃H; CF₃—CH₂—CH₂—S—CH₂CHBr—(CH₂)₁₃—O—PO₃H;
CF₃—CH₂—CH₂—S—CH₂CHBr—(CH₂)₁₃—OH; CF₃—CH₂—CH₂—S—CH₂CHBr—(CH₂)₁₃—SH
CF₃—CH₂—CH₂—S—CH₂CHBr—(CH₂)₁₄—COOH; CF₃—CH₂—CH₂—S—CH₂CHBr—(CH₂)₁₄—SO₃H;
CF₃—CH₂—CH₂—S—CH₂CHBr—(CH₂)₁₄—O—SO₃H; CF₃—CH₂—CH₂—S—CH₂CHBr—(CH₂)₁₄—O—PO₃H;
CF₃—CH₂—CH₂—S—CH₂CHBr—(CH₂)₁₄—OH; CF₃—CH₂—CH₂—S—CH₂CHBr—(CH₂)₁₄—SH;
CF₃—CH₂—CH₂—S—CH₂CHBr—(CH₂)₁₅—COOH; CF₃—CH₂—CH₂—S—CH₂CHBr—(CH₂)₁₅—SO₃H;
CF₃—CH₂—CH₂—S—CH₂CHBr—(CH₂)₁₅—O—SO₃H; CF₃—CH₂—CH₂—S—CH₂CHBr—(CH₂)₁₅—O—PO₃H;
CF₃—CH₂—CH₂—S—CH₂CHBr—(CH₂)₁₅—OH; CF₃—CH₂—CH₂—S—CH₂CHBr—(CH₂)₁₅—SH;
CF₃—CH₂—CH₂—S—CH₂CHBr—(CH₂)₁₆—COOH; CF₃—CH₂—CH₂—S—CH₂CHBr—(CH₂)₁₆—SO₃H;
CF₃—CH₂—CH₂—S—CH₂CHBr—(CH₂)₁₆—O—SO₃H; CF₃—CH₂—CH₂—S—CH₂CHBr—(CH₂)₁₆—O—PO₃H;
CF₃—CH₂—CH₂—S—CH₂CHBr—(CH₂)₁₆—OH; CF₃—CH₂—CH₂—S—CH₂CHBr—(CH₂)₁₆—SH
C₂F₅—S—CH₂CHBr—(CH₂)₄—COOH; C₂F₅—S—CH₂CHBr—(CH₂)₄—SO₃H; C₂F₅—S—CH₂CHBr—(CH₂)₄—O—SO₃H;
C₂F₅—S—CH₂CHBr—(CH₂)₄—O—PO₃H; C₂F₅—S—CH₂CHBr—(CH₂)₄—OH;
C₂F₅—S—CH₂CHBr—(CH₂)₄—SH
C₂F₅—S—CH₂CHBr—(CH₂)₅—COOH;
C₂F₅—S—CH₂CHBr—(CH₂)₅—SO₃HC₂F₅—S—CH₂CHBr—(CH₂)₅—O—SO₃HC₂F₅—S—CH₂CHBr—(CH₂)₅—O—PO₃H;
C₂F₅—S—CH₂CHBr—(CH₂)₅—OH;
C₂F₅—S—CH₂CHBr—(CH₂)₅—SH;
C₂F₅—S—CH₂CHBr—(CH₂)₆—COOH; C₂F₅—S—CH₂CHBr—(CH₂)₆—SO₃H; C₂F₅—S—(CH₂)₆—O—SO₃H;
C₂F₅—S—CH₂CHBr—(CH₂)₆—O—PO₃H; C₂F₅—S—CH₂CHBr—(CH₂)₆—OH; C₂F₅—S—CH₂CHBr—(CH₂)₆—SH
C₂F₅—S—CH₂CHBr—(CH₂)₇—COOH; C₂F₅—S—CH₂CHBr—(CH₂)₇—SO₃H; C₂F₅—S—(CH₂)₇—O—SO₃H;
C₂F₅—S—CH₂CHBr—(CH₂)₇—O—PO₃H; C₂F₅—S—CH₂CHBr—(CH₂)₇—OH; C₂F₅—S—CH₂CHBr—(CH₂)₇—SH
C₂F₅—S—CH₂CHBr—(CH₂)₈—COOH; C₂F₅—S—CH₂CHBr—(CH₂)₈—SO₃H; C₂F₅—S—CH₂CHBr—(CH₂)₈—O—SO₃H;
C₂F₅—S—CH₂CHBr—(CH₂)₈—O—PO₃H; C₂F₅—S—CH₂CHBr—(CH₂)₈—OH;
C₂F₅—S—CH₂CHBr—(CH₂)₈—SH
C₂F₅—S—CH₂CHBr—(CH₂)₉—COOH; C₂F₅—S—CH₂CHBr—(CH₂)₉—SO₃H; C₂F₅—S—CH₂CHBr—(CH₂)₉—O—SO₃H;
C₂F₅—S—CH₂CHBr—(CH₂)₉—O—PO₃H; C₂F₅—S—CH₂CHBr—(CH₂)₉—OH;
C₂F₅—S—CH₂CHBr—(CH₂)₉—SH
C₂F₅—S—CH₂CHBr—(CH₂)₁₀—COOH; C₂F₅—S—CH₂CHBr—(CH₂)₁₀—SO₃H; C₂F₅—S—CH₂CHBr—(CH₂)₁₀—O—SO₃H;
C₂F₅—S—CH₂CHBr—(CH₂)₁₀—O—PO₃H; C₂F₅—S—CH₂CHBr—(CH₂)₁₀—OH;
C₂F₅—S—CH₂CHBr—(CH₂)₁₀—SH
C₂F₅—S—CH₂CHBr—(CH₂)₁₁—COOH; C₂F₅—S—CH₂CHBr—(CH₂)₁₁—SO₃H; C₂F₅—S—CH₂CHBr—(CH₂)₁₁—O—SO₃H;
C₂F₅—S—CH₂CHBr—(CH₂)₁₁—O—PO₃H; C₂F₅—S—CH₂CHBr—(CH₂)₁₁—OH;
C₂F₅—S—CH₂CHBr—(CH₂)₁₁—SH
C₂F₅—S—CH₂CHBr—(CH₂)₁₂—COOH; C₂F₅—S—CH₂CHBr—(CH₂)₁₂—SO₃H; C₂F₅—S—CH₂CHBr—(CH₂)₁₂—O—SO₃H;
C₂F₅—S—CH₂CHBr—(CH₂)₁₂—O—PO₃H; C₂F₅—S—CH₂CHBr—(CH₂)₁₂—OH;
C₂F₅—S—CH₂CHBr—(CH₂)₁₂—SH;
C₂F₅—S—CH₂CHBr—(CH₂)₁₃—COOH; C₂F₅—S—CH₂CHBr—(CH₂)₁₃—SO₃H; C₂F₅—S—CH₂CHBr—(CH₂)₁₃—O—SO₃H;
C₂F₅—S—CH₂CHBr—(CH₂)₁₃—O—PO₃H; C₂F₅—S—CH₂CHBr—(CH₂)₁₃—OH;
C₂F₅—S—CH₂CHBr—(CH₂)₁₃—SH
C₂F₅—S—CH₂CHBr—(CH₂)₁₄—COOH; C₂F₅—S—CH₂CHBr—(CH₂)₁₄—SO₃H; C₂F₅—S—CH₂CHBr—(CH₂)₁₄—O—SO₃H;
C₂F₅—S—CH₂CHBr—(CH₂)₁₄—O—PO₃H; C₂F₅—S—CH₂CHBr—(CH₂)₁₄—OH;
C₂F₅—S—CH₂CHBr—(CH₂)₁₄—SH;
C₂F₅—S—CH₂CHBr—(CH₂)₁₅—COOH; C₂F₅—S—CH₂CHBr—(CH₂)₁₅—SO₃H; C₂F₅—S—CH₂CHBr—(CH₂)₁₅—O—SO₃H;
C₂F₅—S—CH₂CHBr—(CH₂)₁₅—O—PO₃H; C₂F₅—S—CH₂CHBr—(CH₂)₁₅—OH;
C₂F₅—S—CH₂CHBr—(CH₂)₁₅—SH;
C₂F₅—S—CH₂CHBr—(CH₂)₁₆—COOH; C₂F₅—S—CH₂CHBr—(CH₂)₁₆—SO₃H; C₂F₅—S—CH₂CHBr—(CH₂)₁₆—O—SO₃H;
C₂F₅—S—CH₂CHBr—(CH₂)₁₆—O—PO₃H; C₂F₅—S—CH₂CHBr—(CH₂)₁₆—OH;
C₂F₅—S—CH₂CHBr—(CH₂)₁₆—SH
(CF₃)₂N—CH₂CHBr—(CH₂)₄—COOH; (CF₃)₂N—CH₂CHBr—(CH₂)₄—SO₃H; (CF₃)₂N—CH₂CHBr—(CH₂)₄—O—SO₃H;
(CF₃)₂N—CH₂CHBr—(CH₂)₄—O—PO₃H
(CF₃)₂N—CH₂CHBr—(CH₂)₅—COOH; (CF₃)₂N—CH₂CHBr—(CH₂)₅—SO₃H; (CF₃)₂N—CH₂CHBr—(CH₂)₅—O—SO₃H;
(CF₃)₂N—CH₂CHBr—(CH₂)₅—O—PO₃H
(CF₃)₂N—CH₂CHBr—(CH₂)₆—COOH; (CF₃)₂N—CH₂CHBr—(CH₂)₆—SO₃H; (CF₃)₂N—CH₂CHBr—(CH₂)₆—O—SO₃H;
(CF₃)₂N—CH₂CHBr—(CH₂)₆—O—PO₃H
(CF₃)₂N—CH₂CHBr—(CH₂)₇—COOH; (CF₃)₂N—CH₂CHBr—(CH₂)₇—SO₃H; (CF₃)₂N—CH₂CHBr—(CH₂)₇—O—SO₃H;
(CF₃)₂N—CH₂CHBr—(CH₂)₇—O—PO₃H
(CF₃)₂N—CH₂CHBr—(CH₂)₈—COOH; (CF₃)₂N—CH₂CHBr—(CH₂)₈—SO₃H; (CF₃)₂N—CH₂CHBr—(CH₂)₈—O—SO₃H;
(CF₃)₂N—CH₂CHBr—(CH₂)₈—O—PO₃H
(CF₃)₂N—CH₂CHBr—(CH₂)₉—COOH; (CF₃)₂N—CH₂CHBr—(CH₂)₉—SO₃H; (CF₃)₂N—CH₂CHBr—(CH₂)₉—O—SO₃H;
(CF₃)₂N—CH₂CHBr—(CH₂)₉—O—PO₃H
(CF₃)₂N—CH₂CHBr—(CH₂)₁₀—COOH; (CF₃)₂N—CH₂CHBr—(CH₂)₁₀—SO₃H; (CF₃)₂N—CH₂CHBr—(CH₂)₁₀—O—SO₃H;
(CF₃)₂N—CH₂CHBr—(CH₂)₁₀—O—PO₃H
(CF₃)₂N—CH₂CHBr—(CH₂)₁₁—COOH; (CF₃)₂N—CH₂CHBr—(CH₂)₁₁—SO₃H; (CF₃)₂N—CH₂CHBr—(CH₂)₁₁—O—SO₃H;
(CF₃)₂N—CH₂CHBr—(CH₂)₁₁—O—PO₃H
(CF₃)₂N—CH₂CHBr—(CH₂)₁₂—COOH; (CF₃)₂N—CH₂CHBr—(CH₂)₁₂—SO₃H; (CF₃)₂N—CH₂CHBr—(CH₂)₁₂—O—SO₃H;
(CF₃)₂N—CH₂CHBr—(CH₂)₁₂—O—PO₃H
(CF₃)₂N—CH₂CHBr—(CH₂)₁₃—COOH; (CF₃)₂N—CH₂CHBr—(CH₂)₁₃—SO₃H; (CF₃)₂N—CH₂CHBr—(CH₂)₁₃—O—SO₃H;
(CF₃)₂N—CH₂CHBr—(CH₂)₁₃—O—PO₃H
(CF₃)₂N—CH₂CHBr—(CH₂)₁₄—COOH; (CF₃)₂N—CH₂CHBr—(CH₂)₁₄—SO₃H; (CF₃)₂N—CH₂CHBr—(CH₂)₁₄—O—SO₃H;
(CF₃)₂N—CH₂CHBr—(CH₂)₁₄—O—PO₃H
(CF₃)₂N—CH₂CHBr—(CH₂)₁₅—COOH; (CF₃)₂N—CH₂CHBr—(CH₂)₁₅—SO₃H; (CF₃)₂N—CH₂CHBr—(CH₂)₁₅—O—SO₃H;
(CF₃)₂N—CH₂CHBr—(CH₂)₁₅—O—PO₃H
(CF₃)₂N—CH₂CHBr—(CH₂)₁₆—COOH; (CF₃)₂N—CH₂CHBr—(CH₂)₁₆—SO₃H; (CF₃)₂N—CH₂CHBr—(CH₂)₁₆—O—SO₃H;
(CF₃)₂N—CH₂CHBr—(CH₂)₁₆—O—PO₃H
CF₃—S—(CH₂)₄—OH; CF₃—S—(CH₂)₄—SH; CF₃—S—(CH₂)₄—(OCH₂CHR)ₘ—OH; CF₃—S—(CH₂)₄—(OCH₂CHR)ₘ—SH;
CF₃—S—(CH₂)₄—(OCH₂CHR)ₘ—SO₂—CH═CH₂
CF₃—S—(CH₂)₅—OH; CF₃—S—(CH₂)₅—SH; CF₃—S—(CH₂)₅—(OCH₂CH₂)ₘ—OH; CF₃—S—(CH₂)₅—(OCH₂CH₂)ₘ—SH;
CF₃—S—(CH₂)₅—(OCH₂CH₂)ₘ—SO₂—CH═CH₂
CF₃—S—(CH₂)₆—OH; CF₃—S—(CH₂)₆—SH; CF₃—S—(CH₂)₆—(OCH₂CH₂)ₘ—OH; CF₃—S—(CH₂)₆—(OCH₂CH₂)ₘ—SH;
CF₃—S—(CH₂)₆—(OCH₂CH₂)ₘ—SO₂—CH═CH₂
CF₃—S—(CH₂)₇—OH; CF₃—S—(CH₂)₇—SH; CF₃—S—(CH₂)₇—(OCH₂CH₂)ₘ—OH; CF₃—S—(CH₂)₇—(OCH₂CH₂)ₘ—SH;
CF₃—S—(CH₂)₇—(OCH₂CH₂)ₘ—SO₂—CH═CH₂
CF₃—S—(CH₂)₈—OH; CF₃—S—(CH₂)₈—SH; CF₃—S—(CH₂)₈—(OCH₂CH₂)ₘ—OH; CF₃—S—(CH₂)₈—(OCH₂CH₂)ₘ—SH;
CF₃—S—(CH₂)₈—(OCH₂CH₂)ₘ—SO₂—CH═CH₂
CF₃—S—(CH₂)₉—OH; CF₃—S—(CH₂)₉—SH; CF₃—S—(CH₂)₉—(OCH₂CH₂)ₘ—OH; CF₃—S—(CH₂)₉—(OCH₂CH₂)ₘ—SH;
CF₃—S—(CH₂)₉—(OCH₂CH₂)ₘ—SO₂—CH═CH₂
CF₃—S—(CH₂)₁₀—OH; CF₃—S—(CH₂)₁₀—SH; CF₃—S—(CH₂)₁₀—(OCH₂CH₂)ₘ—OH; CF₃—S—(CH₂)₁₀—(OCH₂CH₂)ₘ—SH;
CF₃—S—(CH₂)₁₀—(OCH₂CH₂)ₘ—SO₂—CH═CH₂
CF₃—S—(CH₂)₁₁—OH; CF₃—S—(CH₂)₁₁—SH; CF₃—S—(CH₂)₁₁—(OCH₂CH₂)ₘ—OH; CF₃—S—(CH₂)₁₁—(OCH₂CH₂)ₘ—SH;
CF₃—S—(CH₂)₁₁—(OCH₂CH₂)ₘ—SO₂—CH═CH₂
CF₃—S—(CH₂)₁₂—OH; CF₃—S—(CH₂)₁₂—SH; CF₃—S—(CH₂)₁₂—(OCH₂CH₂)ₘ—OH; CF₃—S—(CH₂)₁₂—(OCH₂CH₂)ₘ—SH;
CF₃—S—(CH₂)₁₂—(OCH₂CH₂)ₘ—SO₂—CH═CH₂
CF₃—S—(CH₂)₁₃—OH; CF₃—S—(CH₂)₁₃—SH; CF₃—S—(CH₂)₁₃—(OCH₂CH₂)ₘ—OH; CF₃—S—(CH₂)₁₃—(OCH₂CH₂)ₘ—SH;
CF₃—S—(CH₂)₁₃—(OCH₂CH₂)ₘ—SO₂—CH═CH₂
CF₃—S—(CH₂)₁₄—OH; CF₃—S—(CH₂)₁₄—SH; CF₃—S—(CH₂)₁₄—(OCH₂CH₂)ₘ—OH; CF₃—S—(CH₂)₁₄—(OCH₂CH₂)ₘ—SH;
CF₃—S—(CH₂)₁₄—(OCH₂CH₂)ₘ—SO₂—CH═CH₂
CF₃—S—(CH₂)₁₅—OH; CF₃—S—(CH₂)₁₅—SH; CF₃—S—(CH₂)₁₅—(OCH₂CH₂)ₘ—OH; CF₃—S—(CH₂)₁₅—(OCH₂CH₂)ₘ—SH;
CF₃—S—(CH₂)₁₅—(OCH₂CH₂)ₘ—SO₂—CH═CH₂
CF₃—S—(CH₂)₁₆—OH; CF₃—S—(CH₂)₁₆—SHCF₃—S—(CH₂)₁₆—(OCH₂CH₂)ₘ—OH; CF₃—S—(CH₂)₁₆—(OCH₂CH₂)ₘ—SH;
CF₃—S—(CH₂)₁₆—(OCH₂CH₂)ₘ—SO₂—CH═CH₂
(CF₃)₂N—(CH₂)₄—OH; (CF₃)₂N—(CH₂)₄—SH; (CF₃)₂N—(CH₂)₄—(OCH₂CH₂)ₘ—OH; (CF₃)₂N—(CH₂)₄—(OCH₂CH₂)ₘ—SH;
(CF₃)₂N—(CH₂)₄—(OCH₂CH₂)ₘ—SO₂—CH═CH_{2;} (CF₃)₂N—(CH₂)₄—O—PO₃H
(CF₃)₂N—(CH₂)₅—OH; (CF₃)₂N—(CH₂)₅—SH; (CF₃)₂N—(CH₂)₅—(OCH₂CH₂)ₘ—OH; (CF₃)₂N—(CH₂)₅—(OCH₂CH₂)ₘ—SH;
(CF₃)₂N—(CH₂)₅—(OCH₂CH₂)ₘ—SO₂—CH═CH_{2;} (CF₃)₂N—(CH₂)₅—O—PO₃H
(CF₃)₂N—(CH₂)₆—OH; (CF₃)₂N—(CH₂)₆—SH; (CF₃)₂N—(CH₂)₆—(OCH₂CH₂)ₘ—OH; (CF₃)₂N—(CH₂)₆—(OCH₂CH₂)ₘ—SH;
(CF₃)₂N—(CH₂)₆—(OCH₂CH₂)ₘ—SO₂—CH═CH_{2;} (CF₃)₂N—(CH₂)₆—O—PO₃H
(CF₃)₂N—(CH₂)₇—OH; (CF₃)₂N—(CH₂)₇—SH; (CF₃)₂N—(CH₂)₇—(OCH₂CH₂)ₘ—OH; (CF₃)₂N—(CH₂)₇—(OCH₂CH₂)ₘ—SH;
(CF₃)₂N—(CH₂)₇—(OCH₂CH₂)ₘ—SO₂—CH═CH_{2;} (CF₃)₂N—(CH₂)₇—O—PO₃H
(CF₃)₂N—(CH₂)₈—OH; (CF₃)₂N—(CH₂)₈—SH; (CF₃)₂N—(CH₂)₈—(OCH₂CH₂)ₘ—OH; (CF₃)₂N—(CH₂)₈—(OCH₂CH₂)ₘ—SH;
(CF₃)₂N—(CH₂)₈—(OCH₂CH₂)ₘ—SO₂—CH═CH_{2;} (CF₃)₂N—(CH₂)₈—O—PO₃H
(CF₃)₂N—(CH₂)₉—OH; (CF₃)₂N—(CH₂)₉—SH; (CF₃)₂N—(CH₂)₉—(OCH₂CH₂)ₘ—OH; (CF₃)₂N—(CH₂)₉—(OCH₂CH₂)ₘ—SH;
(CF₃)₂N—(CH₂)₉—(OCH₂CH₂)ₘ—SO₂—CH═CH_{2;} (CF₃)₂N—(CH₂)₉—O—PO₃H
(CF₃)₂N—(CH₂)₁₀—OH; (CF₃)₂N—(CH₂)₁₀—SH; (CF₃)₂N—(CH₂)₁₀—(OCH₂CH₂)ₘ—OH;
(CF₃)₂N—(CH₂)₁₀—(OCH₂CH₂)ₘ—SH; (CF₃)₂N—(CH₂)₁₀—(OCH₂CH₂)ₘ—SO₂—CH═CH_{2;}
(CF₃)₂N—(CH₂)₁₀—O—PO₃H
(CF₃)₂N—(CH₂)₁₁—OH; (CF₃)₂N—(CH₂)₁₁—SH; (CF₃)₂N—(CH₂)₁₁—(OCH₂CH₂)ₘ—OH;
(CF₃)₂N—(CH₂)₁₁—(OCH₂CH₂)ₘ—SH; (CF₃)₂N—(CH₂)₁₁—(OCH₂CH₂)ₘ—SO₂—CH═CH_{2;}
(CF₃)₂N—(CH₂)₁₁—O—PO₃H
(CF₃)₂N—(CH₂)₁₂—OH; (CF₃)₂N—(CH₂)₁₂—SH; (CF₃)₂N—(CH₂)₁₂—(OCH₂CH₂)ₘ—OH;;
(CF₃)₂N—(CH₂)₁₂—(OCH₂CH₂)ₘ—SH; (CF₃)₂N—(CH₂)₁₂—(OCH₂CH₂)ₘ—SO₂—CH═CH_{2;}
(CF₃)₂N—(CH₂)₁₂—O—PO₃H
(CF₃)₂N(CH₂)₁₃—OH; (CF₃)₂N(CH₂)₁₃—SH; (CF₃)₂N—(CH₂)₁₃—(OCH₂CH₂)ₘ—OH;
(CF₃)₂N—(CH₂)₁₃—(OCH₂CH₂)ₘ—SH; (CF₃)₂N—(CH₂)₁₃—(OCH₂CH₂)ₘ—SO₂—CH═CH_{2;}
(CF₃)₂N(CH₂)₁₃—O—PO₃H
(CF₃)₂N—(CH₂)₁₄—OH; (CF₃)₂N—(CH₂)₁₄—SH; (CF₃)₂N—(CH₂)₁₄—(OCH₂CH₂)ₘ—OH;
(CF₃)₂N—(CH₂)₁₄—(OCH₂CH₂)ₘ—SH; (CF₃)₂N—(CH₂)₁₄—(OCH₂CH₂)ₘ—SO₂—CH═CH_{2;}
(CF₃)₂N—(CH₂)₁₄—O—PO₃H
(CF₃)₂N—(CH₂)₁₅—OH; (CF₃)₂N—(CH₂)₁₅—SH; (CF₃)₂N—(CH₂)₁₅—(OCH₂CH₂)ₘ—OH;
(CF₃)₂N—(CH₂)₁₅—(OCH₂CH₂)ₘ—SH; (CF₃)₂N—(CH₂)₁₅—(OCH₂CH₂)ₘ—SO₂—CH═CH_{2;}
(CF₃)₂N—(CH₂)₁₅—O—PO₃H
(CF₃)₂N—(CH₂)₁₆—OH; (CF₃)₂N—(CH₂)₁₆—SH; (CF₃)₂N—(CH₂)₁₆—(OCH₂CH₂)ₘ—OH;
(CF₃)₂N—(CH₂)₁₆—(OCH₂CH₂)ₘ—SH; (CF₃)₂N—(CH₂)₁₆—(OCH₂CH₂)ₘ—SO₂—CH═CH_{2;}
(CF₃)₂N—(CH₂)₁₆—O—PO₃H
(CF₃)₂N—CH₂—CH═CH—(CH₂)₄—OH; (CF₃)₂N—CH₂—CH═CH—(CH₂)₄—(OCH₂CH₂)ₘ—OH;
(CF₃)₂N—CH₂—CH═CH—(CH₂)₄—(OCH₂CH₂)ₘ—SO₂—CH═CH₂
(CF₃)₂N—CH₂—CH═CH—(CH₂)₅—OH; (CF₃)₂N—CH₂—CH═CH—(CH₂)₅—(OCH₂CH₂)ₘ—OH;
(CF₃)₂N—CH₂—CH═CH—(CH₂)₅—(OCH₂CH₂)ₘ—SO₂—CH═CH₂
(CF₃)₂N—CH₂—CH═CH—(CH₂)₆—OH; (CF₃)₂N—CH₂—CH═CH—(CH₂)₆—(OCH₂CH₂)ₘ—OH;
(CF₃)₂N—CH₂—CH═CH—(CH₂)₆—(OCH₂CH₂)ₘ—SO₂—CH═CH₂
(CF₃)₂N—CH₂—CH═CH—(CH₂)₇—OH; (CF₃)₂N—CH₂—CH═CH—(CH₂)₇—(OCH₂CH₂)ₘ—OH;
(CF₃)₂N—CH₂—CH═CH—(CH₂)₇—(OCH₂CH₂)ₘ—SO₂—CH═CH₂
(CF₃)₂N—CH₂—CH═CH—(CH₂)₈—OH; (CF₃)₂N—CH₂—CH═CH—(CH₂)₈—(OCH₂CH₂)ₘ—OH;
(CF₃)₂N—CH₂—CH═CH—(CH₂)₈—(OCH₂CH₂)ₘ—SO₂—CH═CH₂
(CF₃)₂N—CH₂—CH═CH—(CH₂)₉—OH; (CF₃)₂N—CH₂—CH═CH—(CH₂)₉—(OCH₂CH₂)ₘ—OH;
(CF₃)₂N—CH₂—CH═CH—(CH₂)₉—(OCH₂CH₂)ₘ—SO₂—CH═CH₂
(CF₃)₂N—CH₂—CH═CH—(CH₂)₁₀—OH; (CF₃)₂N—CH₂—CH═CH—(CH₂)₁₀—(OCH₂CH₂)ₘ—OH;
(CF₃)₂N—CH₂—CH═CH—(CH₂)₁₀—(OCH₂CH₂)ₘ—SO₂—CH═CH₂
(CF₃)₂N—CH₂—CH═CH—(CH₂)₁₁—OH; (CF₃)₂N—CH₂—CH═CH—(CH₂)₁₁—(OCH₂CH₂)ₘ—OH;
(CF₃)₂N—CH₂—CH═CH—(CH₂)₁₁—(OCH₂CH₂)ₘ—SO₂—CH═CH₂
(CF₃)₂N—CH₂—CH═CH—(CH₂)₁₂—OH; (CF₃)₂N—CH₂—CH═CH—(CH₂)₁₂—(OCH₂CH₂)ₘ—OH;
(CF₃)₂N—CH₂—CH═CH—(CH₂)₁₂—(OCH₂CH₂)ₘ—SO₂—CH═CH₂
(CF₃)₂N—CH₂—CH═CH—(CH₂)₁₃—OH; (CF₃)₂N—CH₂—CH═CH—(CH₂)₁₃—(OCH₂CH₂)ₘ—OH;
(CF₃)₂N—CH₂—CH═CH—(CH₂)₁₃—(OCH₂CH₂)ₘ—SO₂—CH═CH₂
(CF₃)₂N—CH₂—CH═CH—(CH₂)₁₄—OH; (CF₃)₂N—CH₂—CH═CH—(CH₂)₁₄—(OCH₂CH₂)ₘ—OH;
(CF₃)₂N—CH₂—CH═CH—(CH₂)₁₄—(OCH₂CH₂)ₘ—SO₂—CH═CH₂
(CF₃)₂N—CH₂—CH═CH—(CH₂)₁₅—OH; (CF₃)₂N—CH₂—CH═CH—(CH₂)₁₅—(OCH₂CH₂)ₘ—OH;
(CF₃)₂N—CH₂—CH═CH—(CH₂)₁₅—(OCH₂CH₂)ₘ—SO₂—CH═CH₂
(CF₃)₂N—CH₂—CH═CH—(CH₂)₁₆—OH; (CF₃)₂N—CH₂—CH═CH—(CH₂)₁₆—(OCH₂CH₂)ₘ—OH;
(CF₃)₂N—CH₂—CH═CH—(CH₂)₁₆—(OCH₂CH₂)ₘ—SO₂—CH═CH₂
(CF₃)₂N—CH═CH—(CH₂)₄—SH; (CF₃)₂N—CH═CH—(CH₂)₄—(OCH₂CH₂)ₘ—OH;
(CF₃)₂N—CH═CH—(CH₂)₄—(OCH₂CH₂)ₘ—SO₂—CH═CH₂
(CF₃)₂N—CH═CH—(CH₂)₅—SH; (CF₃)₂N—CH═CH—(CH₂)₅—(OCH₂CH₂)ₘ—OH;
(CF₃)₂N—CH═CH—(CH₂)₅—(OCH₂CH₂)ₘ—SO₂—CH═CH₂
(CF₃)₂N—CH═CH—(CH₂)₆—SH; (CF₃)₂N—CH═CH—(CH₂)₆—(OCH₂CH₂)ₘ—OH;
(CF₃)₂N—CH═CH—(CH₂)₆—(OCH₂CH₂)ₘ—SO₂—CH═CH₂
(CF₃)₂N—CH═CH—(CH₂)₇—SH; (CF₃)₂N—CH═CH—(CH₂)₇—(OCH₂CH₂)ₘ—OH;
(CF₃)₂N—CH═CH—(CH₂)₇—(OCH₂CH₂)ₘ—SO₂—CH═CH₂
(CF₃)₂N—CH═CH—(CH₂)₈—SH; (CF₃)₂N—CH═CH—(CH₂)₈—(OCH₂CH₂)ₘ—OH;
(CF₃)₂N—CH═CH—(CH₂)₈—(OCH₂CH₂)ₘ—SO₂—CH═CH₂
(CF₃)₂N—CH═CH—(CH₂)₉—SH; (CF₃)₂N—CH═CH—(CH₂)₉—(OCH₂CH₂)ₘ—OH;
(CF₃)₂N—CH═CH—(CH₂)₉—(OCH₂CH₂)ₘ—SO₂—CH═CH₂
(CF₃)₂N—CH═CH—(CH₂)₁₀—SH; (CF₃)₂N—CH═CH—(CH₂)₁₀—(OCH₂CH₂)ₘ—OH;
(CF₃)₂N—CH═CH—(CH₂)₁₀—(OCH₂CH₂)ₘ—SO₂—CH═CH₂
(CF₃)₂N—CH═CH—(CH₂)₁₁—SH; (CF₃)₂N—CH═CH—(CH₂)₁₁—(OCH₂CH₂)ₘ—OH;
(CF₃)₂N—CH═CH—(CH₂)₁₁—(OCH₂CH₂)ₘ—SO₂—CH═CH₂
(CF₃)₂N—CH═CH—(CH₂)₁₂—SH; (CF₃)₂N—CH═CH—(CH₂)₁₂—(OCH₂CH₂)ₘ—OH;
(CF₃)₂N—CH═CH—(CH₂)₁₂—(OCH₂CH₂)ₘ—SO₂—CH═CH₂
(CF₃)₂N—CH═CH—(CH₂)₁₃—SH; (CF₃)₂N—CH═CH—(CH₂)₁₃—(OCH₂CH₂)ₘ—OH;
(CF₃)₂N—CH═CH—(CH₂)₁₃—(OCH₂CH₂)ₘ—SO₂—CH═CH₂
(CF₃)₂N—CH═CH—(CH₂)₁₄—SH; (CF₃)₂N—CH═CH—(CH₂)₁₄—(OCH₂CH₂)ₘ—OH;
(CF₃)₂N—CH═CH—(CH₂)₁₄—(OCH₂CH₂)ₘ—SO₂—CH═CH₂
(CF₃)₂N—CH═CH—(CH₂)₁₅—SH; (CF₃)₂N—CH═CH—(CH₂)₁₅—(OCH₂CH₂)ₘ—OH;
(CF₃)₂N—CH═CH—(CH₂)₁₅—(OCH₂CH₂)ₘ—SO₂—CH═CH₂
(CF₃)₂N—CH═CH—(CH₂)₁₆—SH; (CF₃)₂N—CH═CH—(CH₂)₁₆—(OCH₂CH₂)ₘ—OH;
(CF₃)₂N—CH═CH—(CH₂)₁₆—(OCH₂CH₂)ₘ—SO₂—CH═CH₂
(CF₃)₂N—CH₂CHBr—(CH₂)₄—OH; (CF₃)₂N—CH₂CHBr—(CH₂)₄—(OCH₂CH₂)ₘ—OH;
(CF₃)₂N—CH₂CHBr—(CH₂)₄—(OCH₂CH₂)ₘ—SO₂—CH═CH₂
(CF₃)₂N—CH₂CHBr—(CH₂)₅—OH; (CF₃)₂N—CH₂CHBr—(CH₂)₅—(OCH₂CH₂)ₘ—OH;
(CF₃)₂N—CH₂CHBr—(CH₂)₅—(OCH₂CH₂)ₘ—SO₂—CH═CH₂
(CF₃)₂N—CH₂CHBr—(CH₂)₆—OH; (CF₃)₂N—CH₂CHBr—(CH₂)₆—(OCH₂CH₂)ₘ—OH;
(CF₃)₂N—CH₂CHBr—(CH₂)₆—(OCH₂CH₂)ₘ—SO₂—CH═CH₂
(CF₃)₂N—CH₂CHBr—(CH₂)₇—OH; (CF₃)₂N—CH₂CHBr—(CH₂)₇—(OCH₂CH₂)ₘ—OH;
(CF₃)₂N—CH₂CHBr—(CH₂)₇—(OCH₂CH₂)ₘ—SO₂—CH═CH₂
(CF₃)₂N—CH₂CHBr—(CH₂)₈—OH; (CF₃)₂N—CH₂CHBr—(CH₂)₈—(OCH₂CH₂)ₘ—OH;
(CF₃)₂N—CH₂CHBr—(CH₂)₈—(OCH₂CH₂)ₘ—SO₂—CH═CH₂
(CF₃)₂N—CH₂CHBr—(CH₂)₉—OH; (CF₃)₂N—CH₂CHBr—(CH₂)₉—(OCH₂CH₂)ₘ—OH;
(CF₃)₂N—CH₂CHBr—(CH₂)₉—(OCH₂CH₂)ₘ—SO₂—CH═CH₂
(CF₃)₂N—CH₂CHBr—(CH₂)₁₀—OH; (CF₃)₂N—CH₂CHBr—(CH₂)₁₀—(OCH₂CH₂)ₘ—OH;
(CF₃)₂N—CH₂CHBr—(CH₂)₁₀—(OCH₂CH₂)ₘ—SO₂—CH═CH₂
(CF₃)₂N—CH₂CHBr—(CH₂)₁₁—OH; (CF₃)₂N—CH₂CHBr—(CH₂)₁₁—(OCH₂CH₂)ₘ—OH;
(CF₃)₂N—CH₂CHBr—(CH₂)₁₁—(OCH₂CH₂)ₘ—SO₂—CH═CH₂
(CF₃)₂N—CH₂CHBr—(CH₂)₁₂—OH; (CF₃)₂N—CH₂CHBr—(CH₂)₁₂—(OCH₂CH₂)ₘ—OH;
(CF₃)₂N—CH₂CHBr—(CH₂)₁₂—(OCH₂CH₂)ₘ—SO₂—CH═CH₂
(CF₃)₂N—CH₂CHBr—(CH₂)₁₃—OH; (CF₃)₂N—CH₂CHBr—(CH₂)₁₃—(OCH₂CH₂)ₘ—OH;
(CF₃)₂N—CH₂CHBr—(CH₂)₁₃—(OCH₂CH₂)ₘ—SO₂—CH═CH₂
(CF₃)₂N—CH₂CHBr—(CH₂)₁₄—OH; (CF₃)₂N—CH₂CHBr—(CH₂)₁₄—(OCH₂CH₂)ₘ—OH;
(CF₃)₂N—CH₂CHBr—(CH₂)₁₄—(OCH₂CH₂)ₘ—SO₂—CH═CH₂
(CF₃)₂N—CH₂CHBr—(CH₂)₁₅—OH; (CF₃)₂N—CH₂CHBr—(CH₂)₁₅—(OCH₂CH₂)ₘ—OH;
(CF₃)₂N—CH₂CHBr—(CH₂)₁₅—(OCH₂CH₂)ₘ—SO₂—CH═CH₂
(CF₃)₂N—CH₂CHBr—(CH₂)₁₆—OH; (CF₃)₂N—CH₂CHBr—(CH₂)₁₆—(OCH₂CH₂)ₘ—OH;
(CF₃)₂N—CH₂CHBr—(CH₂)₁₆—(OCH₂CH₂)ₘ—SO₂—CH═CH₂
CF₃—S—(CH₂)₄—(OCH₂CH₂)ₘ—OCOCH═CH₂; CF₃—S—(CH₂)₄—(OCH₂CH₂)ₘ—OCH═CH₂;
CF₃—S—(CH₂)₄—(OCH₂CH₂)ₘ—OAr(NCO)ₚ
CF₃—S—(CH₂)₅—(OCH₂CH₂)ₘ—OCOCH═CH₂; CF₃—S—(CH₂)₅—(OCH₂CH₂)ₘ—OCH═CH₂;
CF₃—S—(CH₂)₅—(OCH₂CH₂)ₘ—OAr(NCO)ₚ
CF₃—S—(CH₂)₆—(OCH₂CH₂)ₘ—OCOCH═CH₂; CF₃—S—(CH₂)₆—(OCH₂CH₂)ₘ—OCH═CH₂;
CF₃—S—(CH₂)₆—(OCH₂CH₂)ₘ—OAr(NCO)ₚ
CF₃—S—(CH₂)₇—(OCH₂CH₂)ₘ—OCOCH═CH₂; CF₃—S—(CH₂)₇—(OCH₂CH₂)ₘ—OCH═CH₂;
CF₃—S—(CH₂)₇—(OCH₂CH₂)ₘ—OAr(NCO)ₚ
CF₃—S—(CH₂)₈—(OCH₂CH₂)ₘ—OCOCH═CH₂; CF₃—S—(CH₂)₈—(OCH₂CH₂)ₘ—OCH═CH₂;
CF₃—S—(CH₂)₈—(OCH₂CH₂)ₘ—OAr(NCO)ₚ
CF₃—S—(CH₂)₉—(OCH₂CH₂)ₘ—OCOCH═CH₂; CF₃—S—(CH₂)₉—(OCH₂CH₂)ₘ—OCH═CH₂;
CF₃—S—(CH₂)₉—(OCH₂CH₂)ₘ—OAr(NCO)ₚ
CF₃—S—(CH₂)₁₀—(OCH₂CH₂)ₘ—OCOCH═CH₂; CF₃—S—(CH₂)₁₀—(OCH₂CH₂)ₘ—OCH═CH₂;
CF₃—S—(CH₂)₁₀—(OCH₂CH₂)ₘ—OAr(NCO)ₚ
CF₃—S—(CH₂)₁₁—(OCH₂CH₂)ₘ—OCOCH═CH₂; CF₃—S—(CH₂)₁₁—(OCH₂CH₂)ₘ—OCH═CH₂;
CF₃—S—(CH₂)₁₁—(OCH₂CH₂)ₘ—OAr(NCO)ₚ
CF₃—S—(CH₂)₁₂—(OCH₂CH₂)ₘ—OCOCH═CH₂; CF₃—S—(CH₂)₁₂—(OCH₂CH₂)ₘ—OCH═CH₂;
CF₃—S—(CH₂)₁₂—(OCH₂CH₂)ₘ—OAr(NCO)ₚ
CF₃—S—(CH₂)₁₃—(OCH₂CH₂)ₘ—OCOCH═CH₂; CF₃—S—(CH₂)₁₃—(OCH₂CH₂)ₘ—OCH═CH₂;
CF₃—S—(CH₂)₁₃—(OCH₂CH₂)ₘ—OAr(NCO)ₚ
CF₃—S—(CH₂)₁₄—(OCH₂CH₂)ₘ—OCOCH═CH₂; CF₃—S—(CH₂)₁₄—(OCH₂CH₂)ₘ—OCH═CH₂;
CF₃—S—(CH₂)₁₄—(OCH₂CH₂)ₘ—OAr(NCO)ₚ
CF₃—S—(CH₂)₁₅—(OCH₂CH₂)ₘ—OCOCH═CH₂; CF₃—S—(CH₂)₁₅—(OCH₂CH₂)ₘ—OCH═CH₂;
CF₃—S—(CH₂)₁₅—(OCH₂CH₂)ₘ—OAr(NCO)ₚ
CF₃—S—(CH₂)₁₆—(OCH₂CH₂)ₘ—OCOCH═CH₂; CF₃—S—(CH₂)₁₆—(OCH₂CH₂)ₘ—OCH═CH₂;
CF₃—S—(CH₂)₁₆—(OCH₂CH₂)ₘ—OAr(NCO)ₚ
(CF₃)₂N—(CH₂)₄—(OCH₂CH₂)ₘ—OCOCH═CH₂; (CF₃)₂N—(CH₂)₄—(OCH₂CH₂)ₘ—OCH═CH₂;
(CF₃)₂N—(CH₂)₄—(OCH₂CH₂)ₘ—OAr(NCO)ₚ
(CF₃)₂N—(CH₂)₅—(OCH₂CH₂)ₘ—OCOCH═CH₂; (CF₃)₂N—(CH₂)₅—(OCH₂CH₂)ₘ—OCH═CH₂;
(CF₃)₂N—(CH₂)₅—(OCH₂CH₂)ₘ—OAr(NCO)ₚ
(CF₃)₂N—(CH₂)₆—(OCH₂CH₂)ₘ—OCOCH═CH₂; (CF₃)₂N—(CH₂)₆—(OCH₂CH₂)ₘ—OCH═CH₂;
(CF₃)₂N—(CH₂)₆—(OCH₂CH₂)ₘ—OAr(NCO)ₚ
(CF₃)₂N—(CH₂)₇—(OCH₂CH₂)ₘ—OCOCH═CH₂; (CF₃)₂N—(CH₂)₇—(OCH₂CH₂)ₘ—OCH═CH₂;
(CF₃)₂N—(CH₂)₇—(OCH₂CH₂)ₘ—OAr(NCO)ₚ
(CF₃)₂N—(CH₂)₈—(OCH₂CH₂)ₘ—OCOCH═CH₂; (CF₃)₂N—(CH₂)₈—(OCH₂CH₂)ₘ—OCH═CH₂;
(CF₃)₂N—(CH₂)₈—(OCH₂CH₂)ₘ—OAr(NCO)ₚ
(CF₃)₂N—(CH₂)₉—(OCH₂CH₂)ₘ—OCOCH═CH₂; (CF₃)₂N—(CH₂)₉—(OCH₂CH₂)ₘ—OCH═CH₂;
(CF₃)₂N—(CH₂)₉—(OCH₂CH₂)ₘ—OAr(NCO)ₚ
(CF₃)₂N—(CH₂)₁₀—(OCH₂CH₂)ₘ—OCOCH═CH₂; (CF₃)₂N—(CH₂)₁₀—(OCH₂CH₂)ₘ—OCH═CH₂;
(CF₃)₂N—(CH₂)₁₀—(OCH₂CH₂)ₘ—OAr(NCO)ₚ
(CF₃)₂N—(CH₂)₁₁—(OCH₂CH₂)ₘ—OCOCH═CH₂; (CF₃)₂N—(CH₂)₁₁—(OCH₂CH₂)ₘ—OCH═CH₂;
(CF₃)₂N—(CH₂)₁₁—(OCH₂CH₂)ₘ—OAr(NCO)ₚ
(CF₃)₂N—(CH₂)₁₂—(OCH₂CH₂)ₘ—OCOCH═CH₂; (CF₃)₂N—(CH₂)₁₂—(OCH₂CH₂)ₘ—OCH═CH₂;
(CF₃)₂N—(CH₂)₁₂—(OCH₂CH₂)ₘ—OAr(NCO)ₚ
(CF₃)₂N—(CH₂)₁₃—(OCH₂CH₂)ₘ—OCOCH═CH₂; (CF₃)₂N—(CH₂)₁₃—(OCH₂CH₂)ₘ—OCH═CH₂;
(CF₃)₂N—(CH₂)₁₃—(OCH₂CH₂)ₘ—OAr(NCO)ₚ
(CF₃)₂N—(CH₂)₁₄—(OCH₂CH₂)ₘ—OCOCH═CH₂; (CF₃)₂N—(CH₂)₁₄—(OCH₂CH₂)ₘ—OCH═CH₂;
(CF₃)₂N—(CH₂)₁₄—(OCH₂CH₂)ₘ—OAr(NCO)ₚ
(CF₃)₂N—(CH₂)₁₅—(OCH₂CH₂)ₘ—OCOCH═CH₂; (CF₃)₂N—(CH₂)₁₅—(OCH₂CH₂)ₘ—OCH═CH₂;
(CF₃)₂N—(CH₂)₁₅—(OCH₂CH₂)ₘ—OAr(NCO)ₚ
(CF₃)₂N—(CH₂)₁₆—(OCH₂CH₂)ₘ—OCOCH═CH₂; (CF₃)₂N—(CH₂)₁₆—(OCH₂CH₂)ₘ—OCH═CH₂;
(CF₃)₂N—(CH₂)₁₆—(OCH₂CH₂)ₘ—OAr(NCO)ₚ
(CF₃)₂N—CH₂—CH═CH—(CH₂)₄—(OCH₂CH₂)ₘ—OCOCH═CH₂; (CF₃)₂N—CH₂—CH═CH—(CH₂)₄—(OCH₂CH₂)ₘ—OCH═CH₂;
(CF₃)₂N—CH₂—CH═CH—(CH₂)₄—(OCH₂CH₂)ₘ—OAr(NCO)_{p;}
(CF₃)₂N—CH₂—CH═CH—(CH₂)₄—(OCH₂CH₂)ₘ—SH
(CF₃)₂N—CH₂—CH═CH—(CH₂)₅—(OCH₂CH₂)ₘ—OCOCH═CH₂; (CF₃)₂N—CH₂—CH═CH—(CH₂)₅—(OCH₂CH₂)ₘ—OCH═CH₂;
(CF₃)₂N—CH₂—CH═CH—(CH₂)₅—(OCH₂CH₂)ₘ—OAr(NCO)_{p;}
(CF₃)₂N—CH₂—CH═CH—(CH₂)₅—(OCH₂CH₂)ₘ—SH
(CF₃)₂N—CH₂—CH═CH—(CH₂)₆—(OCH₂CH₂)ₘ—OCOCH═CH₂; (CF₃)₂N—CH₂—CH═CH—(CH₂)₆—(OCH₂CH₂)ₘ—OCH═CH₂;
(CF₃)₂N—CH₂—CH═CH—(CH₂)₆—(OCH₂CH₂)ₘ—OAr(NCO)_{p;}
(CF₃)₂N—CH₂—CH═CH—(CH₂)₆—(OCH₂CH₂)ₘ—SH
(CF₃)₂N—CH₂—CH═CH—(CH₂)₇—(OCH₂CH₂)ₘ—OCOCH═CH₂; (CF₃)₂N—CH₂—CH═CH—(CH₂)₇—(OCH₂CH₂)ₘ—OCH═CH₂;
(CF₃)₂N—CH₂—CH═CH—(CH₂)₇—(OCH₂CH₂)ₘ—OAr(NCO)_{p;}
(CF₃)₂N—CH₂—CH═CH—(CH₂)₇—(OCH₂CH₂)ₘ—SH
(CF₃)₂N—CH₂—CH═CH—(CH₂)₈—(OCH₂CH₂)ₘ—OCOCH═CH₂; (CF₃)₂N—CH₂—CH═CH—(CH₂)₈—(OCH₂CH₂)ₘ—OCH═CH₂;
(CF₃)₂N—CH₂—CH═CH—(CH₂)₈—(OCH₂CH₂)ₘ—OAr(NCO)_{p;}
(CF₃)₂N—CH₂—CH═CH—(CH₂)₈—(OCH₂CH₂)ₘ—SH
(CF₃)₂N—CH₂—CH═CH—(CH₂)₉—(OCH₂CH₂)ₘ—OCOCH═CH₂; (CF₃)₂N—CH₂—CH═CH—(CH₂)₉—(OCH₂CH₂)ₘ—OCH═CH₂;
(CF₃)₂N—CH₂—CH═CH—(CH₂)₉—(OCH₂CH₂)ₘ—OAr(NCO)_{p;}
(CF₃)₂N—CH₂—CH═CH—(CH₂)₉—(OCH₂CH₂)ₘ—SH
(CF₃)₂N—CH₂—CH═CH—(CH₂)₁₀—(OCH₂CH₂)ₘ—OCOCH═CH₂; (CF₃)₂N—CH₂—CH═CH—(CH₂)₁₀—(OCH₂CH₂)ₘ—OCH═CH₂;
(CF₃)₂N—CH₂—CH═CH—(CH₂)₁₀—(OCH₂CH₂)ₘ—OAr(NCO)_{p;}
(CF₃)₂N—CH₂—CH═CH—(CH₂)₁₀—(OCH₂CH₂)ₘ—SH
(CF₃)₂N—CH₂—CH═CH—(CH₂)₁₁—(OCH₂CH₂)ₘ—OCOCH═CH₂; (CF₃)₂N—CH₂—CH═CH—(CH₂)₁₁—(OCH₂CH₂)ₘ—OCH═CH₂;
(CF₃)₂N—CH₂—CH═CH—(CH₂)₁₁—(OCH₂CH₂)ₘ—OAr(NCO)_{p;}
(CF₃)₂N—CH₂—CH═CH—(CH₂)₁₁—(OCH₂CH₂)ₘ—SH
(CF₃)₂N—CH₂—CH═CH—(CH₂)₁₂—(OCH₂CH₂)ₘ—OCOCH═CH₂; (CF₃)₂N—CH₂—CH═CH—(CH₂)₁₂—(OCH₂CH₂)ₘ—OCH═CH₂;
(CF₃)₂N—CH₂—CH═CH—(CH₂)₁₂—(OCH₂CH₂)ₘ—OAr(NCO)_{p;}
(CF₃)₂N—CH₂—CH═CH—(CH₂)₁₂—(OCH₂CH₂)ₘ—SH
(CF₃)₂N—CH₂—CH═CH—(CH₂)₁₃—(OCH₂CH₂)ₘ—OCOCH═CH₂; (CF₃)₂N—CH₂—CH═CH—(CH₂)₁₃—(OCH₂CH₂)ₘ—OCH═CH₂;
(CF₃)₂N—CH₂—CH═CH—(CH₂)₁₃—(OCH₂CH₂)ₘ—OAr(NCO)_{p;}
(CF₃)₂N—CH₂—CH═CH—(CH₂)₁₃—(OCH₂CH₂)ₘ—SH
(CF₃)₂N—CH₂—CH═CH—(CH₂)₁₄—(OCH₂CH₂)ₘ—OCOCH═CH₂; (CF₃)₂N—CH₂—CH═CH—(CH₂)₁₄—(OCH₂CH₂)ₘ—OCH═CH₂;
(CF₃)₂N—CH₂—CH═CH—(CH₂)₁₄—(OCH₂CH₂)ₘ—OAr(NCO)_{p;}
(CF₃)₂N—CH₂—CH═CH—(CH₂)₁₄—(OCH₂CH₂)ₘ—SH
(CF₃)₂N—CH₂—CH═CH—(CH₂)₁₅—(OCH₂CH₂)ₘ—OCOCH═CH₂; (CF₃)₂N—CH₂—CH═CH—(CH₂)₁₅—(OCH₂CH₂)ₘ—OCH═CH₂;
(CF₃)₂N—CH₂—CH═CH—(CH₂)₁₅—(OCH₂CH₂)ₘ—OAr(NCO)_{p;}
(CF₃)₂N—CH₂—CH═CH—(CH₂)₁₅—(OCH₂CH₂)ₘ—SH
(CF₃)₂N—CH₂—CH═CH—(CH₂)₁₆—(OCH₂CH₂)ₘ—OCOCH═CH₂; (CF₃)₂N—CH₂—CH═CH—(CH₂)₁₆—(OCH₂CH₂)ₘ—OCH═CH₂;
(CF₃)₂N—CH₂—CH═CH—(CH₂)₁₆—(OCH₂CH₂)ₘ—OAr(NCO)_{p;}
(CF₃)₂N—CH₂—CH═CH—(CH₂)₁₆—(OCH₂CH₂)ₘ—SH
(CF₃)₂N—CH₂CHBr—(CH₂)₄—(OCH₂CHR)ₘ—OCOCH═CH₂; (CF₃)₂N—CH₂CHBr—(CH₂)₄—(OCH₂CHR)ₘ—OCH═CH₂;
(CF₃)₂N—CH₂CHBr—(CH₂)₄—(OCH₂CHR)ₘ—OAr(NCO)ₚ
(CF₃)₂N—CH₂CHBr—(CH₂)₅—(OCH₂CHR)ₘ—OCOCH═CH₂; (CF₃)₂N—CH₂CHBr—(CH₂)₅—(OCH₂CHR)ₘ—OCH═CH₂;
(CF₃)₂N—CH₂CHBr—(CH₂)₅—(OCH₂CHR)ₘ—OAr(NCO)ₚ
(CF₃)₂N—CH₂CHBr—(CH₂)₆—(OCH₂CHR)ₘ—OCOCH═CH₂; (CF₃)₂N—CH₂CHBr—(CH₂)₆—(OCH₂CHR)ₘ—OCH═CH₂;
(CF₃)₂N—CH₂CHBr—(CH₂)₆—(OCH₂CHR)ₘ—OAr(NCO)ₚ
(CF₃)₂N—CH₂CHBr—(CH₂)₇—(OCH₂CHR)ₘ—OCOCH═CH₂; (CF₃)₂N—CH₂CHBr—(CH₂)₇—(OCH₂CHR)ₘ—OCH═CH₂;
(CF₃)₂N—CH₂CHBr—(CH₂)₇—(OCH₂CHR)ₘ—OAr(NCO)ₚ
(CF₃)₂N—CH₂CHBr—(CH₂)₈—(OCH₂CHR)ₘ—OCOCH═CH₂; (CF₃)₂N—CH₂CHBr—(CH₂)₈—(OCH₂CHR)ₘ—OCH═CH₂;
(CF₃)₂N—CH₂CHBr—(CH₂)₈—(OCH₂CHR)ₘ—OAr(NCO)ₚ
(CF₃)₂N—CH₂CHBr—(CH₂)₉—(OCH₂CHR)ₘ—OCOCH═CH₂; (CF₃)₂N—CH₂CHBr—(CH₂)₉—(OCH₂CHR)ₘ—OCH═CH₂;
(CF₃)₂N—CH₂CHBr—(CH₂)₉—(OCH₂CHR)ₘ—OAr(NCO)ₚ
(CF₃)₂N—CH₂CHBr—(CH₂)₁₀—(OCH₂CHR)ₘ—OCOCH═CH₂; (CF₃)₂N—CH₂CHBr—(CH₂)₁₀—(OCH₂CHR)ₘ—OCH═CH₂;
(CF₃)₂N—CH₂CHBr—(CH₂)₁₀—(OCH₂CHR)ₘ—OAr(NCO)ₚ
(CF₃)₂N—CH₂CHBr—(CH₂)₁₁—(OCH₂CHR)ₘ—OCOCH═CH₂; (CF₃)₂N—CH₂CHBr—(CH₂)₁₁—(OCH₂CHR)ₘ—OCH═CH₂;
(CF₃)₂N—CH₂CHBr—(CH₂)₁₁—(OCH₂CHR)ₘ—OAr(NCO)ₚ
(CF₃)₂N—CH₂CHBr—(CH₂)₁₂—(OCH₂CHR)ₘ—OCOCH═CH₂; (CF₃)₂N—CH₂CHBr—(CH₂)₁₂—(OCH₂CHR)ₘ—OCH═CH₂;
(CF₃)₂N—CH₂CHBr—(CH₂)₁₂—(OCH₂CHR)ₘ—OAr(NCO)ₚ
(CF₃)₂N—CH₂CHBr—(CH₂)₁₃—(OCH₂CHR)ₘ—OCOCH═CH₂; (CF₃)₂N—CH₂CHBr—(CH₂)₁₃—(OCH₂CHR)ₘ—OCH═CH₂;
(CF₃)₂N—CH₂CHBr—(CH₂)₁₃—(OCH₂CHR)ₘ—OAr(NCO)ₚ
(CF₃)₂N—CH₂CHBr—(CH₂)₁₄—(OCH₂CHR)ₘ—OCOCH═CH₂; (CF₃)₂N—CH₂CHBr—(CH₂)₁₄—(OCH₂CHR)ₘ—OCH═CH₂;
(CF₃)₂N—CH₂CHBr—(CH₂)₁₄—(OCH₂CHR)ₘ—OAr(NCO)ₚ
(CF₃)₂N—CH₂CHBr—(CH₂)₁₅—(OCH₂CHR)ₘ—OCOCH═CH₂; (CF₃)₂N—CH₂CHBr—(CH₂)₁₅—(OCH₂CHR)ₘ—OCH═CH₂;
(CF₃)₂N—CH₂CHBr—(CH₂)₁₅—(OCH₂CHR)ₘ—OAr(NCO)ₚ
(CF₃)₂N—CH₂CHBr—(CH₂)₁₆—(OCH₂CHR)ₘ—OCOCH═CH₂; (CF₃)₂N—CH₂CHBr—(CH₂)₁₆—(OCH₂CHR)ₘ—OCH═CH₂;
(CF₃)₂N—CH₂CHBr—(CH₂)₁₆—(OCH₂CHR)ₘ—OAr(NCO)ₚ
CF₃—S—(CH₂)₄—N⁺R¹R²R³Z⁻; CF₃—S—(CH₂)₄—P⁺R¹R²R³Z⁻; CF₃—S—(CH₂)₄—O-glucoside
CF₃—S—(CH₂)₅—N⁺R¹R²R³Z⁻; CF₃—S—(CH₂)₅—P⁺R¹R²R³Z⁻; CF₃—S—(CH₂)₅—O-glucoside
CF₃—S—(CH₂)₆—N⁺R¹R²R³Z⁻; CF₃—S—(CH₂)₆—P⁺R¹R²R³Z⁻; CF₃—S—(CH₂)₆—O-glucoside
CF₃—S—(CH₂)₇—N⁺R¹R²R³Z⁻; CF₃—S—(CH₂)₇—P⁺R¹R²R³Z⁻; CF₃—S—(CH₂)₇—O-glucoside
CF₃—S—(CH₂)₈—N⁺R¹R²R³Z⁻; CF₃—S—(CH₂)₈—P⁺R¹R²R³Z⁻; CF₃—S—(CH₂)₈—O-glucoside
CF₃—S—(CH₂)₉—N⁺R¹R²R³Z⁻; CF₃—S—(CH₂)₉—P⁺R¹R²R³Z⁻; CF₃—S—(CH₂)₉—O-glucoside
CF₃—S—(CH₂)₁₀—N⁺R¹R²R³Z⁻; CF₃—S—(CH₂)₁₀—P⁺R¹R²R³Z⁻; CF₃—S—(CH₂)₁₀—O-glucoside
CF₃—S—(CH₂)₁₁—N⁺R¹R²R³Z⁻; CF₃—S—(CH₂)₁₁—P⁺R¹R²R³Z⁻; CF₃—S—(CH₂)₁₁—O-glucoside
CF₃—S—(CH₂)₁₂—N⁺R¹R²R³Z⁻; CF₃—S—(CH₂)₁₂—P⁺R¹R²R³Z⁻; CF₃—S—(CH₂)₁₂—O-glucoside
CF₃—S—(CH₂)₁₃—N⁺R¹R²R³Z⁻; CF₃—S—(CH₂)₁₃—P⁺R¹R²R³Z⁻; CF₃—S—(CH₂)₁₃—O-glucoside
CF₃—S—(CH₂)₁₄—N⁺R¹R²R³Z⁻; CF₃—S—(CH₂)₁₄—P⁺R¹R²R³Z⁻; CF₃—S—(CH₂)₁₄—O-glucoside
CF₃—S—(CH₂)₁₅—N⁺R¹R²R³Z⁻; CF₃—S—(CH₂)₁₅—P⁺R¹R²R³Z⁻; CF₃—S—(CH₂)₁₅—O-glucoside
CF₃—S—(CH₂)₁₆—N⁺R¹R²R³Z⁻; CF₃—S—(CH₂)₁₆—P⁺R¹R²R³Z⁻; CF₃—S—(CH₂)₁₆—O-glucoside
(CF₃)₂N—(CH₂)₄—N⁺R¹R²R³Z⁻; (CF₃)₂N—(CH₂)₄—P⁺R¹R²R³Z⁻; (CF₃)₂N—(CH₂)₄—O-
glucoside
(CF₃)₂N—(CH₂)₅—N⁺R¹R²R³Z⁻; (CF₃)₂N—(CH₂)₅—P⁺R¹R²R³Z⁻; (CF₃)₂N—(CH₂)₅—O-
glucoside
(CF₃)₂N—(CH₂)₆—N⁺R¹R²R³Z⁻; (CF₃)₂N—(CH₂)₆—P⁺R¹R²R³Z⁻; (CF₃)₂N—(CH₂)₆—O-
glucoside
(CF₃)₂N—(CH₂)₇—N⁺R¹R²R³Z⁻; (CF₃)₂N—(CH₂)₇—P⁺R¹R²R³Z⁻; (CF₃)₂N—(CH₂)₇—O-
glucoside
(CF₃)₂N—(CH₂)₈—N⁺R¹R²R³Z⁻; (CF₃)₂N—(CH₂)₈—P⁺R¹R²R³Z⁻; (CF₃)₂N—(CH₂)₈—O-
glucoside
(CF₃)₂N—(CH₂)₉—N⁺R¹R²R³Z⁻; (CF₃)₂N—(CH₂)₉—P⁺R¹R²R³Z⁻; (CF₃)₂N—(CH₂)₉—O-
glucoside
(CF₃)₂N—(CH₂)₁₀—N⁺R¹R²R³Z⁻; (CF₃)₂N—(CH₂)₁₀—P⁺R¹R²R³Z⁻; (CF₃)₂N—(CH₂)₁₀—O-
glucoside
(CF₃)₂N—(CH₂)₁₁—N⁺R¹R²R³Z⁻; (CF₃)₂N—(CH₂)₁₁—P⁺R¹R²R³Z⁻; (CF₃)₂N—(CH₂)₁₁—O-
glucoside
(CF₃)₂N—(CH₂)₁₂—N⁺R¹R²R³Z⁻; (CF₃)₂N—(CH₂)₁₂—P⁺R¹R²R³Z⁻; (CF₃)₂N—(CH₂)₁₂—O-
glucoside
(CF₃)₂N—(CH₂)₁₃—N⁺R¹R²R³Z⁻; (CF₃)₂N—(CH₂)₁₃—P⁺R¹R²R³Z⁻; (CF₃)₂N—(CH₂)₁₃—O-
glucoside
(CF₃)₂N—(CH₂)₁₄—N⁺R¹R²R³Z⁻; (CF₃)₂N—(CH₂)₁₄—P⁺R¹R²R³Z⁻; (CF₃)₂N—(CH₂)₁₄—O-
glucoside
(CF₃)₂N—(CH₂)₁₅—N⁺R¹R²R³Z⁻; (CF₃)₂N—(CH₂)₁₅—P⁺R¹R²R³Z⁻; (CF₃)₂N—(CH₂)₁₅—O-
glucoside
(CF₃)₂N—(CH₂)₁₆—N⁺R¹R²R³Z⁻; (CF₃)₂N—(CH₂)₁₆—P⁺R¹R²R³Z⁻; (CF₃)₂N—(CH₂)₁₆—O-
glucoside
(CF₃)₂N—CH₂—CH═CH—(CH₂)₄—N⁺R¹R²R³Z⁻; (CF₃)₂N—CH₂—CH═CH—(CH₂)₄—P⁺R¹R²R₃Z⁻;
(CF₃)₂N—CH₂—CH═CH—(CH₂)₄—O-glucoside; (CF₃)₂N—CH₂—CH═CH—(CH₂)₄—OPO₃H
(CF₃)₂N—CH₂—CH═CH—(CH₂)₅—N⁺R¹R²R³Z⁻; (CF₃)₂N—CH₂—CH═CH—(CH₂)₅—P⁺R¹R²R³Z⁻;
(CF₃)₂N—CH₂—CH═CH—(CH₂)₅—O-glucoside;; (CF₃)₂N—CH₂—CH═CH—(CH₂)₅—OPO₃H
(CF₃)₂N—CH₂—CH═CH—(CH₂)₆—N⁺R¹R²R³Z⁻; (CF₃)₂N—CH₂—CH═CH—(CH₂)₆—P⁺R¹R²R³Z⁻;
(CF₃)₂N—CH₂—CH═CH—(CH₂)₆—O-glucoside;; (CF₃)₂N—CH₂—CH═CH—(CH₂)₆—OPO₃H
(CF₃)₂N—CH₂—CH═CH—(CH₂)₇—N⁺R¹R²R³Z⁻; (CF₃)₂N—CH₂—CH═CH—(CH₂)₇—P⁺R¹R²R³Z⁻;
(CF₃)₂N—CH₂—CH═CH—(CH₂)₇—O-glucoside;; (CF₃)₂N—CH₂—CH═CH—(CH₂)₇—OPO₃H
(CF₃)₂N—CH₂—CH═CH—(CH₂)₈—N⁺R¹R²R³Z⁻; (CF₃)₂N—CH₂—CH═CH—(CH₂)₈—P⁺R¹R²R³Z⁻;
(CF₃)₂N—CH₂—CH═CH—(CH₂)₈—O-glucoside;; (CF₃)₂N—CH₂—CH═CH—(CH₂)₈—OPO₃H
(CF₃)₂N—CH₂—CH═CH—(CH₂)₉—N⁺R¹R²R³Z⁻; (CF₃)₂N—CH₂—CH═CH—(CH₂)₉—P⁺R¹R²R³Z⁻;
(CF₃)₂N—CH₂—CH═CH—(CH₂)₉—O-glucoside;; (CF₃)₂N—CH₂—CH═CH—(CH₂)₉—OPO₃H
(CF₃)₂N—CH₂—CH═CH—(CH₂)₁₀—N⁺R¹R²R³Z⁻; (CF₃)₂N—CH₂—CH═CH—(CH₂)₁₀—P⁺R¹R²R³Z⁻;
(CF₃)₂N—CH₂—CH═CH—(CH₂)₁₀—O-glucoside;; (CF₃)₂N—CH₂—CH═CH—(CH₂)₁₀—OPO₃H
(CF₃)₂N—CH₂—CH═CH—(CH₂)₁₁—N⁺R¹R²R³Z⁻; (CF₃)₂N—CH₂—CH═CH—(CH₂)₁₁—P⁺R¹R²R³Z⁻;
(CF₃)₂N—CH₂—CH═CH—(CH₂)₁₁—O-glucoside;; (CF₃)₂N—CH₂—CH═CH—(CH₂)₁₁—OPO₃H
(CF₃)₂N—CH₂—CH═CH—(CH₂)₁₂—N⁺R¹R²R³Z⁻; (CF₃)₂N—CH₂—CH═CH—(CH₂)₁₂—P⁺R¹R²R³Z⁻;
(CF₃)₂N—CH₂—CH═CH—(CH₂)₁₂—O-glucoside;; (CF₃)₂N—CH₂—CH═CH—(CH₂)₁₂—OPO₃H
(CF₃)₂N—CH₂—CH═CH—(CH₂)₁₃—N⁺R¹R²R³Z⁻; (CF₃)₂N—CH₂—CH═CH—(CH₂)₁₃—P⁺R¹R²R³Z⁻;
(CF₃)₂N—CH₂—CH═CH—(CH₂)₁₃—O-glucoside;; (CF₃)₂N—CH₂—CH═CH—(CH₂)₁₃—OPO₃H
(CF₃)₂N—CH₂—CH═CH—(CH₂)₁₄—N⁺R¹R²R³Z⁻; (CF₃)₂N—CH₂—CH═CH—(CH₂)₁₄—P⁺R¹R²R³Z⁻;
(CF₃)₂N—CH₂—CH═CH—(CH₂)₁₄—O-glucoside;; (CF₃)₂N—CH₂—CH═CH—(CH₂)₁₄—OPO₃H
(CF₃)₂N—CH₂—CH═CH—(CH₂)₁₅—N⁺R¹R²R³Z⁻; (CF₃)₂N—CH₂—CH═CH—(CH₂)₁₅—P⁺R¹R²R³Z⁻;
(CF₃)₂N—CH₂—CH═CH—(CH₂)₁₅—O-glucoside;; (CF₃)₂N—CH₂—CH═CH—(CH₂)₁₅—OPO₃H
(CF₃)₂N—CH₂—CH═CH—(CH₂)₁₆—N⁺R¹R²R³Z⁻; (CF₃)₂N—CH₂—CH═CH—(CH₂)₁₆—P⁺R¹R²R³Z⁻;
(CF₃)₂N—CH₂—CH═CH—(CH₂)₁₆—O-glucoside;; (CF₃)₂N—CH₂—CH═CH—(CH₂)₁₆—OPO₃H
CF₃S—CH₂CHBr—(CH₂)₄—N⁺R¹R²R³Z⁻; CF₃S—CH₂CHBr—(CH₂)₄—P⁺R¹R²R³Z⁻; CF₃S—CH₂CHBr—(CH₂)₄—O-
glucoside; CF₃S—CH₂CHBr—(CH₂)₄—SH
CF₃S—CH₂CHBr—(CH₂)₅—N⁺R¹R²R³Z⁻; CF₃S—CH₂CHBr—(CH₂)₅—P⁺R¹R²R³Z⁻; CF₃S—CH₂CHBr—(CH₂)₅—O-
glucoside; CF₃S—CH₂CHBr—(CH₂)₅—SH
CF₃S—CH₂CHBr—(CH₂)₆—N⁺R¹R²R³Z⁻; CF₃S—CH₂CHBr—(CH₂)₆—P⁺R¹R²R³Z⁻; CF₃S—CH₂CHBr—(CH₂)₆—O-
glucoside; CF₃S—CH₂CHBr—(CH₂)₆—SH
CF₃S—CH₂CHBr—(CH₂)₇—N⁺R¹R²R³Z⁻; CF₃S—CH₂CHBr—(CH₂)₇—P⁺R¹R²R³Z⁻; CF₃S—CH₂CHBr—(CH₂)₇—O-
glucoside; CF₃S—CH₂CHBr—(CH₂)₇—SH
CF₃S—CH₂CHBr—(CH₂)₈—N⁺R¹R²R³Z⁻; CF₃S—CH₂CHBr—(CH₂)₈—P⁺R¹R²R³Z⁻; CF₃S—CH₂CHBr—(CH₂)₈—O-
glucoside; CF₃S—CH₂CHBr—(CH₂)₈—SH
CF₃S—CH₂CHBr—(CH₂)₉—N⁺R¹R²R³Z⁻; CF₃S—CH₂CHBr—(CH₂)₉—P⁺R¹R²R³Z⁻; CF₃S—CH₂CHBr—(CH₂)₉—O-
glucoside; CF₃S—CH₂CHBr—(CH₂)₉—SH
CF₃S—CH₂CHBr—(CH₂)₁₀—N⁺R¹R²R³Z⁻; CF₃S—CH₂CHBr—(CH₂)₁₀—P⁺R¹R²R³Z⁻; CF₃S—CH₂CHBr—(CH₂)₁₀—O-
glucoside; CF₃S—CH₂CHBr—(CH₂)₁₀—SH
CF₃S—CH₂CHBr—(CH₂)₁₁—N⁺R¹R²R³Z⁻; CF₃S—CH₂CHBr—(CH₂)₁₁—P⁺R¹R²R³Z⁻; CF₃S—CH₂CHBr—(CH₂)₁₁—O-
glucoside; CF₃S—CH₂CHBr—(CH₂)₁₁—SH
CF₃S—CH₂CHBr—(CH₂)₁₂—N⁺R¹R²R³Z⁻; CF₃S—CH₂CHBr—(CH₂)₁₂—P⁺R¹R²R³Z⁻; CF₃S—CH₂CHBr—(CH₂)₁₂—O-
glucoside; CF₃S—CH₂CHBr—(CH₂)₁₂—SH
CF₃S—CH₂CHBr—(CH₂)₁₃—N⁺R¹R²R³Z⁻; CF₃S—CH₂CHBr—(CH₂)₁₃—P⁺R¹R²R³Z⁻; CF₃S—CH₂CHBr—(CH₂)₁₃—O-
glucoside; CF₃S—CH₂CHBr—(CH₂)₁₃—SH
CF₃S—CH₂CHBr—(CH₂)₁₄—N⁺R¹R²R³Z⁻; CF₃S—CH₂CHBr—(CH₂)₁₄—P⁺R¹R²R³Z⁻; CF₃S—CH₂CHBr—(CH₂)₁₄—O-
glucoside; CF₃S—CH₂CHBr—(CH₂)₁₄—SH
CF₃S—CH₂CHBr—(CH₂)₁₅—N⁺R¹R²R³Z⁻; CF₃S—CH₂CHBr—(CH₂)₁₅—P⁺R¹R²R³Z⁻; CF₃S—CH₂CHBr—(CH₂)₁₅—O-
glucoside; CF₃S—CH₂CHBr—(CH₂)₁₅—SH
CF₃S—CH₂CHBr—(CH₂)₁₆—N⁺R¹R²R³Z⁻; CF₃S—CH₂CHBr—(CH₂)₁₆—P⁺R¹R²R³Z⁻; CF₃S—CH₂CHBr—(CH₂)₁₆—O-
glucoside; CF₃S—CH₂CHBr—(CH₂)₁₆—SH
(CF₃—CH₂—CH₂)₂N—CH₂CHBr—(CH₂)₄—N⁺R¹R²R³Z⁻; (CF₃—CH₂—CH₂)₂N—CH₂CHBr—(CH₂)₄—P⁺R¹R²R³Z⁻;
(CF₃—CH₂—CH₂)₂N—CH₂CHBr—(CH₂)₄—O-glucoside;
(CF₃—CH₂—CH₂)₂N—CH₂CHBr—(CH₂)₅—N⁺R¹R²R³Z⁻; (CF₃—CH₂—CH₂)₂N—CH₂CHBr—(CH₂)₅—P⁺R¹R²R³Z⁻;
(CF₃—CH₂—CH₂)₂N—CH₂CHBr—(CH₂)₅—O-glucoside;
(CF₃—CH₂—CH₂)₂N—CH₂CHBr—(CH₂)₆—N⁺R¹R²R³Z⁻; (CF₃—CH₂—CH₂)₂N—CH₂CHBr—(CH₂)₆—P⁺R¹R²R³Z⁻;
(CF₃—CH₂—CH₂)₂N—CH₂CHBr—(CH₂)₆—O-glucoside;
(CF₃—CH₂—CH₂)₂N—CH₂CHBr—(CH₂)₇—N⁺R¹R²R³Z⁻; (CF₃—CH₂—CH₂)₂N—CH₂CHBr—(CH₂)₇—P⁺R¹R²R³Z⁻;
(CF₃—CH₂—CH₂)₂N—CH₂CHBr—(CH₂)₇—O-glucoside;
(CF₃—CH₂—CH₂)₂N—CH₂CHBr—(CH₂)₈—N⁺R¹R²R³Z⁻; (CF₃—CH₂—CH₂)₂N—CH₂CHBr—(CH₂)₈—P⁺R¹R²R³Z⁻;
(CF₃—CH₂—CH₂)₂N—CH₂CHBr—(CH₂)₈—O-glucoside
(CF₃—CH₂—CH₂)₂N—CH₂CHBr—(CH₂)₉—N⁺R¹R²R³Z⁻; (CF₃—CH₂—CH₂)₂N—CH₂CHBr—(CH₂)₉—P⁺R¹R²R³Z⁻;
(CF₃—CH₂—CH₂)₂N—CH₂CHBr—(CH₂)₉—O-glucoside
(CF₃—CH₂—CH₂)₂N—CH₂CHBr—(CH₂)₁₀—N⁺R¹R²R³Z⁻; (CF₃—CH₂—CH₂)₂N—CH₂CHBr—(CH₂)₁₀—P⁺R¹R²R³Z⁻;
(CF₃—CH₂—CH₂)₂N—CH₂CHBr—(CH₂)₁₀—O-glucoside
(CF₃—CH₂—CH₂)₂N—CH₂CHBr—(CH₂)₁₁—N⁺R¹R²R³Z⁻; (CF₃—CH₂—CH₂)₂N—CH₂CHBr—(CH₂)₁₁—P⁺R¹R²R³Z⁻;
(CF₃—CH₂—CH₂)₂N—CH₂CHBr—(CH₂)₁₁—O-glucoside
(CF₃—CH₂—CH₂)₂N—CH₂CHBr—(CH₂)₁₂—N⁺R¹R²R³Z⁻; (CF₃—CH₂—CH₂)₂N—CH₂CHBr—(CH₂)₁₂—P⁺R¹R²R³Z⁻;
(CF₃—CH₂—CH₂)₂N—CH₂CHBr—(CH₂)₁₂—O-glucoside
(CF₃—CH₂—CH₂)₂N—CH₂CHBr—(CH₂)₁₃—N⁺R¹R²R³Z⁻; (CF₃—CH₂—CH₂)₂N—CH₂CHBr—(CH₂)₁₃—P⁺R¹R²R³Z⁻;
(CF₃—CH₂—CH₂)₂N—CH₂CHBr—(CH₂)₁₃—O-glucoside
(CF₃—CH₂—CH₂)₂N—CH₂CHBr—(CH₂)₁₄—N⁺R¹R²R³Z⁻; (CF₃—CH₂—CH₂)₂N—CH₂CHBr—(CH₂)₁₄—P⁺R¹R²R³Z⁻;
(CF₃—CH₂—CH₂)₂N—CH₂CHBr—(CH₂)₁₄—O-glucoside
(CF₃—CH₂—CH₂)₂N—CH₂CHBr—(CH₂)₁₅—N⁺R¹R²R³Z⁻; (CF₃—CH₂—CH₂)₂N—CH₂CHBr—(CH₂)₁₅—P⁺R¹R²R³Z⁻;
(CF₃—CH₂—CH₂)₂N—CH₂CHBr—(CH₂)₁₅—O-glucoside
(CF₃—CH₂—CH₂)₂N—CH₂CHBr—(CH₂)₁₆—N⁺R¹R²R³Z⁻; (CF₃—CH₂—CH₂)₂N—CH₂CHBr—(CH₂)₁₆—P⁺R¹R²R³Z⁻;
(CF₃—CH₂—CH₂)₂N—CH₂CHBr—(CH₂)₁₆—O-glucoside
CF₃—CH₂—CH₂—S—CH₂CHBr—(CH₂)₄—N⁺R¹R²R³Z⁻; CF₃—CH₂—CH₂—S—CH₂CHBr—(CH₂)₄—P⁺R¹R²R³Z⁻;
CF₃—CH₂—CH₂—S—CH₂CHBr—(CH₂)₄—O-glucoside;
CF₃—CH₂—CH₂—S—CH₂CHBr—(CH₂)₅—N⁺R¹R²R³Z⁻; CF₃—CH₂—CH₂—S—CH₂CHBr—(CH₂)₅—P⁺R¹R²R³Z⁻;
CF₃—CH₂—CH₂—S—CH₂CHBr—(CH₂)₅—O-glucoside;
CF₃—CH₂—CH₂—S—CH₂CHBr—(CH₂)₆—N⁺R¹R²R³Z⁻; CF₃—CH₂—CH₂—S—CH₂CHBr—(CH₂)₆—P⁺R¹R²R³Z⁻;
CF₃—CH₂—CH₂—S—CH₂CHBr—(CH₂)₆—O-glucoside;
CF₃—CH₂—CH₂—S—CH₂CHBr—(CH₂)₇—N⁺R¹R²R³Z⁻; CF₃—CH₂—CH₂—S—CH₂CHBr—(CH₂)₇—P⁺R¹R²R³Z⁻;
CF₃—CH₂—CH₂—S—CH₂CHBr—(CH₂)₇—O-glucoside;
CF₃—CH₂—CH₂—S—CH₂CHBr—(CH₂)₈—N⁺R¹R²R³Z⁻; CF₃—CH₂—CH₂—S—CH₂CHBr—(CH₂)₈—P⁺R¹R²R³Z⁻;
CF₃—CH₂—CH₂—S—CH₂CHBr—(CH₂)₈—O-glucoside
CF₃—CH₂—CH₂—S—CH₂CHBr—(CH₂)₉—N⁺R¹R²R³Z⁻; CF₃—CH₂—CH₂—S—CH₂CHBr—(CH₂)₉—P⁺R¹R²R³Z⁻;
CF₃—CH₂—CH₂—S—CH₂CHBr—(CH₂)₉—O-glucoside
CF₃—CH₂—CH₂—S—CH₂CHBr—(CH₂)₁₀—N⁺R¹R²R³Z⁻; CF₃—CH₂—CH₂—S—CH₂CHBr—(CH₂)₁₀—P⁺R¹R²R³Z⁻;
CF₃—CH₂—CH₂—S—CH₂CHBr—(CH₂)₁₀—O-glucoside
CF₃—CH₂—CH₂—S—CH₂CHBr—(CH₂)₁₁—N⁺R¹R²R³Z⁻; CF₃—CH₂—CH₂—S—CH₂CHBr—(CH₂)₁₁—P⁺R¹R²R³Z⁻;
CF₃—CH₂—CH₂—S—CH₂CHBr—(CH₂)₁₁—O-glucoside
CF₃—CH₂—CH₂—S—CH₂CHBr—(CH₂)₁₂—N⁺R¹R²R³Z⁻; CF₃—CH₂—CH₂—S—CH₂CHBr—(CH₂)₁₂—P⁺R¹R²R³Z⁻;
CF₃—CH₂—CH₂—S—CH₂CHBr—(CH₂)₁₂—O-glucoside
CF₃—CH₂—CH₂—S—CH₂CHBr—(CH₂)₁₃—N⁺R¹R²R³Z⁻; CF₃—CH₂—CH₂—S—CH₂CHBr—(CH₂)₁₃—P⁺R¹R²R³Z⁻;
CF₃—CH₂—CH₂—S—CH₂CHBr—(CH₂)₁₃—O-glucoside
CF₃—CH₂—CH₂—S—CH₂CHBr—(CH₂)₁₄—N⁺R¹R²R³Z⁻; CF₃—CH₂—CH₂—S—CH₂CHBr—(CH₂)₁₄—P⁺R¹R²R³Z⁻;
CF₃—CH₂—CH₂—S—CH₂CHBr—(CH₂)₁₄—O-glucoside
CF₃—CH₂—CH₂—S—CH₂CHBr—(CH₂)₁₅—N⁺R¹R²R³Z⁻; CF₃—CH₂—CH₂—S—CH₂CHBr—(CH₂)₁₅—P⁺R¹R²R³Z⁻;
CF₃—CH₂—CH₂—S—CH₂CHBr—(CH₂)₁₅—O-glucoside
CF₃—CH₂—CH₂—S—CH₂CHBr—(CH₂)₁₆—N⁺R¹R²R³Z⁻; CF₃—CH₂—CH₂—S—CH₂CHBr—(CH₂)₁₆—P⁺R¹R²R³Z⁻;
CF₃—CH₂—CH₂—S—CH₂CHBr—(CH₂)₁₆—O-glucoside
C₂F₅—S—CH₂CHBr—(CH₂)₄—N⁺R¹R²R³Z⁻; C₂F₅—S—CH₂CHBr—(CH₂)₄—P⁺R¹R²R³Z⁻; C₂F₅—S—CH₂CHBr—(CH₂)₄—O-
glucoside;
C₂F₅—S—CH₂CHBr—(CH₂)₅—N⁺R¹R²R³Z⁻; C₂F₅—S—CH₂CHBr—(CH₂)₅—P⁺R¹R²R³Z⁻; C₂F₅—S—CH₂CHBr—(CH₂)₅—O-
glucoside;
C₂F₅—S—CH₂CHBr—(CH₂)₆—N⁺R¹R²R³Z⁻; C₂F₅—S—CH₂CHBr—(CH₂)₆—P⁺R¹R²R³Z⁻; C₂F₅—S—CH₂CHBr—(CH₂)₆—O-
glucoside;
C₂F₅—S—CH₂CHBr—(CH₂)₇—N⁺R¹R²R³Z⁻; C₂F₅—S—CH₂CHBr—(CH₂)₇—P⁺R¹R²R³Z⁻; C₂F₅—S—CH₂CHBr—(CH₂)₇—O-
glucoside;
C₂F₅—S—CH₂CHBr—(CH₂)₈—N⁺R¹R²R³Z⁻; C₂F₅—S—CH₂CHBr—(CH₂)₈—P⁺R¹R²R³Z⁻; C₂F₅—S—CH₂CHBr—(CH₂)₈—O-
glucoside
C₂F₅—S—CH₂CHBr—(CH₂)₉—N⁺R¹R²R³Z⁻; C₂F₅—S—CH₂CHBr—(CH₂)₉—P⁺R¹R²R³Z⁻; C₂F₅—S—CH₂CHBr—(CH₂)₉—O-
glucoside
C₂F₅—S—CH₂CHBr—(CH₂)₁₀—N⁺R¹R²R³Z⁻; C₂F₅—S—CH₂CHBr—(CH₂)₁₀—P⁺R¹R²R³Z⁻;
C₂F₅—S—CH₂CHBr—(CH₂)₁₀—O-glucoside
C₂F₅—S—CH₂CHBr—(CH₂)₁₁—N⁺R¹R²R³Z⁻; C₂F₅—S—CH₂CHBr—(CH₂)₁₁—P⁺R¹R²R³Z⁻;
C₂F₅—S—CH₂CHBr—(CH₂)₁₁—O-glucoside
C₂F₅—S—CH₂CHBr—(CH₂)₁₂—N⁺R¹R²R³Z⁻; C₂F₅—S—CH₂CHBr—(CH₂)₁₂—P⁺R¹R²R³Z⁻;
C₂F₅—S—CH₂CHBr—(CH₂)₁₂—O-glucoside
C₂F₅—S—CH₂CHBr—(CH₂)₁₃—N⁺R¹R²R³Z⁻; C₂F₅—S—CH₂CHBr—(CH₂)₁₃—P⁺R¹R²R³Z⁻;
C₂F₅—S—CH₂CHBr—(CH₂)₁₃—O-glucoside
C₂F₅—S—CH₂CHBr—(CH₂)₁₄—N⁺R¹R²R³Z⁻; C₂F₅—S—CH₂CHBr—(CH₂)₁₄—P⁺R¹R²R³Z⁻;
C₂F₅—S—CH₂CHBr—(CH₂)₁₄—O-glucoside
C₂F₅—S—CH₂CHBr—(CH₂)₁₅—N⁺R¹R²R³Z⁻; C₂F₅—S—CH₂CHBr—(CH₂)₁₅—P⁺R¹R²R³Z⁻;
C₂F₅—S—CH₂CHBr—(CH₂)₁₅—O-glucoside
C₂F₅—S—CH₂CHBr—(CH₂)₁₆—N⁺R¹R²R³Z⁻; C₂F₅—S—CH₂CHBr—(CH₂)₁₆—P⁺R¹R²R³Z⁻;
C₂F₅—S—CH₂CHBr—(CH₂)₁₆—O-glucoside
(CF₃)₂N—CH₂CHBr—(CH₂)₄—N⁺R¹R²R³Z⁻; (CF₃)₂N—CH₂CHBr—(CH₂)₄—P⁺R¹R²R³Z⁻;
(CF₃)₂N—CH₂CHBr—(CH₂)₄—O-glucoside;
(CF₃)₂N—CH₂CHBr—(CH₂)₅—N⁺R¹R²R³Z⁻; (CF₃)₂N—CH₂CHBr—(CH₂)₅—P⁺R¹R²R³Z⁻;
(CF₃)₂N—CH₂CHBr—(CH₂)₅—O-glucoside;
(CF₃)₂N—CH₂CHBr—(CH₂)₆—N⁺R¹R²R³Z⁻; (CF₃)₂N—CH₂CHBr—(CH₂)₆—P⁺R¹R²R³Z⁻;
(CF₃)₂N—CH₂CHBr—(CH₂)₆—O-glucoside;
(CF₃)₂N—CH₂CHBr—(CH₂)₇—N⁺R¹R²R³Z⁻; (CF₃)₂N—CH₂CHBr—(CH₂)₇—P⁺R¹R²R³Z⁻;
(CF₃)₂N—CH₂CHBr—(CH₂)₇—O-glucoside;
(CF₃)₂N—CH₂CHBr—(CH₂)₈—N⁺R¹R²R³Z⁻; (CF₃)₂N—CH₂CHBr—(CH₂)₈—P⁺R¹R²R³Z⁻;
(CF₃)₂N—CH₂CHBr—(CH₂)₈—O-glucoside
(CF₃)₂N—CH₂CHBr—(CH₂)₉—N⁺R¹R²R³Z⁻; (CF₃)₂N—CH₂CHBr—(CH₂)₉—P⁺R¹R²R³Z⁻;
(CF₃)₂N—CH₂CHBr—(CH₂)₉—O-glucoside
(CF₃)₂N—CH₂CHBr—(CH₂)₁₀—N⁺R¹R²R³Z⁻; (CF₃)₂N—CH₂CHBr—(CH₂)₁₀—P⁺R¹R²R³Z⁻;
(CF₃)₂N—CH₂CHBr—(CH₂)₁₀—O-glucoside
(CF₃)₂N—CH₂CHBr—(CH₂)₁₁—N⁺R¹R²R³Z⁻; (CF₃)₂N—CH₂CHBr—(CH₂)₁₁—P⁺R¹R²R³Z⁻;
(CF₃)₂N—CH₂CHBr—(CH₂)₁₁—O-glucoside
(CF₃)₂N—CH₂CHBr—(CH₂)₁₂—N⁺R¹R²R³Z⁻; (CF₃)₂N—CH₂CHBr—(CH₂)₁₂—P⁺R¹R²R³Z⁻;
(CF₃)₂N—CH₂CHBr—(CH₂)₁₂—O-glucoside
(CF₃)₂N—CH₂CHBr—(CH₂)₁₃—N⁺R¹R²R³Z⁻; (CF₃)₂N—CH₂CHBr—(CH₂)₁₃—P⁺R¹R²R³Z⁻;
(CF₃)₂N—CH₂CHBr—(CH₂)₁₃—O-glucoside
(CF₃)₂N—CH₂CHBr—(CH₂)₁₄—N⁺R¹R²R³Z⁻; (CF₃)₂N—CH₂CHBr—(CH₂)₁₄—P⁺R¹R²R³Z⁻;
(CF₃)₂N—CH₂CHBr—(CH₂)₁₄—O-glucoside
(CF₃)₂N—CH₂CHBr—(CH₂)₁₅—N⁺R¹R²R³Z⁻; (CF₃)₂N—CH₂CHBr—(CH₂)₁₅—P⁺R¹R²R³Z⁻;
(CF₃)₂N—CH₂CHBr—(CH₂)₁₅—O-glucoside
(CF₃)₂N—CH₂CHBr—(CH₂)₁₆—N⁺R¹R²R³Z⁻; (CF₃)₂N—CH₂CHBr—(CH₂)₁₆—P⁺R¹R²R³Z⁻;
(CF₃)₂N—CH₂CHBr—(CH₂)₁₆—O-glucoside
C₂F₅—S—(CH₂)₄—COOH; C₂F₅—S—(CH₂)₄—SO₃H; C₂F₅—S—(CH₂)₄—O—SO₃H; C₂F₅—S—(CH₂)₄—OPO₃H
C₂F₅—S—(CH₂)₅—COOH; C₂F₅—S—(CH₂)₅—SO₃H; C₂F₅—S—(CH₂)₅—O—SO₃H; C₂F₅—S—(CH₂)₅—OPO₃H
C₂F₅—S—(CH₂)₆—COOH; C₂F₅—S—(CH₂)₆—SO₃H; C₂F₅—S—(CH₂)₆—O—SO₃H; C₂F₅—S—(CH₂)₆—OPO₃H;
C₂F₅—S—(CH₂)₇—COOH; C₂F₅—S—(CH₂)₇—SO₃H; C₂F₅—S—(CH₂)₇—O—SO₃H; C₂F₅—S—(CH₂)₇—OPO₃H;
C₂F₅—S—(CH₂)₈—COOH; C₂F₅—S—(CH₂)₈—SO₃H; C₂F₅—S—(CH₂)₈—O—SO₃H; C₂F₅—S—(CH₂)₈—OPO₃H
C₂F₅—S—(CH₂)₉—COOH; C₂F₅—S—(CH₂)₉—SO₃H; C₂F₅—S—(CH₂)₉—O—SO₃H; C₂F₅—S—(CH₂)₉—OPO₃H
C₂F₅—S—(CH₂)₁₀—COOH; C₂F₅—S—(CH₂)₁₀—SO₃H; C₂F₅—S—(CH₂)₁₀—O—SO₃H; C₂F₅—S—(CH₂)₁₀—OPO₃H
C₂F₅—S—(CH₂)₁₁—COOH; C₂F₅—S—(CH₂)₁₁—SO₃H; C₂F₅—S—(CH₂)₁₁—O—SO₃H; C₂F₅—S—(CH₂)₁₁—OPO₃H
C₂F₅—S—(CH₂)₁₂—COOH; C₂F₅—S—(CH₂)₁₂—SO₃H; C₂F₅—S—(CH₂)₁₂—O—SO₃H, C₂F₅—S—(CH₂)₁₂—OPO₃H
C₂F₅—S—(CH₂)₁₃—COOH; C₂F₅—S—(CH₂)₁₃—SO₃H; C₂F₅—S—(CH₂)₁₃—O—SO₃H, C₂F₅—S—(CH₂)₁₃—OPO₃H
C₂F₅—S—(CH₂)₁₄—COOH; C₂F₅—S—(CH₂)₁₄—SO₃H; C₂F₅S—(CH₂)₁₄—O—SO₃H; C₂F₅—S—(CH₂)₁₄—OPO₃H
C₂F₅—S—(CH₂)₁₅—COOH; C₂F₅—S—(CH₂)₁₅—SO₃H; C₂F₅—S—(CH₂)₁₅—O—SO₃H; C₂F₅—S—(CH₂)₁₅—OPO₃H
C₂F₅—S—(CH₂)₁₆—COOH; C₂F₅—S—(CH₂)₁₆—SO₃H; C₂F₅—S—(CH₂)₁₆—O—SO₃H; C₂F₅—S—(CH₂)₁₆—OPO₃H
C₂F₅—S—CH₂—Ar—(CH₂)₄—COOH; C₂F₅—S—CH₂—Ar—(CH₂)₄—SO₃H; C₂F₅—S—CH₂—Ar—(CH₂)₄—O—SO₃H;
C₂F₅—S—CH₂—Ar—(CH₂)₄—O—PO₃H
C₂F₅—S—CH₂—Ar—(CH₂)₅—COOH; C₂F₅—S—CH₂—Ar—(CH₂)₅—SO₃H; C₂F₅—S—CH₂—Ar—(CH₂)₅—O—SO₃H,
C₂F₅—S—CH₂—Ar—(CH₂)₅—O—PO₃H
C₂F₅—S—CH₂—Ar—(CH₂)₆—COOH; C₂F₅—S—CH₂—Ar—(CH₂)₆—SO₃H; C₂F₅—S—CH₂—Ar—(CH₂)₆—O—SO₃H,
C₂F₅—S—CH₂—Ar—(CH₂)₆—O—PO₃H
C₂F₅—S—CH₂—Ar—(CH₂)₇—COOH; C₂F₅—S—CH₂—Ar—(CH₂)₇—SO₃H; C₂F₅—S—CH₂—Ar—(CH₂)₇—O—SO₃H;
C₂F₅—S—CH₂—Ar—(CH₂)₇—O—PO₃H
C₂F₅—S—CH₂—Ar—(CH₂)₈—COOH; C₂F₅—S—CH₂—Ar—(CH₂)₈—SO₃H; C₂F₅—S—CH₂—Ar—(CH₂)₈—O—SO₃H,
C₂F₅—S—CH₂—Ar—(CH₂)₈—O—PO₃H
C₂F₅—S—CH₂—Ar—(CH₂)₉—COOH; C₂F₅—S—CH₂—Ar—(CH₂)₉—SO₃H; C₂F₅—S—CH₂—Ar—(CH₂)₉—O—SO₃H,
C₂F₅—S—CH₂—Ar—(CH₂)₉—O—PO₃H
C₂F₅—S—CH₂—Ar—(CH₂)₁₀—COOH; C₂F₅—S—CH₂—Ar—(CH₂)₁₀—SO₃H; C₂F₅—S—CH₂—Ar—(CH₂)₁₀—O—SO₃H,
C₂F₅—S—CH₂—Ar—(CH₂)₁₀—O—PO₃H
C₂F₅—S—CH₂—Ar—(CH₂)₁₁—COOH; C₂F₅—S—CH₂—Ar—(CH₂)₁₁—SO₃H; C₂F₅—S—CH₂—Ar—(CH₂)₁₁—O—SO₃H,
C₂F₅—S—CH₂—Ar—(CH₂)₁₁—O—PO₃H
C₂F₅—S—CH₂—Ar—(CH₂)₁₂—COOH; C₂F₅—S—CH₂—Ar—(CH₂)₁₂—SO₃H; C₂F₅—S—CH₂—Ar—(CH₂)₁₂—O—SO₃H,
C₂F₅—S—CH₂—Ar—(CH₂)₁₂—O—PO₃H
C₂F₅—S—CH₂—Ar—(CH₂)₁₃—COOH; C₂F₅—S—CH₂—Ar—(CH₂)₁₃—SO₃H; C₂F₅—S—CH₂—Ar—(CH₂)₁₃—O—SO₃H,
C₂F₅—S—CH₂—Ar—(CH₂)₁₃—O—PO₃H
C₂F₅—S—CH₂—Ar—(CH₂)₁₄—COOH; C₂F₅—S—CH₂—Ar—(CH₂)₁₄—SO₃H; C₂F₅—S—CH₂—Ar—(CH₂)₁₄—O—SO₃H,
C₂F₅—S—CH₂—Ar—(CH₂)₁₄—O—PO₃H
C₂F₅—S—CH₂—Ar—(CH₂)₁₅—COOH; C₂F₅—S—CH₂—Ar—(CH₂)₁₅—SO₃H; C₂F₅—S—CH₂—Ar—(CH₂)₁₅—O—SO₃H,
C₂F₅—S—CH₂—Ar—(CH₂)₁₅—O—PO₃H
C₂F₅—S—CH₂—Ar—(CH₂)₁₆—COOH C₂F₅—S—CH₂—Ar—(CH₂)₁₆—SO₃H; C₂F₅—S—CH₂—Ar—(CH₂)₁₆—O—SO₃H,
C₂F₅—S—CH₂—Ar—(CH₂)₁₆—O—PO₃H
CF₃—CH₂—CH₂—S—(CH₂)₄—COOH; CF₃—CH₂—CH₂—S—(CH₂)₄—SO₃H; CF₃—CH₂—CH₂—S—(CH₂)₄—O—SO₃H,
CF₃—CH₂—CH₂—S—(CH₂)₄—OH, CF₃—CH₂—CH₂—S—(CH₂)₄—SH; CF₃—CH₂—CH₂—S—(CH₂)₄—(OCH₂CHR)ₘOH
CF₃—CH₂—CH₂—S—(CH₂)₅—COOH; CF₃—CH₂—CH₂—S—(CH₂)₅—SO₃H; CF₃—CH₂—CH₂—S—(CH₂)₅—O—SO₃H,
CF₃—CH₂—CH₂—S—(CH₂)₅—OH, CF₃—CH₂—CH₂—S—(CH₂)₅—SH CF₃—CH₂—CH₂—S—(CH₂)₅—(OCH₂CHR)ₘOH
CF₃—CH₂—CH₂—S—(CH₂)₆—COOH; CF₃—CH₂—CH₂—S—(CH₂)₆—SO₃H; CF₃—CH₂—CH₂—S—(CH₂)₆—O—SO₃H,
CF₃—CH₂—CH₂—S—(CH₂)₆—OH, CF₃—CH₂—CH₂—S—(CH₂)₆—SH CF₃—CH₂—CH₂—S—(CH₂)₆—(OCH₂CHR)ₘOH
CF₃—CH₂—CH₂—S—(CH₂)₇—COOH; CF₃—CH₂—CH₂—S—(CH₂)₇—SO₃H; CF₃—CH₂—CH₂—S—(CH₂)₇—O—SO₃H,
CF₃—CH₂—CH₂—S—(CH₂)₇—OH, CF₃—CH₂—CH₂—S—(CH₂)₇—SH CF₃—CH₂—CH₂—S—(CH₂)₇—(OCH₂CHR)ₘOH
CF₃—CH₂—CH₂—S—(CH₂)₈—COOH; CF₃—CH₂—CH₂—S—(CH₂)₈—SO₃H; CF₃—CH₂—CH₂—S—(CH₂)₈—O—SO₃H,
CF₃—CH₂—CH₂—S—(CH₂)₈—OH, CF₃—CH₂—CH₂—S—(CH₂)₈—SH CF₃—CH₂—CH₂—S—(CH₂)₈—(OCH₂CHR)ₘOH
CF₃—CH₂—CH₂—S—(CH₂)₉—COOH; CF₃—CH₂—CH₂—S—(CH₂)₉—SO₃H; CF₃—CH₂—CH₂—S—(CH₂)₉—O—SO₃H,
CF₃—CH₂—CH₂—S—(CH₂)₉—OH, CF₃—CH₂—CH₂—S—(CH₂)₉—SH CF₃—CH₂—CH₂—S—(CH₂)₉—(OCH₂CHR)ₘOH
CF₃—CH₂—CH₂—S—(CH₂)₁₀—COOH; CF₃—CH₂—CH₂—S—(CH₂)₁₀—SO₃H; CF₃—CH₂—CH₂—S—(CH₂)₁₀—O—SO₃H,
CF₃—CH₂—CH₂—S—(CH₂)₁₀—OH, CF₃—CH₂—CH₂—S—(CH₂)₁₀—SH CF₃—CH₂—CH₂—S—(CH₂)₁₀—(OCH₂CHR)ₘOH
CF₃—CH₂—CH₂—S—(CH₂)₁₁—COOH; CF₃—CH₂—CH₂—S—(CH₂)₁₁—SO₃H; CF₃—CH₂—CH₂—S—(CH₂)₁₁—O—SO₃H,
CF₃—CH₂—CH₂—S—(CH₂)₁₁—OH, CF₃—CH₂—CH₂—S—(CH₂)₁₁—SH CF₃—CH₂—CH₂—S—(CH₂)₁₁—(OCH₂CHR)ₘOH
CF₃—CH₂—CH₂—S—(CH₂)₁₂—COOH; CF₃—CH₂—CH₂—S—(CH₂)₁₂—SO₃H; CF₃—CH₂—CH₂—S—(CH₂)₁₂—O—SO₃H,
CF₃—CH₂—CH₂—S—(CH₂)₁₂—OH, CF₃—CH₂—CH₂—S—(CH₂)₁₂—SH CF₃—CH₂—CH₂—S—(CH₂)₁₂—(OCH₂CHR)ₘOH
CF₃—CH₂—CH₂—S—(CH₂)₁₃—COOH; CF₃—CH₂—CH₂—S—(CH₂)₁₃—SO₃H; CF₃—CH₂—CH₂—S—(CH₂)₁₃—O—SO₃H,
CF₃—CH₂—CH₂—S—(CH₂)₁₃—OH, CF₃—CH₂—CH₂—S—(CH₂)₁₃—SH CF₃—CH₂—CH₂—S—(CH₂)₁₃—(OCH₂CHR)ₘOH
CF₃—CH₂—CH₂—S—(CH₂)₁₄—COOH; CF₃—CH₂—CH₂—S—(CH₂)₁₄—SO₃H; CF₃—CH₂—CH₂—S—(CH₂)₁₄—O—SO₃H,
CF₃—CH₂—CH₂—S—(CH₂)₁₄—OH, CF₃—CH₂—CH₂—S—(CH₂)₁₄—SH CF₃—CH₂—CH₂—S—(CH₂)₁₄—(OCH₂CHR)ₘOH
CF₃—CH₂—CH₂—S—(CH₂)₁₅—COOH; CF₃—CH₂—CH₂—S—(CH₂)₁₅—SO₃H; CF₃—CH₂—CH₂—S—(CH₂)₁₅—O—SO₃H,
CF₃—CH₂—CH₂—S—(CH₂)₁₅—OH, CF₃—CH₂—CH₂—S—(CH₂)₁₅—SH CF₃—CH₂—CH₂—S—(CH₂)₁₅—(OCH₂CHR)ₘOH
CF₃—CH₂—CH₂—S—(CH₂)₁₆—COOH; CF₃—CH₂—CH₂—S—(CH₂)₁₆—SO₃H; CF₃—CH₂—CH₂—S—(CH₂)₁₆—O—SO₃H,
CF₃—CH₂—CH₂—S—(CH₂)₁₆—OH, CF₃—CH₂—CH₂—S—(CH₂)₁₆—SH CF₃—CH₂—CH₂—S—(CH₂)₁₆—(OCH₂CHR)ₘOH
CF₃—CH₂—CH₂—S—(CH₂)₄—(OCH₂CHR)ₘSH; CF₃—CH₂—CH₂—S—(CH₂)₄—(OCH₂CHR)ₘSO₂CH═CH₂;
CF₃—CH₂—CH₂—S—(CH₂)₄—(OCH₂CHR)_{m—}OCOCH═CH₂,
CF₃—CH₂—CH₂—S—(CH₂)₄—(OCH₂CHR)ₘOPO₃H, CF₃—CH₂—CH₂—S—(CH₂)₄—(OCH₂CHR)ₘOAr(NCO)ₚ
CF₃—CH₂—CH₂—S—(CH₂)₅—(OCH₂CHR)ₘSH; CF₃—CH₂—CH₂—S—(CH₂)₅—(OCH₂CHR)ₘSO₂CH═CH₂;
CF₃—CH₂—CH₂—S—(CH₂)₅—(OCH₂CHR)ₘOCOCH═CH₂,
CF₃—CH₂—CH₂—S—(CH₂)₅—(OCH₂CHR)ₘOPO₃H, CF₃—CH₂—CH₂—S—(CH₂)₅—(OCH₂CHR)ₘOAr(NCO)ₚ
CF₃—CH₂—CH₂—S—(CH₂)₆—(OCH₂CHR)ₘSH; CF₃—CH₂—CH₂—S—(CH₂)₆—(OCH₂CHR)ₘSO₂CH═CH₂;
CF₃—CH₂—CH₂S—(CH₂)₆—(OCH₂CHR)ₘOCOCH═CH₂, CF₃—CH₂—CH₂S—(CH₂)₆—(OCH₂CHR)ₘOPO₃H,
CF₃—CH₂—CH₂S—(CH₂)₆—(OCH₂CHR)ₘOAr(NCO)ₚ
CF₃—CH₂—CH₂—S—(CH₂)—(OCH₂CHR)ₘSH; CF₃—CH₂—CH₂S—(CH₂)₇—(OCH₂CHR)ₘSO₂CH═CH₂;
CF₃—CH₂—CH₂S—(CH₂)₇—(OCH₂CHR)ₘOCOCH═CH₂, CF₃—CH₂—CH₂S—(CH₂)₇—(OCH₂CHR)ₘOPO₃H,
CF₃—CH₂—CH₂S—(CH₂)₇—(OCH₂CHR)ₘOAr(NCO)ₚ
CF₃—CH₂—CH₂—S—(CH₂)₈—(OCH₂CHR)ₘSH; CF₃—CH₂—CH₂S—(CH₂)₈—(OCH₂CHR)ₘSO₂CH═CH₂;
CF₃—CH₂—CH₂S—(CH₂)₈—(OCH₂CHR)ₘOCOCH═CH₂, CF₃—CH₂—CH₂S—(CH₂)₈—(OCH₂CHR)ₘOPO₃H,
CF₃—CH₂—CH₂S—(CH₂)₈—(OCH₂CHR)ₘOAr(NCO)ₚ
CF₃—CH₂—CH₂—S—(CH₂)₉—(OCH₂CHR)ₘSH; CF₃—CH₂—CH₂S—(CH₂)₉—(OCH₂CHR)ₘSO₂CH═CH₂;
CF₃—CH₂—CH₂S—(CH₂)₉—(OCH₂CHR)ₘOCOCH═CH₂, CF₃—CH₂—CH₂S—(CH₂)₉—(OCH₂CHR)ₘOPO₃H,
CF₃—CH₂—CH₂S—(CH₂)₉—(OCH₂CHR)ₘOAr(NCO)ₚ
CF₃—CH₂—CH₂—S—(CH₂)₁₀—(OCH₂CHR)ₘSH; CF₃—CH₂—CH₂—S—(CH₂)₁₀—(OCH₂CHR)ₘSO₂CH═CH₂;
CF₃—CH₂—CH₂S—(CH₂)₁₀—(OCH₂CHR)_{m—}OCOCH═CH₂,
CF₃—CH₂—CH₂S—(CH₂)₁₀—(OCH₂CHR)ₘOPO₃H, CF₃—CH₂—CH₂S—(CH₂)₁₀—(OCH₂CHR)ₘOAr(NCO)ₚ
CF₃—CH₂—CH₂—S—(CH₂)₁₁—(OCH₂CHR)ₘSH; CF₃—CH₂—CH₂S—(CH₂)₁₁—(OCH₂CHR)ₘSO₂CH═CH₂;
CF₃—CH₂—CH₂S—(CH₂)₁₁—(OCH₂CHR)_{m—}OCOCH═CH₂,
CF₃—CH₂—CH₂S—(CH₂)₁₁—(OCH₂CHR)ₘOPO₃H, CF₃—CH₂—CH₂S—(CH₂)₁₁—(OCH₂CHR)ₘOAr(NCO)ₚ
CF₃—CH₂—CH₂—S—(CH₂)₁₂—(OCH₂CHR)ₘSH; CF₃—CH₂—CH₂S—(CH₂)₁₂—(OCH₂CHR)ₘSO₂CH═CH₂;
CF₃—CH₂—CH₂S—(CH₂)₁₂—(OCH₂CHR)_{m—}OCOCH═CH₂,
CF₃—CH₂—CH₂S—(CH₂)₁₂—(OCH₂CHR)ₘOPO₃H, CF₃—CH₂—CH₂S—(CH₂)₁₂—(OCH₂CHR)ₘOAr(NCO)ₚ
CF₃—CH₂—CH₂—S—(CH₂)₁₃—(OCH₂CHR)ₘSH; CF₃—CH₂—CH₂S—(CH₂)₁₃—(OCH₂CHR)ₘSO₂CH═CH₂;
CF₃—CH₂—CH₂S—(CH₂)₁₃—(OCH₂CHR)_{m—}OCOCH═CH₂,
CF₃—CH₂—CH₂S—(CH₂)₁₃—(OCH₂CHR)ₘOPO₃H, CF₃—CH₂—CH₂S—(CH₂)₁₃—(OCH₂CHR)ₘOAr(NCO)ₚ
CF₃—CH₂—CH₂—S—(CH₂)₁₄—(OCH₂CHR)ₘSH; CF₃—CH₂—CH₂S—(CH₂)₁₄—(OCH₂CHR)ₘSO₂CH═CH₂;
CF₃—CH₂—CH₂S—(CH₂)₁₄—(OCH₂CHR)_{m—}OCOCH═CH₂,
CF₃—CH₂—CH₂S—(CH₂)₁₄—(OCH₂CHR)ₘOPO₃H, CF₃—CH₂—CH₂S—(CH₂)₁₄—(OCH₂CHR)ₘOAr(NCO)ₚ
CF₃—CH₂—CH₂—S—(CH₂)₁₅—(OCH₂CHR)ₘSH; CF₃—CH₂—CH₂S—(CH₂)₁₅—(OCH₂CHR)ₘSO₂CH═CH₂;
CF₃—CH₂—CH₂S—(CH₂)₁₅—(OCH₂CHR)_{m—}OCOCH═CH₂,
CF₃—CH₂—CH₂S—(CH₂)₁₅—(OCH₂CHR)ₘOPO₃H, CF₃—CH₂—CH₂S—(CH₂)₁₅—(OCH₂CHR)ₘOAr(NCO)ₚ
CF₃—CH₂—CH₂—S—(CH₂)₁₆—(OCH₂CHR)ₘSH; CF₃—CH₂—CH₂S—(CH₂)₁₆—(OCH₂CHR)ₘSO₂CH═CH₂;
CF₃—CH₂—CH₂S—(CH₂)₁₆—(OCH₂CHR)ₘ—OCOCH═CH₂,
CF₃—CH₂—CH₂S—(CH₂)₁₆—(OCH₂CHR)ₘOPO₃H, CF₃—CH₂—CH₂S—(CH₂)₁₆—(OCH₂CHR)ₘOAr(NCO)ₚ
CF₃—CH₂—CH₂—S—CH₂—Ar—(CH₂)₄—COOH; CF₃—CH₂—CH₂—S—CH₂—Ar—(CH₂)₄—SO₃H;
CF₃—CH₂—CH₂—S—CH₂—Ar—(CH₂)₄—O—SO₃H
CF₃—CH₂—CH₂—S—CH₂—Ar—(CH₂)₅—COOH; CF₃—CH₂—CH₂—S—CH₂—Ar—(CH₂)₅—SO₃H;
CF₃—CH₂—CH₂—S—CH₂—Ar—(CH₂)₅—O—SO₃H
CF₃—CH₂—CH₂—S—CH₂—Ar—(CH₂)₆—COOH; CF₃—CH₂—CH₂—S—CH₂—Ar—(CH₂)₆—SO₃H;
CF₃—CH₂—CH₂—S—CH₂—Ar—(CH₂)₆—O—SO₃H
CF₃—CH₂—CH₂—S—CH₂—Ar—(CH₂)₇—COOH; CF₃—CH₂—CH₂—S—CH₂—Ar—(CH₂)₇—SO₃H;
CF₃—CH₂—CH₂—S—CH₂—Ar—(CH₂)₇—O—SO₃H
CF₃—CH₂—CH₂—S—CH₂—Ar—(CH₂)₈—COOH CF₃—CH₂—CH₂—S—CH₂—Ar—(CH₂)₈—SO₃H;
CF₃—CH₂—CH₂—S—CH₂—Ar—(CH₂)₈—O—SO₃H
CF₃—CH₂—CH₂—S—CH₂—Ar—(CH₂)₉—COOH; CF₃—CH₂—CH₂—S—CH₂—Ar—(CH₂)₉—SO₃H;
CF₃—CH₂—CH₂—S—CH₂—Ar—(CH₂)₉—O—SO₃H
CF₃—CH₂—CH₂—S—CH₂—Ar—(CH₂)₁₀—COOH; CF₃—CH₂—CH₂—S—CH₂—Ar—(CH₂)₁₀—SO₃H;
CF₃—CH₂—CH₂—S—CH₂—Ar—(CH₂)₁₀—O—SO₃H
CF₃—CH₂—CH₂—S—CH₂—Ar—(CH₂)₁₁—COOH; CF₃—CH₂—CH₂—S—CH₂—Ar—(CH₂)₁₁—SO₃H;
CF₃—CH₂—CH₂—S—CH₂—Ar—(CH₂)₁₁—O—SO₃H
CF₃—CH₂—CH₂—S—CH₂—Ar—(CH₂)₁₂—COOH; CF₃—CH₂—CH₂—S—CH₂—Ar—(CH₂)₁₂—SO₃H;
CF₃—CH₂—CH₂—S—CH₂—Ar—(CH₂)₁₂—O—SO₃H
CF₃—CH₂—CH₂—S—CH₂—Ar—(CH₂)₁₃—COOH; CF₃—CH₂—CH₂—S—CH₂—Ar—(CH₂)₁₃—SO₃H;
CF₃—CH₂—CH₂—S—CH₂—Ar—(CH₂)₁₃—O—SO₃H
CF₃—CH₂—CH₂—S—CH₂—Ar—(CH₂)₁₄—COOH; CF₃—CH₂—CH₂—S—CH₂—Ar—(CH₂)₁₄—SO₃H;
CF₃—CH₂—CH₂—S—CH₂—Ar—(CH₂)₁₄—O—SO₃H
CF₃—CH₂—CH₂—S—CH₂—Ar—(CH₂)₁₅—COOH; CF₃—CH₂—CH₂—S—CH₂—Ar—(CH₂)₁₅—SO₃H;
CF₃—CH₂—CH₂—S—CH₂—Ar—(CH₂)₁₅—O—SO₃H
CF₃—CH₂—CH₂—S—CH₂—Ar—(CH₂)₁₆—COOHCF₃—CH₂—CH₂—S—CH₂—Ar—(CH₂)₁₆—SO₃H;
CF₃—CH₂—CH₂—S—CH₂—Ar—(CH₂)₁₆—O—SO₃H
(CF₃—CH₂—CH₂)₂N—(CH₂)₄—COOH; (CF₃—CH₂—CH₂)₂N—(CH₂)₄—SO₃H; (CF₃—CH₂—CH₂)₂N—(CH₂)₄—O—SO₃H,
(CF₃—CH₂—CH₂)₂N—(CH₂)₄—OH(CF₃—CH₂—CH₂)₂N—(CH₂)₄—SH;
(CF₃—CH₂—CH₂)₂N—(CH₂)₄—(OCH₂CHR)ₘOH
(CF₃—CH₂—CH₂)₂—N—(CH₂)₅—COOH; (CF₃—CH₂—CH₂)₂N—(CH₂)₅—SO₃H; (CF₃—CH₂—CH₂)₂N—(CH₂)₅—O—SO₃H,
(CF₃—CH₂—CH₂)₂—N—(CH₂)₅—OH; (CF₃—CH₂—CH₂)₂—N—(CH₂)₅—SH;
(CF₃—CH₂—CH₂)₂N—(CH₂)₅—(OCH₂CHR)ₘOH
(CF₃—CH₂—CH₂)₂—N—(CH₂)₆—COOH; (CF₃—CH₂—CH₂)₂N—(CH₂)₆—SO₃H; (CF₃—CH₂—CH₂)₂N—(CH₂)₆—O—SO₃H,
(CF₃—CH₂—CH₂)₂—N—(CH₂)₆—OH(CF₃—CH₂—CH₂)₂—N—(CH₂)₆—SH;
(CF₃—CH₂—CH₂)₂N—(CH₂)₆—(OCH₂CHR)ₘOH
(CF₃—CH₂—CH₂)₂—N—(CH₂)₇—COOH; (CF₃—CH₂—CH₂)₂N—(CH₂)₇—SO₃H; (CF₃—CH₂—CH₂)₂N—(CH₂)₇—O—SO₃H,
(CF₃—CH₂—CH₂)₂N—(CH₂)₇—OH; (CF₃—CH₂—CH₂)₂—N—(CH₂)₇—SH;
(CF₃—CH₂—CH₂)₂N—(CH₂)₇—(OCH₂CHR)ₘOH
(CF₃—CH₂—CH₂)₂—N—(CH₂)₈—COOH; (CF₃—CH₂—CH₂)₂N—(CH₂)₈—SO₃H; (CF₃—CH₂—CH₂)₂N—(CH₂)₈—O—SO₃H,
(CF₃—CH₂—CH₂)₂—N—(CH₂)₈—OH; (CF₃—CH₂—CH₂)₂—N—(CH₂)₈—SH;
(CF₃—CH₂—CH₂)₂N—(CH₂)₈—(OCH₂CHR)ₘOH
(CF₃—CH₂—CH₂)₂—N—(CH₂)₉—COOH; (CF₃—CH₂—CH₂)₂N—(CH₂)₉—SO₃H; (CF₃—CH₂—CH₂)₂N—(CH₂)₉—O—SO₃H,
(CF₃—CH₂—CH₂)₂—N—(CH₂)₉—OH; (CF₃—CH₂—CH₂)₂—N—(CH₂)₉—SH;
(CF₃—CH₂—CH₂)₂N—(CH₂)₉—(OCH₂CHR)ₘOH
(CF₃—CH₂—CH₂)₂—N—(CH₂)₁₀—COOH; (CF₃—CH₂—CH₂)₂N—(CH₂)₁₀—SO₃H; (CF₃—CH₂—CH₂)₂N—(CH₂)₁₀—O—SO₃H,
(CF₃—CH₂—CH₂)₂N—(CH₂)₁₀—OH; (CF₃—CH₂—CH₂)₂—N—(CH₂)₁₀—SH;
(CF₃—CH₂—CH₂)₂N—(CH₂)₁₀—(OCH₂CHR)ₘOH
(CF₃—CH₂—CH₂)₂—N—(CH₂)₁₁—COOH; (CF₃—CH₂—CH₂)₂N—(CH₂)₁₁—SO₃H; (CF₃—CH₂—CH₂)₂N—(CH₂)₁₁—O—SO₃H,
(CF₃—CH₂—CH₂)₂—N—(CH₂)₁₁—OH; (CF₃—CH₂—CH₂)₂—N—(CH₂)₁₁—SH;
(CF₃—CH₂—CH₂)₂N—(CH₂)₁₁—(OCH₂CHR)ₘOH
(CF₃—CH₂—CH₂)₂N—(CH₂)₁₂—COOH; (CF₃—CH₂—CH₂)₂N—(CH₂)₁₂—SO₃H; (CF₃—CH₂—CH₂)₂N—(CH₂)₁₂—O—SO₃H,
(CF₃—CH₂—CH₂)₂N—(CH₂)₁₂—OH; (CF₃—CH₂—CH₂)₂N—(CH₂)₁₂—SH;
(CF₃—CH₂—CH₂)₂N—(CH₂)₁₂—(OCH₂CHR)ₘOH
(CF₃—CH₂—CH₂)₂N—(CH₂)₁₃—COOH; (CF₃—CH₂—CH₂)₂N—(CH₂)₁₃—SO₃H; (CF₃—CH₂—CH₂)₂N—(CH₂)₁₃—O—SO₃H,
(CF₃—CH₂—CH₂)₂N—(CH₂)₁₃—OH; (CF₃—CH₂—CH₂)₂N—(CH₂)₁₃—SH;
(CF₃—CH₂—CH₂)₂N—(CH₂)₁₃—(OCH₂CHR)ₘOH
(CF₃—CH₂—CH₂)₂N—(CH₂)₁₄—COOH; (CF₃—CH₂—CH₂)₂N—(CH₂)₁₄—SO₃H; (CF₃—CH₂—CH₂)₂N—(CH₂)₁₄—O—SO₃H,
(CF₃—CH₂—CH₂)₂N—(CH₂)₁₄—OH; (CF₃—CH₂—CH₂)₂N—(CH₂)₁₄—SH;
(CF₃—CH₂—CH₂)₂N—(CH₂)₁₄—(OCH₂CHR)ₘOH
(CF₃—CH₂—CH₂)₂N—(CH₂)₁₅—COOH; (CF₃—CH₂—CH₂)₂N—(CH₂)₁₅—SO₃H; (CF₃—CH₂—CH₂)₂N—(CH₂)₁₅—O—SO₃H,
(CF₃—CH₂—CH₂)₂N—(CH₂)₁₅—OH; (CF₃—CH₂—CH₂)₂N—(CH₂)₁₅—SH;
(CF₃—CH₂—CH₂)₂N—(CH₂)₁₅—(OCH₂CHR)ₘOH
(CF₃—CH₂—CH₂)₂N—(CH₂)₁₆—COOH; (CF₃—CH₂—CH₂)₂N—(CH₂)₁₆—SO₃H; (CF₃—CH₂—CH₂)₂—N—(CH₂)₁₆—O—SO₃H,
(CF₃—CH₂—CH₂)₂N—(CH₂)₁₆—OH; (CF₃—CH₂—CH₂)₂N—(CH₂)₁₆—SH;
(CF₃—CH₂—CH₂)₂N—(CH₂)₁₆—(OCH₂CHR)ₘOH
(CF₃—CH₂—CH₂)₂N—(CH₂)₄—(OCH₂CHR)ₘSH; (CF₃—CH₂—CH₂)₂N—(CH₂)₄—(OCH₂CHR)ₘSO₂CH═CH₂;
(CF₃—CH₂—CH₂)₂N—(CH₂)₄—(OCH₂CHR)ₘ—OCOCH═CH₂,
(CF₃—CH₂—CH₂)₂N—(CH₂)₄—(OCH₂CHR)ₘOPO₃H, (CF₃—CH₂—CH₂)₂N—(CH₂)₄—(OCH₂CHR)ₘOAr(NCO)ₚ
(CF₃—CH₂—CH₂)₂N—(CH₂)₅—(OCH₂CHR)ₘSH; (CF₃—CH₂—CH₂)₂N—(CH₂)₅—(OCH₂CHR)ₘSO₂CH═CH₂;
(CF₃—CH₂—CH₂)₂N—(CH₂)₅—(OCH₂CHR)ₘ—OCOCH═CH₂,
(CF₃—CH₂—CH₂)₂N—(CH₂)₅—(OCH₂CHR)ₘOPO₃H, (CF₃—CH₂—CH₂)₂N—(CH₂)₅—(OCH₂CHR)ₘOAr(NCO)ₚ
(CF₃—CH₂—CH₂)₂N—(CH₂)₆—(OCH₂CHR)ₘSH; (CF₃—CH₂—CH₂)₂N—(CH₂)₆—(OCH₂CHR)ₘSO₂CH═CH₂;
(CF₃—CH₂—CH₂)₂N—(CH₂)₆—(OCH₂CHR)ₘ—OCOCH═CH₂,
(CF₃—CH₂—CH₂)₂N—(CH₂)₆—(OCH₂CHR)ₘOPO₃H, (CF₃—CH₂—CH₂)₂N—(CH₂)₆—(OCH₂CHR)ₘOAr(NCO)ₚ
(CF₃—CH₂—CH₂)₂N—(CH₂)—(OCH₂CHR)ₘSH; (CF₃—CH₂—CH₂)₂N—(CH₂)₇—(OCH₂CHR)ₘSO₂CH═CH₂;
(CF₃—CH₂—CH₂)₂N—(CH₂)₇—(OCH₂CHR)ₘ—OCOCH═CH₂,
(CF₃—CH₂—CH₂)₂N—(CH₂)₇—(OCH₂CHR)ₘOPO₃H, (CF₃—CH₂—CH₂)₂N—(CH₂)₇—(OCH₂CHR)ₘOAr(NCO)ₚ
(CF₃—CH₂—CH₂)₂N—(CH₂)₈—(OCH₂CHR)ₘSH; (CF₃—CH₂—CH₂)₂N—(CH₂)₈—(OCH₂CHR)ₘSO₂CH═CH₂;
(CF₃—CH₂—CH₂)₂N—(CH₂)₈—(OCH₂CHR)ₘ—OCOCH═CH₂,
(CF₃—CH₂—CH₂)₂N—(CH₂)₈—(OCH₂CHR)ₘOPO₃H, (CF₃—CH₂—CH₂)₂N—(CH₂)₈—(OCH₂CHR)ₘOAr(NCO)ₚ
(CF₃—CH₂—CH₂)₂N—(CH₂)₉—(OCH₂CHR)ₘSH; (CF₃—CH₂—CH₂)₂N—(CH₂)₉—(OCH₂CHR)ₘSO₂CH═CH₂;
(CF₃—CH₂—CH₂)₂N—(CH₂)₉—(OCH₂CHR)ₘ—OCOCH═CH₂,
(CF₃—CH₂—CH₂)₂N—(CH₂)₉—(OCH₂CHR)ₘOPO₃H, (CF₃—CH₂—CH₂)₂N—(CH₂)₉—(OCH₂CHR)ₘOAr(NCO)ₚ
(CF₃—CH₂—CH₂)₂N—(CH₂)₁₀—(OCH₂CHR)ₘSH; (CF₃—CH₂—CH₂)₂N—(CH₂)₁₀—(OCH₂CHR)ₘSO₂CH═CH₂;
(CF₃—CH₂—CH₂)₂N—(CH₂)₁₀—(OCH₂CHR)ₘ—OCOCH═CH₂,
(CF₃—CH₂—CH₂)₂N—(CH₂)₁₀—(OCH₂CHR)ₘOPO₃H, (CF₃—CH₂—CH₂)₂N—(CH₂)₁₀—(OCH₂CHR)ₘOAr(NCO)ₚ
(CF₃—CH₂—CH₂)₂N—(CH₂)₁₁—(OCH₂CHR)ₘSH; (CF₃—CH₂—CH₂)₂N—(CH₂)₁₁—(OCH₂CHR)ₘSO₂CH═CH₂;
(CF₃—CH₂—CH₂)₂N—(CH₂)₁₁—(OCH₂CHR)ₘ—OCOCH═CH₂,
(CF₃—CH₂—CH₂)₂N—(CH₂)₁₁—(OCH₂CHR)ₘOPO₃H, (CF₃—CH₂—CH₂)₂N—(CH₂)₁₁—(OCH₂CHR)ₘOAr(NCO)ₚ
(CF₃—CH₂—CH₂)₂N—(CH₂)₁₂—(OCH₂CHR)ₘSH; (CF₃—CH₂—CH₂)₂N—(CH₂)₁₂—(OCH₂CHR)ₘSO₂CH═CH₂;
(CF₃—CH₂—CH₂)₂N—(CH₂)₁₂—(OCH₂CHR)ₘ—OCOCH═CH₂,
(CF₃—CH₂—CH₂)₂N—(CH₂)₁₂—(OCH₂CHR)ₘOPO₃H, (CF₃—CH₂—CH₂)₂N—(CH₂)₁₂—(OCH₂CHR)ₘOAr(NCO)ₚ
(CF₃—CH₂—CH₂)₂N—(CH₂)₁₃—(OCH₂CHR)ₘSH; (CF₃—CH₂—CH₂)₂N—(CH₂)₁₃—(OCH₂CHR)ₘSO₂CH═CH₂;
(CF₃—CH₂—CH₂)₂N—(CH₂)₁₃—(OCH₂CHR)ₘ—OCOCH═CH₂,
(CF₃—CH₂—CH₂)₂N—(CH₂)₁₃—(OCH₂CHR)ₘOPO₃H, (CF₃—CH₂—CH₂)₂N—(CH₂)₁₃—(OCH₂CHR)ₘOAr(NCO)ₚ
(CF₃—CH₂—CH₂)₂N—(CH₂)₁₄—(OCH₂CHR)ₘSH; (CF₃—CH₂—CH₂)₂N—(CH₂)₁₄—(OCH₂CHR)ₘSO₂CH═CH₂;
(CF₃—CH₂—CH₂)₂N—(CH₂)₁₄—(OCH₂CHR)ₘ—OCOCH═CH₂,
(CF₃—CH₂—CH₂)₂N—(CH₂)₁₄—(OCH₂CHR)ₘOPO₃H, (CF₃—CH₂—CH₂)₂N—(CH₂)₁₄—(OCH₂CHR)ₘOAr(NCO)ₚ
(CF₃—CH₂—CH₂)₂N—(CH₂)₁₅—(OCH₂CHR)ₘSH; (CF₃—CH₂—CH₂)₂N—(CH₂)₁₅—(OCH₂CHR)ₘSO₂CH═CH₂;
(CF₃—CH₂—CH₂)₂N—(CH₂)₁₅—(OCH₂CHR)ₘ—OCOCH═CH₂,
(CF₃—CH₂—CH₂)₂N—(CH₂)₁₀—(OCH₂CHR)ₘOPO₃H, (CF₃—CH₂—CH₂)₂N—(CH₂)₁₅—(OCH₂CHR)ₘOAr(NCO)ₚ
(CF₃—CH₂—CH₂)₂N—(CH₂)₁₆—(OCH₂CHR)ₘSH; (CF₃—CH₂—CH₂)₂N—(CH₂)₁₆—(OCH₂CHR)ₘSO₂CH═CH₂;
(CF₃—CH₂—CH₂)₂N—(CH₂)₁₆—(OCH₂CHR)ₘ—OCOCH═CH₂,
(CF₃—CH₂—CH₂)₂N—(CH₂)₁₆—(OCH₂CHR)ₘOPO₃H, (CF₃—CH₂—CH₂)₂N—(CH₂)₁₆—(OCH₂CHR)ₘOAr(NCO)ₚ
(CF₃—CH₂—CH₂)₂N—CH₂—Ar—(CH₂)₄—COOH; (CF₃—CH₂—CH₂)₂N—CH₂—Ar—(CH₂)₄—SO₃H;
(CF₃—CH₂—CH₂)₂N—CH₂—Ar—(CH₂)₄—O—SO₃H; (CF₃—CH₂—CH₂)₂N—CH₂—Ar—(CH₂)₄—OPO₃H
(CF₃—CH₂—CH₂)₂N—CH₂—Ar—(CH₂)₅—COOH; (CF₃—CH₂—CH₂)₂N—CH₂—Ar—(CH₂)₅—SO₃H;
(CF₃—CH₂—CH₂)₂N—CH₂—Ar—(CH₂)₅—O—SO₃H; (CF₃—CH₂—CH₂)₂N—CH₂—Ar—(CH₂)₅—OPO₃H
(CF₃—CH₂—CH₂)₂N—CH₂—Ar—(CH₂)₆—COOH; (CF₃—CH₂—CH₂)₂N—CH₂—Ar—(CH₂)₆—SO₃H;
(CF₃—CH₂—CH₂)₂N—CH₂—Ar—(CH₂)₆—O—SO₃H; (CF₃—CH₂—CH₂)₂N—CH₂—Ar—(CH₂)₆—OPO₃H
(CF₃—CH₂—CH₂)₂N—CH₂—Ar—(CH₂)₇—COOH; (CF₃—CH₂—CH₂)₂N—CH₂—Ar—(CH₂)₇—SO₃H;
(CF₃—CH₂—CH₂)₂N—CH₂—Ar—(CH₂)₇—O—SO₃H; (CF₃—CH₂—CH₂)₂N—CH₂—Ar—(CH₂)₇—OPO₃H
(CF₃—CH₂—CH₂)₂N—CH₂—Ar—(CH₂)₈—COOH (CF₃—CH₂—CH₂)₂N—CH₂—Ar—(CH₂)₈—SO₃H;
(CF₃—CH₂—CH₂)₂N—CH₂—Ar—(CH₂)₈—O—SO₃H; (CF₃—CH₂—CH₂)₂N—CH₂—Ar—(CH₂)₈—OPO₃H
(CF₃—CH₂—CH₂)₂N—CH₂—Ar—(CH₂)₉—COOH; (CF₃—CH₂—CH₂)₂N—CH₂—Ar—(CH₂)₉—SO₃H;
(CF₃—CH₂—CH₂)₂N—CH₂—Ar—(CH₂)₉—O—SO₃H; (CF₃—CH₂—CH₂)₂N—CH₂—Ar—(CH₂)₉—OPO₃H;
(CF₃—CH₂—CH₂)₂N—CH₂—Ar—(CH₂)₁₀—COOH; (CF₃—CH₂—CH₂)₂N—CH₂—Ar—(CH₂)₁₀—SO₃H;
(CF₃—CH₂—CH₂)₂N—CH₂—Ar—(CH₂)₁₀—O—SO₃H; (CF₃—CH₂—CH₂)₂N—CH₂—Ar—(CH₂)₁₀—OPO₃H
(CF₃—CH₂—CH₂)₂N—CH₂—Ar—(CH₂)₁₁—COOH; (CF₃—CH₂—CH₂)₂N—CH₂—Ar—(CH₂)₁₁—SO₃H;
(CF₃—CH₂—CH₂)₂N—CH₂—Ar—(CH₂)₁₁—O—SO₃H; (CF₃—CH₂—CH₂)₂N—CH₂—Ar—(CH₂)₁₁—OPO₃H
(CF₃—CH₂—CH₂)₂N—CH₂—Ar—(CH₂)₁₂—COOH; (CF₃—CH₂—CH₂)₂N—CH₂—Ar—(CH₂)₁₂—SO₃H;
(CF₃—CH₂—CH₂)₂N—CH₂—Ar—(CH₂)₁₂—O—SO₃H; (CF₃—CH₂—CH₂)₂N—CH₂—Ar—(CH₂)₁₂—OPO₃H
(CF₃—CH₂—CH₂)₂N—CH₂—Ar—(CH₂)₁₃—COOH; (CF₃—CH₂—CH₂)₂N—CH₂—Ar—(CH₂)₁₃—SO₃H;
(CF₃—CH₂—CH₂)₂N—CH₂—Ar—(CH₂)₁₃—O—SO₃H; (CF₃—CH₂—CH₂)₂N—CH₂—Ar—(CH₂)₁₃—OPO₃H;
(CF₃—CH₂—CH₂)₂N—CH₂—Ar—(CH₂)₁₄—COOH; (CF₃—CH₂—CH₂)₂N—CH₂—Ar—(CH₂)₁₄—SO₃H;
(CF₃—CH₂—CH₂)₂N—CH₂—Ar—(CH₂)₁₄—O—SO₃H; (CF₃—CH₂—CH₂)₂N—CH₂—Ar—(CH₂)₁₄—OPO₃H
(CF₃—CH₂—CH₂)₂N—CH₂—Ar—(CH₂)₁₅—COOH; (CF₃—CH₂—CH₂)₂N—CH₂—Ar—(CH₂)₁₅—SO₃H;
(CF₃—CH₂—CH₂)₂N—CH₂—Ar—(CH₂)₁₅—O—SO₃H; (CF₃—CH₂—CH₂)₂N—CH₂—Ar—(CH₂)₁₅—OPO₃H
(CF₃—CH₂—CH₂)₂N—CH₂—Ar—(CH₂)₁₆—COOH; (CF₃—CH₂—CH₂)₂N—CH₂—Ar—(CH₂)₁₆—SO₃H;
(CF₃—CH₂—CH₂)₂N—CH₂—Ar—(CH₂)₁₆—O—SO₃H; (CF₃—CH₂—CH₂)₂N—CH₂—Ar—(CH₂)₁₆—OPO₃H
C₂F₅—S—CH₂—CH═CH—(CH₂)₄—OH; C₂F₅—S—CH₂—CH═CH—(CH₂)₄—(OCH₂CHR)ₘ—OH;
C₂F₅—S—CH₂—CH═CH—(CH₂)₄—(OCH₂CHR)ₘ—SO₂—CH═CH₂
C₂F₅—S—CH₂—CH═CH—(CH₂)₅—OH; C₂F₅—S—CH₂—CH═CH—(CH₂)₅—(OCH₂CHR)ₘ—OH;
C₂F₅—S—CH₂—CH═CH—(CH₂)₅—(OCH₂CHR)ₘ—SO₂—CH═CH₂
C₂F₅—S—CH₂—CH═CH—(CH₂)₆—OH; C₂F₅—S—CH₂—CH═CH—(CH₂)₆—(OCH₂CHR)ₘ—OH;
C₂F₅—S—CH₂—CH═CH—(CH₂)₆—(OCH₂CHR)ₘ—SO₂—CH═CH₂
C₂F₅—S—CH₂—CH═CH—(CH₂)₇—OH; C₂F₅—S—CH₂—CH═CH—(CH₂)₇—(OCH₂CHR)ₘ—OH;
C₂F₅—S—CH₂—CH═CH—(CH₂)₇—(OCH₂CHR)ₘ—SO₂—CH═CH₂
C₂F₅—S—CH₂—CH═CH—(CH₂)₈—OH; C₂F₅—S—CH₂—CH═CH—(CH₂)₈—(OCH₂CHR)ₘ—OH;
C₂F₅—S—CH₂—CH═CH—(CH₂)₈—(OCH₂CHR)ₘ—SO₂—CH═CH₂
C₂F₅—S—CH₂—CH═CH—(CH₂)₉—OH; C₂F₅—S—CH₂—CH═CH—(CH₂)₉—(OCH₂CHR)ₘ—OH;
C₂F₅—S—CH₂—CH═CH—(CH₂)₉—(OCH₂CHR)ₘ—SO₂—CH═CH₂
C₂F₅—S—CH₂—CH═CH—(CH₂)₁₀—OH; C₂F₅—S—CH₂—CH═CH—(CH₂)₁₀—(OCH₂ CHR)ₘ—OH;
C₂F₅—S—CH₂—CH═CH—(CH₂)₁₀—(OCH₂CHR)ₘ—SO₂—CH═CH₂
C₂F₅—S—CH₂—CH═CH—(CH₂)₁₁—OH; C₂F₅—S—CH₂—CH═CH—(CH₂)₁₁—(OCH₂ CHR)ₘ—OH;
C₂F₅—SCH₂—CH═CH—(CH₂)₁₁—(OCH₂CHR)ₘ—SO₂—CH═CH₂
C₂F₅—S—CH₂—CH═CH—(CH₂)₁₂—OH; C₂F₅—S—CH₂—CH═CH—(CH₂)₁₂—(OCH₂ CHR)ₘ—OH;
C₂F₅—S—CH₂—CH═CH—(CH₂)₁₂—(OCH₂CHR)ₘ—SO₂—CH═CH₂
C₂F₅—S—CH₂—CH═CH—(CH₂)₁₃—OH; C₂F₅—S—CH₂—CH═CH—(CH₂)₁₃—(OCH₂ CHR)ₘ—OH;
C₂F₅—S—CH₂—CH═CH—(CH₂)₁₃—(OCH₂CHR)ₘ—SO₂—CH═CH₂
C₂F₅—S—CH₂—CH═CH—(CH₂)₁₄—OH; C₂F₅—S—CH₂—CH═CH—(CH₂)₁₄—(OCH₂ CHR)ₘ—OH;
C₂F₅—S—CH₂—CH═CH—(CH₂)₁₄—(OCH₂CHR)ₘ—SO₂—CH═CH₂
C₂F₅—S—CH₂—CH═CH—(CH₂)₁₅—OH; C₂F₅—S—CH₂—CH═CH—(CH₂)₁₅—(OCH₂ CHR)ₘ—OH;
C₂F₅—S—CH₂—CH═CH—(CH₂)₁₅—(OCH₂CHR)ₘ—SO₂—CH═CH₂
C₂F₅—S—CH₂—CH═CH—(CH₂)₁₆—OH; C₂F₅—S—CH₂—CH═CH—(CH₂)₁₆—(OCH₂ CHR)ₘ—OH;
C₂F₅—S—CH₂—CH═CH—(CH₂)₁₆—(OCH₂CHR)ₘ—SO₂—CH═CH₂
C₂F₅—S—CH₂—CH═CH—(CH₂)₄—COOH; C₂F₅—S—CH₂—CH═CH—(CH₂)₄—SO₃H; C₂F₅—S—CH₂—CH═CH—(CH₂)₄—OSO₃H;
C₂F₅—S—CH₂—CH═CH—(CH₂)₄—OPO₃H
C₂F₅—S—CH₂—CH═CH—(CH₂)₅—COOH; C₂F₅—S—CH₂—CH═CH—(CH₂)₅—SO₃H; C₂F₅—S—CH₂—CH═CH—(CH₂)₅—OSO₃H;
C₂F₅—S—CH₂—CH═CH—(CH₂)₅—OPO₃H
C₂F₅—S—CH₂—CH═CH—(CH₂)₆—COOH; C₂F₅—S—CH₂—CH═CH—(CH₂)₆—SO₃H; C₂F₅—S—CH₂—CH═CH—(CH₂)₆—OSO₃H;
C₂F₅—S—CH₂—CH═CH—(CH₂)₆—OPO₃H
C₂F₅—S—CH₂—CH═CH—(CH₂)₇—COOH; C₂F₅—S—CH₂—CH═CH—(CH₂)₇—SO₃H; C₂F₅—S—CH₂—CH═CH—(CH₂)₇—OSO₃
C₂F₅—S—CH₂—CH═CH—(CH₂)₇—OPO₃H
C₂F₅—S—CH₂—CH═CH—(CH₂)₈—COOH; C₂F₅—S—CH₂—CH═CH—(CH₂)₈—SO₃H; C₂F₅—S—CH₂—CH═CH—(CH₂)₈—OSO₃H;
C₂F₅—S—CH₂—CH═CH—(CH₂)₈—OPO₃H
C₂F₅—S—CH₂—CH═CH—(CH₂)₉—COOH; C₂F₅—S—CH₂—CH═CH—(CH₂)₉—SO₃H; C₂F₅—S—CH₂—CH═CH—(CH₂)₉—OSO₃H;
C₂F₅—S—CH₂—CH═CH—(CH₂)₉—OPO₃H
C₂F₅—S—CH₂—CH═CH—(CH₂)₁₀—COOH; C₂F₅—S—CH₂—CH═CH—(CH₂)₁₀—SO₃HC₂F₅—S—CH₂—CH═CH—(CH₂)₁₀—OSO₃H;
C₂F₅—S—CH₂—CH═CH—(CH₂)₁₀—OPO₃H
C₂F₅—S—CH₂—CH═CH—(CH₂)₁₁—COOH; C₂F₅—S—CH₂—CH═CH—(CH₂)₁₁—SO₃H; C₂F₅—SCH₂—CH═CH—(CH₂)₁₁—OSO₃H;
C₂F₅—SCH₂—CH═CH—(CH₂)₁₁—OPO₃H
C₂F₅—S—CH₂—CH═CH—(CH₂)₁₂—COOH; C₂F₅—S—CH₂—CH═CH—(CH₂)₁₂—SO₃H; C₂F₅—S—CH₂—CH═CH—(CH₂)₁₂—OSO₃H;
C₂F₅—S—CH₂—CH═CH—(CH₂)₁₂—OPO₃H
C₂F₅—S—CH₂—CH═CH—(CH₂)₁₃—COOH; C₂F₅—S—CH₂—CH═CH—(CH₂)₁₃—SO₃H; C₂F₅—S—CH₂—CH═CH—(CH₂)₁₃—OSO₃H;
C₂F₅—S—CH₂—CH═CH—(CH₂)₁₃—OPO₃H
C₂F₅—S—CH₂—CH═CH—(CH₂)₁₄—COOH; C₂F₅—S—CH₂—CH═CH—(CH₂)₁₄—SO₃H; C₂F₅—S—CH₂—CH═CH—(CH₂)₁₄—OSO₃H;
C₂F₅—S—CH₂—CH═CH—(CH₂)₁₄—OPO₃H
C₂F₅—S—CH₂—CH═CH—(CH₂)₁₅—COOH; C₂F₅—S—CH₂—CH═CH—(CH₂)₁₅—SO₃H; C₂F₅—S—CH₂—CH═CH—(CH₂)₁₅—OSO₃H;
C₂F₅—S—CH₂—CH═CH—(CH₂)₁₅—OPO₃H;
C₂F₅—S—CH₂—CH═CH—(CH₂)₁₆—COOH; C₂F₅—S—CH₂—CH═CH—(CH₂)₁₆—SO₃H; C₂F₅—S—CH₂—CH═CH—(CH₂)₁₆—OSO₃H;
C₂F₅—S—CH₂—CH═CH—(CH₂)₁₆—OPO₃H
C₂F₅—S—CH₂—CH═CH—(CH₂)₄—N⁺R¹R²R³Z⁻; C₂F₅—S—CH₂—CH═CH—(CH₂)₄—P⁺R¹R²R³Z⁻;
C₂F₅—S—CH₂—CH═CH—(CH₂)₄—O-glucoside; C₂F₅—S—CH₂—CH═CH—(CH₂)₄—SH
C₂F₅—S—CH₂—CH═CH—(CH₂)₅—N⁺R¹R²R³Z⁻; C₂F₅—S—CH₂—CH═CH—(CH₂)₅—P⁺R¹R²R³Z⁻;
C₂F₅—S—CH₂—CH═CH—(CH₂)₅—O-glucoside; C₂F₅—S—CH₂—CH═CH—(CH₂)₅—SH
C₂F₅—S—CH₂—CH═CH—(CH₂)₆—N⁺R¹R²R³Z⁻; C₂F₅—S—CH₂—CH═CH—(CH₂)₆—P⁺R¹R²R³Z⁻;
C₂F₅—S—CH₂—CH═CH—(CH₂)₆—O-glucoside;
C₂F₅—S—CH₂—CH═CH—(CH₂)₇—N⁺R¹R²R³Z⁻; C₂F₅—S—CH₂—CH═CH—(CH₂)₇—P⁺R¹R²R³Z⁻;
C₂F₅—S—CH₂—CH═CH—(CH₂)₇—O-glucoside;
C₂F₅—S—CH₂—CH═CH—(CH₂)₈—N⁺R¹R²R³Z⁻; C₂F₅—S—CH₂—CH═CH—(CH₂)₈—P⁺R¹R²R³Z⁻;
C₂F₅—S—CH₂—CH═CH—(CH₂)₈—O-glucoside
C₂F₅—S—CH₂—CH═CH—(CH₂)₉—N⁺R¹R²R³Z⁻; C₂F₅—S—CH₂—CH═CH—(CH₂)₉—P⁺R¹R²R³Z⁻;
C₂F₅—S—CH₂—CH═CH—(CH₂)₉—O-glucoside
C₂F₅—S—CH₂—CH═CH—(CH₂)₁₀—N⁺R¹R²R³Z⁻; C₂F₅—S—CH₂—CH═CH—(CH₂)₁₀—P⁺R¹R²R³Z⁻;
C₂F₅—S—CH₂—CH═CH—(CH₂)₁₀—O-glucoside
C₂F₅—S—CH₂—CH═CH—(CH₂)₁₁—N⁺R¹R²R³Z⁻; C₂F₅—S—CH₂—CH═CH—(CH₂)₁₁—P⁺R¹R²R³Z⁻;
C₂F₅—S—CH₂—CH═CH—(CH₂)₁₁—O-glucoside
C₂F₅—S—CH₂—CH═CH—(CH₂)₁₂—N⁺R¹R²R³Z⁻; C₂F₅—S—CH₂—CH═CH—(CH₂)₁₂—P⁺R¹R²R³Z⁻;
C₂F₅—S—CH₂—CH═CH—(CH₂)₁₂—O-glucoside
C₂F₅—S—CH₂—CH═CH—(CH₂)₁₃—N⁺R¹R²R³Z⁻; C₂F₅—S—CH₂—CH═CH—(CH₂)₁₃—P⁺R¹R²R³Z⁻;
C₂F₅—S—CH₂—CH═CH—(CH₂)₁₃—O-glucoside
C₂F₅—S—CH₂—CH═CH—(CH₂)₁₄—N⁺R¹R²R³Z⁻; C₂F₅—S—CH₂—CH═CH—(CH₂)₁₄—P⁺R¹R²R³Z⁻;
C₂F₅—S—CH₂—CH═CH—(CH₂)₁₄—O-glucoside
C₂F₅—S—CH₂—CH═CH—(CH₂)₁₅—N⁺R¹R²R³Z⁻; C₂F₅—S—CH₂—CH═CH—(CH₂)₁₅—P⁺R¹R²R³Z⁻;
C₂F₅—S—CH₂—CH═CH—(CH₂)₁₅—O-glucoside
C₂F₅—S—CH₂—CH═CH—(CH₂)₁₆—N⁺R¹R²R³Z⁻; C₂F₅—S—CH₂—CH═CH—(CH₂)₁₆—P⁺R¹R²R³Z⁻;
C₂F₅—S—CH₂—CH═CH—(CH₂)₁₆—O-glucoside
CF₃—CH₂—CH₂—S—CH₂—CH═CH—(CH₂)₄—OH; CF₃—CH₂—CH₂——S—CH₂—CH═CH—(CH₂)₄—(OCH₂CHR)ₘ—OH;
CF₃—CH₂—CH₂—S—CH₂—CH═CH—(CH₂)₄—(OCH₂CHR)ₘ—SO₂—CH═CH₂
CF₃—CH₂—CH₂—S—CH₂—CH═CH—(CH₂)₅—OH; CF₃—CH₂—CH₂——S—CH₂—CH═CH—(CH₂)₅—(OCH₂CHR)ₘ—OH;
CF₃—CH₂—CH₂—S—CH₂—CH═CH—(CH₂)₅—(OCH₂CHR)ₘ—SO₂—CH═CH₂
CF₃—CH₂—CH₂—S—CH₂—CH═CH—(CH₂)₆—OH; CF₃—CH₂—CH₂—S—CH₂—CH═CH—(CH₂)₆—(OCH₂CHR)ₘ—OH;
CF₃—CH₂—CH₂—S—CH₂—CH═CH—(CH₂)₆—(OCH₂CHR)ₘ—SO₂—CH═CH₂
CF₃—CH₂—CH₂—S—CH₂—CH═CH—(CH₂)₇—OH; CF₃—CH₂—CH₂—S—CH₂—S—CH₂—CH═CH—(CH₂)₇—(OCH₂CHR)ₘ—OH;
CF₃—CH₂—CH₂—S—CH₂—CH═CH—(CH₂)₇—(OCH₂CHR)ₘ—SO₂—CH═CH₂
CF₃—CH₂—CH₂—S—CH₂—CH═CH—(CH₂)₈—OH; CF₃—CH₂—CH₂—S—CH₂—CH═CH—(CH₂)₈—(OCH₂CHR)ₘ—OH;
CF₃—CH₂—CH₂—S—CH₂—CH═CH—(CH₂)₈—(OCH₂CHR)ₘ—SO₂—CH═CH₂
CF₃—CH₂—CH₂—S—CH₂—CH═CH—(CH₂)₉—OH; CF₃—CH₂—CH₂—S—CH₂—CH═CH—(CH₂)₉—(OCH₂CHR)ₘ—OH;
CF₃—CH₂—CH₂—S—CH₂—CH═CH—(CH₂)₉—(OCH₂CHR)ₘ—SO₂—CH═CH₂
CF₃—CH₂—CH₂—S—CH₂—CH═CH—(CH₂)₁₀—OH; CF₃—CH₂—CH₂—S—CH₂—CH═CH—(CH₂)₁₀—(OCH₂CHR)ₘ—OH;
CF₃—CH₂—CH₂—S—CH₂—CH═CH—(CH₂)₁₀—(OCH₂CHR)ₘ—SO₂—CH═CH₂
CF₃—CH₂—CH₂—S—CH₂—CH═CH—(CH₂)₁₁—OH; CF₃—CH₂—CH₂—S—CH₂—CH═CH—(CH₂)₁₁—(OCH₂CHR)ₘ—OH;
CF₃—CH₂—CH₂—S—CH₂—CH═CH—(CH₂)₁₁—(OCH₂CHR)ₘ—SO₂—CH═CH₂
CF₃—CH₂—CH₂—S—CH₂—CH═CH—(CH₂)₁₂—OH; CF₃—CH₂—CH₂—S—CH₂—CH═CH—(CH₂)₁₂—(OCH₂CHR)ₘ—OH;
CF₃—CH₂—CH₂—S—CH₂—CH═CH—(CH₂)₁₂—(OCH₂CHR)ₘ—SO₂—CH═CH₂
CF₃—CH₂—CH₂—S—CH₂—CH═CH—(CH₂)₁₃—OH; CF₃—CH₂—CH₂—S—CH₂—CH═CH—(CH₂)₁₃—(OCH₂CHR)ₘ—OH;
CF₃—CH₂—CH₂—S—CH₂—CH═CH—(CH₂)₁₃—(OCH₂CHR)ₘ—SO₂—CH═CH₂
CF₃—CH₂—CH₂—S—CH₂—CH═CH—(CH₂)₁₄—OH; CF₃—CH₂—CH₂—S—CH₂—CH═CH—(CH₂)₁₄—(OCH₂CHR)ₘ—OH;
CF₃—CH₂—CH₂—S—CH₂—CH═CH—(CH₂)₁₄—(OCH₂CHR)ₘ—SO₂—CH═CH₂
CF₃—CH₂—CH₂—S—CH₂—CH═CH—(CH₂)₁₅—OH; CF₃—CH₂—CH₂—S—CH₂—CH═CH—(CH₂)₁₅—(OCH₂CHR)ₘ—OH;
CF₃—CH₂—CH₂—S—CH₂—CH═CH—(CH₂)₁₅—(OCH₂CHR)ₘ—SO₂—CH═CH₂
CF₃—CH₂—CH₂—S—CH₂—CH═CH—(CH₂)₁₆—OH; CF₃—CH₂—CH₂—S—CH₂—CH═CH—(CH₂)₁₆—(OCH₂CHR)ₘ—OH;
CF₃—CH₂—CH₂—S—CH₂—CH═CH—(CH₂)₁₆—(OCH₂CHR)ₘ—SO₂—CH═CH₂
(CF₃—CH₂—CH₂)₂N—CH₂—CH═CH—(CH₂)₄—OH; (CF₃—CH₂ —CH₂)₂N—CH₂—CH═CH—(CH₂)₄—(OCH₂CHR)ₘ—OH;
(CF₃—CH₂—CH₂)₂N—CH₂—CH═CH—(CH₂)₄—(OCH₂CHR)ₘ—SO₂—CH═CH₂
(CF₃—CH₂—CH₂)₂N—CH₂—CH═CH—(CH₂)₅—OH; (CF₃—CH₂ —CH₂)₂N—CH₂—CH═CH—(CH₂)₅—(OCH₂CHR)ₘ—OH;
(CF₃—CH₂—CH₂)₂N—CH₂—CH═CH—(CH₂)₅—(OCH₂CHR)ₘ—SO₂—CH═CH₂
(CF₃—CH₂—CH₂)₂N—CH₂—CH═CH—(CH₂)₆—OH; (CF₃—CH₂ —CH₂)₂N—CH₂—CH═CH—(CH₂)₆—(OCH₂CHR)ₘ—OH;
(CF₃—CH₂—CH₂)₂N—CH₂—CH═CH—(CH₂)₆—(OCH₂CHR)ₘ—SO₂—CH═CH₂
(CF₃—CH₂—CH₂)₂N—CH₂—CH═CH—(CH₂)₇—OH; (CF₃—CH₂ —CH₂)₂N—CH₂—CH═CH—(CH₂)₇—(OCH₂CHR)ₘ—OH;
(CF₃—CH₂—CH₂)₂N—CH₂—CH═CH—(CH₂)₇—(OCH₂CHR)ₘ—SO₂—CH═CH₂
(CF₃—CH₂—CH₂)₂N—CH₂—CH═CH—(CH₂)₈—OH; (CF₃—CH₂ —CH₂)₂N—CH₂—CH═CH—(CH₂)₈—(OCH₂CHR)ₘ—OH;
(CF₃—CH₂—CH₂)₂N—CH₂—CH═CH—(CH₂)₈—(OCH₂CHR)ₘ—SO₂—CH═CH₂
(CF₃—CH₂—CH₂)₂N—CH₂—CH═CH—(CH₂)₉—OH; (CF₃—CH₂ —CH₂)₂N—CH₂—CH═CH—(CH₂)₉—(OCH₂CHR)ₘ—OH;
(CF₃—CH₂—CH₂)₂N—CH₂—CH═CH—(CH₂)₉—(OCH₂CHR)ₘ—SO₂—CH═CH₂
(CF₃—CH₂—CH₂)₂N—CH₂—CH═CH—(CH₂)₁₀—OH; (CF₃—CH₂ —CH₂)₂N—CH₂—CH═CH—(CH₂)₁₀—(OCH₂CHR)ₘ—OH;
(CF₃—CH₂—CH₂)₂N—CH₂—CH═CH—(CH₂)₁₀—(OCH₂CHR)ₘ—SO₂—CH═CH₂
(CF₃—CH₂—CH₂)₂N—CH₂—CH═CH—(CH₂)₁₁—OH; (CF₃—CH₂—CH₂)₂N—CH₂—CH═CH—(CH₂)₁₁—(OCH₂CHR)ₘ—OH;
(CF₃—CH₂—CH₂)₂N—CH₂—CH═CH—(CH₂)₁₁—(OCH₂CHR)ₘ—SO₂—CH═CH₂
(CF₃—CH₂—CH₂)₂N—CH₂—CH═CH—(CH₂)₁₂—OH; (CF₃—CH₂—CH₂)₂N—CH₂—CH═CH—(CH₂)₁₂—(OCH₂CHR)ₘ—OH;
(CF₃—CH₂—CH₂)₂N—CH₂—CH═CH—(CH₂)₁₂—(OCH₂CHR)ₘ—SO₂—CH═CH₂
(CF₃—CH₂—CH₂)₂N—CH₂—CH═CH—(CH₂)₁₃—OH; (CF₃—CH₂—CH₂)₂N—CH₂—CH═CH—(CH₂)₁₃—(OCH₂CHR)ₘ—OH;
(CF₃—CH₂—CH₂)₂N—CH₂—CH═CH—(CH₂)₁₃—(OCH₂CHR)ₘ—SO₂—CH═CH₂
(CF₃—CH₂—CH₂)₂N—CH₂—CH═CH—(CH₂)₁₄—OH; (CF₃—CH₂—CH₂)₂N—CH₂—CH═CH—(CH₂)₁₄—(OCH₂CHR)ₘ—OH;
(CF₃—CH₂—CH₂)₂N—CH₂—CH═CH—(CH₂)₁₄—(OCH₂CHR)ₘ—SO₂—CH═CH₂
(CF₃—CH₂—CH₂)₂N—CH₂—CH═CH—(CH₂)₁₅—OH; (CF₃—CH₂—CH₂)₂N—CH₂—CH═CH—(CH₂)₁₅—(OCH₂CHR)ₘ—OH;
(CF₃—CH₂—CH₂)₂N—CH₂—CH═CH—(CH₂)₁₅—(OCH₂CHR)ₘ—SO₂—CH═CH₂
(CF₃—CH₂—CH₂)₂N—CH₂—CH═CH—(CH₂)₁₆—OH; (CF₃—CH₂—CH₂)₂N—CH₂—CH═CH—(CH₂)₁₆—(OCH₂CHR)ₘ—OH;
(CF₃—CH₂—CH₂)₂N—CH₂—CH═CH—(CH₂)₁₆—(OCH₂CHR)ₘ—SO₂—CH═CH₂
CF₃—CF₂—S—(CH₂)₄—N⁺R¹R²R³Z⁻; CF₃—CF₂—S—(CH₂)₄—P⁺R¹R²R³Z⁻; CF₃—CF₂—S—(CH₂)₄—O-
glucoside
CF₃—CF₂—S—(CH₂)₅—N⁺R¹R²R³Z⁻; CF₃—CF₂—S—(CH₂)₅—P⁺R¹R²R³Z⁻; CF₃—CF₂—S—(CH₂)₅—O-
glucoside
CF₃—CF₂—S—(CH₂)₆—N⁺R¹R²R³Z⁻; CF₃—CF₂—S—(CH₂)₆—P⁺R¹R²R³Z⁻; CF₃—CF₂—S—(CH₂)₆—O-
glucoside
CF₃—CF₂—S—(CH₂)₇—N⁺R¹R²R³Z⁻; CF₃—CF₂—S—(CH₂)₇—P⁺R¹R²R³Z⁻; CF₃—CF₂—S—(CH₂)₇—O-
glucoside
CF₃—CF₂—S—(CH₂)₈—N⁺R¹R²R³Z⁻; CF₃—CF₂—S—(CH₂)₈—P⁺R¹R²R³Z⁻; CF₃—CF₂—S—(CH₂)₈—O-
glucoside
CF₃—CF₂—S—(CH₂)₉—N⁺R¹R²R³Z⁻; CF₃—CF₂—S—(CH₂)₉—P⁺R¹R²R³Z⁻; CF₃—CF₂—S—(CH₂)₉—O-
glucoside
CF₃—CF₂—S—(CH₂)₁₀—N⁺R¹R²R³Z⁻; CF₃—CF₂—S—(CH₂)₁₀—P⁺R¹R²R³Z⁻; CF₃—CF₂—CF₂—S—(CH₂)₁₀—O-
glucoside
CF₃—CF₂—S—(CH₂)₁₁—N⁺R¹R²R³Z⁻; CF₃—CF₂—S—(CH₂)₁₁—P⁺R¹R²R³Z⁻; CF₃—CF₂—S—(CH₂)₁₁—O-
glucoside
CF₃—CF₂—S—(CH₂)₁₂—N⁺R¹R²R³Z⁻; CF₃—CF₂—S—(CH₂)₁₂—P⁺R¹R²R³Z⁻; CF₃—CF₂—S—(CH₂)₁₂—O-
glucoside
CF₃—CF₂—S—(CH₂)₁₃—N⁺R¹R²R³Z⁻; CF₃—CF₂—S—(CH₂)₁₃—P⁺R¹R²R³Z⁻; CF₃—CF₂—S—(CH₂)₁₃—O-
glucoside
CF₃—CF₂—S—(CH₂)₁₄—N⁺R¹R²R³Z⁻; CF₃—CF₂—S—(CH₂)₁₄—P⁺R¹R²R³Z⁻; CF₃—CF₂—S—(CH₂)₁₄—O-
glucoside
CF₃—CF₂—S—(CH₂)₁₅—N⁺R¹R²R³Z⁻; CF₃—CF₂—S—(CH₂)₁₅—P⁺R¹R²R³Z⁻; CF₃—CF₂—S—(CH₂)₁₅—O-
glucoside
CF₃—CF₂—S—(CH₂)₁₆—N⁺R¹R²R³Z⁻; CF₃—CF₂—S—(CH₂)₁₆—P⁺R¹R²R³Z⁻; CF₃—CF₂—S—(CH₂)₁₆—O-
glucoside
CF₃—CH₂—CH₂—S—(CH₂)₄—N⁺R¹R²R³Z⁻; CF₃—CH₂—CH₂—S—(CH₂)₄—P⁺R¹R²R³Z⁻; CF₃—CH₂—CH₂—S—(CH₂)₄—O-
glucoside
CF₃—CH₂—CH₂—S—(CH₂)₅—N⁺R¹R²R³Z⁻; CF₃—CH₂—CH₂—S—(CH₂)₅—P⁺R¹R²R³Z⁻; CF₃—CH₂—CH₂—S—(CH₂)₅—O-
glucoside
CF₃—CH₂—CH₂—S—(CH₂)₆—N⁺R¹R²R³Z⁻; CF₃—CH₂—CH₂—S—(CH₂)₆—P⁺R¹R²R³Z⁻; CF₃—CH₂—CH₂—S—(CH₂)₆—O-
glucoside
CF₃—CH₂—CH₂—S—(CH₂)₇—N⁺R¹R²R³Z⁻; CF₃—CH₂—CH₂—S—(CH₂)₇—P⁺R¹R²R³Z⁻; CF₃—CH₂—CH₂—S—(CH₂)₇—O-
glucoside
CF₃—CH₂—CH₂—S—(CH₂)₈—N⁺R¹R²R³Z⁻; CF₃—CH₂—CH₂—S—(CH₂)₈—P⁺R¹R²R³Z⁻; CF₃—CH₂—CH₂—S—(CH₂)₈—O-
glucoside
CF₃—CH₂—CH₂—S—(CH₂)₉—N⁺R¹R²R³Z⁻; CF₃—CH₂—CH₂—S—(CH₂)₉—P⁺R¹R²R³Z⁻; CF₃—CH₂—CH₂—S—(CH₂)₉—O-
glucoside
CF₃—CH₂—CH₂—S—(CH₂)₁₀—N⁺R¹R²R³Z⁻; CF₃—CH₂—CH₂—S—(CH₂)₁₀—P⁺R¹R²R³Z⁻; CF₃—CH₂—CH₂—S—(CH₂)₁₀—O-
glucoside
CF₃—CH₂—CH₂—S—(CH₂)₁₁—N⁺R¹R²R³Z⁻; CF₃—CH₂—CH₂—S—(CH₂)₁₁—P⁺R¹R²R³Z⁻; CF₃—CH₂—CH₂—S—(CH₂)₁₁—O-
glucoside
CF₃—CH₂—CH₂—S—(CH₂)₁₂—N⁺R¹R²R³Z⁻; CF₃—CH₂—CH₂—S—(CH₂)₁₂—P⁺R¹R²R³Z⁻; CF₃—CH₂—CH₂—S—(CH₂)₁₂—O-
glucoside
CF₃—CH₂—CH₂—S—(CH₂)₁₃—N⁺R¹R²R³Z⁻; CF₃—CH₂—CH₂—S—(CH₂)₁₃—P⁺R¹R²R³Z⁻; CF₃—CH₂—CH₂—S—(CH₂)₁₃—O-
glucoside
CF₃—CH₂—CH₂—S—(CH₂)₁₄—N⁺R¹R²R³Z⁻; CF₃—CH₂—CH₂—S—(CH₂)₁₄—P⁺R¹R²R³Z⁻; CF₃—CH₂—CH₂—S—(CH₂)₁₄—O-
glucoside
CF₃—CH₂—CH₂—S—(CH₂)₁₅—N⁺R¹R²R³Z⁻; CF₃—CH₂—CH₂—S—(CH₂)₁₅—P⁺R¹R²R³Z⁻; CF₃—CH₂—CH₂—S—(CH₂)₁₅—O-
glucoside
CF₃—CH₂—CH₂—S—(CH₂)₁₆—N⁺R¹R²R³Z⁻; CF₃—CH₂—CH₂—S—(CH₂)₁₆—P⁺R¹R²R³Z⁻; CF₃—CH₂—CH₂—S—(CH₂)₁₆—O-
glucoside
CF₃—CH₂—CH₂—S—CH₂—CH═CH—(CH₂)₄—N⁺R¹R²R³Z⁻; CF₃—CH₂—CH₂—S—CH₂—CH═CH—(CH₂)₄—P⁺R¹R²R³Z⁻;
CF₃—CH₂—CH₂—S—CH₂—CH═CH—(CH₂)₄—O-glucoside; CF₃—CH₂—CH₂—S—CH₂—CH═CH—(CH₂)₄—SH
CF₃—CH₂—CH₂—S—CH₂—CH═CH(CH₂)₅—N⁺R¹R²R³Z⁻; CF₃—CH₂—CH₂—S—CH₂—CH═CH—(CH₂)₅—P⁺R¹R²R³Z⁻;
CF₃—CH₂—CH₂—S—CH₂—CH═CH—(CH₂)₅—O-glucoside; CF₃—CH₂—CH₂—S—CH₂—CH═CH—(CH₂)₅—SH
CF₃—CH₂—CH₂—S—CH₂—CH═CH(CH₂)₆—N⁺R¹R²R³Z⁻; CF₃—CH₂—CH₂—S—CH₂—CH═CH—(CH₂)₆—P⁺R¹R²R³Z⁻;
CF₃—CH₂—CH₂—S—CH₂—CH═CH(CH₂)₆—O-glucoside; CF₃—CH₂—CH₂—S—CH₂—CH═CH(CH₂)₆—SH
CF₃—CH₂—CH₂—S—CH₂—CH═CH(CH₂)₇—N⁺R¹R²R³Z⁻; CF₃—CH₂—CH₂—S—CH₂—CH═CH—(CH₂)₇—P⁺R¹R²R³Z⁻;
CF₃—CH₂—CH₂—S—CH₂—CH═CH(CH₂)₇—O-glucoside; CF₃—CH₂—CH₂—S—CH₂—CH═CH(CH₂)₇—SH
CF₃—CH₂—CH₂—S—CH₂—CH═CH(CH₂)₈—N⁺R¹R²R³Z⁻; CF₃—CH₂—CH₂—S—CH₂—CH═CH—(CH₂)₈—P⁺R¹R²R³Z⁻;
CF₃—CH₂—CH₂—S—CH₂—CH═CH(CH₂)₈—O-glucoside; CF₃—CH₂—CH₂—S—CH₂—CH═CH(CH₂)₈—SH
CF₃—CH₂—CH₂—S—CH₂—CH═CH(CH₂)₉—N⁺R¹R²R³Z⁻; CF₃—CH₂—CH₂—S—CH₂—CH═CH—(CH₂)₉—P⁺R¹R²R³Z⁻;
CF₃—CH₂—CH₂—S—CH₂—CH═CH(CH₂)₉—O-glucoside; CF₃—CH₂—CH₂—S—CH₂—CH═CH(CH₂)₉—SH
CF₃—CH₂—CH₂—S—CH₂—CH═CH(CH₂)₁₀—N⁺R¹R²R³Z⁻; CF₃—CH₂—CH₂—S—CH₂—CH═CH—(CH₂)₁₀—P⁺R¹R²R³Z⁻;
CF₃—CH₂—CH₂—S—CH₂—CH═CH(CH₂)₁₀—O-glucoside; CF₃—CH₂—CH₂—S—CH₂—CH═CH(CH₂)₁₀—SH
CF₃—CH₂—CH₂—S—CH₂—CH═CH(CH₂)₁₁—N⁺R¹R²R³Z⁻; CF₃—CH₂—CH₂—S—CH₂—CH═CH—(CH₂)₁₁—P⁺R¹R²R³Z⁻;
CF₃—CH₂—CH₂—S—CH₂—CH═CH—(CH₂)₁₁—O-glucoside; CF₃—CH₂—CH₂—S—CH₂—CH═CH—(CH₂)₁₁—SH
CF₃—CH₂—CH₂—S—CH₂—CH═CH(CH₂)₁₂—N⁺R¹R²R³Z⁻; CF₃—CH₂—CH₂—S—CH₂—CH═CH—(CH₂)₁₂—P⁺R¹R²R³Z⁻;
CF₃—CH₂—CH₂—S—CH₂—CH═CH(CH₂)₁₂—O-glucoside; CF₃—CH₂—CH₂—S—CH₂—CH═CH(CH₂)₁₂—SH
CF₃—CH₂—CH₂—S—CH₂—CH═CH(CH₂)₁₃—N⁺R¹R²R³Z⁻; CF₃—CH₂—CH₂—S—CH₂—CH═CH—(CH₂)₁₃—P⁺R¹R²R³Z⁻;
CF₃—CH₂—CH₂—S—CH₂—CH═CH(CH₂)₁₃—O-glucoside; CF₃—CH₂—CH₂—S—CH₂—CH═CH(CH₂)₁₃—SH
CF₃—CH₂—CH₂—S—CH₂—CH═CH—(CH₂)₁₄—N⁺R¹R²R³Z⁻; CF₃—CH₂—CH₂—S—CH₂—CH═CH—(CH₂)₁₄—P⁺R¹R²R³Z⁻;
CF₃—CH₂—CH₂—S—CH₂—CH═CH—(CH₂)₁₄—O-glucoside; CF₃—CH₂—CH₂—S—CH₂—CH═CH—(CH₂)₁₄—SH
CF₃—CH₂—CH₂—S—CH₂—CH═CH—(CH₂)₁₅—N⁺R¹R²R³Z⁻; CF₃—CH₂—CH₂—S—CH₂—CH═CH—(CH₂)₁₅—P⁺R¹R²R³Z⁻;
CF₃—CH₂—CH₂—S—CH₂—CH═CH—(CH₂)₁₅—O-glucoside; CF₃—CH₂—CH₂—S—CH₂—CH═CH—(CH₂)₁₅—SH
CF₃—CH₂—CH₂—S—CH₂—CH═CH—(CH₂)₁₆—N⁺R¹R²R³Z⁻; CF₃—CH₂—CH₂—S—CH₂—CH═CH—(CH₂)₁₆—P⁺R¹R²R³Z⁻;
CF₃—CH₂—CH₂—S—CH₂—CH═CH—(CH₂)₁₆—O-glucoside; CF₃—CH₂—CH₂—S—CH₂—CH═CH—(CH₂)₁₆—SH
(CF₃—CH₂—CH₂)₂N—(CH₂)₄—N⁺R¹R²R³Z⁻; (CF₃—CH₂—CH₂)₂N—(CH₂)₄—P⁺R¹R²R³Z⁻; (CF₃—CH₂—CH₂)₂N—(CH₂)₄—O-
glucoside,
(CF₃—CH₂—CH₂)₂N—(CH₂)₅—N⁺R¹R²R³Z⁻; (CF₃—CH₂—CH₂)₂N—(CH₂)₅—P⁺R¹R²R³Z⁻; (CF₃—CH₂—CH₂)₂N—(CH₂)₅—O-
glucoside
(CF₃—CH₂—CH₂)₂N—(CH₂)₆—N⁺R¹R²R³Z⁻; (CF₃—CH₂—CH₂)₂N—(CH₂)₆—P⁺R¹R²R³Z⁻; (CF₃—CH₂—CH₂)₂N—(CH₂)₆—O-
glucoside
(CF₃—CH₂—CH₂)₂N—(CH₂)₇—N⁺R¹R²R³Z⁻; (CF₃—CH₂—CH₂)₂N—(CH₂)₇—P⁺R¹R²R³Z⁻; (CF₃—CH₂—CH₂)₂N—(CH₂)₇—O-
glucoside
(CF₃—CH₂—CH₂)₂N—(CH₂)₈—N⁺R¹R²R³Z⁻; (CF₃—CH₂—CH₂)₂N—(CH₂)₈—P⁺R¹R²R³Z⁻; (CF₃—CH₂—CH₂)₂N—(CH₂)₈—O-
glucoside
(CF₃—CH₂—CH₂)₂N—(CH₂)₉—N⁺R¹R²R³Z⁻; (CF₃—CH₂—CH₂)₂N—(CH₂)₉—P⁺R¹R²R³Z⁻; (CF₃—CH₂—CH₂)₂N—(CH₂)₉—O-
glucoside
(CF₃—CH₂—CH₂)₂N—(CH₂)₁₀—N⁺R¹R²R³Z⁻; (CF₃—CH₂—CH₂)₂N—(CH₂)₁₀—P⁺R¹R²R³Z⁻;
(CF₃—CH₂—CH₂)₂N—(CH₂)₁₀—O-glucoside
(CF₃—CH₂—CH₂)₂N—(CH₂)₁₁—N⁺R¹R²R³Z⁻; (CF₃—CH₂—CH₂)₂N—(CH₂)₁₁—P⁺R¹R²R³Z⁻;
(CF₃—CH₂—CH₂)₂N—(CH₂)₁₁—O-glucoside
(CF₃—CH₂—CH₂)₂N—(CH₂)₁₂—N⁺R¹R²R³Z⁻; (CF₃—CH₂—CH₂)₂N—(CH₂)₁₂—P⁺R¹R²R³Z⁻;
(CF₃—CH₂—CH₂)₂N—(CH₂)₁₂—O-glucoside
(CF₃—CH₂—CH₂)₂N—(CH₂)₁₃—N⁺R¹R²R³Z⁻; (CF₃—CH₂—CH₂)₂N—(CH₂)₁₃—P⁺R¹R²R³Z⁻;
(CF₃—CH₂—CH₂)₂N—(CH₂)₁₃—O-glucoside
(CF₃—CH₂—CH₂)₂N—(CH₂)₁₄—N⁺R¹R²R³Z⁻; (CF₃—CH₂—CH₂)₂N—(CH₂)₁₄—P⁺R¹R²R³Z⁻;
(CF₃—CH₂—CH₂)₂N—(CH₂)₁₄—O-glucoside
(CF₃—CH₂—CH₂)₂N—(CH₂)₁₅—N⁺R¹R²R³Z⁻; (CF₃—CH₂—CH₂)₂N—(CH₂)₁₅—P⁺R¹R²R³Z⁻;
(CF₃—CH₂—CH₂)₂N—(CH₂)₁₅—O-glucoside
(CF₃—CH₂—CH₂)₂N—(CH₂)₁₆—N⁺R¹R²R³Z⁻; (CF₃—CH₂—CH₂)₂N—(CH₂)₁₆—P⁺R¹R²R³Z⁻;
(CF₃—CH₂—CH₂)₂N—(CH₂)₁₆—O-glucoside
(CF₃—CH₂—CH₂)₂N—CH₂—CH═CH—(CH₂)₄—N⁺R¹R²R³Z⁻; (CF₃—CH₂—CH₂)₂N—CH₂—CH═CH—(CH₂)₄—P⁺R¹R²R³Z⁻;
(CF₃—CH₂—CH₂)₂N—CH₂—CH═CH—(CH₂)₄—O-glucoside,
(CF₃—CH₂—CH₂)₂N—CH₂—CH═CH—(CH₂)₄—SH
(CF₃—CH₂—CH₂)₂N—CH₂—CH═CH—(CH₂)₅—N⁺R¹R²R³Z⁻; (CF₃—CH₂—CH₂)₂N—CH₂—CH═CH—(CH₂)₅—P⁺R¹R²R³Z⁻;
(CF₃—CH₂—CH₂)₂N—CH₂—CH═CH—(CH₂)₅—O-glucoside;
(CF₃—CH₂—CH₂)₂N—CH₂—CH═CH—(CH₂)₅—SH
(CF₃—CH₂—CH₂)₂N—CH₂—CH═CH—(CH₂)₆—N⁺R¹R²R³Z⁻; (CF₃—CH₂—CH₂)₂N—CH₂—CH═CH—(CH₂)₆—P⁺R¹R²R³Z⁻;
(CF₃—CH₂—CH₂)₂N—CH₂—CH═CH—(CH₂)₆—O-glucoside;
(CF₃—CH₂—CH₂)₂N—CH₂—CH═CH—(CH₂)₆—SH
(CF₃—CH₂—CH₂)₂N—CH₂—CH═CH—(CH₂)₇—N⁺R¹R²R³Z⁻; (CF₃—CH₂—CH₂)₂N—CH₂—CH═CH—(CH₂)₇—P⁺R¹R²R³Z⁻;
(CF₃—CH₂—CH₂)₂N—CH₂—CH═CH—(CH₂)₇—O-glucoside;
(CF₃—CH₂—CH₂)₂N—CH₂—CH═CH—(CH₂)₇—SH
(CF₃—CH₂—CH₂)₂N—CH₂—CH═CH—(CH₂)₈—N⁺R¹R²R³Z⁻; (CF₃—CH₂—CH₂)₂N—CH₂—CH═CH—(CH₂)₈—P⁺R¹R²R³Z⁻;
(CF₃—CH₂—CH₂)₂N—CH₂—CH═CH—(CH₂)₈—O-glucoside;
(CF₃—CH₂—CH₂)₂N—CH₂—CH═CH—(CH₂)₈—SH
(CF₃—CH₂—CH₂)₂N—CH₂—CH═CH—(CH₂)₉—N⁺R¹R²R³Z⁻; (CF₃—CH₂—CH₂)₂N—CH₂—CH═CH—(CH₂)₉—P⁺R¹R²R³Z⁻;
(CF₃—CH₂—CH₂)₂N—CH₂—CH═CH—(CH₂)₉—O-glucoside;
(CF₃—CH₂—CH₂)₂N—CH₂—CH═CH—(CH₂)₉—SH
(CF₃—CH₂—CH₂)₂N—CH₂—CH═CH—(CH₂)₁₀—N⁺R¹R²R³Z⁻; (CF₃—CH₂—CH₂)₂N—CH₂—CH═CH—(CH₂)₁₀—P⁺R¹R²R³Z⁻;
(CF₃—CH₂—CH₂)₂N—CH₂—CH═CH—(CH₂)₁₀—O-glucoside;
(CF₃—CH₂—CH₂)₂N—CH₂—CH═CH—(CH₂)₁₀—SH
(CF₃—CH₂—CH₂)₂N—CH₂—CH═CH—(CH₂)₁₁—N⁺R¹R²R³Z⁻; (CF₃—CH₂—CH₂)₂N—CH₂—CH═CH—(CH₂)₁₁—P⁺R¹R²R³Z⁻;
(CF₃—CH₂—CH₂)₂N—CH₂—CH═CH—(CH₂)₁₁—O-glucoside;
(CF₃—CH₂—CH₂)₂N—CH₂—CH═CH—(CH₂)₁₁—SH
(CF₃—CH₂—CH₂)₂N—CH₂—CH═CH—(CH₂)₁₂—N⁺R¹R²R³Z⁻; (CF₃—CH₂—CH₂)₂N—CH₂—CH═CH—(CH₂)₁₂—P⁺R¹R²R³Z⁻;
(CF₃—CH₂—CH₂)₂N—CH₂—CH═CH—(CH₂)₁₂—O-glucoside;
(CF₃—CH₂—CH₂)₂N—CH₂—CH═CH—(CH₂)₁₂—SH
(CF₃—CH₂—CH₂)₂N—CH₂—CH═CH—(CH₂)₁₃—N⁺R¹R²R³Z⁻; (CF₃—CH₂—CH₂)₂N—CH₂—CH═CH—(CH₂)₁₃—P⁺R¹R²R³Z⁻;
(CF₃—CH₂—CH₂)₂N—CH₂—CH═CH—(CH₂)₁₃—O-glucoside;
(CF₃—CH₂—CH₂)₂N—CH₂—CH═CH—(CH₂)₁₃—SH
(CF₃—CH₂—CH₂)₂N—CH₂—CH═CH—(CH₂)₁₄—N⁺R¹R²R³Z⁻; (CF₃—CH₂—CH₂)₂N—CH₂—CH═CH—(CH₂)₁₄—P⁺R¹R²R³Z⁻;
(CF₃—CH₂—CH₂)₂N—CH₂—CH═CH—(CH₂)₁₄—O-glucoside;
(CF₃—CH₂—CH₂)₂N—CH₂—CH═CH—(CH₂)₁₄—SH
(CF₃—CH₂—CH₂)₂N—CH₂—CH═CH—(CH₂)₁₅—N⁺R¹R²R³Z⁻; (CF₃—CH₂—CH₂)₂N—CH₂—CH═CH—(CH₂)₁₅—P⁺R¹R²R³Z⁻;
(CF₃—CH₂—CH₂)₂N—CH₂—CH═CH—(CH₂)₁₅—O-glucoside;
(CF₃—CH₂—CH₂)₂N—CH₂—CH═CH—(CH₂)₁₅—SH
(CF₃—CH₂—CH₂)₂N—CH₂—CH═CH—(CH₂)₁₆—N⁺R¹R²R³Z⁻; (CF₃—CH₂—CH₂)₂N—CH₂—CH═CH—(CH₂)₁₆—P⁺R¹R²R³Z⁻;
(CF₃—CH₂—CH₂)₂N—CH₂—CH═CH—(CH₂)₁₆—O-glucoside;
(CF₃—CH₂—CH₂)₂N—CH₂—CH═CH—(CH₂)₁₆—SH
C₂F₅—S—(CH₂)₄—OH; C₂F₅—S—(CH₂)₄—SH; C₂F₅—S—(CH₂)₄—(OCH₂CHR)ₘSO₂CH═CH_{2;}
C₂F₅—S—(CH₂)₄—(OCH₂CHR)ₘ—OH; C₂F₅—S—(CH₂)₄—(OCH₂CHR)ₘ—SH;
C₂F₅—S—(CH₂)₅—OH; C₂F₅—S—(CH₂)₅—SH; C₂F₅—S—(CH₂)₅—(OCH₂CHR)ₘSO₂CH═CH_{2;}
C₂F₅—S—(CH₂)₅—(OCH₂CHR)ₘ—OH; C₂F₅—S—(CH₂)₅—(OCH₂CHR)ₘ—SH;
C₂F₅—S—(CH₂)₆—OH; C₂F₅—S—(CH₂)₆—SH; C₂F₅—S—(CH₂)₆—(OCH₂CHR)ₘSO₂CH═CH_{2;}
C₂F₅—S—(CH₂)₆—(OCH₂CHR)ₘ—OH; C₂F₅—S—(CH₂)₆—(OCH₂CHR)ₘ—SH
C₂F₅—S—(CH₂)₇—OH; C₂F₅—S—(CH₂)₇—SH; C₂F₅—S—(CH₂)₇—(OCH₂CHR)ₘSO₂CH═CH_{2;}
C₂F₅—S—(CH₂)₇—(OCH₂CHR)ₘ—OH; C₂F₅—S—(CH₂)₇—(OCH₂CHR)ₘ•SH
C₂F₅—S—(CH₂)₈—OH; C₂F₅—S—(CH₂)₈—SH; C₂F₅—S—(CH₂)₈—(OCH₂CHR)ₘSO₂CH═CH_{2;}
C₂F₅—S—(CH₂)₈—(OCH₂CHR)ₘ—OH; C₂F₅—S—(CH₂)₈—(OCH₂CHR)ₘ—SH
C₂F₅—S—(CH₂)₉—OH; C₂F₅—S—(CH₂)₉—SH; C₂F₅—S—(CH₂)₉—(OCH₂CHR)ₘSO₂CH═CH_{2;}
C₂F₅—S—(CH₂)₉—(OCH₂CHR)ₘ—OH; C₂F₅—S—(CH₂)₉—(OCH₂CHR)ₘ—SH
C₂F₅—S—(CH₂)₁₀—OH; C₂F₅—S—(CH₂)₁₀—SH; C₂F₅—S—(CH₂)₁₀—(OCH₂CHR)ₘSO₂CH═CH_{2;}
C₂F₅—S—(CH₂)₁₀—(OCH₂CHR)ₘ—OH; C₂F₅—S—(CH₂)₁₀—(OCH₂CHR)ₘ—SH
C₂F₅—S—(CH₂)₁₁—OH; C₂F₅—S—(CH₂)₁₁—SH; C₂F₅—S—(CH₂)₁₁—(OCH₂CHR)ₘSO₂CH═CH_{2;}
C₂F₅—S—(CH₂)₁₁—(OCH₂CHR)ₘ—OH; C₂F₅—S—(CH₂)₁₁—(OCH₂CHR)ₘ—SH
C₂F₅—S—(CH₂)₁₂—OH; C₂F₅—S—(CH₂)₁₂—SH; C₂F₅—S—(CH₂)₁₂—(OCH₂CHR)ₘSO₂CH═CH_{2;}
C₂F₅—S—(CH₂)₁₂—(OCH₂CHR)ₘ—OH; C₂F₅—S—(CH₂)₁₂—(OCH₂CHR)ₘ—SH
C₂F₅—S—(CH₂)₁₃—OH; C₂F₅—S—(CH₂)₁₃—SH; C₂F₅—S—(CH₂)₁₃—(OCH₂CHR)ₘSO₂CH═CH_{2;}
C₂F₅—S—(CH₂)₁₃—(OCH₂CHR)ₘ—OH; C₂F₅—S—(CH₂)₁₃—(OCH₂CHR)ₘ—SH
C₂F₅—S—(CH₂)₁₄—OH; C₂F₅—S—(CH₂)₁₄—SH; C₂F₅—S—(CH₂)₁₄—(OCH₂CHR)ₘSO₂CH═CH_{2;}
C₂F₅—S—(CH₂)₁₄—(OCH₂CHR)ₘ—OH; C₂F₅—S—(CH₂)₁₄—(OCH₂CHR)ₘ—SH
C₂F₅—S—(CH₂)₁₅—OH; C₂F₅—S—(CH₂)₁₅—SH; C₂F₅—S—(CH₂)₁₅—(OCH₂CHR)ₘSO₂CH═CH_{2;}
C₂F₅—S—(CH₂)₁₅—(OCH₂CHR)ₘ—OH; C₂F₅—S—(CH₂)₁₅—(OCH₂CHR)ₘ—SH
C₂F₅—S—(CH₂)₁₆—OH; C₂F₅—S—(CH₂)₁₆—SH; C₂F₅—S—(CH₂)₁₆—(OCH₂CHR)ₘSO₂CH═CH_{2;}
C₂F₅—S—(CH₂)₁₆—(OCH₂CHR)ₘ—OH; C₂F₅—S—(CH₂)₁₆—(OCH₂CHR)ₘ—SH
C₂F₅—S—(CH₂)₄—(OCH₂CH₂)ₘ—OCOCH═CH₂; C₂F₅—S—(CH₂)₄—(OCH₂CH₂)ₘ—OCH═CH₂;
C₂F₅—S—(CH₂)₄—(OCH₂CH₂)ₘ—OAr(NCO)ₚ
C₂F₅—S—(CH₂)₅—(OCH₂CH₂)ₘ—OCOCH═CH₂; C₂F₅—S—(CH₂)₅—(OCH₂CH₂)ₘ—OCH═CH₂;
C₂F₅—S—(CH₂)₅—(OCH₂CH₂)ₘ—OAr(NCO)ₚ
C₂F₅—S—(CH₂)₆—(OCH₂CH₂)ₘ—OCOCH═CH₂; C₂F₅—S—(CH₂)₆—(OCH₂CH₂)ₘ—OCH═CH₂;
C₂F₅—S—(CH₂)₆—(OCH₂CH₂)ₘ—OAr(NCO)ₚ
C₂F₅—S—(CH₂)₇—(OCH₂CH₂)ₘ—OCOCH═CH₂; C₂F₅—S—(CH₂)₇—(OCH₂CH₂)ₘ—OCH═CH₂;
C₂F₅—S—(CH₂)₇—(OCH₂CH₂)ₘ—OAr(NCO)ₚ
C₂F₅—S—(CH₂)₈—(OCH₂CH₂)ₘ—OCOCH═CH₂; C₂F₅—S—(CH₂)₈—(OCH₂CH₂)ₘ—OCH═CH₂;
C₂F₅—S—(CH₂)₈—(OCH₂CH₂)ₘ—OAr(NCO)ₚ
C₂F₅—S—(CH₂)₉—(OCH₂CH₂)ₘ—OCOCH═CH₂; C₂F₅—S—(CH₂)₉—(OCH₂CH₂)ₘ—OCH═CH₂;
C₂F₅—S—(CH₂)₉—(OCH₂CH₂)ₘ—OAr(NCO)ₚ
C₂F₅—S—(CH₂)₁₀—(OCH₂CH₂)ₘ—OCOCH═CH₂; C₂F₅—S—(CH₂)₁₀—(OCH₂CH₂)ₘ—OCH═CH₂;
C₂F₅—S—(CH₂)₁₀—(OCH₂CH₂)ₘ—OAr(NCO)ₚ
C₂F₅—S—(CH₂)₁₁—(OCH₂CH₂)ₘ—OCOCH═CH₂; C₂F₅—S—(CH₂)₁₁—(OCH₂CH₂)ₘ—OCH═CH₂;
C₂F₅—S—(CH₂)₁₁—(OCH₂CH₂)ₘ—OAr(NCO)ₚ
C₂F₅—S—(CH₂)₁₂—(OCH₂CH₂)ₘ—OCOCH═CH₂; C₂F₅—S—(CH₂)₁₂—(OCH₂CH₂)ₘ—OCH═CH₂;
C₂F₅—S—(CH₂)₁₂—(OCH₂CH₂)ₘ—OAr(NCO)ₚ
C₂F₅—S—(CH₂)₁₃—(OCH₂CH₂)ₘ—OCOCH═CH₂; (C₂F₅—S—(CH₂)₁₃—(OCH₂CH₂)ₘ—OCH═CH₂;
C₂F₅—S—(CH₂)₁₃—(OCH₂CH₂)ₘ—OAr(NCO)ₚ
C₂F₅—S—(CH₂)₁₄—(OCH₂CH₂)ₘ—OCOCH═CH₂; C₂F₅—S—(CH₂)₁₄—(OCH₂CH₂)ₘ—OCH═CH₂;
C₂F₅—S—(CH₂)₁₄—(OCH₂CH₂)ₘ—OAr(NCO)ₚ
C₂F₅—S—(CH₂)₁₅—(OCH₂CH₂)ₘ—OCOCH═CH₂; C₂F₅—S—(CH₂)₁₅—(OCH₂CH₂)ₘ—OCH═CH₂;
C₂F₅—S—(CH₂)₁₅—(OCH₂CH₂)ₘ—OAr(NCO)ₚ
C₂F₅—S—(CH₂)₁₆—(OCH₂CH₂)ₘ—OCOCH═CH₂; C₂F₅—S—(CH₂)₁₆—(OCH₂CH₂)ₘ—OCH═CH₂;
C₂F₅—S—(CH₂)₁₆—(OCH₂CH₂)ₘ—OAr(NCO)ₚ
(CF₃—CH₂—CH₂)₂—N—(CH₂)ₙ—S—(CH₂)_{n′}—N⁺R¹R²R³Z⁻; (CF₃—CH₂—CH₂)₂—N—(CH₂)ₙ—S—(CH₂)_{n′}—P⁺R¹R²R³Z⁻;
(CF₃—CH₂—CH₂)₂—N—(CH₂)ₙ—S—(CH₂)_{n′}—O-glucoside; (CF₃—CH₂—CH₂)₂—N—(CH₂)ₙ—S—(CH₂)_{n′}—COOH;
(CF₃—CH₂—CH₂)₂—N—(CH₂)ₙ—S—(CH₂)_{n′}SO₃H; (CF₃—CH₂—CH₂)₂—N—(CH₂)ₙ—S—(CH₂)_{n′}—O—SO₃H;
(CF₃—CH₂—CH₂)₂—N—(CH₂)ₙ—S—(CH₂)_{n′}—OH; (CF₃—CH₂—CH₂)₂—N—(CH₂)ₙ—S—(CH₂)_{n′}—SH;
(CF₃—CH₂—CH₂)₂—N—(CH₂)ₙ—S—(CH₂)_{n′}—O—PO₃H;
CF₃—CH₂—CH₂—S—(CH₂)ₙ—S—(CH₂)_{n′}—N⁺R¹R²R³Z⁻; CF₃—CH₂—CH₂—S—(CH₂)ₙ—S—(CH₂)_{n′}—P⁺R¹R²R³Z⁻;
CF₃—CH₂—CH₂—S—(CH₂)ₙ—S—(CH₂)_{n′}—O-glucoside; CF₃—CH₂—CH₂—S—(CH₂)ₙ—S—(CH₂)_{n′}—COOH;
CF₃—CH₂—CH₂—S—(CH₂)ₙ—S—(CH₂)_{n′}—SO₃H; CF₃—CH₂—CH₂—S—(CH₂)ₙ—S—(CH₂)_{n′}—S—SO₃H;
CF₃—CH₂—CH₂—S—(CH₂)ₙ—S—(CH₂)_{n′}—OH; CF₃—CH₂—CH₂—S—(CH₂)ₙ—S—(CH₂)_{n′}—SH;
CF₃—CH₂—CH₂—S—(CH₂)ₙ—S—(CH₂)_{n′}—O—PO₃H;
(CF₃)₂—N—(CH₂)ₙ—S—(CH₂)_{n′}—N⁺R¹R²R³Z⁻; (CF₃)₂—N—(CH₂)ₙ—S—(CH₂)_{n′}—P⁺R¹R²R³Z⁻;
(CF₃)₂—N—(CH₂)ₙ—S—(CH₂)_{n′}—O-glucoside; (CF₃)₂—N—(CH₂)ₙ—S—(CH₂)_{n′}COOH; (CF₃)₂—N—(CH₂)ₙ—S—(CH₂)_{n′}—SO₃H;
(CF₃)₂—N—(CH₂)ₙ—S—(CH₂)_{n′}—O—SO₃H; (CF₃)₂—N—(CH₂)ₙ—S—(CH₂)_{n′}—OH;
(CF₃)₂—N—(CH₂)ₙ—S—(CH₂)_{n′}—SH; (CF₃)₂—N—(CH₂)ₙ—S—(CH₂)_{n′}—O—PO₃H;
C₂F₅—S—(CH₂)ₙ—S—(CH₂)_{n′}—N⁺R¹R²R³Z⁻; C₂F₅—S—(CH₂)ₙ—S—(CH₂)_{n′}—P⁺R¹R²R³Z⁻; C₂F₅—S—(CH₂)ₙ—S—(CH₂)_{n′}—O-glucoside;
C₂F₅—S—(CH₂)ₙ—S—(CH₂)_{n′}—COOH; C₂F₅—S—(CH₂)ₙ—S—(CH₂)_{n′}—SO₃H;
C₂F₅—S—(CH₂)ₙ—S—(CH₂)_{n′}—O—SO₃H; C₂F₅—S—(CH₂)ₙ—S—(CH₂)_{n′}—OH;
C₂F₅—S—(CH₂)ₙ—S—(CH₂)_{n′}—SH; C₂F₅—S—(CH₂)ₙ—S—(CH₂)_{n′}—O—PO₃H;
CF₃—S—(CH₂)ₙ—S—(CH₂)_{n′}—N⁺R¹R²R³Z⁻; CF₃—S—(CH₂)ₙ—S—(CH₂)_{n′}—P⁺R¹R²R³Z⁻; CF₃—S—(CH₂)ₙ—S—(CH₂)_{n′}—O-glucoside;
CF₃—S—(CH₂)ₙ—S—(CH₂)_{n′}—COOH; CF₃—S—(CH₂)ₙ—S—(CH₂)_{n′}—SO₃H;
CF₃—S—(CH₂)ₙ—S—(CH₂)_{n′}—O—SO₃H; CF₃—S—(CH₂)ₙ—S—(CH₂)_{n′}—OH; CF₃—S—(CH₂)ₙ—S—(CH₂)_{n′}—SH;
CF₃—S—(CH₂)ₙ—S—(CH₂)_{n′}—O—PO₃H;

The compounds which can be used in accordance with the invention as surfactants are particularly suitable for use as hydrophobicising agents or oleophobicising agents.

Areas of use are, for example, the surface modification of textiles, paper, glass, porous building materials or adsorbents. In paints, surface coatings, photographic coatings (for photographic plates, films and papers), special coatings for semiconductor photolithography (photoresists, top antireflective coatings, bottom antireflective coatings) or other compositions for surface coating, the compounds according to the invention and the compounds to be employed in accordance with the invention can advantageously be employed with one or more of the following functions: antifogging agent, dispersant, emulsion stabiliser, antifoam, deaerator, antistatic, flameproofing agent, gloss enhancer, lubricant, pigment or filler compatibility enhancer, scratch resistance enhancer, substrate adhesion enhancer, surface adhesion reducer, skin preventer, hydrophobicising agent, oleophobicising agent, UV stabiliser, wetting agent, flow-control agent, viscosity reducer, migration inhibitor, drying accelerator. In printing inks, the compounds according to the invention and the compounds to be employed in accordance with the invention can likewise advantageously be employed and have one or more of the following functions: antifoam, deaerator, friction control agent, wetting agent, flow-control agent, pigment compatibility enhancer, print resolution enhancer, drying accelerator.

The present invention therefore furthermore relates to the use of the compounds according to the invention or the compounds to be employed in accordance with the invention as additives in compositions for surface coating, such as printing inks, paints, surface coatings, photographic coatings, special coatings for semiconductor photolithography, such as photoresists, top antireflective coatings, bottom antireflective coatings, or in additive compositions for addition to corresponding compositions.

A further use according to the invention of compounds according to the invention or compounds to be employed in accordance with the invention is the use as interface promoter or emulsifier. These properties can be used advantageously, in particular, for the preparation of fluoropolymers by emulsion polymerisation.

Compounds according to the invention and compounds to be employed in accordance with the invention can be employed as foam stabiliser, in particular in compositions which are known as “fire-extinguishing foams”. The invention therefore furthermore relates to the use of compounds according to the invention or compounds to be employed in accordance with the invention as foam stabiliser and/or for supporting film formation, in particular in aqueous film-forming fire-extinguishing foams, both synthetic and also protein-based and also for alcohol-resistant formulations (AFFF and AFFF-AR, FP, FFFP and FFFP-AR fire-extinguishing foams).

Compounds according to the invention and compounds to be employed in accordance with the invention can also be used as antistatics. The antistatic action is important, in particular, in the treatment of textiles, in particular clothing, carpets and carpeting, upholstery in furnishings and automobiles, nonwoven textile materials, leather goods, papers and cardboards, wood and wood-based materials, mineral substrates, such as stone, cement, concrete, plaster, ceramics (glazed and unglazed tiles, stoneware, porcelain) and glasses, and for plastics and metallic substrates. The present application relates to the corresponding use.

For metallic substrates, the present invention additionally also relates to the use of compounds according to the invention in anticorrosion compositions.

The present invention furthermore also relates to the use thereof as mould-release agents in plastics processing.

In general, compounds according to the invention and compounds to be employed in accordance with the invention are suitable as antispot and antisoiling compositions, stain releases, antifogging agents, lubricants, and as abrasion resistance and mechanical wear resistance enhancers.

Compounds according to the invention and compounds to be employed in accordance with the invention can advantageously be employed as additives in cleaning compositions and spot removers for textiles (in particular clothing, carpets and carpeting, upholstery in furnishings and automobiles) and hard surfaces (in particular kitchen surfaces, sanitary ware, tiles, glass) and in polishes and waxes (in particular for furnishings, floorcoverings and automobiles) with one or more of the following functions: wetting agent, flow-control agent, hydrophobicising agent, oleophobicising agent, antispot and antisoiling agent, lubricant, antifoam, deaerator, drying accelerator. In the case of cleaning compositions and spot removers, an advantageous embodiment of the present invention is additionally also the use as detergent or dirt emulsifier and dispersant. The invention therefore furthermore relates to the use of compounds according to the invention or compounds to be employed in accordance with the invention in cleaning compositions and spot removers or as wetting agents, flow-control agents, hydrophobicising agents, oleophobicising agents, antispot and antisoiling compositions, lubricants, antifoams, deaerators or drying accelerators.

The compounds according to the invention and compounds to be employed in accordance with the invention can also advantageously be used as additives in polymeric materials (plastics) with one or more of the following functions: lubricant, internal friction reducer, UV stabiliser, hydrophobicising agent, oleophobicising agent, antispot and antisoiling agent, coupling agent for fillers, flameproofing agent, migration inhibitor (in particular against migration of plasticisers), antifogging agent.

On use as additives in liquid media for cleaning, etching, reactive modification and/or substance deposition on metal surfaces (in particular also electroplating and anodising) or semiconductor surfaces (in particular for semiconductor photolithography), compounds according to the invention and compounds to be employed in accordance with the invention act as developer, stripper, edge bead remover, etchant and cleaning agent, as wetting agent and/or deposited film quality enhancer. In the case of electroplating processes (in particular chrome-plating), the present invention additionally also relates to the function as evaporation inhibitor with or without foaming action.

In addition, the compounds which can be used in accordance with the invention as surfactants are suitable for washing and cleaning applications, in particular of textiles. The cleaning and polishing of hard surfaces is also a possible area of application of the compounds which can be used in accordance with the invention as surfactants. Furthermore, the compounds which can be used in accordance with the invention as surfactants can advantageously be employed in cosmetic products, such as, for example, foam baths and hair shampoos, or as emulsifiers in creams and lotions. The compounds according to the invention and the compounds to be employed in accordance with the invention can likewise advantageously be employed as additives in hair and body care products (for example hair rinses and hair conditioners) with one or more of the following functions: wetting agent, foaming agent, lubricant, antistatic, skin grease resistance enhancer.

Compounds according to the invention and compounds to be employed in accordance with the invention act as additives in herbicides, pesticides and fungicides with one or more of the following functions: substrate wetting agent, adjuvant, foam inhibitor, dispersant, emulsion stabiliser.

Compounds according to the invention and compounds to be employed in accordance with the invention can likewise beneficially be employed as additives in adhesives with one or more of the following functions: wetting agent, penetrant, substrate adhesion enhancer, antifoam.

Compounds according to the invention and compounds to be employed in accordance with the invention can also serve as additives in greases and hydraulic fluids with one or more of the following functions: wetting agent, corrosion inhibitor. In the case of greases, the use as dispersant (in particular for fluoropolymer particles) is additionally also an important aspect.

On use as additives in putties and fillers, compounds according to the invention and compounds to be employed in accordance with the invention can have one or more of the following functions: hydrophobicising agent, oleophobicising agent, antisoiling agent, weathering resistance enhancer, UV stabiliser, silicone bleeding preventer.

A further area of application of the compounds which can be used in accordance with the invention as surfactants is flotation, i.e. the recovery and separation of ores and minerals from dead rock. To this end, they are employed as additives in compositions for ore processing, in particular flotation and leaching solutions, with one or more of the following functions: wetting agent, foaming agent, foam inhibitor. A related use is also as additives in compositions for the stimulation of oil wells, having one or more of the following functions: wetting agent, foaming agent, emulsifier.

In addition, they can be employed as additives in de-icing compositions or anti-icing compositions.

In addition, preferred compounds of those which can be used in accordance with the invention as surfactants can also be employed as emulsifiers or dispersion aids in foods. Further areas of application are in metal treatment, as leather assistants, construction chemistry and in crop protection.

Surfactants according to the invention are furthermore also suitable as antimicrobial active ingredient, in particular as reagents for antimicrobial surface modification. For this use, it is particularly advantageous to use compounds of the formulae I to IV where X stands for a cationic polar group or a polymerisable group.

The present invention relates to all the uses mentioned here of compounds to be employed in accordance with the invention. The respective use of surfactants for the said purposes is known to the person skilled in the art, and consequently the use of compounds to be employed in accordance with the invention presents no problems.

For use, the compounds to be employed in accordance with the invention are usually incorporated into correspondingly designed compositions. Corresponding compositions, to which the present invention likewise relates, comprise at least one surface-active compound containing at least one end group Y, where Y stands for CF₃(CH₂)ₐS— or CF₃CF₂S— or [CF₃—(CH₂)ₐ]₂N—, where a stands for an integer selected from the range from 0 to 5, and a vehicle which is suitable for the particular application and optionally further specific active substances and optionally assistants.

Preferred compositions here are paint and coating compositions, fire-extinguishing compositions, greases, washing and cleaning compositions, deicers or hydrophobicising agents for the treatment of textiles or the treatment of glass. In a preferred variant of the invention, the compositions are hydrophobicising compositions for the treatment of textiles and carpets.

The hydrophobic treatment of textiles is generally carried out using hydrophobicising compositions based on polysiloxanes, fluorinated hydrocarbons or mixtures of aluminium salts or zirconium salts with paraffins (cf. in this respect “Handbuch der Textilhilfsmittel” [Handbook of Textile Assistants], A. Chwala, V. Anger, Verlag Chemie, New York 1977, Chapter 3.24 “Phobiermittel” [Phobicising Compositions], page 735 ff). The hydrophobic treatment of textiles, in particular in weather-protection clothing, serves to make the latter either water-repellent or waterproof. The hydrophobicising composition is applied to the fibres of the textiles, where it arranges itself in such a way that the hydrophobic moieties are perpendicular to the fibre surface. In this way, the attempts by water to spread over the entire surface are greatly reduced. Owing to cohesive forces, the water adopts the spherical shape and runs off the textile surface in the form of beads.

Further areas of application of compositions according to the invention are paint and coating compositions, fire-extinguishing compositions (powders and foams), greases, washing and cleaning compositions, and deicers.

The compositions can be prepared here by methods known per se; for example by mixing the compounds according to the invention with a vehicle which is suitable for the particular application and optionally further specific active substances and optionally assistants. The compounds to be used in accordance with the invention can be prepared here by methods known per se to the person skilled in the art from the literature.

The aliphatic CF₃S and CF₂CF₃S groups as well as the aliphatic (CF₃(CH₂)ₐ)₂N groups can be introduced into allyl halides by means of corresponding tetramethylammonium salts: the respective tetramethylammonium salts can be obtained as indicated in EP 1 081 129 A or DE 199 41 566 A. The corresponding disclosure of the said method in the cited references is thus expressly also part of the disclosure content of the present application.

In the following synthesis schemes, Rf stands for fully or partially fluorinated hydrocarbon radicals, as present in the end groups Y which are essential to the invention. The variable a stands for 0 to 5.

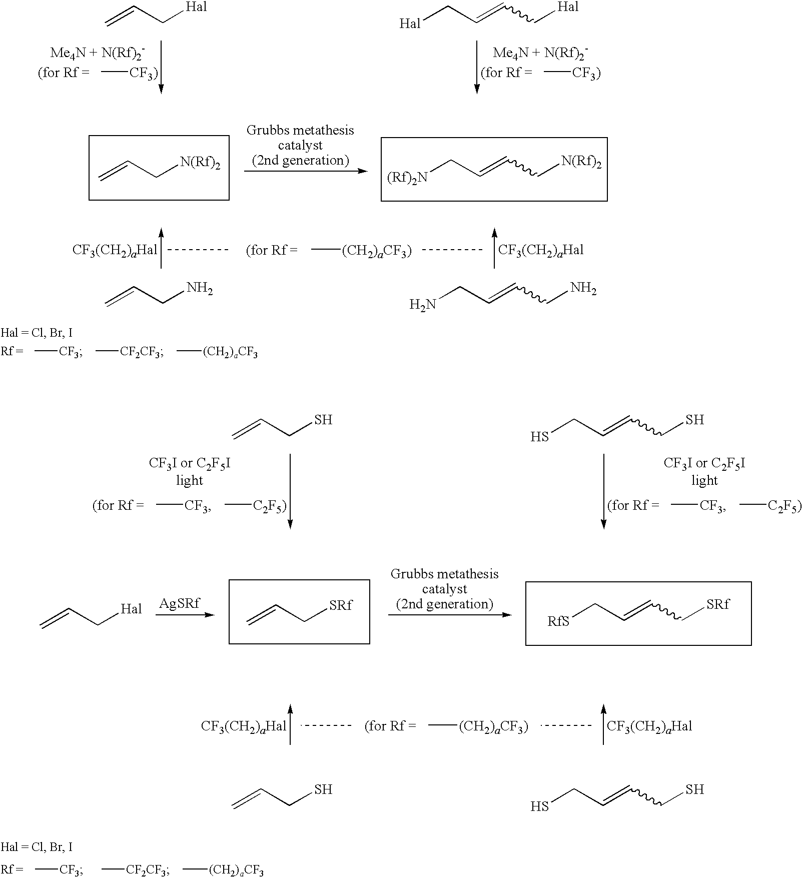

In the same way, the reaction can also be carried out with the starting materials CH₂═C(CH₂G)₂ or GCH₂CH═C(CH₂G)₂, where G stands for -Hal or —SH as shown in the above scheme, to give the corresponding products:

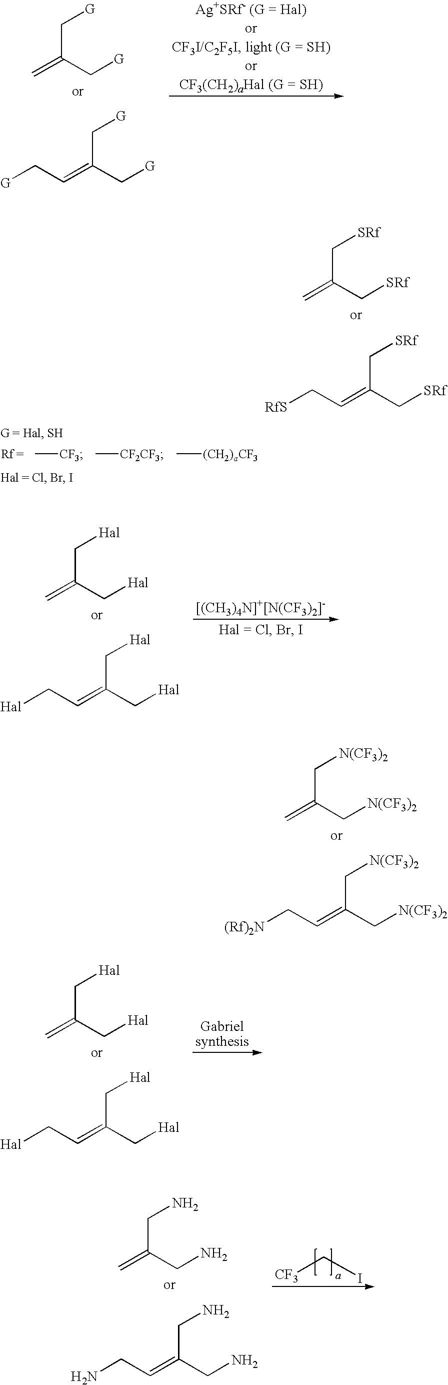

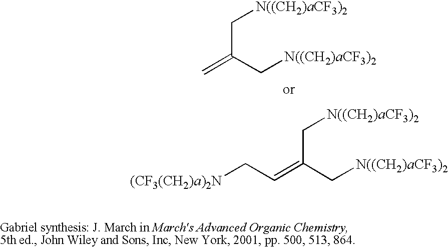

The end groups Rf=CF₃(CH₂)ₐS— or CF₃CF₂S— can preferably also be introduced into suitable molecules or intermediates by reaction of the corresponding Rf bromide with a thiolate, as described in N. V. Ignatiev et al., Zh. Organich. Khim. 1985, 21(3), p. 653, or analogously to the experimental part of this patent by reaction of a thiol with a corresponding Rf iodide in ammonia with irradiation with light. It is also possible to react the thiol directly with Rf-Br or Rf-I in the presence of a base, as described in N. V. Ignatiev, Ukr. Khim. Zh. 2001, No. 10, pp. 98-102. According to N. V. Kondartenko et al. Ukr. Khim. Zh. 1975, 41(6), pp. 516 ff, CF₃S groups can also be introduced by means of AgSCF₃ or, according to W. A. Sheppard, J. Org. Chem. 1964, 29(4), 895 ff, by means of CF₃SCl. The corresponding disclosure of the said method in the cited references is thus expressly also part of the disclosure content of the present application.

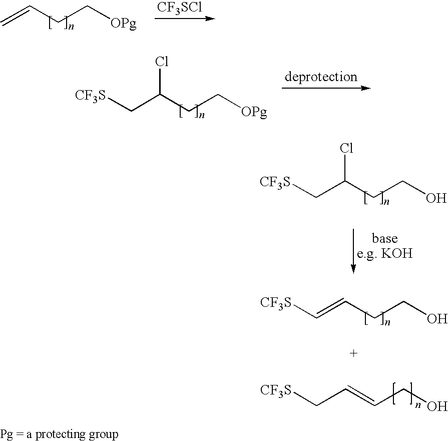

A preferred synthesis of CH₂═CH—CH₂—SH starts in this way and leads, by reaction with Rf-I in the presence of a base, to CH₂═CH—CH₂−Y, where Y stands for CF₃(CH₂)ₐS— or CF₃CF₂S—. In the same way, the reaction can also be carried out with the starting materials CH₂═C(CH₂SH)₂ or HSCH₂CH═C(CH₂SH)₂ or HSCH₂CH═CCH₂SH to give the corresponding products.

The amine moiety [CF₃—(CH₂)ₐ]₂N—, where a stands for an integer selected from the range from 1 to 5, can be introduced with the aid of the Gabriel synthesis (Organikum: Organisch-Chemisches Grundpraktikum [Practical Organic Chemistry: Basic Practical Organic Chemistry], 16th Edn, VEB Deutscher Verlag der Wissenschaften, Berlin, 1986), followed by liberation of the primary amine by reaction with hydrazine. Subsequent alkylation of this amine using CF₃(CH₂)ₐHal and debenzylation gives the tertiary amino alcohol as key building block.

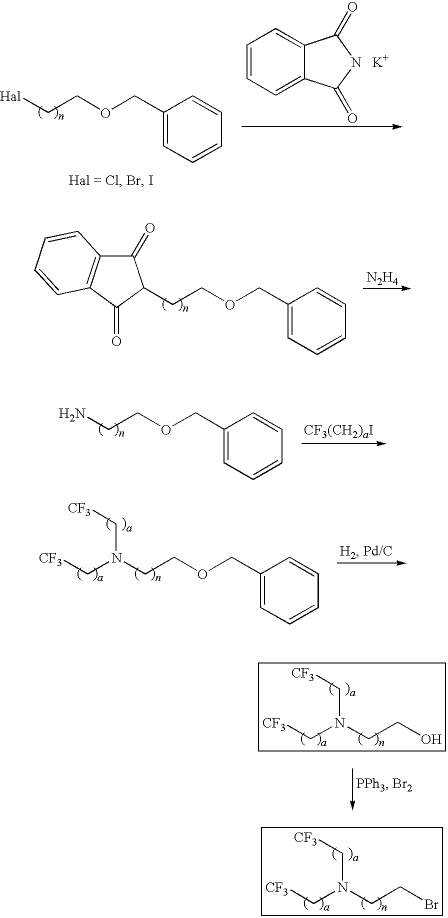

The hydrophilic, anionic, cationic, reactive or polymerisable component can be introduced via the corresponding ω-fluorinated compounds, such as, for example, alcohols, aldehydes, carboxylic acids or alkenes, by methods known to the person skilled in the art. Examples are shown in the following schemes:

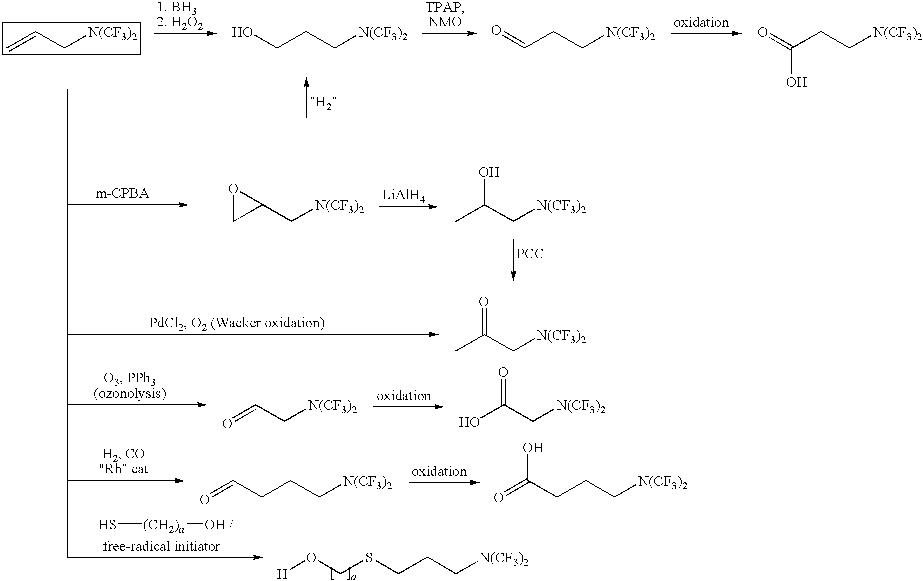

Chain Extension by Cross-Metathesis:

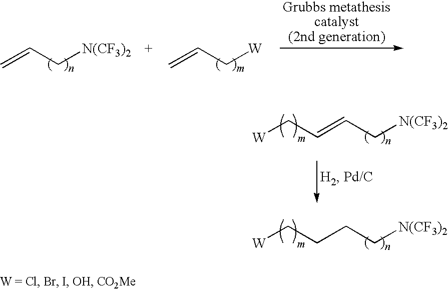

Chain Extension by Wittig Reaction:

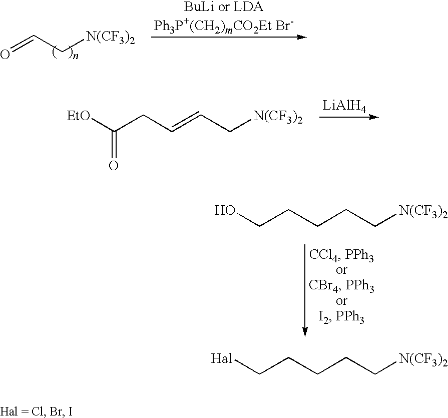

TPAP: tetra-n-propylammonium perruthenate

NMO: N-morpholine N-oxide

mCPBA: meta-chloroperbenzoic acid

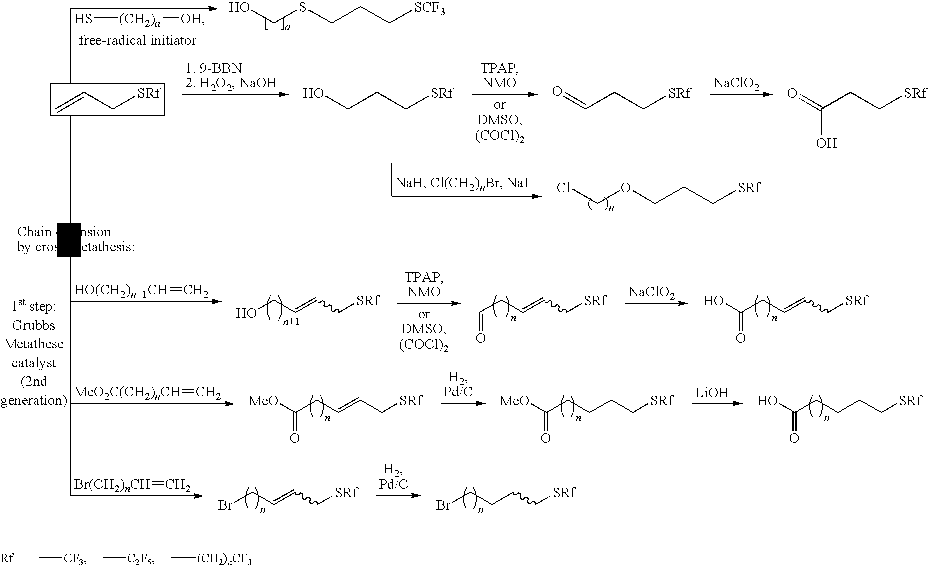

Starting from the allyl thioether, the C₃ alcohol can be obtained by hydroboration using 9-borabicyclo[3.3.1]nonane (9-BBN) and subsequent oxidation using H₂O₂ and 3 N NaOH (Ref.: Nelson, D. J. et al. J. Am. Chem. Soc. 1989, 111, 1414-1418).

The subsequent oxidation of the alcoholic C₃ building block to the aldehyde is carried out with the aid of the Ley oxidation (TPAP, NMO):
ref.: Ley-Oxidation: Griffith, W. P.; Ley, S. V. Aldrichim. Acta 1990, 23, 13. or under standard Swern conditions (DMSO, (COCl)₂):
ref.: Mancuso, A. J.; Huang, S.-L.; Swern, D. J. Org. Chem. 1987, 43, 2480.

The aldehyde obtained in this way can be converted in a Kraus oxidation (oxidation using sodium chlorite: NaClO₂) into the corresponding acid:
ref.: Kraus, G. A.; Taschner, M. J. J. Org. Chem. 1980, 45, 1175, or Crimmins, M. T. et al. J. Am. Chem. Soc. 1996, 118, 7513-7528.

A corresponding situation applies to the chain-extended aldehydes obtained by cross-metathesis (see above diagram).

The free-radical-initiated addition of a hydroxythiolate onto an Rf-substituted olefin derivative is carried out under, for example, conditions as described in Azov, V. A.; Skinner, P. J.; Yamakoshi, Y.; Seiler, P.; Gramlich, V.; Diederich, F., Helv. Chim. Acta 2003, 86, 3648.

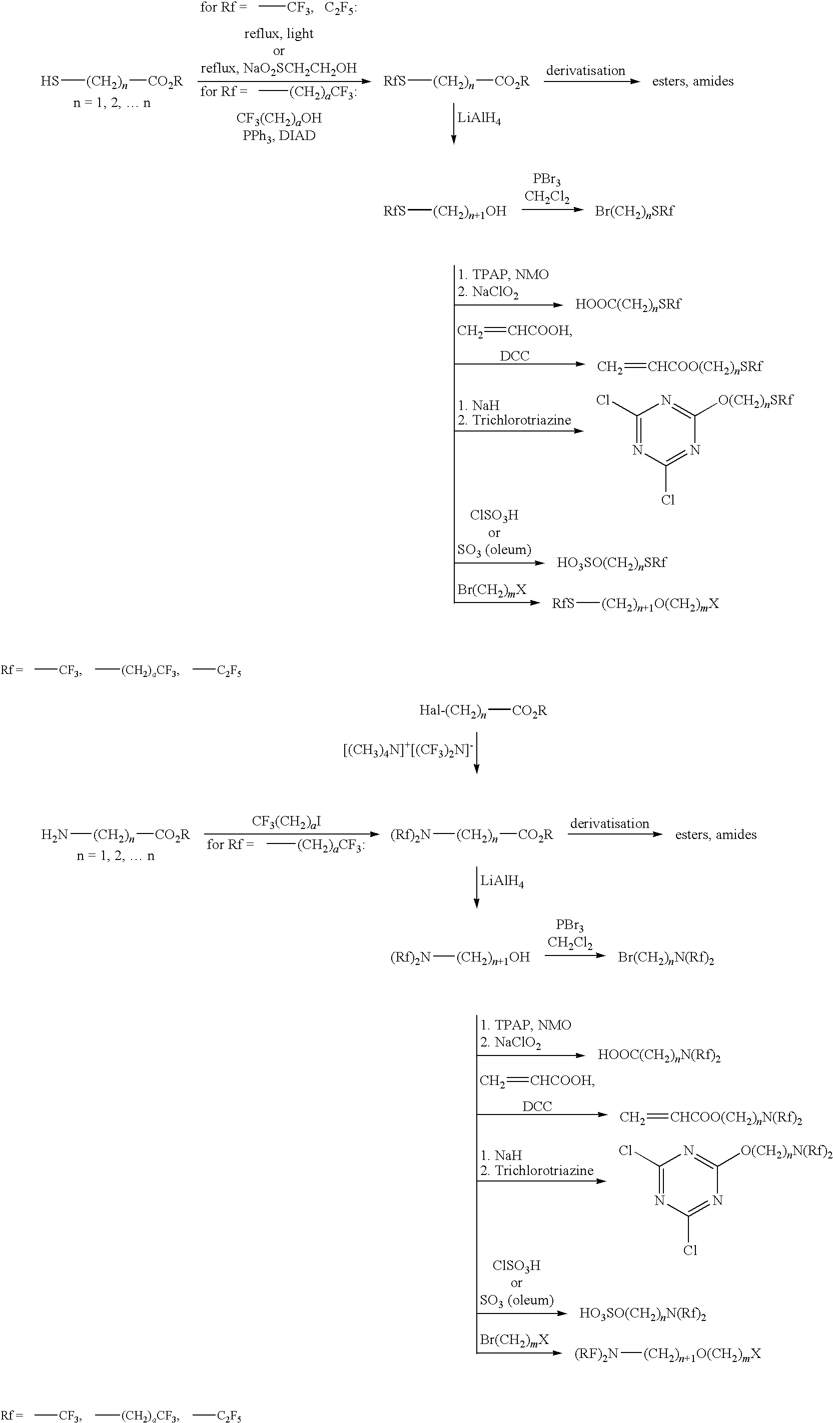

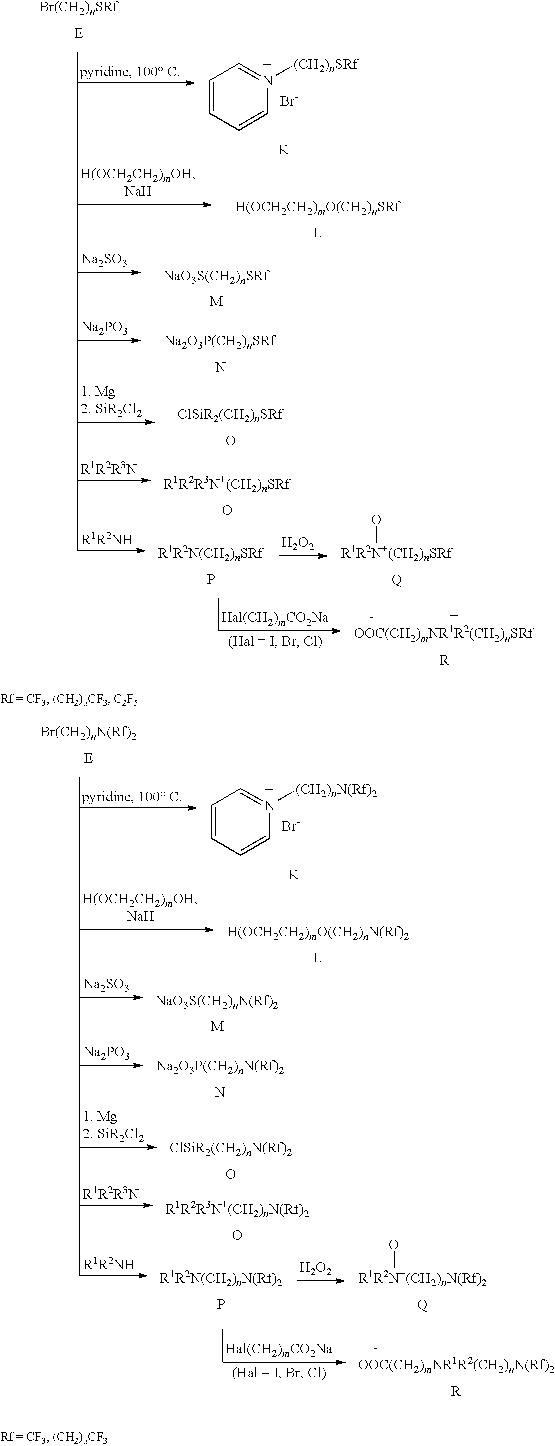

The same reaction steps can also be carried out analogously with the respective other fluorinated end groups according to the invention. The selection of suitable solvents and reaction conditions does not present the person skilled in the art with any difficulties (Organikum: Organisch-Chemisches Grundpraktikum [Basic Practical Organic Chemistry], 16th Edn, VEB Deutscher Verlag der Wissenschaften, Berlin, 1986).

The present invention therefore furthermore relates to a process for the preparation of a compound of the formula I, characterised in that firstly a compound of the formula V

Y—CH₂—CR²═CR³R⁴ V

where R², R³ and R⁴ each, independently of one another, stand for H or C₁₋₄-alkyl or Y—CH₂—, is prepared by reaction of an allyl halide Hal-CH₂—CR⁵═CR⁶R⁷, where R⁵, R⁶ and R⁷ each, independently of one another, stand for H, C₁₋₄-alkyl or Hal-CH₂—, and Hal stands for Cl, Br or I, with a tetraalkylammonium Y⁻, and this is then, if X in the compound of the formula I is not CR²═CR³R⁴ or n is >1, converted into the compound of the formula I by modification of the double bond in a manner known per se, and to a process for the preparation of a compound of the formula I, characterised in that firstly a compound of the formula V in which Y stands for CF₃(CH₂)ₐS— or CF₃CF₂S—, where a stands for an integer selected from the range from 0 to 5, is prepared by reaction of an allyl thiol HS—CH₂—CR⁵═CR⁶R⁷, where R⁵, R⁶ and R⁷ each, independently of one another, stand for H, C₁₋₄-alkyl or HS—CH₂—, with a fluorinated alkyl halide CF₃(CH₂)ₐ-Hal or CF₃CF₂-Hal in the presence of a base, where Hal stands for Br or I, and this is then, if X in the compound of the formula I is not CR²═CR³R⁴ or n is >1, converted into the compound of the formula I by modification of the double bond in a manner known per se.

Apart from the preferred compounds mentioned in the description, the use thereof, compositions and processes, the claims disclose further preferred combinations of the subject-matters according to the invention.

The disclosures in the cited references are thus expressly also part of the disclosure content of the present application.

The following examples explain the present invention in greater detail without restricting the scope of protection. In particular, the features, properties and advantages, described in the examples, of the compounds on which the relevant examples are based can also be applied to other substances and compounds which are not described in detail, but fall within the scope of protection, unless stated otherwise elsewhere. In addition, the invention can be carried out throughout the range claimed and is not restricted to the examples mentioned here.

### EXAMPLES

Index of abbreviations used:

Bn: benzyl

DBH: 1,3-dibromo-5,5-dimethylhydantoin

DCM: dichloromethane

DMAP: 4-(dimethylamino)pyridine

Me: methyl

MTB or MTBE: methyl tert-butyl ether

RT: room temperature (20° C.)

THF: tetrahydrofuran

PE: petroleum ether

THP: tetrahydropyranyl

DMF: N,N-dimethylformamide

TLC: thin-layer chromatography

DCC: dicyclohexylcarbodiimide

DI: deionised

AIBN: azoisobutyronitrile

### Example 1

### Preparation of N(CF₃)₂ Compounds

### Example 1a

### Preparation of the Ammonium Salt

(CH₃)₄N⁺F⁻+CF₃SO₂N(CF₃)₂→(CH₃)₄N+N(CF₃)₂⁻

1.05 g (11.33 mmol) of tetramethylammonium fluoride are suspended in 30 ml of dry dichloromethane at −40° C. (temperature in the bath) under an inert atmosphere. The condenser is cooled to −20° C. and kept at this temperature throughout the reaction. 3.4 g (11.93 mmol) of N,N-bis(trifluoromethyl)trifluoromethanesulfonamide, CF₃SO₂N(CF₃)₂, are subsequently slowly added dropwise, during which gas evolution is observed in the solution. The cooling bath of the reaction flask is removed 30 minutes after the addition is complete. When the reaction flask has reached room temperature, 30 ml of dry acetonitrile are added. A clear, colourless solution forms. The solvent distilled off, and the residue is dried in an oil-pump vacuum (1·10⁻³ mbar) for 60 minutes, giving tetramethylammonium bis-(trifluoromethyl)imide, [(CH₃)₄N]⁺[N(CF₃)₂]⁻, as a white solid.

Melting point: 120-125° C.

¹⁹F NMR (solvent: CD₃CN, standard: CCl₃F), ppm, δ: −43.4 s.

### Example 1b

### Preparation of the Allyl Compounds

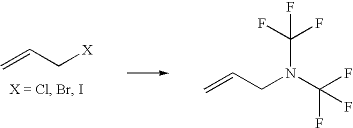

A mixture of 0.837 g (2.12 mmol) of [(CH₃)₄N]⁺[N(CF₃)₂]⁻ and 0.196 g (1.62 mmol) of allyl bromide is refluxed for a few hours under an argon atmosphere. When conversion is complete, the product is distilled off and characterised by means of NMR.

¹⁹F NMR (solvent: CD₃CN, standard: CCl₃F), ppm, δ: −55.7 s.

¹H NMR (solvent: CD₃CN, standard: TMS), ppm, δ: 3.96 d,m (CH₂), 5.27 d,m (CH), 5.35 d,m (CH), 5.91 d,d,t (CH), ³J_{H,H}=5.8 Hz, ³J_{H,H}=10.3 Hz, ³J_{H,H}=17.0 Hz.

### Example 1c

### Preparation of Further Allyl Compounds

[(C₄H₉)₄N]⁺[N(CF₃)₂]⁻+C₆H₅—CH═CH—CH₂Br→C₆H₅—CH═CH—CH₂N(CF₃)₂

A mixture of 3.27 g (8.29 mmol) of [(C₄H₉)₄N]⁺[N(CF₃)₂]⁻ and 1.47 g (7.46 mmol) of Ph-CH═CH—CH₂Br in 5 ml of dried CH₂Cl₂ is stirred at room temperature for four hours. When conversion is complete, the volatile products are condensed off in vacuo (13 Pa) and collected in a trap cooled with liquid nitrogen. After warming to room temperature, dichloromethane is distilled off, and 1-phenyl-3-bis(trifluoromethyl)aminoprop-1-ene is isolated. The substance is characterised by means of NMR.

¹⁹F NMR (solvent: CD₃CN, standard: CCl₃F), ppm, δ: −56.3 s.

¹H NMR (solvent: CD₃CN, standard: TMS), ppm, δ: 4.09 d (CH₂), 6.26 d,t (CH), 6.66 d (CH), 7.26-7.43 m (5H, Ph), ³J_{H,H}=6 Hz, ³J_{H,H}=16 Hz.

### Example 1d

### Preparation of the Alkyl Compounds

Alkyl compounds can be prepared analogously to the nucleophilic substitution reactions at the allylic centre, as described in Example 1c, by substitution of a halide, mesylate, tosylate or triflate on saturated alkyl chains using [(CH₃)₄N]⁺[N(CF₃)₂]⁻.

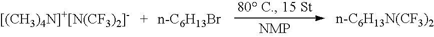

A mixture of 5.17 g (22.9 mmol) of [(CH₃)₄N]⁺[N(CF₃)₂]⁻ and 3.62 g (21.9 mmol) of hexyl bromide in 20 ml of dried N-methylpyrrolidone is stirred at 80° C. (temperature in the oil bath) for 15 hours. When conversion is complete, the product is distilled off and collected in a trap cooled with ice. N,N-Bis(trifluoromethyl)aminoheptane can be purified further by distillation. The boiling point is 124-125° C. The substance is characterised by means of NMR.

¹⁹F NMR (solvent: CD₃CN, standard: CCl₃F), ppm, δ: −56.7 s.

¹H NMR (solvent: CD₃CN, standard: TMS), ppm, δ: 0.88 m (CH₃), 1.30 m (3CH₂), 1.62 m (CH₂), 3.28 t (CH₂), ³J_{H,H}=7 Hz.

### Example 1e

### 6-Benzyloxy-1-bistrifluoromethylamine

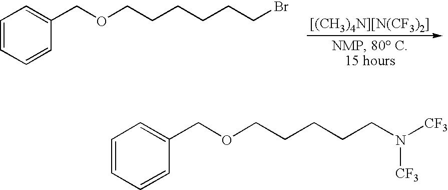

A mixture of 5 g (22.1 mmol) of [(CH₃)₄N]⁺[N(CF₃)₂]⁻ and 5.83 g (21.5 mmol) of 1-benzyloxy-6-bromohexane in 20 ml of dried N-methylpyrrolidone is stirred at 80° C. (temperature in the oil bath) for 15 hours. When conversion is complete, the product is isolated directly by column chromatography and purified. The substance is characterised by means of NMR.

¹⁹F NMR (solvent: CD₃CN, standard: CCl₃F), ppm, δ: −56.7 s.

¹H NMR (solvent: CD₃CN, standard: TMS), ppm, δ: 7.34-7.25 (m, 5H); 4.50 (s, 2H), 3.42 (t, 2H); 3.30 (dt, 2H); 1.62 (m, 2H); 1.56 (m, 2H); 1.43-1.31 (m, 4H) ppm.

### Example 1f

### 3-Bistrifluoromethylaminopropan-1-ol

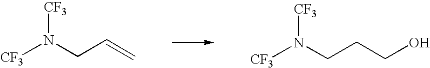

A solution of the bistrifluoroallylamine (3 g; 16 mmol; 1 eq) in 18 ml of THF is added dropwise to a stirred solution of 9-BBN in THF (0.5 molar solution; 16 mmol; 1 eq) at RT. After 24 hours, the reaction mixture (cloudy like milk) is cooled to 0° C., and 5 ml of 32% aqueous sodium hydroxide solution (evolution of heat) and 6 ml of 30% hydrogen peroxide solution (considerable evolution of heat, countercooled using a dry ice/acetone bath, reaction mixture very cloudy like milk) are then successively added dropwise. The mixture is heated at 50° C. for 1 hour (the colourless solid re-dissolves) and then cooled to RT.

For work-up, saturated sodium sulfite solution and MTB are added, the phases are separated, and the organic phase is washed with saturated NaCl solution, dried over sodium sulfate and filtered. The organic phase is employed directly in the next step.

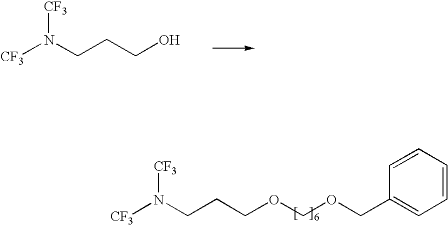

### 1-Benzyloxy-6-(3-bistrifluoromethylaminopropyl)hexane

A reaction mixture consisting of 0.8 g of tetrabutylammonium hydrogen-sulfate (2.3 mmol, 0.15 eq), a crude solution of 3-bistrifluoromethylaminopropan-1-ol (see previous step), 6.5 g of 45% NaOH solution (78.2 mmol, 5 eq) and 5.6 g of 1-bromo-6-benzyloxyhexane (20.65 mmol; 1.3 eq) is heated under reflux. After 4 days, the mixture is cooled to room temperature, diluted with water and extracted three times with MTB. The combined organic phases are dried over NaSO₄ and filtered, and the solvent is distilled off in a rotary evaporator. A slightly yellowish liquid is obtained. Purification by column chromatography gives a colourless liquid.

C₁₈H₂₅F₆NO₂; M=401.4 g/mol
¹H-NMR: (300 MHz, CDCl₃)
   7.34-7.25 (m, 5H); 4.50 (s, 2H), 3.47 (t, 2H); 3.42 (t, 2H); 3.38 (t, 2H); 3.34 (dt, 2H); 1.87 (quint., 2H); 1.62 (quint, 2H); 1.56 (quint, 2H); 1.43-1.31 (m, 4H) ppm.
¹³C-NMR (75 MHz, CDCl₃):
   138.4, 128.0, 127.2, 120.7 (q), 72.5, 70.7, 70.0, 66.6, 41.6, 29.5, 29.4, 29.3, 25.7, 25.7 ppm
¹⁹F-NMR (282 MHz, CDCl₃):
   −57.5 (s, —N(CF₃)₂) ppm
IR (KBr) 3069, 3033, 2935, 2858, 1470, 1450, 1382, 1338, 1320, 1320, 1207, 1153, 1122, 953, 737, 693 cm⁻¹

### Example 1g

### 6-(3-Bistrifluoromethylaminopropyl)hexan-1-ol

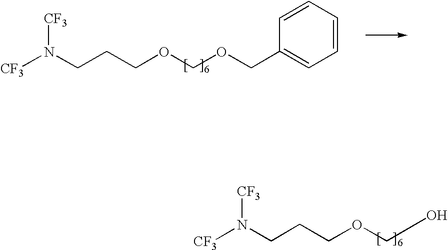

2.6 g of 1-benzyloxy-6-(3-bistrifluoromethylaminopropyl)hexane (5.7 mmol, 1 eq) are dissolved in 25 ml of THF, 2.3 g of Pd on active carbon (comprises 50.5% of water) are subsequently added, and the reaction mixture is hydrogenated at 50° C. under a hydrogen atmosphere (5 bar). Uptake of about 300 ml of hydrogen is noted. When the reaction is complete, the mixture is cooled to RT, and the catalyst is filtered off. Evaporation of the solution gives the product, which is employed directly in the next step without further purification.

C₁₁H₁₉F₆NO₂; M=311.26 g/mol
¹H-NMR: (300 MHz, CDCl₃)
   3.64 (t, 2H); 3.44 (t, 2H); 3.39 (t, 2H); 3.35 (dt, 2H); 1.88 (dt, 2H); 1.60-1.53 (m., 4H); 1.40-1.35 (m, 4H) ppm
¹³C-NMR (75 MHz, CDCl₃):
   120.5 (q), 71.0, 67.1, 42.1, 32.8, 30.3, 29.8, 29.6, 26.0, 25.6 ppm
¹⁹F-NMR (282 MHz, CDCl₃):
   −57.5 (s, —N(CF₃)₂) ppm
IR (KBr) 3372, 2942, 2868, 1477, 1385, 1338, 1321, 1207, 1153, 1123, 955, 688 cm⁻¹

### Example 1h

### 1-Bromo-6-(3-bistrifluoromethylaminopropyl)hexane

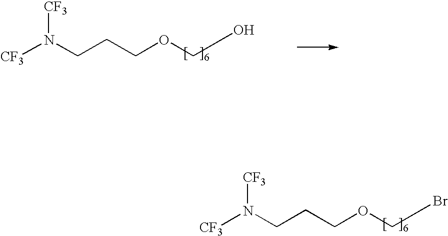

2.3 g of 6-(3-bistrifluoromethylaminopropyl)hexan-1-ol (7.4 mmol, 1 eq) are initially introduced in 60 ml of dry dichloromethane at room temperature, and 2.9 g of triphenylphosphine (11.1 mmol, 1.5 eq) are added. A solution of 4.2 g of tetrabromomethane (12.6 mmol, 1.7 eq) in 16 ml of DCM is subsequently added dropwise at 0° C. with controlled metering (internal temperature: 0-10° C.). When the addition is complete, the reaction mixture is warmed to RT, stirred for half an hour (complete conversion) and then worked up. To this end, the reaction mixture is quenched using NaHCO₃-saturated solution, and the phases are separated. The organic phase is dried over Na₂SO₄ and evaporated on a rotary evaporator together with 10 g of silica gel. The crude product on silica gel is purified by column chromatography using heptane, giving a yellowish liquid.

C₁₁H₁₈BrF₆NO; M=374.16 g/mol
¹H-NMR: (300 MHz, CDCl₃)
   3.44 (t, 2H); 3.40 (t, 2H); 3.39 (t, 2H); 3.35 (dt, 2H); 1.91-1.83 (m., 4H); 1.57 (m, 2H); 1.50-1.42 (m, 2H); 1.41-1.33 (m, 2H) ppm.
¹³C-NMR (75 MHz, CDCl₃):
   120.5 (q), 70.5, 66.7, 41.6, 33.4, 32.4, 29.4, 29.1, 27.6, 25.0 ppm
¹⁹F-NMR (282 MHz, CDCl₃):
   −57.5 (s, —N(CF₃)₂) ppm
IR (KBr) 2940, 2858, 1475, 1377, 1344, 1323, 1248, 1205, 1158, 1122, 961, 690 cm⁻¹
EI-MS M⁺=373

### Example 1i

### 6-(3-Bistrifluoromethylaminopropyl)hexane-1-sulfonic acid sodium salt

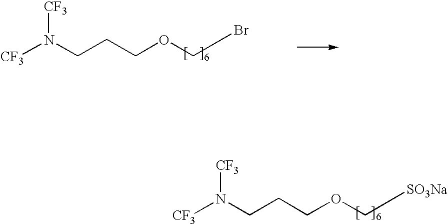

2.5 g of 1-bromo-6-(3-bistrifluoromethylaminopropyl)hexane (6.7 mmol; 1 eq) and 1.03 g of sodium sulfite (8.1 mmol; 1.2 eq) are refluxed for 24 hours in 14 ml of distilled water and 14 ml of ethanol. The reaction mixture is cooled to RT and adjusted to pH 14 using 32% NaOH. The solution is washed by shaking with a little PE/MTB 1:1 (removal of nonpolar impurities). The aqueous phase is acidified using concentrated sulfuric acid and then extracted 10 times with MTB ether. The combined organic phases are evaporated in a rotary evaporator, and 20 ml of methanol and 0.33 g of NaOH pellets are added to the residue (the sulfonic acid), the mixture is boiled up for 30 min and then cooled. The suspension is evaporated in a rotary evaporator, and the resultant residue is filtered through silica gel (MTB/MeOH 1:1). Removal of the solvent gives a colourless solid after drying in a drying cabinet at 50° C. over the weekend.

C₁₁H₁₈F₆NO₄SNa; M=397.31
¹H-NMR: (300 MHz, CDCl₃)
   3.51 (t, 2H); 3.46 (t, 2H); 3.39 (t, 2H); 2.88 (dt, 2H); 1.88 (quint., 2H); 1.74 (quint., 2H); 1.58 (m, 2H); 1.48-1.33 (m, 4H) ppm.
¹³C-NMR (75 MHz, CDCl₃):
   120.8 (q), 70.8, 66.9, 51.3, 41.5, 29.1, 28.7, 27.9, 25.1, 24.2 ppm
¹⁹F-NMR (282 MHz, CDCl₃):
   −57.8 (s, —N(CF₃)₂) ppm
IR (KBr) 3476, 2945, 2873, 1480, 1390, 1349, 1320, 1207, 1153, 1058, 958, 798, 690 cm⁻¹
ESI M⁻=374; [2M⁻H]=749; [2M⁻Na]=771

### Example 1j

### Chain Extension

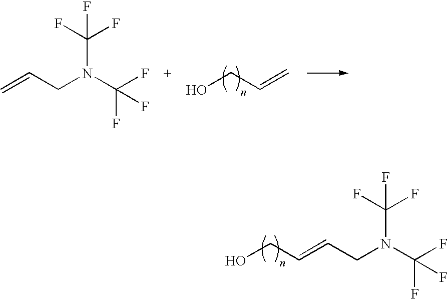

14.4 g of allylbistrifluoromethylamine (74.7 mmol, 0.77 eq) and subsequently the 2nd generation Grubbs catalyst (2.5 g; 2.9 mmol; 0.03 eq) are added to a solution of 13.8 g of 8-nonenol (n=7) (97 mmol; 1 eq) in 175 ml of dichloromethane.

The mixture is refluxed for 17 hours.

The mixture is then evaporated in a rotary evaporator and purified via a column. In order completely to remove the catalyst, the mixture is again chromatographed using PE/MTB 9/1, giving a colourless liquid (slightly brownish due to Ru residues, mixture of various homologues).

C₁₂H₁₉F₆NO; M=307.3
¹H-NMR: (300 MHz, CDCl₃)
   5.57-5.26 (m, 2H); 3.64(t, 2H); 3.24-3.18 (m, 2H); 2.10-1.95 (m, 2H); 1.64-1.53 (m., 4H); 1.43-1.27 (m., 6H) ppm.
¹⁹F-NMR (282 MHz, CDCl₃):
   −57.3 (s, —N(CF₃)₂) ppm
IR (KBr) 3372, 2930, 2853, 1470, 1382, 1320, 1158, 1074, 968, 842, 685 cm⁻¹
MALDI-MS M⁺=307

### Example 1k

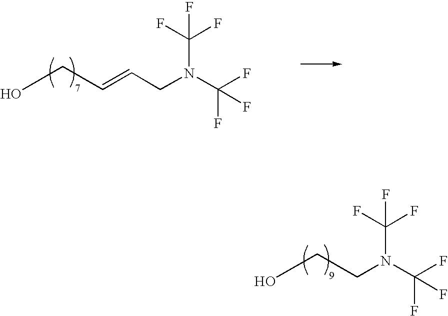

14.3 g of the alkene (46.5 mmol; 1 eq) are hydrogenated in the presence of 2.8 g of palladium (5%, dry) on active carbon in 290 g of tetrahydrofuran under a 3 bar hydrogen atmosphere. When the reaction is complete, the catalyst is filtered off, and the filtrate is evaporated, giving a slightly brownish oil.

C₁₂H₂₁F₆NO; M=309.3
¹H-NMR: (300 MHz, CDCl₃)
   3.64(t, 2H); 3.20 (t, 2H); 1.65-1.53 (m., 4H); 1.38-1.26 (m., 12H) ppm.
¹⁹F-NMR (282 MHz, CDCl₃):
   −57.3 (s, —N(CF₃)₂) ppm
IR (KBr) 3346, 2930, 2848, 1467, 1390, 1326, 1243, 1156, 1058, 960, 685 cm⁻¹

### Example 1l

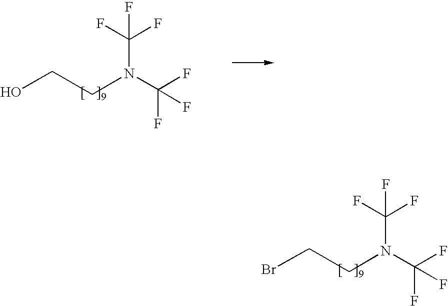

12.6 g of the alcohol (40.9 mmol; 1 eq) are initially introduced in 200 ml of dry DCM, and 21.5 g of triphenylphosphine (82 mmol; 2 eq) and subsequently, in portions, 29.8 g of tetrabromomethane (90 mmol; 2.2 eq) are added. The reaction is stirred at RT for 24 hours and then quenched using saturated NaHCO₃ solution. The phases are separated, and the organic phase is dried over sodium sulfate and evaporated in a rotary evaporator. The resultant crude product is chromatographed using petroleum ether, giving a colourless oil.

C₁₂H₂₀BrF₆N; M=372.2 g/mol
¹H-NMR: (300 MHz, CDCl₃)
   3.40 (t, 2H); 3.19 (t, 2H); 1.86 (quint., 4H); 1.63 (m, 2H), 1.43 (m, 2H), 1.34-1.26 (m., 8H) ppm.
¹⁹F-NMR (282 MHz, CDCl₃):
   −57.3 (s, —N(CF₃)₂) ppm
IR (KBr) 2935, 2858, 1472, 1382, 1331, 1254, 1156, 1102, 950, 726, 693 cm⁻¹

### Example 1m

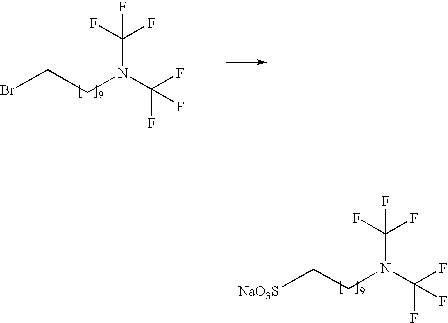

4 g of the bromide (10.7 mmol, 1 eq) and 1.76 g of sodium sulfite in 20 ml of ethanol and 20 ml of water are combined in a 100 ml flask and stirred under reflux at 100° C. for 30 hours under a nitrogen atmosphere. When the reaction is complete (TLC monitoring), the mixture is washed by shaking with PE/MTB 1:1 (removal of lipophilic by-products), and the aqueous phase is adjusted to pH=0 using concentrated sulfuric acid and washed by shaking 10 times with MTB. The combined organic phases are evaporated in a rotary evaporator, the residue is taken up in 30 ml of methanol, and the mixture is refluxed for 1 hour in the presence of 0.44 g (1 eq) of NaOH. After cooling, the mixture is purified via a silica-gel column using MTB/−MeOH 1:1. Drying in a drying cabinet in vacuo gives a colourless solid. Melting point: decomposition >160° C.

C₁₂H₂₀BrF₆NO₃S; M=395.3 g/mol
¹H-NMR: (300 MHz, CDCl₃)
   3.18 (t, 2H); 2.84 (t, 2H); 1.78-1.69 (m, 2H), 1.63-1.55 (m, 2H), 1.41-1.25 (m, 12H) ppm.
¹⁹F-NMR (282 MHz, CDCl₃):
   −57.4 (s, —N(CF₃)₂) ppm
IR (KBr) 3521, 2935, 2868, 1475, 1390, 1338, 1179, 1053, 948, 793, 724, 693 cm⁻¹
ESI M⁻=372
   [2M⁻+Na]=767

### Example 2

### (CF₃(CH₂)₂)₂N—(CH₂)₆—SO₃Na

### Example 2a

### N-Alkylation

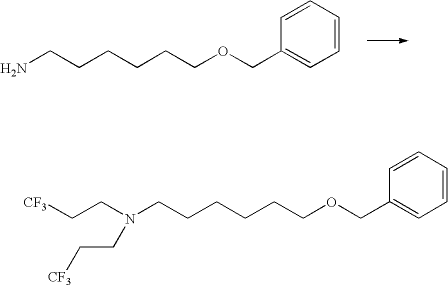

6.7 g of 1,1,1-trifluoro-3-iodopropane and 4 g of K₂CO₃ are added with stirring to 2.07 g of 6-benzyloxy-1-aminohexane (synthesis from 1,6-dibromohexane: Alvarez, M.; Granados, R.; Mauleon, D.; Rosell, G.; Salas, M.; Salles, J.; Valls, N., J. Med. Chem., 1987, 30, 1186) in 30 ml of DMF. The reaction mixture is warmed at 60° C. for 6 h under a nitrogen atmosphere and, after cooling, added to ice/water. The aqueous phase is extracted a number of times with dichloromethane, the combined organic phases are dried, and the crude product is purified by column chromatography. Yellow oil, decomposition>128° C.

H-NMR (CDCl₃, 400 MHz):

7.37(2H, m), 7.20-7.26(3H, m), 4.83(2H, m), 3.68(2H, t), 2.81(4H, t), 2.38(2H, m), 1.93(4H, m), 1.66(2H, m), 1.50(2H, m), 1.37(4H, m)

### Example 2b

### Benzyl Ether Cleavage

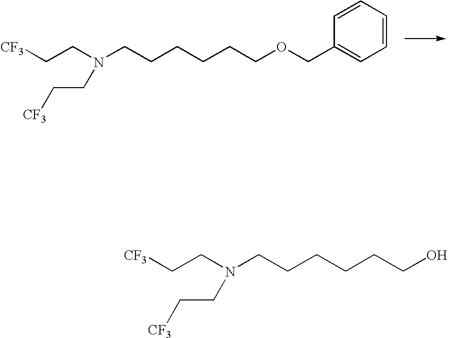

0.5 g of Pd/C (5%) are added to 3.9 g of the benzyl ether in 30 ml of ethanol in a pressure reactor, and the mixture is hydrogenated using 2.5 bar of hydrogen with stirring. The suspension is filtered, and the solvent is evaporated.

H-NMR (CDCl₃, 400 MHz):

3.62 (2H, m), 2.81 (4H, m), 2.24 (2H, t), 2.07 (4H, m), 1.34 (4H, m), 1.04 (4H, m)

### Example 2c

### Bromination

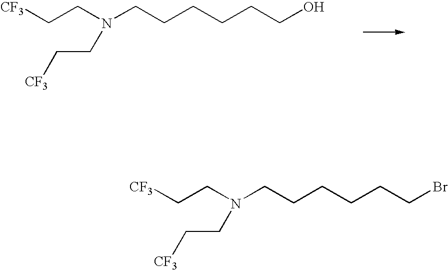

3.0 g of the amino alcohol (9.7 mmol; 1 eq) are initially introduced in 40 ml of dry DCM, and 5.1 g of triphenylphosphine (19.4 mmol; 2 eq) and subsequently, in portions, 7.1 g of tetrabromomethane (21.3 mmol; 2.2 eq) are added. The reaction is stirred at RT for 24 hours and then quenched using saturated NaHCO₃ solution. The phases are separated, and the organic phase is dried over sodium sulfate and evaporated in a rotary evaporator. The resultant crude product is purified by column chromatography.

H-NMR (CDCl₃, 400 MHz):

3.40 (2H, m); 2.81 (4H, m), 2.23 (2H, t), 2.06 (4H, m), 1.74 (2H, m), 1.32 (2H, m), 1.02-1.07 (4H, m)

### Example 2d

### Sulfonylation/Salt Formation

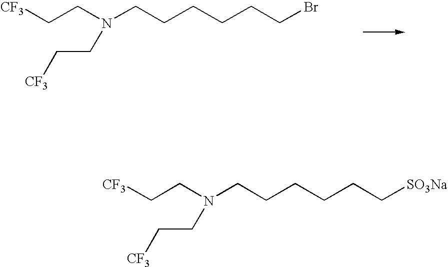

2.0 g of NaSO₃ are added to 3.2 g of the aminobromohexane in 20 ml of ethanol and 20 ml of water. The reaction mixture is stirred at 100° C. for 20 hours under nitrogen. After cooling, the suspension is filtered, and the solvent is removed by freeze-drying.

H-NMR (CDCl₃, 400 MHz): 2.83 (4H, m), 2.42-2.50 (4H, m), 2.10-2.15 (6H, m), 1.49-1.58 (6H, m)

Melting point: decomposition >134° C.

CF₃S or CF₃CF₂S or CF₃CH₂S end groups can also be introduced instead of (CF₃)₂N end groups analogously to Examples 1a-1c. In the case of the sulfur-containing compounds, Pt or Ru catalysts are employed instead of Pd catalysts.

### Example 3

### Synthesis of trifluoromethyl thioethers

### Example 3a

Methyl[(trifluoromethyl)thio]butanoate (synthesis as described in: E. Anselmi et al. J. Fluorine Chem. 2000, 105, 41-44) from methyl 4-mercaptobutyrate

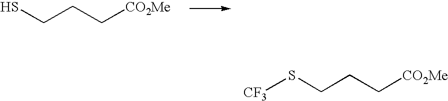

A mixture of methyl mercaptobutanoate (6.3 g; 47 mmol) and sodium phosphate (7.72 g) in 100 ml of DMF is stirred for 15 min. 4.5 ml of water and sodium hydroxymethanesulfinate (10.9 g) are subsequently added rapidly. The flask is evacuated (5 mmHg) and then kept under 4.5 bar of bromotrifluoromethane for 8 hours with shaking. The apparatus is subsequently aerated, and the supernatant liquid is diluted with 50 ml of water. The salts which remain are taken up in diethyl ether, filtered and washed with diethyl ether. The combined organic phases are washed with water (5×100 ml) and saturated NaCl. After drying over Na₂SO₄, the solvent is removed, and the residue is purified by a short-path distillation.

### Example 3b

### Reduction

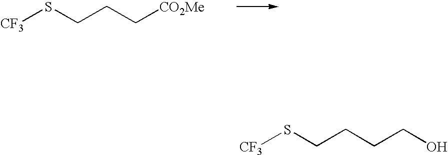

Methyl[(trifluoromethyl)thio]butanoate (5.5 g; 27 mmol) is dissolved in 300 ml of THF and cooled to 0° C. Lithium aluminium hydride (1.14 g; 30 mmol) is subsequently added in portions. The mixture is stirred at 0° C. for 1 h and subsequently warmed to RT. When conversion is complete, the mixture is re-cooled to 0° C., and 4.7 ml of ethyl acetate, 2.1 ml of water, 2.1 ml of 2 N NaOH and finally 6.2 ml of water are successively slowly added dropwise. The mixture is warmed to RT and stirred for a further hour. After this time, a little sodium sulfate is added and filtered off, the solvent is stripped off, and the residue is purified by distillation.

### Example 3c

### Protection Using THP

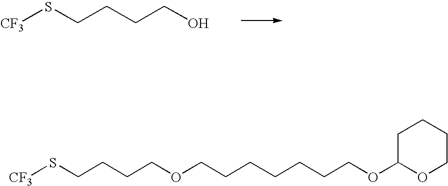

Bu₄NHSO₄ (0.74 g; 2.2 mmol) and 15 ml of a 5% NaOH solution are added to a solution of 7-tetrahydropyranyloxy-1-bromoheptane (2.79 g; 10 mmol) [for monoprotection of 1,n-difunctional compounds (for example diols) see: F. Zaragoza Dörwald: Side Reactions in Organic Synthesis, Wiley-VCH-Verlag Weinheim; 2005 pp. 333 ff] and 4-trifluoromethylthiobutan-1-ol (1.57 g; 9 mmol) in 100 ml of dichloromethane. The reaction mixture is stirred under reflux. After the two-phase mixture has been cooled, the aqueous phase is neutralised using HCl solution (1 N), the phases are separated, and the organic phase is washed with saturated NaCl solution and dried over sodium sulfate. After the solvent has been stripped off, the residue is purified by column chromatography.

### Example 3d

### Etherification and Deprotection

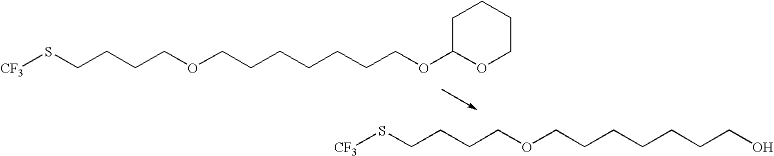

The tetrahydropyranyl acetal (2.98 g; 8 mmol) is dissolved in 80 ml of THF, a catalytic amount of p-toluenesulfonic acid is added, and the mixture is subsequently stirred at RT until conversion is complete. The mixture is added to saturated sodium hydrogencarbonate solution, and the phases are separated. After re-extraction of the aqueous phase, the collected organic phases are washed with saturated NaCl and dried over Na₂SO₄. After removal of the solvent, the residue is purified by column chromatography.

### Example 3e

### Bromination

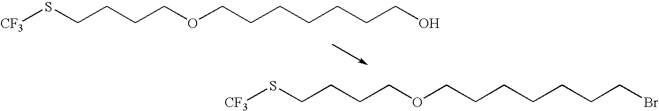

The alcohol (2.79 g; 9.7 mmol) is initially introduced in dry DCM (0.1 molar solution), and triphenylphosphine (3.8 g; 15 mmol) and subsequently, in portions, tetrabromomethane (CBr₄: 5.5 g; 16.5 mmol) are added. The reaction is stirred for 12 hours and then quenched using saturated NaHCO₃ solution. The phases are separated, and the organic phase is dried over sodium sulfate. The resultant crude product is chromatographed using petroleum ether.

### Example 3f

### Surfactant synthesis (pyridine alkylation)

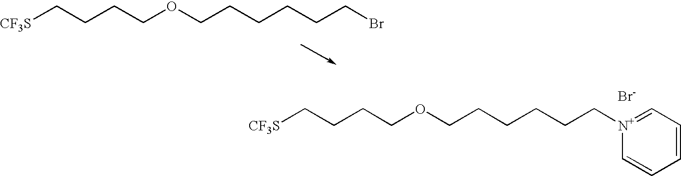

The bromide(1-bromo-7-(4-trifluoromethylsulfanylbutoxy)heptane (1.75 g; 5 mmol)) is refluxed for 2 days in 30 ml of pyridine. When the reaction is complete, the excess pyridine is stripped off. The surfactant product (1-[7-(4-trifluoromethylsulfanylbutoxy)heptyl]pyridinium bromide) obtained in this way can, if necessary, be purified by recrystallisation.

H-NMR (CDCl₃; 400 MHz):

7.06 (2H, m), 6.84 (2H, m), 6.75 (1H, m), 4.23 (2H, m), 3.47 (2H, m), 3.29 (2H, t), 2.75 (2H, m), 1.34-1.48 (12H, m).

### Example 3g

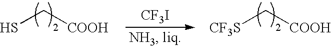

50 g (0.47 mol) of 3-mercaptopropionic acid are initially introduced into a round-bottomed flask, and about 300 ml of liquid ammonia and subsequently 120 g (0.61 mol) of trifluoroiodomethane are condensed in at −78° C. The reaction mixture is irradiated with a UV lamp with stirring for 2 hours at −50° C. and for 2 hours at −15° C. (temperature in the bath) (the reflux condenser is kept at −78° C.). After removal of volatile substances (ammonia and excess CF₃I) at room temperature, the residue is diluted with 150 ml of water and acidified using concentrated hydrochloric acid. The mixture is extracted twice with 150 ml of dichloromethane. The extract is washed with water and dried using sodium sulfate. The dichloromethane is distilled off, and the residue is dried at room temperature for 3 hours under a vacuum of 7 Pa, giving a highly viscous liquid substance. The substance is characterised by means of NMR spectra.

¹H NMR (solvent: DMSO-D₆, standard: TMS), ppm, δ: 2.66 t (CH₂), 3.11 t (CH₂), 12.53 s (OH), ³J_{H,H}=6.8 Hz.

¹⁹F NMR (solvent: DMSO-D₆, standard: CCl₃F), ppm, δ: −43.2 s (SCF₃).

### Example 3h

### Further synthesis of trifluoromethyl thioether

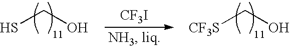

11.2 g (54.8 mmol) of 11-mercaptoundecan-1-ol are initially introduced into a round-bottomed flask, and about 40 ml of liquid ammonia and subsequently 15.0 g (76.6 mmol) of trifluoroiodomethane are condensed in at −78° C. The reaction mixture is irradiated with a UV lamp with stirring for 3 hours at −15° C. (temperature in the bath) (the reflux condenser is kept at −78° C.). After removal of volatile substances (ammonia and excess CF₃I) at room temperature, the residue is diluted with 10 ml of water and acidified using concentrated hydrochloric acid. The mixture is extracted twice with 25 ml of dichloromethane. The extract is washed with water and dried using sodium sulfate. The dichloromethane is distilled off, and the residue is dried at room temperature for 2 hours under a vacuum of 7 Pa, giving a highly viscous substance. S-trifluoromethyl-11-mercaptoundecan-1-ol is characterised by means of NMR spectra.

¹H NMR (solvent: DMSO-D₆, standard: TMS), ppm, δ: 1.23 m (5CH₂), 1.36 m (3CH₂), 1.62 m (CH₂), 2.96 t (CH₂), 3.36 m (CH₂), 4.28 t (OH), ³J_{H,H}=7.4 Hz, ³J_{H,H}=4.1 Hz.

¹⁹F NMR (solvent: DMSO-D₆, standard: CCl₃F), ppm, δ: −42.3 s (SCF₃).

### Example 3i

### 11-(S-trifluoromethyl)mercapto-1-bromoundecane

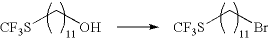

13 g of 11-(S-trifluoromethyl)mercapto-1-undecanol (47.7 mmol, 1 eq) are initially introduced in 300 ml of dry dichloromethane at room temperature, and 18.8 g of triphenylphosphine (71.6 mmol, 1.5 eq) are added. A solution of 26.9 g of tetrabromomethane (81.1 mmol, 1.7 eq) in 50 ml of DCM is subsequently added dropwise at 0° C. at a controlled rate (internal temperature: 0-10° C.). When the addition is complete, the reaction mixture is warmed to RT, stirred for half an hour (complete conversion) and then worked up. To this end, the reaction mixture is quenched using NaHCO₃— sat. solution, and the phases are separated. The organic phase is dried over Na₂SO₄ and evaporated in a rotary evaporator together with 50 g of silica gel. The crude product on silica gel is purified by column chromatography using heptane, giving a colourless liquid.

M=335.27
¹H-NMR: (300 MHz, CDCl₃)
   3.41 (t, 2H); 2.88 (t, 2H); 1.85 (quint., 2H); 1.68 (dt, 2H); 1.47-1.35 (m, 4H); 1.35-1.25 (m, 10H) ppm.
¹³C-NMR (75 MHz, CDCl₃):
   131.2 (q), 34.0, 32.8, 29.9, 29.9, 29.4, 29.4, 29.3, 28.9, 28.7, 28.5, 28.2 ppm
¹⁹F-NMR (282 MHz, CDCl₃):
   −41.2 (s, —SCF₃) ppm
IR (KBr) 2935, 2853, 1462, 1441, 1295, 1251, 1220, 1150, 1107, 760, 724 cm⁻¹
EI-MS M⁺−H=333; M⁺−H+2=335

### Example 3j

### 11-(S-trifluoromethyl)mercaptoundecane-1-sulfonic acid sodium salt

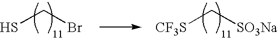

11-(S-trifluoromethyl)mercapto-1-bromoundecane (34.9 mmol; 1 eq) and 4.8 g of sodium sulfite (38.4 mmol; 1.1 eq) are dissolved in 70 ml of distilled water and 70 ml of EtOH in a 250 ml single-necked flask and heated at 100° C. for 19 hours.

The reaction mixture is cooled and extracted with a little MTB/heptane (1:1) (removal of the starting material and nonpolar impurities).

The aqueous phase is acidified using a few drops of concentrated H₂SO₄ (pH=0) and then extracted 10 times with MTB. Evaporation of the combined organic phases in a rotary evaporator gives the moist sulfonic acid. This is taken up in 70 ml of MeOH, 1.34 ml of 32% NaOH are added, and the mixture is boiled for 1 hour (65° C.). After the reaction mixture has been cooled, the resultant suspension is concentrated and filtered through silica gel with MeOH/MTB: 1/1. Evaporation of the solvent in a rotary evaporator gives a colourless solid.

M=358.4

Melting point: decomposition >145° C.

¹H-NMR: (300 MHz, CDCl₃)
2.89 (t, 2H); 2.83 (t, 2H); 1.78-1.64 (m., 4H); 1.43-1.29(m, 14H) ppm.
¹³C-NMR (75 MHz, CDCl₃):
   131.2 (q), 51.5.0, 29.7, 29.7, 29.6, 29.5, 29.4, 29.3, 29.0, 28.8, 28.4, 24.5 ppm
¹⁹F-NMR (282 MHz, CDCl₃):
   −41.3 (s, —SCF₃) ppm
IR (KBr) 3437, 2920, 2860, 1657, 1577, 1466, 1417, 1249, 1219, 1204, 1135, 1062, 792, 762, 724 cm⁻¹
Substance: CF₃—S—(CH₂)₁₁—SO₃Na: concentration: 99.8 mg/100 ml surface tension: 31.29 mN/m

### Example 4

### Synthesis of pentafluoroethyl thioethers

11-Mercaptoundecan-1-ol, for example, or other thiols can be converted into C₂F₅S compounds as described in Examples 3g and 3h by using CF₃CF₂I instead of CF₃I. The subsequent reactions to give the surfactant end product are carried out analogously to the preparation of CF₃—S—(CH₂)₁₁—SO₃Na (Example 3j).

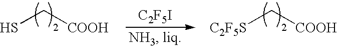

S-pentafluoroethyl-3-mercaptopropionic acid is synthesised by the same route (see above) from 8.3 g (78 mmol) of 3-mercaptopropionic acid and 25.0 g (102 mmol) of pentafluoroethyl iodide in about 30 ml of liquid ammonia. The S-pentafluoroethyl-3-mercaptopropionic acid obtained is characterised by means of NMR spectra.

¹H NMR (solvent: DMSO-D₆, standard: TMS), ppm, δ: 2.68 t (CH₂), 3.14 t (CH₂), 12.58 s (OH), ³J_{H,H}=6.7 Hz.

¹⁹F NMR (solvent: DMSO-D₆, standard: CCl₃F), ppm, δ: −85.5 t (CF₃), −94.2 q (SCF₂), ³J_{F,F}=3.5 Hz.

### Example 5

### Compounds containing a 2,2,2-trifluoropropylmercapto end group

### Example 5a

### (3,3,3-Trifluoropropyl)thiouronium iodide

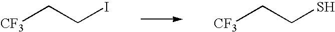

41.8 g of thiourea are introduced into a 250 ml round-bottomed flask, 23.75 ml of anhydrous denatured ethanol and 1.25 ml of water are added. 113 g of 3,3,3-trifluoropropyl iodide are added to the resultant suspension by means of a syringe and balance, the flask is sealed using a reflux condenser, and the mixture is refluxed for 6 h (oil-bath temperature: 90° C.). On the following day, crystallisation of the yellow, somewhat oily reaction mixture is initiated by scratching the glass wall, the mixture is cooled to ˜5° C. in the refrigerator, and the deposited crystals are washed with 40 ml of EtOH at −78° C., giving the product in the form of white, air-stable crystals. The intermediate is employed directly in the subsequent step.

C₄H₈F₃IN₂S; M=302.01

### Example 5b

### 3,3,3-Trifluoropropyl thiol

50 g of the thiouronium salt are introduced into a 250 ml three-necked flask and flushed with N₂, and the reflux condenser is attached. 20 g of NaOH are introduced into the dropping funnel and likewise flushed with N₂, and 100 ml of deionised water are added. The stream of nitrogen effects mixing and protection against ingressing O₂. The 2nd wash bottle after the flask is filled with alkaline permanganate solution in order to bind discharged CF₃CH₂CH₂SH, which has an intense odour. After dissolution, the sodium hydroxide solution is added to the thiouronium salt under a continuous stream of nitrogen, and the flask contents are refluxed for 2 h with stirring. Brown coloration and flocculation of the wash bottle contents indicate the formation and discharge of thiol. The thiol is subsequently liberated in the reaction mixture, cooled using an ice bath, by addition of the requisite amount of HCl for conversion of the resultant Na₂CO₃ (27.7 ml of conc. HCl) (pH then ˜1), and is deposited as the lower phase of greater specific gravity, which is contaminated with a few black specks. After filtration of the entire mixture through a G3 frit, the lower phase is separated off at low temperature by means of a pipette and transferred into an N₂-flushed, cooled Schlenk tube. 2-3 g of MgSO₄ are added to the separated-off liquid in order to bind water, and the mixture is frozen using liquid nitrogen and connected via a Schlenk cross to a tared second Schlenk tube, into which the product is then transferred. A water-pale, readily mobile, very volatile liquid with a strong odour having a density of (calculated) 1.2±0.06 g/ml is obtained.

C₃H₅F₃S; M=130.13
¹H-NMR: (300 MHz, CDCl₃)
   2.88-2.79 (m, 2H); 2.62-2.43 (m, 2H) ppm.
¹³C-NMR (75 MHz, CDCl₃):
   126.0 (q); 34.1; 29.8 ppm

### Example 5c

### 11-(3,3,3-Trifluoropropylsulfanyl)undecanoic acid

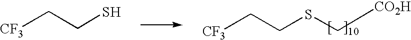

8 g of 3,3,3-trifluoropropyl thiol are transferred from the stock Schlenk vessel into the reaction vessel by means of a Schlenk cross by recondensation (liq. N₂). 60 ml of ethanol in which 10 g of undecenoic acid are dissolved are added to the still-frozen thiol, and 80 mg of AIBN are subsequently added. The vessel is sealed using a ground-glass cap and secured joint clamp. Dissolved oxygen is removed by means of three freeze-pump-thaw cycles. The reaction mixture is left in an 80° C. oil bath for 2 h. The success of the reaction is determined by means of ¹H- and ¹³C-NMR. To this end, all volatile constituents (=excess thiol, ethanol) are removed on the inert-gas unit in vacuo at a water-bath temperature of about 40° C. The white residue which remains, which is crystalline at room temperature, is dissolved in methanol at RT, and water is added to the filtered solution until turbidity remains. The mixture is cooled using acetone/dry ice, during which white crystalline agglomerates of the product form, which are filtered off with suction and dried.

C₁₄H₂₅F₃O₂S; M=314.41
¹H-NMR: (300 MHz, CDCl₃)
   2.64 (m, 2H); 2.51 (t, 2H); 2.36 (m., 2H); 2.28 (t, 2H); 1.60-1.52 (m, 4H); 1.48-1.24 (m, 12H) ppm.

Surface tension of an aqueous solution of the sodium salt (2 eq of NaOH/eq of carboxylic acid): 33.4 mN/m at 0.016 mol/l

### Example 5e

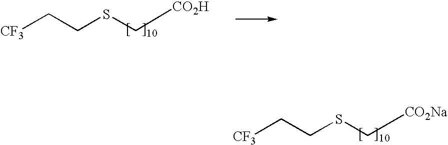

3 g of 11-(3,3,3-trifluoropropylsulfanyl)undecanoic acid (9.5 mmol, 1 eq) are dissolved in 18 ml of methanol, and 0.3 ml of NaOH (32% in water) is subsequently added at RT. The mixture is refluxed for 30 min and then cooled and evaporated and dried in a drying cabinet at 0.1 bar under a stream of nitrogen. The product is a pale-yellowish solid.

C₁₄H₂₄F₃O₂SNa; M=336.39
¹H-NMR: (300 MHz, DMSO)
   2.7-2.48 (m, 6H); 2.18 (t, 2H); 1.58-1.45 (m., 4H); 1.46-1.20 (m, 12H) ppm.
¹³C-NMR (75 MHz, CDCl₃):
   174.5; 126.6 (q), 35.0, 33.7 (q), 31.0, 28.8, 28.8, 28.7, 28.5, 28.5, 28.1, 25.1, 23.1, 23.0 ppm
¹⁹F-NMR (282 MHz, CDCl₃):
   −64.6 (t, —CH₂CF₃) ppm
IR (KBr) 3418, 2929, 2843, 1711, 1560, 1465, 1430, 1382, 1310, 1277, 1236, 1210, 1164, 1076, 960, 845, 721 cm⁻¹
MS ESI: M⁻−Na=313

### Example 5f

### 11-(3,3,3-TrifluoropropyIsulfanyl)undecan-1-ol

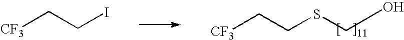

7.1 g of sodium hydride (60% suspension in mineral oil; 178 mmol, 1.5 eq) are initially introduced in 100 ml of THF (dried) in a 250 ml three-necked flask and cooled to 0° C. 25 g of 11-mercaptoundecan-1-ol (119 mmol, 1 eq) in 40 ml of THF are subsequently slowly added dropwise, and the reaction mixture is warmed to RT. The solution is stirred at RT for 2.5 hours and then re-cooled to 0° C. 29.2 g of trifluoroiodopropane (130.5 mmol, 1.1 eq) are then added with the aid of a syringe, and the reaction mixture is stirred at RT for 20 hours. After this time, complete conversion is determined by a TLC check, and the reaction can be worked up. To this end, the mixture is quenched using 300 ml of ice-water, the organic phase is separated off, and the aqueous phase is extracted twice with MTB ether. The combined organic phases are washed twice with saturated NaCl solution, dried using sodium sulfate, filtered and evaporated in a rotary evaporator. The residue is purified by column chromatography using PE/MTB 10:1. The product is a colourless solid.

C₁₄H₂₇F₃OS; M=300.43 g/mol
¹H-NMR: (300 MHz, CDCl₃)
   3.64 (m, 2H); 3.22 (s, 1H, —OH; 2.70-2.65 (t., 2H); 2.54 (t, 2H); 2.45-2.29 (m, 2H); 1.64-1.52 (m, 4H); 1.43-1.23 (m, 14H) ppm.
¹⁹F-NMR (282 MHz, CDCl₃):
   −66.3 (t, —CH₂CF₃) ppm

### Example 5g

### 1-Bromo-11-(3,3,3-trifluoropropylsulfanyl)undecane

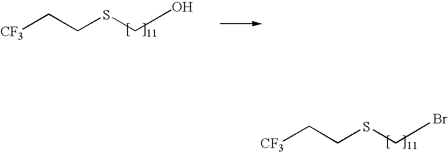

30 g of 11-(3,3,3-trifluoropropylsulfanyl)undecan-1-ol (98.9 mmol, 1 eq) are initially introduced in 500 ml of dry dichloromethane at RT, and 39.7 g of triphenylphosphine (148 mmol, 1.5 eq) are added. The reaction mixture is subsequently cooled to 0° C., and 55.7 g of tetrabromomethane (168 mmol, 1.7 eq) dissolved in 100 ml of dichloromethane are added via a dropping funnel at a controlled rate such that the internal temperature does not exceed 10° C. When the addition is complete, the reaction mixture is slowly warmed to RT. When the reaction is complete (TLC check), the reaction mixture is quenched using saturated NaHCO₃ solution. The organic phase is dried over Na₂SO₄ and evaporated in a rotary evaporator. A brown residue forms, which is adsorbed onto silica gel and purified by column chromatography (PE). The product is a pale-yellowish oil.

C₁₄H₂₆BrF₃S; M=363.32 g/mol
¹H-NMR: (300 MHz, CDCl₃)
   3.40 (t, 2H); 2.68 (m, 2H); 2.53 (t., 2H); 2.45-2.29 (m, 2H); 1.85 (quint, 2H); 1.69-1.53 (m, 2H); 1.45-1.35 (m, 2H); 1.35-1.26 (m, 12H) ppm.
¹³C-NMR (75 MHz, CDCl₃):
   126.0 (q), 34.9 (q), 33.9, 32.8, 32.2, 29.4, 29.4, 29.1, 28.8, 28.7, 28.0, 24.0, 23.9 ppm
¹⁹F-NMR (282 MHz, CDCl₃):
   −66.2 (t, —CH₂CF₃) ppm

### Example 5h

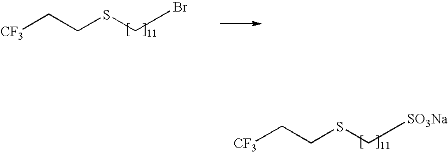

24.4 g of 1-bromo-11-(3,3,3-trifluoropropylsulfanyl)undecane (67 mmol, 1 eq) and 10 g of sodium sulfite (79 mmol, 1.2 eq) in 140 ml of ethanol and 140 ml of water are initially introduced into a 500 ml single-necked flask and heated to 100° C. The mixture is stirred under reflux overnight (20 hours), and the conversion is monitored by TLC. When the reaction is complete, the mixture is washed by shaking with a little MTBE/heptane 1:1 in order to remove the starting material and impurities. The aqueous phase is then acidified to pH=0 using a little sulfuric acid and extracted with 15×100 ml of MTBE. The combined organic phases are dried over Na₂SO₄ and filtered, and the solvent is subsequently removed in vacuo, giving 85 g of still-moist sulfonic acid, which is taken up in 120 ml of methanol, treated with 32% NaOH and refluxed for 1 hour. After the reaction mixture has been cooled, the suspension formed is evaporated, re-dissolved in MeOH/MTBE 1:1 and filtered through silica gel. The solvent is removed in vacuo. The product is a colourless solid.
¹H-NMR: (300 MHz, DMSO)
   2.69-2.35 (m, 8H); 1.68-1.41 (m, 4H); 1.39-1.15 (m., 14H) ppm.
¹³C-NMR (75 MHz, DMSO):
   126.6 (q), 51.4, 33.5, 31.0, 28.9, 28.9, 28.9, 28.8, 28.5, 28.4, 28.1, 25.0, 23.1, 23.0 ppm
¹⁹F-NMR (282 MHz, DMSO):
   −64.7 (t, —CH₂CF₃) ppm
   C₁₄H₂₆BrF₃O₃S₂; M=386.47 g/mol
CF₃—CH₂—CH₂—S—(CH₂)₁₁—SO₃Na Concentration: 100.7 mg/100 ml Surface tension: 38.55 mN/m

### Example 6

### Determination of the Biochemical Degradability

The biochemical degradability of the compounds is determined by the Zahn-Wellens test in accordance with the publication by the European Commission: Classification, Packaging and Labelling of Dangerous Substances in the European Union, Part II-Test Methods, Annex V-Methods for Determining the Physical-Chemical Properties, the Toxicity and the Ecotoxicity, Part B, Biochemical Degradability-Zahn-Wellens test (C.9.), January 1997, pages 353-357.
Batch volume: 1.5 I
Activated sludge concentration: 1 g of TS/I
Origin of the sludge: water treatment plant at Merck KGaA, Darmstadt (not adapted)
Amount of test substances used: about 100 to 200 mg/l as DOC
Aeration: with purified air
Processing of the samples: filtration (medium-hard filter)
Determination of the DOC: by the difference method using a Dimatec instrument

Further details on the method are given in the above-mentioned publication and in the OECD Guideline for the Testing of Chemicals, Section 3, Degradation and Accumulation, method 302 B, pages 1-8, adopted: 17.07.92, the contents of which in this respect are expressly part of the disclosure content of the present application.

In addition, besides degradation of the compound per se in the test, degradation of the fluorine-containing groups is also observed via fluoride determination:
Method: ion chromatography
Instrument: Dionex 120
Detector type: conductivity detector
Column: AS9HC
Eluent: sodium carbonate solution, 9 mmol/l
Flow rate: 1 ml/min
Literature: EN ISO 10304-2

### Example 7

### Determination of the Surface Tension

Instrument: Kruss tensiometer (model K12)
Temperature of the measurement solutions: 20° C.
Measurement module employed: ring
Concentration of the measurement solutions: about 0.5 to 3.0 g/l in deionised water

Further details on the method are given in the European Commission publication: Classification, Packaging and Labelling of Dangerous Substances in the European Union, Part II-Test Methods, Annex V-Methods for Determining the Physical-Chemical Properties, the Toxicity and the Ecotoxicity, Part A, Surface Tension (A.5), January 1997, pages 51-57, and also the OECD Guideline for the Testing of Chemicals, Section 1, Physical-Chemical Properties, method 115, pages 1-7, adopted: 27.07.95, the contents of which in this respect are expressly part of the disclosure content of the present application.

1. A surface-active compound comprising an end group Y, wherein said end group Y is CF₃(CH₂)ₐS— or CF₃CF₂S— or [CF₃—(CH₂)ₐ]₂N—, where a stands for an integer selected from the range from 0 to 5.

2. A compound according to claim 1, characterised in that the end group Y in the surface-active compounds is bonded to a saturated or unsaturated, optionally aromatic, branched or unbranched, optionally substituted hydrocarbon unit optionally containing heteroatoms.

3. A compound according to claim 1, characterised in that the end group Y occurs a number of times in the surface-active compound and the surface-active compound is preferably an oligomer or a polymer.

4. A compound according to claim 1, characterised in that the end group Y occurs only once, twice or three times in the surface-active compound and the surface-active compound is preferably a low-molecular-weight compound of the formula I
Y-spacer-X I
where
Y stands for CF₃(CH₂)ₐS— or CF₃CF₂S— or [CF₃—(CH₂)ₐ]₂N—, where a stands for an integer selected from the range from 0 to 5,
spacer stands for a saturated or unsaturated, optionally aromatic, branched or unbranched, optionally substituted hydrocarbon unit optionally containing heteroatoms,
X stands for a cationic, nonionic, amphoteric or anionic polar group or a polymerisable group or a functional group.

5. A compound according to claim 1, characterised in that the end group Y is present in compounds of one of the formulae Ia to Ig
Y—(CH₂)ₙ—X Ia
Y—CH₂—CH(Hal)—(CH₂)₍ₙ₋₁₎—X Ib
Y—CH═CH—(CH₂)₍ₙ₋₁₎—X Ic
Y—CH₂CH═CH—(CH₂)₍ₙ₋₁₎—X Id
Y—CH₂—Ar—(CH₂)₍ₙ₋₁₎—X Ie
Y—(CH₂)ₙ₋₁—C≡C—(CH₂)ₙ—X If
Y—(CH₂)ₙ-Q-(CH₂)_{n′}—X Ig
in which Y stands for CF₃(CH₂)ₐS— or CF₃CF₂S— or [CF₃—(CH₂)₂]₂N—, where a stands for an integer selected from the range from 0 to 5,
n and n′ stand, independently of one another, for an integer from the range 1 to 30, and
X stands for a cationic, nonionic, amphoteric or anionic polar group or a polymerisable group,
(Hal) stands for F, Cl, Br or I,
Q stands for O, S or N,
and corresponding salts of the compounds of one of the formulae Ia to Ig.

6. A compound according to claim 1, characterised in that the surface-active compound is used as surfactant.

7. A compound according to claim 1, characterised in that the surface-active compound is employed as hydrophobicising agent or oleophobicising agent, in particular in the surface modification of textiles, paper, glass, porous building materials or adsorbents.

8. A compound according to claim 1, characterised in that the surface-active compound is employed as additive in compositions for surface coating, such as printing inks, coatings, paints, lacquers, surface coatings, photographic coatings, special coatings for semiconductor photolithography, such as photoresists, top antireflective coatings, bottom antireflective coatings, or in additive compositions for addition to corresponding compositions.

9. A compound according to claim 1, characterised in that the surface-active compound is used as antistatic, in particular in the treatment of textiles, such as clothing, carpets and carpeting, upholstery in furnishings and automobiles, nonwoven textile materials, leather goods, papers and cardboards, wood and wood-based materials, mineral substrates, such as stone, cement, concrete, plaster, ceramics, such as glazed and unglazed tiles, stoneware, porcelain, and glasses, and for plastics and metallic substrates.

10. A compound according to claim 1, characterised in that the surface-active compound is used as foam stabiliser and/or for supporting film formation, in particular in fire-extinguishing foams.

11. A compound according to claim 1, characterised in that the surface-active compound is used as interface promoter or emulsifier, in particular for the preparation of fluoropolymers.

12. A compound according to claim 1, characterised in that the surface-active compound is used as antimicrobial active ingredient, in particular as reagent for antimicrobial surface modification.

13. A compound according to claim 1, characterised in that X stands for an anionic polar group selected from —COOM, —SO₃M, —OSO₃M, —PO₃M₂, —OPO₃M₂, —(OCH₂CHR)ₘ—O—(CH₂)ₒ—COOM, —(OCH₂CHR)ₘ—O—(CH₂)ₒ—SO₃M, —(OCH₂CHR)ₘ—O—(CH₂)ₒ—OSO₃M, —(OCH₂CHR)ₘ—O—(CH₂)ₒ—PO₃M₂, —(OCH₂CHR)ₘ—O—(CH₂)ₒ—OPO₃M₂, where M stands for H or an alkali metal ion, preferably Li⁺, Na⁺ or K⁺ or NH₄⁺ or tetra-C₁₋₆-alkylammonium or tetra-C₁₋₆-alkylphosphonium,
R stands for H or C₁₋₄-alkyl,
m stands for an integer from the range from 1 to 1000, and
o stands for an integer selected from 1, 2, 3 and 4.

14. A compound according to claim 1, characterised in that X stands for a cationic polar group selected from —NR¹R²R³⁺Z⁻, —PR¹R²R^{3 +}Z⁻, 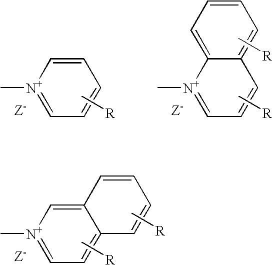
where R stands for H or C₁₋₄-alkyl in any desired position,
Z⁻ stands for Cl⁻, Br⁻, I⁻, CH₃SO₃⁻, CF₃SO₃⁻, CH₃PhSO₃⁻, PhSO₃⁻,
R¹, R² and R³ each, independently of one another, stand for H, C₁₋₃₀-alkyl, Ar or —CH₂Ar, and
Ar stands for an unsubstituted or mono- or polysubstituted aromatic ring or fused ring system having 6 to 18 C atoms, in which, in addition, one or two CH groups may be replaced by N.

15. A compound according to claim 1, characterised in that X stands for a nonionic polar group selected from —Cl, —Br, —I, —(OCH₂CHR)ₘ—OH, —(OCH₂CHR)ₘ—SH, —O-(glycoside)ₒ, —(OCH₂CHR)ₘ—OCH₂—CHOH—CH₂—OH, —(OCH₂CHR)ₘ—OCH₂Ar(—NCO)ₚ, —(OCH₂CHR)ₘ—OAr(—NCO)ₚ, —SiR¹R²Z, —SiR¹Z₂, —SiZ₃, —R²—COZ, —(OCH₂CHR)ₘ—SO₂CH═CH₂, —SO₂Z, 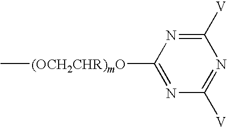
m stands for an integer from the range from 0 to 1000,
n stands for 0 or 1,
o stands for an integer from the range from 1 to 10,
p stands for 1 or 2,
R stands for H or C₁₋₄-alkyl,
R¹ and R² each, independently of one another, stand for C₁₋₃₀-alkyl, Ar or —CH₂Ar, and
Ar stands for an unsubstituted or mono- or polysubstituted aromatic ring or fused ring system having 6 to 18 C atoms, in which, in addition, one or two CH groups may be replaced by N, and
glycoside stands for an etherified carbohydrate, preferably for a mono-, di-, tri- or oligoglucoside,
all Z each, independently of one another, stand for —H, —Cl, —F, —NR¹R², —OR¹, —N-imidazolyl, and
V stands for Cl or F.

16. A compound according to claim 1, characterised in that X stands for a polymerisable group selected from —(OCH₂CHR)ₘOCOCR═CH₂, —(OCH₂CHR)ₘ—OCR═CH₂, 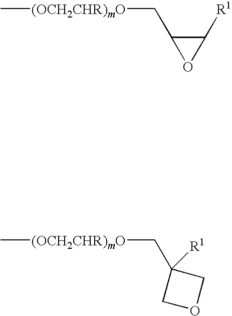
where m stands for an integer from the range from 0 to 1000, R stands for H or C₁₋₄-alkyl and R¹ stands for H or C₁₋₄-alkyl or Y-spacer-(OCH₂CHR)ₘ—OCH₂.

17. A compound according to claim 1, characterised in that X stands for a functional group selected from —CR²═CR³R⁴, —C≡CR², —CHO, —C(═O)CH₃, —COOH, —COOR, —OH, —SH, —SO₂Cl, —Cl, —Br, —I, where R², R³ and R⁴ each, independently of one another, stand for H or Y-spacer- or C₁₋₄-alkyl, and R stands for C₁₋₃₀-alkyl, Ar or —CH₂Ar.

18. A compound according to claim 1, characterised in that X stands for an amphoteric group selected from the functional groups of the acetyldiamines, the N-alkylamino acids, the betaines, the amine oxides and corresponding derivatives thereof.

19. Compound of one of the formulae Ia to Ig
Y—(CH₂)ₙ—X Ia
Y—CH₂—CH(Hal)—(CH₂)₍ₙ₋₁₎—X Ib
Y—CH═CH—(CH₂)₍ₙ₋₁₎—X Ic
Y—CH₂CH═CH—(CH₂)₍ₙ₋₁₎—X Id
Y—CH₂—Ar—(CH₂)₍ₙ₋₁₎—X Ie
Y—(CH₂)ₙ₋₁—C≡C—(CH₂)ₙ—X If
Y—(CH₂)ₙ-Q-(CH₂)_{n′}—X Ig
in which Y stands for CF₃(CH₂)ₐS— or CF₃CF₂S— or [CF₃—(CH₂)ₐ]₂N—, where a stands for an integer selected from the range from 0 to 5,
n and n′ stand, independently of one another, for an integer from the range 1 to 30, and
X stands for a cationic, nonionic, amphoteric or anionic polar group or a polymerisable group or a functional group,
Q stands for O, S or N,
(Hal) stands for F, Cl, Br or I,
and corresponding salts of the compounds of the formulae Ia to Ig.

20. Compound according to claim 19, characterised in that X stands for an anionic polar group selected from —COOM, —SO₃M, —OSO₃M, —PO₃M₂, —OPO₃M₂, —(OCH₂CHR)ₘ—O—(CH₂)ₒ—COOM, —(OCH₂CHR)ₘ—O—(CH₂)ₒ—SO₃M, (OCH₂CHR)ₘ—O—(CH₂)ₒ—OSO₃M, —(OCH₂CHR)ₘ—O—(CH₂)ₒ—PO₃M₂, (OCH₂CHR)ₘ—O—(CH₂)ₒ—OPO₃M₂, where M stands for H or an alkali metal ion, preferably Li⁺, Na⁺ or K⁺, or NH₄⁺ or tetra-C₁₋₆-alkylammonium or tetra-C₁₋₆-alkylphosphonium,
R stands for H or C₁₋₄-alkyl,
m stands for an integer from the range from 1 to 1000, and
o stands for an integer selected from 1, 2, 3 and 4.

21. Compound according to claim 19, characterised in that X stands for a cationic polar group selected from —NR¹R²R³⁺Z⁻, —PR¹R²R³⁺Z⁻, 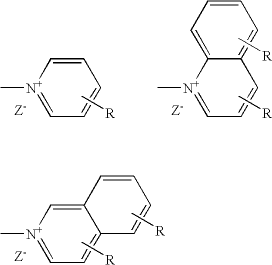
where R stands for H or C₁₋₄-alkyl in any desired position,
Z⁻ stands for Cl⁻, Br⁻, I⁻, CH₃SO₃⁻, CF₃SO₃⁻, CH₃PhSO₃⁻, PhSO₃⁻,
R¹, R² and R³ each, independently of one another, stand for H, C₁₋₃₀-alkyl, Ar or —CH₂Ar, and
Ar stands for an unsubstituted or mono- or polysubstituted aromatic ring or fused ring system having 6 to 18 C atoms, in which, in addition, one or two CH groups may be replaced by N.

22. Compound according to claim 19, characterised in that X stands for a nonionic polar group selected from —Cl, —Br, —I, —(OCH₂CHR)ₘ—OH, —(OCH₂CHR)ₘ—SH, —O-(glycoside)ₒ, —(OCH₂CHR)ₘ—OCH₂—CHOH—CH₂—OH, —(OCH₂CHR)ₘ—OCH₂Ar(—NCO)ₚ, —(OCH₂CHR)ₘ—OAr(—NCO)ₚ, —SiR¹R²Z, —SiR¹Z₂, —SiZ₃, —COZ, —(OCH₂CHR)ₘ—SO₂CH═CH₂, —SO₂Z, 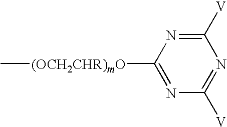
m stands for an integer from the range from 0 to 1000,
n stands for 0 or 1,
o stands for an integer from the range from 1 to 10,
p stands for 1 or 2,
R stands for H or C₁₋₄-alkyl,
R¹ and R² each, independently of one another, stand for C₁₋₃₀-alkyl, Ar or —CH₂Ar, and
Ar stands for an unsubstituted or mono- or polysubstituted aromatic ring or fused ring system having 6 to 18 C atoms, in which, in addition, one or two CH groups may be replaced by N, and
glycoside stands for an etherified carbohydrate, preferably for a mono-, di-, tri- or oligoglucoside,
all Z each, independently of one another, stand for —H, —Cl, —F, —NR¹R², —OR¹, —N-imidazolyl, and
V stands for Cl or F.

23. Compound according to claim 19, characterised in that X stands for a polymerisable group selected from —(OCH₂CHR)ₘOCOCR²═CH₂, —(OCH₂CHR)ₘ—OCR²═CH₂, 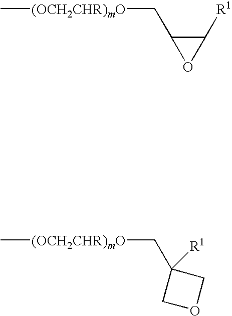
where m stands for an integer from the range from 0 to 1000, R stands for H or C₁₋₄-alkyl and R¹ stands for H or C₁₋₄-alkyl or Y-spacer-(OCH₂CHR)ₘ—OCH₂—.

24. Compound according to claim 19, characterised in that X stands for a functional group selected from —CR²═CR³R⁴, —C≡CR², —CHO, —C(═O)CH₃, —COOH, —COOR, —OH, —SH, —SO₂Cl, —Cl, —Br, —I, where R², R³ and R⁴ each, independently of one another, stand for H or Y-spacer- or C₁₋₄-alkyl and R stands for C₁₋₃₀-alkyl, Ar or —CH₂Ar.

25. Compound according to claim 19, characterised in that X stands for an amphoteric group selected from the functional groups of the acetyldiamines, the N-alkylamino acids, the betaines, the amine oxides and corresponding derivatives thereof.

26. Compound according to claim 19, characterised in that n stands for a number from the range 4 to 28, preferably from the range 4 to 16.

27. Process for the preparation of a compound of the formula I according to claim 4, characterised in that firstly a compound of the formula V
Y—CH₂—CR²=CR³R⁴ V
where R², R³ and R⁴ each, independently of one another, stand for H or C₁₋₄-alkyl or Y—CH₂—, is prepared by reaction of an allyl halide Hal-CH₂—CR⁵═CR⁶R⁷, where R⁵, R⁶ and R⁷ each, independently of one another, stand for H, C₁₋₄-alkyl or Hal-CH₂—, and Hal stands for Cl, Br or I, with a tetraalkylammonium Y⁻, and this is then, if X in the compound of the formula I is not CR²═CR³R⁴ or n is >1, converted into the compound of the formula I by modification of the double bond in a manner known per se.

28. Process for the preparation of a compound of the formula I according to claim 4, characterised in that firstly a compound of the formula V
Y—CH₂—CR²═CR³R⁴ V
where R², R³ and R⁴ each, independently of one another, stand for H or C₁₋₄-alkyl or Y—CH₂— and Y stands for CF₃(CH₂)ₐS— or CF₃CF₂S—, where a stands for an integer selected from the range from 0 to 5, is prepared by reaction of an allyl thiol HS—CH₂—CR⁵═CR⁶R⁷, where R⁵, R⁶ and R⁷ each, independently of one another, stand for H, C₁₋₄-alkyl or HS—CH₂—, with a fluorinated alkyl halide CF₃(CH₂)ₐ-Hal or CF₃CF₂-Hal, where Hal stands for Br or I, and this is then, if X in the compound of the formula I is not CR²═CR³R⁴ or n is >1, converted into the compound of the formula I by modification of the double bond in a manner known per se.

29. Composition comprising at least one surface-active compound containing at least one end group Y, where Y stands for CF₃(CH₂)ₐS— or CF₃CF₂S— or [CF₃—(CH₂)ₐ]₂N—, where a stands for an integer selected from the range from 0 to 5, and a vehicle which is suitable for the particular application and optionally further specific active substances.

30. Composition according to claim 29, characterised in that the composition is a paint or coating composition, fire-extinguishing composition, grease, washing or cleaning composition, deicer or hydrophobicising composition for the treatment of textiles or the treatment of glass, printing ink, paint, photographic coating, special coating for semiconductor photolithography, such as photoresist, top antireflective coating, bottom antireflective coating, or an additive composition for addition to corresponding compositions.

31. Process for the preparation of a composition according to claim 29, characterised in that a surface-active compound containing at least one end group Y, where Y stands for CF₃(CH₂)ₐS— or CF₃CF₂S— or [CF₃—(CH₂)ₐ]₂N—, where a stands for an integer selected from the range from 0 to 5, is mixed with a carrier which is suitable for the particular application and optionally further specific active substances.
